SEQUENCE LISTING (32 sequences)

SEQ ID NO: 1 - RNA, 19 nt, Artificial Sequence
  source: /note=“Description of Artificial Sequence Synthetic oligonucleotide”
ccuucauguucagaugguu

SEQ ID NO: 2 - RNA, 19 nt, Artificial Sequence
  source: /note=“Description of Artificial Sequence Synthetic oligonucleotide”
ugguuuacauguuguguga

SEQ ID NO: 3 - DNA, 213552 bp, Homo sapiens
gtccagcgagcggtctcctcctcctgctagtgctgctgcggcgtcccgcggcctccccgagtcgggcgggaggggagagcgggtgtggatttgtcttgacggtaattgttgcgtttccacgtctcggaggcctgcgcgctgggttgctccttcttcgggagcgagctgttctcagcgatcccactcccagccggggctccccacacacactgggctgcgtgcgtgtggagtgggacccgcgcacacgcgtgtctctggacagctacggcgccgaaaggcaagaagggaaggggtcgccggggaggctccacacttcttgccggcccgggggcccggggtagccaggcgcgggccaaggaggaagaggggacagccctggctctcagctgcctttgccgccgccgtccagtttgaagtggggcatttaaagaccagaacccgcaggccggggaggagggagagggcgtccaggcagacggcggtgaggactgcagcaggcggccgggaggcgcgtccacccaggcgccgagcccacagtgtccctcccgcgccctagacggacggtgctgcgggcacgggagggacactttgtcccctcccagtccaccttcctcctgcagctcagacccgaggggccgggaaggggcctgagcagggaagggacacaggcgtcctgaaacaaagctttgggttgagggcagaggcgaggagccagagaagtgaactcctcctttccgtggccgctcttgtcatttttctcttttctcttttcaaatgggctatttgtgtgttaatttcatttggttcttgggaataacgactcaccgcgttgatttaaagaaaggtgttcgtgtgtccgtacttggtggttttagtgtgggacatgccctgcgcttttgttgacaacagagctgtcccttacgtttgtgtaggataatctttatgtttatgtaacttggagaaatctaacagagtgaaagtgaagtttctgcgtaaggctcccaaaatgtatgataggaccttcagttgttctggtattttgaattggaattaaggcaggaggggaagaacattctgtttttatctaggtgggggaaacctgtttaagttttgtttgttcttttttttttttttatgagttgagtattagttcagccttagctacttgaaacgtgagcaggatgcatctttaactcttagtattgtaaaagcaaatctaggatcactgctcttaaaactactgtgtgtaaagattgagagtgtaaattcacaagcttattgataaagatgaatccggaacttttggttttgctctaatcgatggaggaagatgcagtggtacaagttagtctctagttttctggtgggtaaatattttcaaaaccaactaacttttctttttttaaatctgccttttaaagaactaaaattccagatggcaaactcaatgaatggcagaaaccctggtggtcgaggaggaaatccccgaaaaggtcgaattttgggtattattgatgctattcaggatgcagttggaccccctaagcaagctgccgcagatcgcaggaccgtggagaagacttggaagctcatggacaaagtggtaagttctgtctgacgttgcctgtgcgaaacactattcactttgtctgtggtttctccagatttatttatactgttaattaataccaaccattgttatggcaacaatcttttgatttcaattcatcatttttcttcaactctgtgttttttgaaaagttggattcaaagacttttttgttttttaacctttcatcctttctactctttaaggttcagagttcctctacatgactcagtatgtaaagttttaccattggctgctttttgttggtgtatggccatagaatgcttggaagtgtttgttctgtcttctgtgaggttgtgttgctcttagataacaaaagagtaccagcaggttatttaagtctttgaccactcatgacaatcatctggtatgatactgaaaatatttactggggaaagaaacagattagttcttaagtgaagaagcagtagttgagggagtttccattttcatctttgagaattcatcttcattaagaaactttttttgttgttaatgggagagttaacagtttctttcatctactattccattttgaaacaggaaacctttcgatggcacattgtttattgtgaaaggaaaatttatgttgggggtggtgaggctgagagtatcagaaacctctgtggtttctgcatgttgctttcagacaaactataatttcacaagaagaaatcatgatgggtgtcagtatccagagaagtcaatctgaataccttacagagatttactcagaaactgtagctgtcagtatcctaccttactaagtatgattataaaataaatttaagatgtagcttgctgctttgcaaaaattaataattcttttttttgaagcagtagcactgtcctggatttattagtaaatgtttggaacacattatgactttatttcatagatatctggggccaattataagtttagttgaggcaagactcagtatctttataactgaagaaatggaggttttaaaaacaaatttctgtgtaaggatgtgccaaagcatccatgaaatcatgtgataacattcatctttgctttgatttgctcctgtctgtaaatattggctataaaagccactatcagtagtagtagcaacatatgaatgtgacaagatttccattattttcatttattctgatggaagggacacacagatgaatgactggcctgtcattctacaggcaatggtgctaaatgcagagccagaatatttttgccagataaatagccttagttttgggcttcagtgtgacctatttacgcttctgccaatttattcaaattgttatattaaagtgctccagacagaatgatgtcatagtgtcagaatattgcaacttgctgcttggggttcagttttgattttagcattggatactttagaataaggactattcaattggaaaaatagcatatatttgtcaagcacaatgctgcctatcttgtctttgctaaagagtgtgcatttgaaggtcactctcactgcctaaatcattagttggggtaagagaaggaattcaaggtttttctctaacttatctgctacgtaagtggacagcaaatggttcagattctataaaggggctgttctttttcccccaccctccaactttgagtggtcagtgtttgtacatttgtgttaggacatcaaatcaaaaagatttatataaggatgtggtgagctatagttactataaaatcattatttttgaaacattaaaaatgatgtacacaattttggtctgtaactggttgttaacataagttttattcacttttaattatttcaccatgagttatggtaaaatagatggttatttgatagttaaacatctgctattgtttattttattaacccttctgtgggtttaggtttagtctctccaattgagaaacttgacccaacaaggaaatctattatcaaaagtctctacttagtgggatgtgggtgcgggtgttggcaaatgtacattgcctgtttgtcttcattatttgtcaaagatgaaaatggttgtgttcatttgtgaatgtacccaggggctttgtggagagaaattgcataaattattatgaaagaattcagtatgtttatttttactgtaagatacagtaaatagaaaggcattgataattactaattggcagtatataagtgtgtagtacagaaacatccccctcttttgtttatcttttcttatttgttagtggtgaaggaagtggctcataatatcaacattgtcctgatgattacatcaggaactttattattagttctgaagttaatcccttgacacattacctttttaaaaaaataactgcaatagcatgaatttagtttgtgaataacagaaaatagcaattttcaagtctaattggtaggtatgtaaaaggcagagttaagtgtatagttaatatttttttcttatgaagcctctttgcaagggtagatcttggaagtataaatatgaacacaagcttcaatttcagtgtgtttcagctaccaataaagtaaaataactatttttacagggaataatgaagcagtcttgggttgtaggggaaatatctagtgagtatgtagttttaccatttcttggcctctggaatttaaaatggtcaattgaaattctttccaattaatgttctgttttcagagggcaggcttttgctctaaggtcactgagaataggagagcgtaattgctcagttgtgttccttataagatgtagagtgttttgtaacagaaaatcagtagcacagagttagcatagtctatgttttgaggaaaagaatagtagatagtagcatatcccaaaacatcccattacagattttctggccttagctctgtgtatgtgtaatttgatagccgacccagaagccctcacatcagtggatgcttatttgatacagtgatagaagtattaatcatgaatacatatctaggttcaagtgcgccagaatgaaatgtggagtctgttctgaagctactcaagagacaaggtttcttaaacacttaataaacattttttgagggcctactgtgtttccactgagtgctcgttactggggatatgaagctagacaagacataatcccgcctttgacctgtcaaggaactcagagaacagtatcagagccaaacacataaatggataattgccatgtagtatggaaagggctatattacaaaccccaagtacagtgggaacagagtagagaacatcctccttgtttttctggccagtgttgagacagttttctgtctggattctgctggccattgccttttaggaacagcattattgtgttcttgttggaaatgctgaggtgagagtaaggaaatgattactaatcagtatggttggaatgataatttattaatgtgaggttcctgagcatggttttcataagaatcttagccaggtgatggaaaatagaagaaacaattttccatctaatagatactaaatgtactgagcatacatggatgcttaggaaattagaaaataattaattgatgctgaagagcaaagcactctgagtacctgcctcaaatttttgttttgctgtcctcgtcatttattttaaattacattaccaaaagaatttaaaaagcatttaggaagtgatgatgcccagtaggcttttcctgccttttgccctcagtctatttagtacatttttcaagcttgaaatataatttacactccttttaatttaattgtgatcataatatattttaaagtttgtcaatcaaggatgcttggattatatataaatattttaaagaatacaagtcaactttcttttctaagctgagttcctcatatctctgaaagatattccatgtggactaagacaggttttagatgcctgttgtttgtgtttactaagttggagactcttacattcctagagccttaaaaccagggatatcattcatttgaattatacttcttgacgtatttccttttcctcactgaagttgttccaccttagcagaatcatgggttgtggtagacttggttaggggataataacaaattgtctggacagttttgtttagtactcagttcagttttgaatttgtgttgtgataaatgaagtgatcccaaagtttggtacatgacttcttagaaaagctattgataaagtgaaagatttaaaggaatcatatgaagacttaccttggtttatccctgtgatataaggcattcatataatgcaggccttaatcagttagtagtcttttaaatattacagaatttttagcattgagattttgaatctatttctttcttctttttagaaggtgctttatgtgctatctgttgatttcatcattcatttatcaaatctcaactatggaaagacagacatttccaaatagctatgccatagagtaaaatgtagtaagtgctttaaggtagtataaataaagtgctgagatgtagtacgtggtagaggtgaaccgttgcttgctaagggtgaaaagttggtgtagaatgaagcatggaggaggtgctggccagctcatgggttggagtccagtgagatggaggacatgttaggttgaaggaatgatagattgttgagtaagagaggggaagttagggtaagatgatagaggttttgattgcttttgtcaaataattttgtcttaatttatttggtagaaaaccattgaaggctgttaatcagggagggaaaacttcagggctgtgctttgatagaatacttacttgactctgtaaagcttgaattaagagaaaaaaactgaaagcaagagtaatgatcaagaggaattaaaacagtctgggcaagaaataatgaaatgctggcctagatacttggaagtaggtgtagcaagtgttggttcagatgtgggaggcattttggaggtaaaatctacaggtcttggtgaccccttagaattagaggatgaaagagaaggtgcagtgaaagtgactcttgaggttttcagcctagttgactccgaaggcagtgatgcacttaatgaggtaaggaacgtaagaaggaaaagaggaagtgtgcgggttagtttttggccatgctaagttcagtgtaccagagtgaatttcatttaaaatcagaaaattaagtgtacagttcaagagagaagtagaaacaagagacacatatttggtagatattctcatagagatagcatttcaagctatataactgtcagagatttcggaggaagaatagagggagcaatgcagagaaaaagtcagataattgaattctggggagattgaggggtgtaattaaataccagagaaggaaagagaatcctacatagattagtgatgagaaaggtagaattaaaccataatgacatcctaggtacaaacagctttaagaagaagataaaatctgaagttctgataaaactaagttcagattcaagtagagaaaaccaaacagggtgaaaaattaagaagtcattgaattgtatgattagaggccattattgacctttgtggagcagtttaacttatgtgatggggtgagattaaaatgagtatgtggattatttaaaaattgttctagaactgtaacaacaatgaaaaggaagggagttctgttagcttgaggatttgaacatcattgaaataactctgtgtgtgtgtgtgtgtatgtatgtgtgtgtgtgtgtgtgtgtgtatgtgtgcacacaccataggagaggttcaaagattggtgggaaaaaaaaaagattgatgggaaaggatgtagttagggaatggcctaagatgcaagggaaaaagaaatagttgaagcagtagcattctagattaaactgtgggagggaaagagtaggaacaagtagaagggttaatcttggcgaataaatgtaacttccacttacacaggaggaaagcaagggaatatacagagaagttttgagggaatttgtaaattttatattgggttgtcaggaagtgatttaaggcataatatttataatgtttaaaccatgtttttaatttttatatcatgaaatatgcaatatatgtattttaactagtcttctagcatttaagtgggtaaataagtaaaaaattaaaatggaaatcaattctcatctttaatattaagtttatcttattatacacttgctgtgacttagaggatgccacagtgctatttttatctactctgcttgggcatatattaataatattattacaattttactaaaggttttttttgtttttaagtatttgtaggttgctaatttttttcaagtaatgaaagacacattttgaaaatatccaacacattttataggtttttaacatagttacatttagatgattgttgagcttatattatcttacagtatcaatgaaaaaagtattaatagttataatattctacctgttaaaagatttaaaaaaatattaaagccctacgtcttttcctgagagcctcttgggtttgaagaaaccagaggattccatgtcagctgacaggaacactgacctgggcttagccttatttcctgcttttctgcacagaacattccaggctttttaagggttttaggcagcatgagtaactttatgagctaaaagagcttctaaaaatcatgatcctcaacaaactgttcagtgcccttggtctaattttcatacctttgcaaaatctgcagggattttgccttgtgatggaatgtaaaatatggaagggagaagaaatattaagaaggagatagggctaactatgttctggacacagtgcatagcactttacatttcctcttgtaagaattataatatcctaagaggtttcccgaagaagaaccctgcccaaggttgtaaagctagtaaatgttagggccagatcctaggtttgcttgattctaatgtctgtgttcacagtatgaagagtgaagcacactgctccatggaataatatgatgcttcctttttccaaaggaagtaaatcaaaagggcatttcccctcctccacttccatatttgatattatcaaattcttctccaaactttgtactcatttttaggcagctttattcaatgtagttgtggatatgctatttgtaatataattttcctgagaggttcgtccttaccttgtttgcttcctcctcctcttcctcctttctgccatcaaatatttgttgcacatctactatatgcaaagttccatattaggtgatagagagagaccaatgagaaagactgttcttcactttcaaggaagttccattctcatttggaagaaagagagtgaacagaatacacaattgatattttaattacaactagtcagttctttaatggtgcttcttaatgtcaatcttctgaagacatcttcgtggaaaaccataaaggtcttggtaattttctgtaaattcagctgtgctggactcatgatcaattttatatgccaactttgttttgcccaattaataatctattaaaaaagtgtatttttatacttttttacactgtacatttttatacttagtaaaatacatttttataatttttatatttagtaattatttgctgcattaaaatggaaagaacccaaagaactcttcggtacctgttattataaagagagagatatatatatactttaggtcactgtaataagaaacctcttctaacatttataggaggaggacagggctttcttttttttttttttttttttgacacggagtctcactctgtggcccaggttggagtgctggagtgcagtggcacgatctcggctcactgcaagctccgtctcctgggttcacgccattctcctgcctcagcctcccaagttcctgggactacaggcgcctgccaccacaccctgctaattttgtttttgtatttttagtagaaacagggtttcaccgtgttagccaagatgttctcgatctcctgacctcgtgatccgctcgcctcggcctcccaaagtgctgggattacaggcgtgagccaccgcgcctagcccagggctttctatattgtttttaagaatgaggcagttcacttgagagtatatgacttgattcagtgtaactgaatgctccatgaagtgaggcgtgttttataatgaaagtcttatagtataggcacaattccttactgttgaaattttcattttatttggtaagagttctttcctatgtttcacaggctggtttgaaccttgtttgcaaggaatgtactatctgatctaatacatttttattaccttttggctggaagttttaattaaggcgcagtcagaaaactctttaaatgcaagattattccaagaataagtaatgactttgaaaattcttatagcattagtaaatcctgtttttgtatttgtgcttttgctttgatatggttgaatatatgtatctaaatacatatttatgtattggaatgcatagccaatatattaataatatatttttattcatgtgaaacaatgcatgtacagagagagtctccttaaatatttctaatacatttaagcaaatacaaaactcagttgtatattggtatggagacagttcctgattctcaagtccagctatcagggttctgagcagtaaccttagttaggttctagttctgtatttcaatattgacacgtcaaaaggaattggtcctggtaatttccaaattttttgagtcaatggctatagtggaattttcagcctaattccaataataaatgttttgccttttagttcaacagaaaagagcaatattgctattgaaccaattcttttatgcaccaatcggtgccctacaaagttcatcattttataatgtgactacagatttgttctttgtcaaagctcttctgtgttagtaccaagcttcattctaggaagcaatttaggtatttctagaagaggtgtaaccatttgaatctgaacttgaccctggtggtgagcttgattttctctctaatatactgcctttagaaaagcaatttttctttcctggcttttgcagctccttgcacatagcaggtattcaataaatatttattgaatggaattgaatttgacctgcttagaagtcatagtcctgatgtagggttattcatttttggcagaagctgtcataaatcacagggtttaatagtaatccaactgatgaactgcgcacccagtggcataccgtagttcagcattgtatgttgcactttcccaagtgcaaatttagaaaacagtttagcctacagggccttgatgaactgactctggcctatttcagatctctcgtactactcactcacagccttccttcctatgctctgatcttttagaataactgcatttacttctctcttaacatgcttgctctctggtgtctcttaagccttagcacactcctttgcctagaatactccttttggcaaatttaagtatgtatttcctgacactcttatgtggagatgagtgagctgctgcttttctcctgtagcagtttgtacttgcctccatcatagcactaaattctcctgtaattcttttcctaagccaccagtctttcccactaggcagctgcttgagggcaggtgtgtgatttattcctttctgtgttccctagagcctgaaacaatgcctgccacataaatgtttgttgaattaacaaatgggtcctgaagtttacgtaaatttgcaaatggaataaaataaaaatgtaaatgagtttagcagggatcattaattatatgcctaattttaattaaattgtgatacagttagaaagatcagcccgagattcagaagatacaagctcttgtattggattaatgatattttaatggtcccttaatctttctagttcttagtttttttaaatctggaaatgaatggattgggtggaatctttactaagatcccttagagcaagaattacattgctcttaagttaattatagagaactacgtctcttctgccagactctttgcatctctttgaatcaggaagtatgttctagtcatctataatttcctagctctaagctcagtacctggcatatagtagttcaagcaatttccacctgataaagcaataaattcccacatggaattacagttttaaataaagcaaatgagaaaaaagttcagataaagaatagtgaattaaatatttgtaaaacataaacgtttctgtcaaagaatatttatgtggaatgtctggaaaaaaaatacctgacagaaattttgtatgtatcagttaaaaagttctctagtagaggaaatgaatttgaagctacctggtccccaaactagaagaaaataatgtatcagtctgtgacatgttacaccaagttttatgttgctgacattaaactatgcctgctgtagaatgttggtggcaatattcttggtatttatgtatacttgtgatagtatttttggtttaaataatttaaaaaccatattctttttcaagcaggatttgttaaataattggcatatttgggacttaatctttagggcagaatgaactctgattatactaacgtatcacgtattaaaaagggggtaaaaaaagctctgtcagccattttcactatactttgtaaagatttgctgttatttagtaaatgagatgacatcttacctttccaaggactttcttggagataaaaatactattattactttttaactaagcctatacctttggattattaatttgaatacttgatactttagagttcaattgtaaaaataaaggaaataatacaatcaaactatttgtgaaaaaaaaatagcagtcaaccaatccattaatttgttggttatgtactcagcaattctgctttaaggcattgtttacaaagaggaagaatataagatcatgccctgggttaatgtgaaaaacatcttaaattagaggctgatggttggaaatttgaaccagatttttctctccatctactccctctgaaaaaaaccctgatccccagtgatttcttttttttttccttttcttttttttttttttttttttgagatggagtcttgctttcttgtccaggctgagtgcagtgatgtgatctcagctcactgcaacctctgcctcccgggttcaagtgattctcctgcctcagcctccttagtagctgggattacaggcacctgccaccacacccagctactttttgtatttttagtagagacagggtttcaccatgttggccaggctggtctcgaactcctgacctcatgatctgcctgtctcggcctcccaaagtgctggggttacaggtgtgagccactgcgcctggccaatttctttaagaaagtaagtgtgatagtgtgtataagagtaccctttaaatttgttagttccttgatttattgtgttcgttatgcttgaagttattatagttcgattgtttatttgtcccaggcacttttatgggtgcccagaggcagatccatgttttatgtcccctgaaacttatatgatttgaaagaatctttctaaatataaagaatacgaaagtacaaacatcaaattgtttatgaaagtgaattttttttagaatgagaaaaatccacaaagcactatagtttttaaaaagccggtatatctacaagtgattagagaaataatctagactagtttttggctctgtacattttatactttgattatcttccgttaccgcatatacttccagttttgggtgtcatgaaacacattcaaattgccagatttgaattggatttcactgttcccatggtaggctagtcagcattgtgggcacctcaggtatttctaaaaccatcttttaaaaaaagttctattacagatttaaaaaatattgtatttatttaaggtgtataacatgatgctttgatatatatacacatagtgaaatggttactacatcaaataaattaacatatccttcaccttccataattacctttctgtggtaagaccatttaaactctgttctcttagcaaattttcagaatacagtattattagctataggcctcatgctgtacattagatctccagtattaattatcctacataacttcaaatttgtactctttgaccttcttctctccatttcttcccctccccatccctggggcccacccactgttctactctctgtttctttgttgcttaactccttcttttaagattttgcatgtgaaaatgagatcatatattaatagtatattctttctttgcctgccttatttcagtttcattgggattgctgacagtaattaaaccattttacaggagtgactgtatgcctcacatataccttccattaaacccaaagtaaatgtgtcttcaattcaaatttccattagccagattccaaaaatacccacagccactccagtgccacccaacaggaggctttagccaattatgtttaagatattatactcttaggccgggtacggtggcgcacacctgtaatcccagcactttgggaggccgaggcgggtggatcacaaggtcaggagttcgagaccagcctgacaaagatggtgaaatcccgtctctactaaaaatataaaaattagccaggtgcggtggcaggcacctgtaatcccagctacttgggaggctgaggcaggagaatcacttgaacccgggaggcagaggttgcagtgagctgagatcgtgtcactacactccagcctgggtgacagaatgagactccgtcttaaaaaaacaataaaataaaataagatatactcttgacaatttgacaaaaaaaaaaaatacaacgagggccttagaagagagccatgtatctttatgatctttatgtgaacatttaaccttttgtaagagtgctgtcccattacactatataatgctactgtgcaattctcaattatttcatttgaaagttatttatgtaaagagttgattatgccctttctggagggataagaatgtggtggactaacttactcttggctaaatcacatgaacatgtagaagtgaatacattagttggattactaccttctctggccggcagaaggtactttcagttctgcaggcagcccctgaacgccccagtggttgggcagcacctgtgatgggatgactgaaccgttatatgccacatgtttataggcgtggtagacagtatggggaggtggaaaagactggttctgacctgacacacatttactacttaataactcagttattcaaattgcacctctttgttttattgtcctcggggttcagaacagtacaccaaaacgctaattaacaaactatgagacagcaacagctgggaattcagtgtagtaaaataacatataaatgtgagacattggttgtcaacagagaaggcccatttcctcatttttatttaaaaaaaaagactatcagtgatatacagtgattagcatactggagttcttttaagctatgcacaatgaggtatcttagaattaaactttagaggttcaaaataaggatatgatatagaaaagattgaatggtaggagaacagaataggatttatttatattatgacaaacgtgactatcagttaataaattggagatagtaagttaagatcattatttatattatggaaaaatgaagaaataggacatggttctctaactttttaatttttctgtgaaataaaatattatgacttattttatttttatctttgtttttaggtaagactgtgccaaaatcccaaacttcagttgaaaaatagcccaccatatatacttgatattttgcctgatacatatcagcatttacgacttatattgagtaaatatgatgacaaccagaaacttgcccaactcagtgagaatgagtactttaaaatctacattgatagccttatgaaaaagtcaaaacgggcaataagactctttaaagaaggcaaggagagaatgtatgaagaacagtcacaggacaggtaagaagaatatttcagatgttttggtgtaaaggtcatttatgttgctttttacttaatagttaacctaaacgtcaccatgtaattgttttggggtaaatgtgagtcgctttgtatatagtcatgtggtacttaacgatggggatactttacaagaaatgcatttttagacaatttcattgttgtgtggacatgatagagtgtacttacacagagctagatggtattacctaccgcacacctaggctgtatgatgtagcctattgctcctaggctgcaaatctatacagtatgttactgtactgaatactgtgggcagttgtaacacaatggtaagtatgtgtgtatctaaatatacctaaacatagaaaaggtatggtaaaaatactgtataaaaggccgggcgtggtgggtcatgcctgtaatcccagcactttgagaggctgaggtgggcggatcacttgaggtcgggagttcgagaccagcctgaccaacatggagaaaccctgtctctactaaaaatacaaaattagcccggcgtggtggcacacgcctgtaatcccagctactcgggaggctgaggcaggagaatcgcttgaacccaggaggtggaggttgtggtgagctgagattgtgccattgcactccagcctggacaaaaaaaaaaatactgtataaaagataaaaatggataacctgtaataacctgtataacatgcatggagcttgccagactggaagtgagtgagtgaacggtgagtgaatgtgaaggcctaggactgttactgtacactactgcagacttcagaaacattgtacacttaggctacactaaatttataaaaaaaatttcttttccaatatgaacagcctactgtcacatttaaacttgataaacttttaattttttttaactttttgactctggtattaacaattagcttaacaaaaatattttctttctatattaccctataagctttaaaaaatgttaaaaaattaagttttaaaaaaaattttataaactttttaaaaactaagacacaaacacacacattagcctaggcctgcacagtgtcaggatcatcaatattattgtcttccacctccacatcttattccactggaaggtcacacaatatgtaactttttcagattggcttctttcacctaacaatttgcattttagcatggagcaataacatgcatggagctatcatctgtgataatagtgccttcaggaatacctcctgaagggcctgcctgaggcccttttactgttagctgttttttaataagtagaaggaataccctataaaataatgttaaaaagtttatagtatagtaaatacatcaactagtaatatagtcacttattataatatcattatcaagtatgtgttatgcataattgtatgtgctatgcttttatgtgactggtggcacagtagatttgtttacaccagcatcaccacaaacatgtaatatgttgcactatgaccttgtgatgactatgacatcactaggtgataggaatttttcagttccattataatcttatgaaagcactaacataaatgcagtctgtcattaactgaaatgtccttgactgaaatgtcattacggggttcatgactgtatttgcttttaacattaatagctttatgtgaatttacttatgatagtagctagttcatgagacagtattctcaatgttttaatagacttcattttcagagtcattttaggtttacaggaaaattattcagaaattacagttcccataaatctcctctgcccaagcagtttctcctgttcacatcttgcattggtgtagtgtgttatagttgataaactattgttgatacattattattaactaaagcctagagtttacattagggtccactttttgtgttgtgctgttctatgctccataaatgcataatgtaatatatccaccattacagtgttatatagaattgtttcactgccctaaaaatgccccgtgatctacttactgtctcacttccccccctgaacccatggcaaccgctgatctttttactgtctgtgcagttttgccttctccacagtgttacatagttggactcacacaatatgtaactttttcagattggcttctgtcacctaacaatttgcattttaggttcctccatgtcttttcataacttggtagctcatttctttatgccactgaataatactccattgtgtggttgcatcacagtttggtttttcactcacaccttgaagtacatctcggttgcttccattttttgggtaatcatgtataaagttgcaataaatatttgtgtgtaggtattcatgtggatttaggcttttaactcatttgggtaaaatacctagcaagattgctagattttcagggtatgcataagtttagaaagcacataagaaactttcaagaaagtttgtaagaaaccccttgactggttccaaagtggctctaccctttttcattcccaccagcaacgaatgacagttcttggtgctccacatccacaccagaatttggtattggagacaagtgttttaaagacatagtttgattgaatctgcggatgtggtagatgtgggtgggagatgtaacttccatctgtgtattttgacttaagtgagaaactgaactgggctggcaaagactgaattgacatcaaccaaatcctaattttatagccaggattcttcatttagatatcctcattcaagtttattgtttggccattctaatgttaaatttcaggatctatcttaaacaaaataaggaaaatgatagagttttccatttctgggctattttaaatatatatggaatattcatgatagtgatagtttatttagatttccaaattggaaaaccaatattaatgaaaatatatagtgtcttaacatgttagttaaattttagtgtgagactcttaacctctctgtatttagttttctgacataggaaaacctttaagaaacctctaatatttctttcaggtcagaaaaactggcctcatagggttgtaaaatagaaaattcagttatggaggtatgtgaactctaaattttctatgaatactggccaaatacccataacaagtaaaaccatcaaattcaaacataaaaaaccctcttccaggtgtaaaatggcataattatatatttataatttataattttctctctcaattagagatgaacccaaacatactcgtagacgtctaaccaattatgtaactttgggctcgtcacttaatattcttgggccacagtttattcatcttgagggtggtgaatcttcctgttgccttttagcattaattaatgctttaaaattctccaggaataacaagaggaatttgagttgccacagtgtgacagaagtaaggtagagaaatattatagtgattgtttattcttgtacacttttcaagaagtgctaacatcttgggaagtcagatatttatgttaaatttttatttttcacattgtatttctgccattgacctaataaaactaagttctttacaaatgtctttataattcttttttcaaatttagtagttttgaaattcttcctgatccagccaagtcatccattaaattttactctttagggtatcagtacattgacaaggcatataactcctttgactaggaatttgagtggatacagtgttttaattggatgtaatcatatatgatgtggtaaacttgtatcccacttttacatcttctgtggaaacttgaaacattttctaactcagtagatgttttataatttccatttgataattttgatagttttgcttatgtggaaacatttcatgggagacatttaagttttgtttgataatgtatagattcccatagattttgatagctctcataatgctgactattcacaatgaggtcactcattctgtgtattcttgcctgcattttaagtgtccaagtgaattttgttcactgtcctaaatgcaaccctaagacaaaatttattggtttctatgctgatatagcatatccttaattggatattactgttatccttgttctgtttactgacaatgttctgtggccagctgtgtttatgcagaatttacatttgataaatgtttcataacaattcataataatgtaccattcttggtacaccataacaggcacataaactttcgaactgtgtttctttttctagtaatatatttggacacattcacttctaggttcccttaggattccaaatgcaaactgagtctaaaatgtgccattgcacatggagctctgaggcattttgctctacagaacacagaagagtcttcttagcatgttttcatttacagagtggcctcattctgaatagtcagcattatgagatctattgatctatgaaaatctgtacattaccaatcagaacctaaaagtctcccatgaaatgtttctacataagcaaaacaatcaaaggtagttaggcaacaaagtatatggtttagtagaatgactccacctaatcggtatgacaacgctggttactgggaatattataaattcactacagaattgtttctttgattctgtaagtagctcactagggacattactataattgagctaaaaatgaaggacagtaatagtagaaacagtaatttggagttaattgacttaatcagtattacttcatgtgattctgcccgctttattgaagaaattgtgctggatgagtacatgaatcaaaatgcatattttaatgttcctgcacctctgttgtgtcaacaataaatgcggattgttatacatttaaggggagatgagaaaaaatttaggtgtcaattctgaactgattgctttttaaaattttagttgattgttaatgatgaatgagtagcccgaattttaaagcaattttacataaatgtaataaatagaaagataaagaagtaaatggcattcaatacatttcctagttacttaggtcctgtgtagaaggagagaggtagaaattatagtcacataattgtatatgctactgaagtagatatttagggcacagtgaaagtcttttgccttgagagtagataatgatttctagtagagaaaaggcatgagcaggctgcatgcctcccccgactgatacagtaaaccttcatcttttatatcatttttcccaaattgttgttgttgctggtgctctgttgttttgttctttttctttctttctcttttcctgcaaacagttcaatgtcacactgagttggtgctactccagctgggtgtctaaaccattctgagctgacatttctgggaagagtaattcagtgatttctgacaagcataaagcttgcatgtgtatgtgtgtgtatgtgtggagagaaggtaaagaacatgtggagaagtctgtagtttgaagcatagagaaaaggagaaactctaagtattatattacttaaagttttagtggcgcaagtaaaaatggatgtgaaggttgtcattgctaacaaatgtataattgataggaggcatttctatgattttactggcagtttcatcagttaaccattttaatagtaaaatttatttcttctgaaaagtgtgtattaatcagctgctctgcaaggtctggtgttggccactgtgttttttaaaacactcagatgatggccgaaggatactgagattttccctatttttgaactgcttgagtgagccagggtgctacaacccattttcccctcctacacactgctagacaaccactggaaaaaaagtcaactgaggtggaaaaaatagctctgcctttgatagactgtgaagaaaataaagattaacaaaagctctggccagagaggaaaacatagatgttgccttaacattaacattaatgtcatttcagagcctttgaatcatgccttaataaccagtgttgtagaaaaaataaatgacaaaatggggtgtgtgtgtgtgtgtgtgtgtgtgtgtgtgtgtgtggtggagggttacatgttctgtagttaaaaaaagtatttgacatagaaaaattagctctagaagaagctagcctgtatttaaaaggcttcaggagcatccggcagaaatatttttctccattgactgtttggctaaacaaagaataatttgtaatctgctccattaataaagtgcctttccccttaaaggactacagatcactatgcttgctgtttctacttgtgttggttatttacaagatagtgtaccaaaaaacattttgcaattggtacgttgaagatacatggttaaaagttgaatcaaaagcaacagcagccacaataataacaagagcaacaacaaaacaaaagctgcaaacaaacctactttctttccttatgttactgtatgacatcattgctttgagtaaagaatacagtagtattaatttaaatgcttaaatttatatttatacaaatgttgatagcatggtcattgaaatagttttgcaagtcctcacaggttttcagtttaccatataaagagattgatctgtatgtgtaatttgtgctttaagtggtagttttaatagttactggacctcacagtttaatgggcatgtttataatgctacatcaattaccacattttctcatagttctgtatattgtgagaagaaaatgaaaaaagtgctgtttaactaagtacaggaatgtggagccaggaaatctcatgctctgatgccatttcttctcccagtgttctttgtaactttggttggcttcctgactccaatttacattttttcagttattaaattattttctggttattcataacattttcacctcagacatttcaatgtcacaaatgaaaattaagttgtttcagactaaatgctactgttgctacctcagtctgttaatatttcaactttagtagtagtttctatagtaaaaatattgtttttgttattataacaattatttgcaagagaatcttaagaaggtttgtcttttataaataaattatactatattggatatctatgaaatatgtaatgatacatgaaagggaatggcttatattcatcattaaaataattttacatttaaaaaattaacctagtttttctgtttctcctttttttgtttactaatggtgaaaattgcaattattttgaatttgatgaggacaaaggaccaattgcaagtagaaaatatttctaaataaatcaaaataggcaactaatttcttttgaacacaagtattttgatataaacatatacatttactcattttttctatatttgtttgcattcaaggcattttttccttttaaatgggaaactagaggaatgaaaatatgaatttttgatacttcaaaataataatttttatcttggattcagaagacaaatagtaatcaatctcttatgtagaagtatgtctgccagataggaagtagaatttgctttagaaagcttgaaaaatcttctgagattgatttattgtaaatgaataaaaagtttattttatcttgatgagaatgtttttatgcatatttcaagcatctgagtggtgtgaatagacttttctaaagtttggaaatgaaggggctcatttctttaacacaccatgaaagtagaggccacaagaaagttatttagacaaatgttttcaaagttaaacaaagggagtctgttaggggaggcaggtgagagcttgcagaatctttgactgttaaggactttatggccctgtgtatttggctaactgcacccctgctgcttagctagctagatacaatgtaattttcttgttgatttttcaatgaaaagtgaaagttttttttttttaatagcagtttcctctctagggtatattttacaaaatacaaaagaaaacgaaattattcagatgtaattaaaatgaattccaccattagctttttaaattttaataaaatgcttacttgtaatacatttatttgctatttatttcatttatttagtttagtttttttttttttttctaagccccatgttaatactgttcagcccccaactcctggtttcttagtaactaaaacttattgcaagtattttaatttcttctaaatcagattatatattatgcacatatgatatttggatattaaaattgaggacttttttcccccacagatgagcatttgttaaatgtttatatattcctgagaatgataccaattattatatatattaagcaaactcacaagactcaagtgaacttggctttgcagtggaataccgtttatgtttttcagtcttaggtagcttattaaaattgatgttttcctttcagtaaatatctggtgacattaaaatttgataattaaaatccagaagcactcaagaaagaatgcctgcttttagtttcataataacaatactcccacaaagaaagttgatgaatgatgtctttcaagtggcagagttgttatttagttaaagaaagctcctttggcagtttcttacaaaactaaattaaagtttgtttataaaacagacaagctacagtgataatcctatagcctcaagagtgcttggaatctagcccaagaagagtcttgtttttcagtgatttaacaaagaagtggagtttccaagaatccaaacagattagcattcagtgctaattgggtagggtgtaaaatagtattcatgtctgattgttttgatgtgaagtacttcataaccattccgttttctaaatcttttaaaaatattaatcttttgtctcttcagatttattgtttgagcatgagtttttgtttgaggatatatgtatacatcaatggttcatagatgtgctgtaaacagcaagacacatgagatcattacaaaaagtgccctttatagattaaaataataaagagagattgaggctctgttgttcagtggaaacattgaagaacaaattaaactgaaagataatttagatgttctattgaaatgtattactttgaacatctgatcatgtgattataatataccactttcacatataatcacaaatgtgtgactgtatttgtttcttcgggctgcggtaacgaagtaccacatactgggtggcttaaacgagagaaatttattctctcacatttctggaggcaagaaatctgaaatcagggtgtcagccagcagggccttgcccctcagaaccctctagaataggtaatttctttgtctgtctcagtttctggtagctccaggaattccttagcttatggcagcgtaagtctgttctctgctctgtcatcacacagtgttctctgtgtatgtacgtgtgtgtccaaatttccctcttacaaaaacaccagtgattttggatcagagcctacccttgtgaccccatcttgacttcattacagctgcaaagatgctattttgaaacaaggtcatattgacaggtaggattgaggttagaatttaaacatatctttttaggggacaccattcagtccctaacagtgaccaaatatggcagtgtacatttatactaaggaggggaagaaagttgaaacccattagaacatgtcatggaggtttgaaaaacttggattaatagaatataatagcaaacaaaattacttgtaattattttttgtaaaggtctgtagctgaaataagtttttttcaggtgtgtaatgcatggttaatttatgcattagtttttataaatatattggaaaacatggcatattctgcatttcccaataatacatttttgtatttattgatataattgaatttgtaagtacctttttttaacattcttaccgattttaatgtataaagtgccatctctctttagaaattattagtaatacaaatatgtacatttaatttacatttataatattaatagccctggttatttcttaacaatccctgttttctttaattttgtgataatctctggaggattaattagaataattataacttattaaatctgtttataaacttttccctattgcacatagaaaataaataattgggaagtgttttccatgttggtgacaaagagaaggcaacaaacaccacattcattttgtctttaatattaaaatatggagtattaaacatgatttcaattcaaattttaggaaatcaatttgaggattttagtttaaacaagagtaaatgattgtttctgaaagtggcatgttgaatggtttaaaccatgggtcagcaaactgtacagagccagatagtaaatagtttagactttgccagatggtctctggcaattagtcagcgttgcctctgtactaggaaagcagacatagacagtctgtaaattaattgacatggctgtattttgataaaaatttatttacaaaaatgagccagaccaaatttgaccattgggtggtccttcgccaacccttggcttaaagtacctatctaatttttggagagtaaaattttaaaactattttatcaacaccaatccatcatataagttgcattatatgtaagtttcatcaacacagactgagtatataagttggctaaaagtaacaatacccatctaacagtacaatgctgtcagagacccaggctctttctggcttattgtaattcatttccttagcatgttgggttttatcttcattctgttcccttcacagttgtggaattcctgttgcagcttcattttttaaggacacaaggcaggaaaggggaagggcaactccacacgtgtctgtcttcttatcttgaattgcaaagctgtcccagtaccttaccacctacttgcttctctagcagattctcttccatattatttaagccactgggtcactccaggttacaaaggtagcggtatattgaaactttgaaatttcagcctccatagtaaagaagggcaagggagaaacggtgtttgtttagtcagtctaaattgtcaaaggagatagccagatatctctttttgagagataaacagacactcttcatttaaacatggtataacttggctttaaggcatatttctttaaaaatatattgtcaaggactgcgaagagcctgaagctactttgccatactttcagggctagcagaagacaggagaatatttggtcggggaaaatgaaccatttattagtcacagagggacagcattaggcaaggggtcatcacttgtgccaggtcaccaaccctcagtttctataggacagtataaagggcaggagacacctgcatgtgcagtggctggcattacagatgaatcccaagcctaaggaccctgaatttttataatggacagtaaacctgcctgactcttgctctggaaggagacattatctttattgaactggactgtaaccctctttttttttttttctccagaggaaaacactgtccctattatctaaggctgtttgatacacaaatgttcctgaaaggataatttgaaaggctggcagtatctggttgcaagacatgaaaaaatgtgggagacctatagatacttctccccaaaatatttatgttctgttctttcttaaaaaccataatgtatctcatgttctcagttttgaatgctacttaagagtaaggatatttactctattttggattattggtaagcttaaaatcttgggttctgggctgggcgtggtggctcacacctgtaatcccagcactttgggaggctgaggtgggcagatcatgaggtcaggagatcgagaccatcctggctaacatggtgaaaccctgtctgtaataaaaatacaaaaaaattagctgggcgtggtggcgggtgcctgtagtcccagctactcgggaggctgaggcaggagaatggcgtgaatccgggaggcggagcttgcagtgagccgagatcacaccactgcactccagcctgggcaatacagtgagaccccgtctcaaaaaatatatatatatcttggtttctgccattttaatatatataacaaattatgtatttgatatatctaatatgtatattagaaatctcacatatatgtgaaataatgtgatctccagaacaactgtggaaactttgtggtgtgaacatttttaattcctttttttttttttttgaaatgaagtcttgctctgttgcccaggctggagtgcagtggcacgatctcggctcactgcaacctccgcctcccaggttcaagcgattcttctgcctcagcttcccaagtagctgggactacagatgcacgtcaccatgcagagctaattgtttttgtatttttgtagagatagggtttcaccatgttggccagactggtctcaaactcctgaccttaggtgatctgcccacctcggcctcccaaagtgctgggcttacaggcatgagccactgtgcttggcctacattttttattcttgtttgcatattagaaaaaaaactttaataattaaagaaggaacagttttacaaagacttttcatttcaagctcattatcctttaaaagagaagacgattatattttcttctttttttttttttcttgagtaatacttcattcacttttcaactttgactactgtaactttaggtttagacccaaataattggtttcagtaggtaaggaaggttgaaattatgtgatataagaatgatattgtttatctttgttatcagtgtctaaacctcagagatgagcaccaggtagtccaattccattttcctgtgtggagaagctaaatagattggaggttacatgacttgacacactggggtgtgcatgtaatattcatgtgtgatatatttctaaatgaagtttcgttaccagttggcatgtctgagcctacagctgttggcatatgaagggtatcagaattataatgcctttttcaactttgtttcgggaggactcatttgaacaattagcacattttatggttgtgaaaatgaactgttgttttttctcttcattaaaccagaattaatattgtatgtcatctggtcagtcgatgcaacttttttttttttctaacacgatgcttgtattcattaaattcagaaagactatttaaagctttatttttcaagtatacctttttctgtaaatatctcttcctttctcactctgtttatgtatatacatgtgtgaaatattacttgtaactttttaagcatttaatttgtcttgatgggcacagatttttaaaaaggaagtaagaaagattacctagttttttctgaactatattaatgataaagcatctcagctcattttacagatgctgaaaacccttcgcgttttgttctgaaggtttttgtagggcatactgattttctatgaagtatgtggagctgtcaattttattcttttttccagagatttcagaaaattttcatttaactaaagatttccatcctggtcagctgggtctcatttacctgattaaaataggtttgcatggataaagtcttaaagcaaattctccttaccacattttgtgtgaattttttcctctttaatactaattttagtttgttctcattacaattgcatatgtaaaaaatactttttgataaagcaactgaaactttgaagttgataatttatcacaatacttttttccccattatatcaacacttggcaaactacagactgtgagcctaatccaggttgtatttccttctgtgagtaaattttcaatggcggagccatgctcatttatttacttactgtctgtggctacaatggcagaggtaagtagttccatagagactatctggcctgcagagctgaaaacttttactctctgggcctttactgcaaatgtttgctgattcctgcattactgatatcattttcataacagtcttaaaaacttggcatttttaaacttaaatacttttttctttttgtcgctttcttctctaccctatctctgccagcagttctttgtgaattaccattgtgatcttctaaaggcaaaaaaaatggtaggtagtcaatgactgatagctatagacctatgaaactaacatttcctatcttgtctcaatattctgatgtatataatcattttaaaacataataaatttaggccttttttgtttttgtttttaacacaagatatgccactaatgtctgacaagcatttaatctagtggctttcctgcattttccatggatcagatgcattctaatcctctgaaatgattattaaatacagattgccagcctttatataatgcctgctaaatttaccagttgggatctgcacatatattggtaaagagtgccctagcagtttctgaccttctgtctgctttcagaatccacatgagtcaaattatctccttttatgtataatcttatcaaggactattgaggtggcacaggtggggatggaatcctggatgtgttcagaactcctttcattttactaagcttcctttatactaccagaggaatccacttccagtctcgagtcacttagaagagggggccgggcgtgatggctcatgcctgtaatcccagcactttgagaggtcaggagatcaagaccatcctggccaacatggtgaaaccctgtctctactaaaaatgcaaaaattagctgggtgtggtggcatgcgcctgtaatcaggaggctgaggcaggagaatggcttgaacctggtaggtggagattgcagtgagccgagttcacactactacactccagcctggcgacagaaaaaaaaaaaaaaaaagaaggatacatttctgtaaggtttttaaagttttgttggaaaagatggattgcaattctcttcttatgtttttctgagattagcctaatcactatccttggaatttacctttgcttgccagccttttatcctgcccctttctcctccctttcctatttccagtttatgcagatattctttcttattactggcatagctaacattcccactctatccccatgcccatcttggctctgattttctgatcattctagctaaaatgtgtcttttccttctgaccatagcatttatcaaggcctttcatgtggcagttaatgatatattgtcaaatattgttatctttttaaaatggtgttaggagcctgggtaacagagagaccccatctctacaaaaaaaaaaaaaaaaaaaaaaaaaaaatagccaggtgtgttggcccatgcctgtggtcccagctactctggaggctgaaatgggtgagattgcttgagccaaggaggttgaggctactgtgagctgaggtcatggcgctgcactccagcctgggtgacagagtgagaacgtgtctcagaaaaaaaaaaaaaaagaaaaaaaggaaaaaaaaaggtaataggctatgtctcttctgtgtctcccatggtatccagaacagtgctttatacttggcaaacactcataataatatatggttatttctttagaattactataggatgatttgaaaatcctgatgtattcctagattacgagctgctggggcctggagggaggcagaagagagatgttacaatggaaagtgttgaagtgtctgaaggcttgtttttagaatttgtaggtacactctgaatgtcagcttcttcacatttgggaatgagtactggcgattttagaagaaaagaaaatgcttataaggatagtaatatcattattatttatgttttaattataaatatatttcaaagcaactcttaactgtatgtattctcaaaatagctttactgtaataacattcttggacaaagttcatgaaattactacagtttacaagtacttctttaatggagtgtccttgtagctacacttagtacctgtaagataaattcctaaaaaattcacatcattctgcctactctcacaaactactcaaagcaatgagcatattgcatagctttcttattgacttattagttctgtatctgagctccaatgagtgttttcacttcctcttattcttatattaacatttcttcaaagactgtttttgtttctgtgatgtgttgtacttatgaccctgaatacacaggacttttcggcaactggggaatcctccaaaaatctatgctttcgacatgaatagtccagtttcatttggtatgtattattgacagaagtgcttcatcctctttatagcttgctgtgcagcagatggttgttatattattcaggactcttgaggcagtgcattaatacgcgtgcacaatgttttttaagtcttattgtgggaattgtcttgtgtccttgttgacggaattccttaatacggatgaagatttccaaacttgaaaactagtttttaaattttagctaaggctattttgtatttatgtattgcttaactgctatctctttttctttccttttaaagtaaaggtcggagggttggatctaatattcttgctataaatgatttttttttttttttggtcttgttttgttttgatgtgagcatatcagctttgagggacatctgagatattttgaatatgtaggcagggcttttaaagaaaggaattgctgttttcttacatccaccatgcagacttagacttgacaggaaaacagaggcatgtgtgttctcactgtgtattatcgttgcgccccctcacatagctgttagggtttgtcagcagtttgttttgactcactagaccaagggtgggggtggttttgtgtgtttttgtaagttaactagtgtgggatgtcagtaatctcagctttaccattttagttggccatgatcccttcatgtaagaaaccgtcatatttgccctatttctcaggccatactgtggttccgaaacttgcccgtaaatatttgtgaaatagtttttcttttcttataactctggaggtcaaaacccagccctaagataaactcctgtttcatattgcctctgcggggtgctcatggtatgtccccagactaatttgggaacctccttgaattactttttaaaatttatctggatagttcttacacaattaaatttaatttttatttattatagcaagattgaggacactcccaaatccacacttcatttcctgggagatcatgttagacacagataactatgctaaattattttgatccaggtaagcgtgttagaaagagaggatggggctgctggacttcattgagaggatcttttgatttcagaaaccattagtgaaggaaaggtgtactagacagagctgtcataacctttttactatttcttaagagaagtggtacctctgagagttgtgtaacatggtggtcttcccacatctttatcaagagattaattaaggttgtgaagtaaagctttagaggtgaggcacaaaaggcaagcagtgtttacctctaatcactcgaggtaacctttatattggaagatcagtgtagatgtattgtgtaccagataacattccattatgtaactattctttatttacttcctctactgtatgtgtggcatcattccagcatcaagtttaatccttctttgtatgcatttattgtaaactgtcatttatgtgggattaaaataactggaaagcctaaataactggaattcttgtagcatgtttggtacttgacatttaaaagagagacagatggcaagtaaacaaggtgagagcaggactttatttgctgtactgcaatctcaaactgctgtcaagaattttaattagatgttattttaggaagctttcaatttactgcaggtcattgaagcttagaaagaagcagaatgtacaccaagtattttaatatgttagcaatccactgcaattagttaatctggagctagtaggttgtatttttaagagtgtaaacattttactgggtttcagtgtgacttagagtaaaataatttttctgtttaattttaaatcatactattttaaatttctaagttgggcttcagtatcaagttggttgaattaaagatttaaatatttctcctgatgtcttcccttctccttcctactaaactggtgttcatttttctttatggtgatcatttctaatttcccacagcttccctggacatttccttcactgtgagatagtagttttacacatcattcaacatccagttgttcagttttcacaaaaagatttctgagaaatctgacttcaaagttactcttttcaaactgaagctcgtttttagaactattcatgatataggaaaatacattcataactgaaagctttttttgggttattctttgtaacattacatgccgcttttcaccaatgaaaagaatcctcaagaacctggaaaaagcaattcctttttgcagttaaaatgaagataaagagccttgaaaaatggctgatgctaatagttcatggagattaataagtgatatgtgaaaggcagactgtcctgtagttgcaaaatgttacatagtttgtacaccttgtcagcgttgtgataacatgaagtcatagtttaattggtaaacaagaaaagcttccaataaagtgtcccatggatgatcttttcctttgagactgcagcatttctgaagaatatagggtatcttgtgatacagcaaggtaaaagcagggcacatcttccctatgataaatctgtgctcttattgtcggagtttaacctagtccttcatattttgtttctgaatacttgagttcatttatgtatagtggggaagattttgtttttgattttgacttctgtcatgcttgcttgcttacttgattttcttttcttttttgagatggagttttgctctgtcgctcaggctggagtgcagtggcgcaatcttgtctcactgcaagctccgcctccctggttcatgccattctcctgcctcagcctcctgagtagctgggactacaggtgcctgccaccacgcccggctaattttttgtatttttagtagagacggggtttcaccgtgttagccaggatggtctccatctcctgacctcgtgatccacccgcctcggcctcccaaagtgctgggattacaggcatgagctaccgtgtccagccctatttctttatttaaaaatgacaaatacatttatggttgtactacatgatgtttctgtatatgtatacattgtggaatggctaaatcaagctaattaacatagccattacttcccatacttactttttttcttttttaactggtgagaacatttaaaatataccctcttagcaattttcaatatacagtacattgttaacctcttgaacttatgtctccggtctaactgaaatttcgtatcctttgaccaatagctcttcatccactgcctcaactgcccacccccaccccaggaaattgtgattgatatattttttaaggttgttgaattaattgtcttttttgcagttaaaaaatatattaatagtaaaatgggctaggtagagattttttcagagggcaagttaattctggacctgacaaccttaaggtctggaaaaatagttactctcaaatttttttttttaaaacgaaaggttttacttaattatcaggttctaattcccttaggatactggtcttcagtgattgtttaaatgtctcagaggaaacttatataaagtttggaattttcattttaaaatttgtgttgttccaagatttcaaaacagatctaatctagattattattgatgttaaatgatgtaaaataaaagtatatttaattttagaaatttccattaataataaaattattcaaattataaggaatatacccaagaatgtgagttcttttcaaaccagaattaaactcatttatatttataatacccctaagtactgaaaataaatactaacagcatttcttattcagaaaacaaaactggttgcaagcagcaagagaaaatatcttgctgagtgtcgctttgaaagccaaatgagttgtaaaaaaaaagtaaaagttcctagtttttgttttacaataaagtttctaattttgtctttagttttaactgatcatagaaattaataagcctgtctgtctcagagagagatatttgttctgccttcactattctgcctgaagcaggattctggaatgttcttcagaatcatgcagaatgagttgtaattatttgctacagaatgagttgcaatatttgctagtcttcaaattcattctcattattcaagattatatgatcagaatgaatggaaactgcgatggctataacctagagaagtaataagacaaaaagcattatttactcatatgaagggtttatcattcatatttcttcatcatctacctttctagattcatttcacgacactctccctcaggtcttccttctttatccactcacatcatttttcagtttttcaggcctttaggacctccacatctgaggcttcatgtgcctgatttttttccaactacctaacacatctttcgtatcataacttaaatgtcatttcctcagagaggccttccctgagtgatcattaaatgttttgacacattattttatctcatggtattctgctgtgttctttccagtacttgtcacagtttctgtttcttcgttttcttcaccactgtcaccacagtgcctacttcatttccaacacggtgtaggtactcaatacagattgaaggaatgaaggatagataatgcactcatcttgagctacatcctatttagtgagcaccataaaacttggcaggcattgttagacctttatatggacaagagctttgatggttcgtttttgttttgttcagttttgttttctttctggagatggaaaaagaaatttaatagtgataaagccagagaaacttaagctggacctgtcaatctggtatggttgagaggtatttattttaatcagctgtggattgtattttcatgtgacattttaaaaaggatgaaaagggtcttacagccacatgcatcattcttttgcctgctgatccataattagaatcagcatctagaatctgtttttgtatataactgaagtatcagagttacaagtaaaaccactttccacgttatttgttgtccattaactgctgaagattagcatatttaaaagttttactgagaagcacataattggaacctgatgtttgttatttatcctgattcatgttatattttcttcttagtggacattaaaatttcaaaattgctataacagaggtgctgttggggtagtttcttagttgtatatgggtcgtatattgtatatgggaatctttttatggtacacatctgttttcatcaaataggtgagtagtattcgggcattaccttataccttctttaaagaattttcatccattgctttaataattatattatgccagctctatttaatataattaaatttttttttaaaatttcctgttgtatcaacctgaaactactttgactagcattttggctgcaaccttcttttcttaactgttcagtatccctctgtgtatttttatttggttcccttaattggaaataatttaaaaaacataatgtgtgactttcttgcttactcttgaataacatttaaacttcagcctcacttgtctaatttcagttctgaggtatgttctacttgttgcctcctcaaattgctttgccttcctgaaaagtttttcttttgattctacatattttcaggggagggataaagatctaagcataaatatcacctccttagatgggtcttttttgaccatcctatcaaataggttgtcacagttagtattgtgaatattttctgatgatttattttctcccccaacaaattttaactgtctttcaaagtataattgaagttctcattcttgaagcattttctgaataatcaagtgctagaacttgagcattttttagcttttactttttgtcagaacattgtagttggtggtgtagagacagttctatcttgatgcttcaggttctcatttctacttgagggaagctcacaattttcaggaatcacggaggatggcatttgagcccgcctggcggtctgagcccacctggagatccccatcctttcaatggggaaatgtatttgagctattccccaagctaatgctcaggtaggagatacaggcaggaaaagatgtcatggaagatattttaaaggaatgggagtatacgcctagcttagaatacaatatgtgaaagcatattgtattgtggttctttggtgctggatatctcaggagtggaggaattgaaggattgtgttttcatttttacactttttctcttctgctcttctgatttttcaaccatgaacatggttatgtgtatggtctggtttgggggaagtatatatcagaaaaaataaaacaagaaaggaagttactctggttaccatgcaatttcgtatttaaatgacaaggttttgcattttggatatttcatgtcagccgtgtctcctcagctaagttctagtcacttagaagtttggtgtcactgaattataattttggtgcttcccatgttttatatttacactagactacatgctatatggacaatgtgcaatcagttaatatctgaatgaatgaattgaccaacttttgagaacagagtttgacataaatttggtggtttttggttcattgtgctttaattgaaggagaagtagacattgtacttcacattaggctactctttagataagctttccattacatggcatgtgtcttactttcttacttgataatggctctgtttctgaaattatataaacagatttttactgtttttaaactttacaacttaatattagactgtaaattatttacttatttattggaaacaaacttttagatacagaaattggtcttttaagacaacaacaaaaaatacctagtttttaatctggctcctgtctttcatgactaagtctgactgatctttcttgttatatgatgctttataagattgattctgtatccgggttaccaacgcttcctgtaacagaagaaaaacattcagatccttatagtgctgagtcaggggaaattattacatacaacaaaggaaaagtctttgataggaaatattgcttctggtaggaaattaagtacagagtacaaggatctaaatgtctgtcatataaacaatgggcaatcttattaggcaattttggacacctattacgtggctttatttatttattaatagtaggcttttctcatcatggttcatggggatttgtaaggtaacgataccctggacagccttccaagatgttttgatgttggcagcaatgcattactggggcaggggcaggggaggaggtagaaagtgtgataactctgcatcactccttcttctttgttctcatttataacacctaaggcattggaattccccagagtaagaattctagtgaaaagggaagcaaataaaatacatctgacagttgggatccttgggtttatacatgtgctgactaattacatatgaaatgagatatccccttaaattaaaactatttgaagataccacttcaaatatatgtaatagatgatttacacttcttatcatggctttcaatagcaagaatttctcttgcagtaggataattaactagaaatataaagaaatgtttcaaaagtataataatgtagaagaaaaatagaaaaatttgattttggaagaaatcttcctccattattatgaagatagctgcagtttggtttttttttttttttttttttttttttttttttttgggacggtcttgctgtgtcgctctggctatggtgtggtggcatgaacacagctcactgcagcctgtacttactaggttgagccatcctcctgcctcagtctccaagtaggctcccaagtaggtgggactacaggtacatgccactatgcctggctgatttttaaattttttgtagagacagggtctcgccatgttgcctaggctggtcttgaactcctgggctcaggcatttctcctgccttggcctcccaaagcgtcaagatcacaggcatgagtcaccacacccagctgatagctgcagttttgtgtgtgagagttgatcttcttgcctttactgtgacaaaacatctttatggaaagacaaacagtattcgttacgtttattttacctgtgtattcaagaatgtttatttttttcataagctacaaaaccagattagaaaagtaacttgtatttcctaaaaggaggcatattttaggagctatatgaactgggcatttttaattttttttcattactttggggaatatttcaggatctcatcagcattcattacatgggaggacaggggattaaaaaaacaaaattgattgttaatgaatttttttttttttgtatttaggaagaagaaagactgtaacttaaaaagaattgtggcaaattatatatgttaaatattctggtctagaaatgtggtatagaggttccctagaagcttctgttccactgtggttaaaatgttttctcaccttcttgaactgtctttgtaggtgatgtcatgaaatgtcccaagagaacgttgtataaaacttcctttgacctttctggactcagggctgagctccttagacctacagtttcttgttaaaatcgagtaggttgtgtttgaaaaataaagtttatttgtaaaattatttcagtttatattttatgtataatcatctctcttacaaatatatgactcaaggaaagatagtattttgccctagggtcaagagtgaaattatttaaacttattagtgcatatttatttcttttctattttatgctaaaaaaatgtgtggaactaaaatttgaaaagatacttattttgttagtttagaaaatgtttagtatttcatataatcttaactttcaagtattttggcgtctatcttaatattttctcctttaagaaaagacaaaaattaaaaatcttattctaacttagtttagtccactcagtgaaacagcatttaatatttaatgaaaattgttttgtcgtttcctttgacatctgtattaaagattgctaattgcctccaaaatccattttgtccctttcctatcattatatatttccccattgttatccatccatagtaaagatagcatttcctgggttcacatgttgctgatgtggccctgtgagcaagttttgatgaattggatgtgagtagaagtgagctgtgcagcttctagatcatgcccttaaagggaagccacatgcccgctcattcccctttttcctgctccctgctggctagaatgtgaacagggttatgagtcatctcaaccatgtgaaggagggtgatggaacagggatgggagaaggagcctaagtccttgacttctccatagactagagctgctttgcttaggcgagaaggaaataaacatcagcatcagatagtattattctgtcacacttagccaaaactgtgtataaaatacattaccatgctgacagaattcaccatttctgctgtgtttttttgaggtttaagtttcaaatttgcataatatgtgaaggcattttcatgtgaaataaaacattattaactatatcttgttttcagagcaaccactggattcatgctaatactgcttttatattacagttattcagtattaaaccattttgtgactatctgtaactgttagatgtatgacccatttgaggcgtaaacagagtgctgtctcttgataatctaaatttcctgtcttaaatttttgtgtctgctttgcctccctttctttgtcttcctccctcatgtctttccctcccctttctgcctttggctaatgtctgggccagtgctggtaaagtgctccctggccagtccatgggagtttaaggaccttaacagagaccaggttcctggcgttacttctgaacacacacacacacacatacacactggtaaaattcagttacttgcttggggagtttatgagtttatattgtaatggatggaaaaatgccctgtgctttgcaaccttttagactaagtaataacacttacaagaaattgtgagtgttatgggaaccaacttctgtgtgggagatgggtacgagggaaaaaaaaagtttcataatgatgaatgagaaattttcagaaagcattgtttggtttgtcattttcagtccagaaggttacttacgtcatttactctggggatggaatatagcttcactgaatgacagcatttggttccaagttatttagttatgtttcattgtgttgaaataccctacctttgccagaatcgttgtaaactatgtgtgaagctctaggtggggaaaaattgaaaggagcgccatattatattacaggaacaattcatgtcctactcttggtttcctttcaaagccctgtatttgtggctgtgctaataaattccatcacattcattatgcatagctgtaagtgtatgtaaataccacgcatcttgcattttgaagaaaaccagggagtgaacacacatagtctttcttgcctgggattcatttattttttaaaattcattttatatatgaatgagaaagttgagggaacatttcctttcataccaacatagaaaatcctctaataccacatcccctgtctcctggtcctgggagcagtaatgggattttctgggtgcatttgcatcatgtgtatcattgcttttctgtttatgttcagaagccttttctgccaaggcttccacaatcttctgaccccagcagcaggtacatccttgtagatgtcagtgtaagtctttatattatgaagagtatttgagagaatggtacaagaggatgaaagggtgaatggaagttgggggcaaaataatggagggctaaagaatatagatttgatcttacaaaagctgtcgagaaaccattgtgcattcttaagcagtgaagtggcaccatttaaaatgcacatgaatgattagtctaatatagtttgtctgacggcaaagactggagagtggagtaagaaatttgttggattttataccaaaaagctaaagtgtttctaggatagcatgtcttgtttaatattttatcttttttcaaaatggagatcatgttttatactgccttgttatatggttttattacttctttatgaatgacttaatagagcattttgtgtatgtggtaaatatttgtatgttcgtttctctgatcaagaagctaatggattattttgaagtatctactattttgtagcactacatgagttgtacaattaacttgagtccccataaacaaaattggggacatgctggatccatgtcatgctgtcatgctggagccgtgtcatgctggagccatttcaaactccagtgtgcttgacgttaaatttcaaaagtcaaggaaattgttgttaaggagcttagttgaagctatcttttcaagaatttaatctactaattcattttgtttaatttttaggaatttgtatgtcattaaaatactctcaaaaatgctgttctgatttgaaatgaaatacgagtataaaaattggggtcgatctttggtacattgtttattgtactggaaaatgaatgcttaacattagtctgtgatagtgatatcatgctattttgtattcaagcctttatatgttttcatcaggaagagtcaggcaaaagcagacaaaaatttcaactaatggcaaagcactttgtacctttaaaacaatttaatgaggagttgtttttactctaaaagctgaggcaagagatacatggcagtaaaatctgctattgttaatctactgtagtctcaaatttttattcattaatttatttattcatcagttccttcagtgatatttgttgagcgcctactctgtcctggcactgctgtagtcacagaggacaattatgagttactgagcctgactcctctcctatatggattagagagtgaacataaaatatcaggaaaactaaacagtttttacattttgctttgaatgtttttttactttcctctccgcttcctttccccccttctcttctttctctttccatcccaggtcctcctttttctcactatctgagagtcggaggaaacaaggagatgggaatttggagtggaagataggtttaaggagatagttatcccactctggttctaaattcttgcacaatatctgatatggcattagctgaagaaccatgacatgattgatttctttttttcatcccactagtatatgttacaggctctcagtgtgccaaccttgtttgagacacaaaggatttagtagtgaataacctagatgcagtcctcatggagcagacgctgtgatggagaaggtagaccacaaggaaatcagcatatacaaacatgctataatttcagttagtgataaatcctataaagaggaataatctaggataggaggagagaaaaggagagatgcctttatagtaacgctctaggtactcttctgattacactctaggattgtgtatatggctctctcatgtcctaacacttttgcaaagaatggggatatgtttcttttagtaggtctaagtctaaacaagagaaggtggaatgatgaggtctccttctcagtggctggagtgaagtggaagggtataggaggctcccctttcgtgaatgcccttgctgtctcatcagggacaccagatgaggctgagctgttagcttccctctccaaattgtcatttctgccaccttgaatcccacagttggcataaaaatcgtattttaagatgttattactataatttagttgtgaataggaatgggttatgtgataggcatcagggaagggcacaaagaagaaaggggcccagagtggggagattgcaattcgctaaagtcaggctgactctactccacagccctcctgacctaaggctgtgtttatgatattcagtcctcttacgtgacagaaatatgtggtacttggtttaggttccaggtgcatatttgttaactaaagttgccttgcttgggtggcaaaaaactagcctgtagtccattttacaagcataagcggtgaaggtgtttaagatttgactttgcatccatggtaacagtctccaccaatagtacatgttacagagccctttaggcctacaattgtactgtaaagacagactgtagtgacttgtctttcttatcaggaccccaccctacatggatccttgaagagtgagcaggactgcagaaggagctcaggtggatttgacagcttataaacctggagtttggggaatgctgaggcttcaggaagaaaacaccagtaagcttaagtttgggattcccactactcaggaggactgatgatggggaccaaagttaggaaagcagaggagttttggagaacctatgagttcaaaagggtgaagtaggaaaggtgagaatgtgagggagaaatgtcagagtttatttggattcttttaaagaataaaatagaacacagtggggagattcagagtcagcttggtagggttgagtttgagggcctaagaaggaagagagtgatggagacattataagaatggaagctcaagagaaccatgtaggtgctttgtaccctggtatggagaatttctgggtgcagagttcatggaccctggcgagtaagagctcaacacaagagattagttgctacttgaaataaacatgataattgtggcccagacagaaaagcctcctctttttcaaaggtcaagattgctgaggtacatgactcagaccttgcatagcagtaggaagagtaaccagcagcaaagcaataagttgtttatcaaagggtgctggtgactcagtcaggaggatttggaatagaatgattgatttggaagtaggttaagctaaccaaataatatgaatctcctatgtgtttcttaaactgattattttgaactcaagattctcttctagtgttgtgaaaatagtttgtgatatgaaaatgtatatgctcaattgctgtgaccactaagggagacaggtatagcaagagaaataatggttcagtctggaggggaagccatcccttctcgcccttcccctacgttgtgtggatggaaggtctccaagagaagcatctatgagattaggccagggagccttctgtttcagggccaccccattctgtgtggttttatttgtcagagacgctgatcctaacagaaacagctgtttcagtgaagttcaagcttgccatctattttcagtagaagcaggaagaatgtcagagacaatgtttgcattttagggcctttttataggagcctaagataaagctgtttaaaccttctgtcctctgtcatttaacttatcaaattcaagcagagccttttaatttcaacacacttaacttctagggatgaatagggcattgaacggattctataaacaagtacttttctgagcgataaacaggaacaaaagcagcacaaagatagcatgtcaacaaggtagggtgtattgtgagaagggcaaggcttgagagcctaatctatgctattctggggaagcaactatgtttgtttgtttgatttaaggagcagtgaagttacagagagcagaaaatgctgatgtacctgaaggccattattcacttctgtagacagtgctactgggtgagaagtgaataatattagtaatatatgttcattaattaaatgtagcttttaaaagaaaacacaagatggaaaagtgaaacaaaatggatagagttgtttgggtactaagagtagtagagtagattgaagaccatttaaatgggaggcagattttcccatgcatatctaaaaaggtcctagtgttatcccttcactccattttagatttgtttgtttagtttggccctgagtatgctagaacagataataacctcccagcaaatgtaactgagtgggaacattattttagttcactttcaaaagaagagggagatccaattcataatacccaaaatagagagtaccggttagttccagaattgaccagccatggaaagacatagaaactttgcctgttagaatgctcatcctgagaataggacctgagcttttagaggtgttagatgactagaaggccttagaaccattaaggcattttatttctttcacattcagccagctttccttgagcagcttttctatgcctcttgcatttataaatacttaaacatgttatcccattcaaaagtgtaatttgttgatacattggttcaacactgccattgtatatgtgtgtgtgtgcaccatatatgttcatattatagttgcagatgaataaaagataatgataaataataatataagttatttataatagtgtcaattggttaaataacttatacatccatatgatgaaacatttccatttttaaatataatgagccacatttatatgtgctggtatggaacaagcaaaaaaaatgttaagatgcagaaagatgtgtttagtatcataacattgaggcaagcagagatacacacagatgcagacttcacatctctgttcatttatatttgaactatgagaagaagtgagaagggatgaaatcatgaccttgaaaataagaagctgtttcttcctctgaggtaggagggaaaagaagttgggtaaagatatgaaatggtgaaacattagttgaggaagaaaggacaaatcagtgagcaaaaatcctgtttttgtttttataaacatttcaatcccgggcaatcttgagaaagccagggtcataggttggatgattgcagtactttcttgtgtgttcaaagaggaaagtccaaatgcgcatttggcactggcacttctgaaaagccctgacctttgtaatcaggttctttttctacctgccctgcaaagatttctttttgttttgttttggcagtagtgtttatgtgaacctgtacccttactgtgaaattttaacttactgtatagactggtttgtgtgatgggctcctacctattttaggactagaaaggactgattagatcatttgctctggcagttggcttttaggaaggtgaatgtgtaagcctttgaactgttcggcattgaaagatttatggatctcttaggcagttatatcaccatttgaatagtaggtgatagatagatagatagatagatagatagatatagatatagatatctgatctttaaaggaccaggattgtagaataatgttagctaggtatgtaaattatgttgtgtttggatgtattttgtttttggatgtaagggacagaacaagtacatggatcaattttcttcatcctataaatttggtaaaacagaaacaatatgatgtttatttcttattttgtatttgtataaaaaatgtaagtgttggggatcattgtctcttatggtacctcacaacccatcactttatatcctaccttcaatgatataggtaaaaggtatattttttaaaccagattttacaaatttccattaaaatgttgaatttttagtttttataaaactaaaattttgagtaaagaacaaaaaggaacagaaaatagtgaagaaacagaaatagagaacgaacttcctaggactttttaagcaaaatgaatattcaggctaatagttctatttatttcctagaagtagtacacagtctagtttttcttttatacattttcttgattttagggtataaaattaaaaaaaattctgaagaatttctttctgtgctctttgaggttttgcatttttatctggtgtttgtgtaaaattaattcaacttaattatgggaggggtttaaaacaaatgatactctttctaagctgggacaagttattttccttagtcactataggaacaaagaaggaatgagtggcagagtagaattgcttttgttgagcatgagctctgatatcttggctgtactgttcatttacacattacaaatattttcttagattctaagtaaatgttaccccctgcccccgaaccttgcttctactattttaaattctttctgctagctgcttctttctgaaccctaacacaggggtccttaacccctgggccacagaccgataccgatatcaggaactggacgtacagcatgaagcaagtggtgggcgaatgaaggcagcttcatctgtattgacagctgcccccacattgctcacatgaccacctgggctccgcctcctgtcagctcagtggtggcattagattctcataggagtgtgaaccctattgtgaactgtgcatgtgagggatctaggttgtgctgtccttaagagaatctagtgcctgatgatctgtcactgtgtcccatcacccccagatgggaccatctagtttcaggaaaacaagttcagggttcccactgattctacattatggtaagttgtataattatttcattatatattacaatgtaataataatagaaatcaagtgcacaataaatgtagtgcacttggatcatcctgaaaccatccccatgcccctggtccatggaaaaattgtcttccatgaaaccggtctctggtgccaaaatagttggggactgctgccctaacacatgtagggtaagtgggatttcacatacaggtataaagataacattttcattttaggaatatatttttccatataataataggaaaattgtgtatcaatgaagttttggaatggtttgagactctctatgtaagacagtgagtccaactgaaaggaagcatggtgtccgttgaatgaattttgcttcttatcaattcctgcaaagttctgatgatatcacatttgtagaaatgagagcaaagatacaaacaaacaaaacaaaacaacaaaatactacttgtcacttttgaggagaaattgaattgtttctatacatcttactttcccttcattatggtttttctttctttctttttttttttttgagaggtggagtcttgctctgttgcccaggctggagttcagtggcacaatctcagctcactgcaacttcctcctcctgggttcaagtgattctcctgcctcagcctcctgagtagctgagactataggcatgtgccaccacgcctggctaattttcgcatttttattggagacagggtcttacagtgttggccaggctggtctcgaactccttgcctcaggggatccaccccccttggcctcccaaagtgctgggattacaggcgtgagccactgcacccggcctcattatgtgttttgatgaatctgttaatttatgctccaacttaaatattcatatttacttttctcttcaagtgttttcaccattgttgactatatttaactgatatatgaagctagatttaagttatatctaacattttgcaaagtatgcttgaagcagaatggagttattgtataactatacataagcagaagttctatttgcagattcacagatgcaattatgaagttaagtcagggatagttgagttgaaacaaactttttcactatagttttgcttcctcatgtagccattttatttttttttacaaattaatctaaatctctagactttcatttttctatatcctatattcataaattactaaaggttttaaagtaatagaataggttaaaatagctgcatactcttcttagaattttgcataaaactaatcttcacaatctatttttctgaacatctgtatgcttttcgtatttaatttattgtttaaagaatttttgcggtttctcttgtgtttagagttacgtaaacagcatttgcattagattcagtggtaatatacagttgcattataatatacttactataaaaagtaaaaattattcatgatttttttgctggtaagtttgagtttttaacgtaaattaagttgccttaatttcataaatttcagtgcaatttcttagcatttttattgaagtgaagaagctatgtagtcattgtgccttatttttaaattgctctggtactttcagtagggaatattattaacatgaaccccatttcattactttttaaatgtccttctagttgccctaaatgtattgtaagtacctggagattttatgtctcaagccttctttttatcagtgtggatgtataatctccataaatgtcaattggtaatgatgatgagccattaaaattaatagcttatttgctaaatttgatgcttttaagtctaggattcgattttacatgattagacctataaatacattttattattacaaatttatttgtgtccattctttggaataatcattctgagaagtttctttgggataagttttcattaattttgatgaaatttgtggtgtttttgaaacagccactttaatattattgtgacatatttaaaggaaagcacatggatttctggaaaccagaattttaaacaagataatataattacatcttttgggaaaaaaattgtttattttcactagtgtgttttattccaggaagcaagtttcatcatcatgatttaaaataataaattgaaagacataaaacttaataatgcaaattagcttttgttggttgtgatgatgtactttgctttacgtgtatttatataacacctcggatacccaaaatatttattaaataccagcatctttctcacattaacctgaaaacttacagtagtcacttttctgcttgcctttattgccagactagtggcatacacaactgtcttttcttacaactaatgccttcagttatttcttagatatgagctttggtatgttacagttttcagaacttgctcaagtaaggcctcattcggcctcattgttcaactcagtggcttgggctctgttgcatctcccgatagttttttagaatattcactactctagttttcagtgacatcgtctgtcttggttcttctgaactacttaatgtgcattcctcttgtagggttctcatcctcctaaatgccggtattctccaaggctgcttcttttagtgtcctcaagcttcaagtcacccatggctactcatccttttcttctatttaccatcttcagttagtcccaagtcctagaatctcctctcccttaaaaataatctttgctgctcctccttgtcccctgttcttgcaactgtgttatttcatccttcccttactcatgataagattactgataatggctggccttctaatatcttgctccccaatgtgctaaagctagagaagtctttaacactttaatcctcttctgggcatttgcttttcttgcaaaagattaattatcttctttttaatctggtttattttacatatgccgtcttatttctcactactacggacttgtcttcttctcattttcatgccttcataattctaaaaacatgtcatgctaattctcccactcatctggaatcctacacacccagttcctgtcccagctcttccataagcctttagaccttgctgatttctccttttgctgaacctgtcttcacttttaatagcatgcatactgatgtatatatatcaaaaacccttttttttttaaacattacttagaattgtggtggagttgtcactaaagattaaaagtaggttccattaataagagaccatatactttttggtatccaccgcagcagtgctgggcccatattttggtgctcagtaaagttgatgattgatttaattgattaaataggtgatacttcaactttcaaatagagactttgtttttaaaaagtacttagctttatattagttgaacagctgtgtacatgaacatgtaaagtagaacttactttatgcactatttactcctgtagttctaaaaatactgcattttgcttctctttttttgtaaactactttctggagtgcttttattatttaaaaatgtacttcttatcatagggattagtgagaagtatttatgagagttctgttaatgtacctgtaaccttaggtatacccaggagcttaacattgcaaaaatcaagcttttgaagaaagaatagcttaagtgctaaatttgaaattgcctgcttagtagttttctaaatggtttagaaaaaattagttgacacgtttgtttcaagtgattttggagcagccaatttgttcgttttttaaaattaataagggcagtcaatttactaatttttactaatttcatggttttgcaaagagaaaaacaagatcagatgacatcatgcaattttttttttcttttttaaaaaatatactaagttctgggatacatgttcagaatgtgcaggtttgttacataggtatgcatgtgccatgatggttaactgcacccatcaacccatcatctacattagatatttctcctaatgctatccctccccttgccccctacctcccgacaggccccagtgtgtgatgttcccctccctgtgcccatatgttctcattgttcaactcccacttatgagcgagaacatgcggtatttggttttttgcttttctcttcctgtgttagtttgctgagaatgatggtttccagcttcacccatgtccctgcaaaggacatgaactcattcttttttatggctgcatagtattccatggtgtatatgtgccacattttctttatctagtctaacattgatgggcatttgggttggttccaagtctttgccattgtgaatagtgctgcaataaacatgtgtgcatgtgtctttatagtagaatgatttataatcctttgggtatatacccagtaatgggattatggggtcaaatggtatttctggttctatatccttgaggaatcgccacactgtcttccacaatggctgaactaattcacactcccaccaacagtgtgtaagcattcctatttttccacatcgtctccagcatctgttttttcctgactttttagtgatcaccattctaactggcgtgagatggtatgtcattgtggttttgatttgcatttctctaatggccagtgatacatcatgaaatttctttcagtttcattggaactgatagaaattgtcattgatattccattgtaatataaagccattaacagcctttcttcctgtgttttttgctaatcatcctttgaacagaacggatgcttttgtctttcctttcactactacattctaaaagaacaaagatatttttatcattcattcaataaagagtaggtacctacaggtgtacaaagcattttttgtagatttaaatctcaatctgcactgaaggagttcttaaattagggcaataatgtaggtttacaaaagacagtatgattttgcatgaaaatgcactgggaagtgctttcatagatgtataaacacattctgaagagagagaatttcttatgcctgacagggctaattacagataattttgtgaaggagctagcatttgaattcatcttcggaagcagttgggatattagtggctaaggatggaagtggagagactgtactgtgtgagacacaggggcaggagatacagggtgtgtggcattagagggtggggggtgggtacaaaatttggctctggagataagtgggaccatatctgggaggtgactgaatcttagactatggattttcttctcatattcattggacatgagggataattgaagagaattttcatgaccagaggtgtgtttatgaaaatttgtcagcactgttgagtataaattggagcatcatgaaattggtaatgaggacggtcctaaacaagacgttgtagtagtataagtaagaaataattgaagcctaaactgaagataatggcagtaggaatggcagtaggcaaaagaaggaatggacacaagagtgatttcaagagactcagcataacttgctgccatattcatttatatttccatgaatcctttttgtcattccttaagtacatattgtacatgtacgctattttccttcaatatattcagtggtggaagttaattgattaaacaattttagttttgtagcatcaaagtctgtttactcttgtgacaaataactgagttcaggtttgttacagaatttaaaagagttaaatataccatttgcataaacaaaacaaaagggaatcaacagtctcagtaatactgattaatgatttaggtatattgataaatcctcttatctgggttctatgttgttagttttattttccgtcacattttgtttttcttatctctgatgcacgtataaacataaagcacacaaaagctttagttgggagaatttaattctttctgaaattctattcggattttgagtctcactctttgatttctcaagttctgagtcatgagtgtttgggcataaaaacctgatgatctttttttaaattttcattagaaagaattaaacaagcaaacaatcagaattgcttaattgaactacatttgaaaccaaattatagaggtagttataattttatcaactttttctgtcctacgatctttcttattagacaagtctgataggctttgtaagcagtatatatgacaggttttcattgaaatgctgccagtgttgaagaggagaaaagctgattttccagaagtgagaaagacaaggaaacagtcacaattttttaagattagttgaggaataccacacttggcttcttgttattgaagttttttggtgtaggtctaacaataaaaatcttgtaatattggttccatatgtatagctgcttacttgtttcatgtaatactttcatcacaaaaaatatatatctaatatttctgtgataaattataaatacagtattggttttggtccttggaggaataaccttacaaacctttaaagacttttaattttaatttttattttctttccagctttattgaagtataattgacaactgaaagactagttggtaattgaaattaggactcatttttatagtcagacaatgttaatatttaggaggagaaatgatgatttattaataactttactcaaaataccattgcataaaaattatacatataataaagtgattaaggggcacccactttttatttactaataacttagaatattcagtaactattgtttgtaaaatgaattttcttttatccccatgtctgtcattctccattcttaaaatattttcctctgtgaaacaagccagcagggataagccccatgttttgtttgatctagttaatttgacttccttctgtttgcaatgtgttattataaggtgaaatattttctggagttgtttcttaatcattttattactcttccttcttgtagggaaatggcgtgccattgtttccttcttttgtttgctaagttacctgaattttatctaagccatagtttaggtgggttagctttaatagtatttgaaatactgcttttgaattaacatactgcttgtttactttatcctttaaaatgtactttaccatttaattcttttagtagcactgagacataaatagggctgaggatgattttatttacatttcctagataagaaaattgatagactatcagtttaagtaacggcctaagatgactaagttgaaatgagaaattaggactgacattaagatattgattttatttcaaacagcattcaagaagaaggttagtagttattttagataaaattaatatagaaataggaagcacaaagaataagcaccccacatttctgatagataccacatctaaatatcacctataagatcttcctgagtctagcccttcttacttttgtatcttcatctcacagggtttcccttgtttctttgctgcatccatgctgactcttttttccagtccctttgcaattgctctgcctccttttaaaacaaaacttttgcccttgctcatttctacttggagctgtcatttcttcctcttagcatcaataactccattttgctgtttagatcgtatcttcctcagaaaagatgttccagacttcactaagtcaaattcctttgcatagctcttggcacagttgtggtgttacttttgtgtgcatgttagttttattccttcctttatcccactgatctgagtttacagatcagtgggatgtgactggtctgtttttattcattcttgtgccctaggtgactaatagcatagccattgagacatagtaggtgctcagtaaatatttgttggatgagtgtttaggtttaagaaagtatctttgcaaacatttgcagcattatcttttaatatttatagctagccagtcatatatgccaatttctgcatctacctatttgtcctctttaactgtatatgccattagcagcaaagcagtaatatggatattactggcaggtttaagtcattgacaggtaagttaaaacaacaaacaaacaagaagggaagggagaccaagtatttaaaaaaacaaaacaaaaaaaaaggaaatggggccaggtgcggtggctcatgcctgtaatcctagcactttgggagggcaaggtggcagatcacctgagatcaggagttcaagaccagcctggtcaacatggtgaaacctcgtctctactaaaaatacaaaaattagccagtggcaggcgcctgtaatcccagctacttgggaggctgaggcagaagaatcacttgaacctgggaggtggaggttgctgtgagccgggttcgtgccgttgcactccagcccggagaacaagagtgaaactccgtctcaaaaaaaaaaaaggaagtaattttcgtagggttgtagaattacagaacttgatggaatactggggtttatctagggaaatctgtctgtctctcttttaaagctttttattttgaaactttacccagtttgcctcagtgttaacgtctgtagcacaatatcaatagtaggaaattgacattactgtcatccacagagctcattcatatttctctagttatgcatgcattgatttgagagtgtatgtgtgtgtgtaggccagtcatttttaagataatagaagtttataaacttgtttccgtgacttactcaaagccaccaggccatgaggtgaaataattggtttttaaactctatattgctgtctgcccaacagccatgaattctgtcataaaattctacagatatcagggattgttcattagcattagtgccagatttagactaatcctacaatattgaaagatactaactataccttttagctaactgtgtttataatgactcaaaaaatttagttctatcaacttggatctatgtcaaaaattagaattaaaatcctacttttgtttttgcccagcaggacttctctactattcagggaaaggcctaattaaggtttcctgaaaaatactctttaatcacttggattagaataaaaaagaaatgcttttaatcacatacatatctgctaatgcttttcttctagtatctaatggctttgataataaaaagtagtaataaaactggagaactaatggattttaaacatgtttctatactgtacatattataaaatatgtatacttagtgcattaagtagtatcttaaacttatacctatgaagtacttacggtctaaaattgatcaggaggcaaccttacttagtatttcctggaagtagcaggcatttactcctaacaagaagtacaaagaaattaatatagagtttgattcagcgttggagtctgatttttaaaaatcaaactaatttctgtcttgcaaataattatatgtaatatttcctactttttataattatagaaacatgacagaactataaattattttctgaatctatggaatattattgatttggtcagtaagttatctatcattggacataaacagaagagtatataggtttcctttgattttttattttgaggtttagaattaaagtcttatgaagtttagctatttcttcgctaaatgctttaagttatacccgtgcaaaaacctctgatttgtttttgtaatttcactaaaataggctagttggtgagctgtggtgtatgtcttaggaatttaaaataaaaataaatattaaactgcttaagtgaatctggttcacacacacaaaaaatccaggtattttaattttttaccaggacatttttttaaaaagattttgggccaagaaacttattttagtaacagcatgctatagtaaatgcaattggaaatgtaagtaattaattttattgaataaaataagagcaaaacatttcccaatagaaaatctctttgaattggcactatttccagcccaattgatttttaaacaggtataaattcatagcagaaccagcttaacttaatttgtagagctcctatcaagcaggtctcagggtgtacacgttctgtctttggtggaagtgttagtttatatttcatcttgaagccagtggacattttaaagtttctttttttcttctgatgtttacaaatatgtttctttagtaattttaggcttgctagtttgctatgctagcttatattttgttattagggagtgctgattccctgtttctaaatgtcccatctttttttctcactgaatttcttcacttcagaaacatgataatgggacattaaaatcacatatgtagtttaccattgctttattgttattataggtattattaaacagtgtacatgacctttcccccatctgatccctcgtctgtttgagaaagtagataaccaaccaggatttgtaaaggaaaacaataagaacagtgtttggcagcttgctggggcagcttggggtttatacatatttctttctctcagactttgagtttatgactccaccaatcataaaaaagagtaagcataaagcagatcttttagtagatattctgggattaaaaacaaatccaagatggtaatgaggttattgttttgcttgtgattcttggtagactgtatgtatgtttaaaaaataagtctcaaggtgctgtttgtagactcatgagtggctggctctgtgatgggagcatctactgagctggtagtatagcctcctgctaatcatctgccagcttctaagctgctcaaggagttaaatgactgttgatacaatttttaaggagtggaattccgttcttgtcattcagtctatgttaataatctcctatgatgaaactcaagtgttgggtatgtttgtttcctgagctcctcatagacagaatatgtgtttatatacataatactttttaaactacttctgatgttattataacaaggttgagtataataaaaccaaagttatgttttaagatttgctttatgtgtgtatttgtctgcttctatctgtttctccgttgcattggtttatccagatatctttctatacattcactgctgtgtcactgttgcccagatgggtgcgtgcaggtagctgaactcaataaatatgagctaagtgaatgaatgaatgttcctaaatggtgggttattttgtcttttgagcagctgacagtgattctgatcttacaatctaaaagctacaattaagttaaataatacatagttttaggaatttttcttaatttattttcagtgctcctatttttgaatatactcttgcatttttcctaaagtgatatttaaaaactgttacaggaaagcatttttatcagttatttctataactggtttctataaatcaaccaaataaacaatgaatacttatttttttgaaatatatgcaatgaataatacctattaatgctattctcttcttcagtgtactttaacctgaatgcatatttctgttggtggctcaaatagtgagatttaagtcatagattttgttatctacaaggaacagttattatatgtttaccatttgcaaaacaaaagaaatatgaaatatgactcctgccttcaagaaacttaagaatatagttggataaatatagtgtaatgacataagacagcttagtatacagcatgaaactggcataaagtttatattatgaaaacacagatcttgtcctgatggaatttcaggtgaatgagtacttggacaacttacctgtaaaataaagactacaaaacaaatacacagacaggtccttagagaaacacatattactaaagatgtgggacaggaatagtttaaagatcttttataagtttttgtattaagctaaagatttttaaaaataaattttcttctttaaagtttgtaggtattttaagaaaacatcattttatatgaaaaatggctttgtagaaggttggaaagttaacttaatattttcttttaaaatttatatggaagatacttttcatcttggttttataaggctcagttgaagacaagtgttagcactgaagcttaaatagttaagctggccaaacagccaaaggatgaagaatattttttaggaaaagttctgtgaacttaggttccttattggtggcttgttttctaattttataacatagagtggttacatatttgttacctacacaatgatagtgtaatgactgagcactcactgacgcatcaagggatcccaattctagtcttgattctatttccaactagttgtatcatgcaagtcctctgagttcctttacttatctaataattcatggagttacaccacagtatgtctaaggtcctttccagctctatgctgatatatacgattctacttcaaaagaagttattccgaataacctttgcacaaactcacagtatagtcttgagaaggatctcagttttgttttgcttttttttctttaaagcaaataaggacttttaaaaataaattctcattatgttttgtgccatatataattacatatataattataaaatgtatataaaaatgtatgattatatccaactatgatgaaattatgtttttccattaaattatatgtgtttctctgaacataggattttacttcacaaataagactatgtattgaactgttaaaagggctcactggacacataccagattctcaggattcttttcaagattatattgctagttagtgcaataatatcactggtaatgcagaatatctctacctacgaggtctggacatgttgcccaacctgttcacgaacgcctgggctcaggtgatcctcccatcttggcctccagaaatgctgggattacaggtgtgagtcactgtgtccggccatatcacagttttttgaggaaaagtagaatctttaagaaaataagtgactaatctttctcaaaaatttaatctggtaaaatttatttaggtgacatttgaatgtattagcagaaatttacttgaaaagttaaagtttcctttcatataatggtattctattggatgtatgtatgatgtactgttcaacgtactaattgacttaaatgtttagtttggtgttactcaaaattaacttattgctatagttagatagcggtgagtgggcctaaaagagcttgttcgtgtccctgtgcagctcaataacagtagtctagcaattcatatggttccatttttagttttgtaacaaaaatgaattgcttaaaaaatttttttaagttttggaagcaacaatgaatgttttacacaaatcagcctcaattaaatttctttaattctggtaaattaagatttagttttctaagtattccatattattgaccagtattttaacagttttcggatatttttgccatatctcctttttgtgcatttctatccatcaatttgccactgaaaattaaaagtaacctgttgcagggtagtatttcaatgaaaaaaaattaaaagtaacttggtatgacatgcctacttcccatggcatgtctgtgttctttgagttggttttcagccagctatttggcaaagcttactggggggttatgactcatcatatctttgtgacctttaaaaagaatatccacttgctgtaacagatagggatctcacacaatttctgttttttttttttttttgaggttttagtattaggtcacaaacccatataaatagttaccacttctatttttgttttgaagtggagttttatgaatatttgagaatatagacagcttcccattgaggattgtattcctcgtccagaggtttcatttcagtggtaaatacggttaagtttgttacaaggtatttacatgagtatgtacagaagtgtattcatttatgcattaattcaacacatactatatgtaatgacgtcttaaagtgagaattaaaaatctctctgaatttctgagtttttaaacccagaatcaatttcattcacattgttctttcttgagcaaaatgtcattttaaagtgagaattaaaaatcccactgaatttctgagtttttaaacccagaatcaatttcattcatcttgttctttctttagcaaacttttcttctgtagagttccccatatgtagaccatgggaggaataggcactgtgggaacatagtggtttatgtgactggcttcctatccttaagagtaatttttttctgttttttttttttttctttcccttttctccctccctctctttcattctttcattcttatttcttaagaatcatgcagtggagatacgtgaatataattcaaggtaataaaagaattaatgtcctactactgatggtggcaaagtggtatgcggcttcagagaatagagtgattaattccaatttcggggactggagatagtttcactaggagatgaatggattttgataaacacccatgtgagggaaggctcttgcagcctgaagccgtaagacatgtaaagggtttttaatgaacctggcagttgagggaataaaatatagtagtataagaatttgagtcatcggctgggtggggtggctcatgcctgtaatcccagcactttgcgaggccaaggcaggcagattgcttgaggctagcagtttgagaccagcctgggcaacatggcaaaaccccgtctctactaaacatacaaaaaattagctgagtgtgatggcacatgcctgtaatcccaactatttgggaagctgaggcacaggaatcgcttgaacctggaaggcagaggttgcagtgagctgagatcatgccactgggcatcccagcctgggtgacagggtgagactccctctcaaaaaaaaaaaaaaaaaattgggggccatatgttttgaggagagatgtaataagaaatgtggctgggattttctcgtttggcctggagagaatgaatgtcatgctcagaaatgcgaactttataaaaattagggtaccattggcatttatttcaggaggtaaatgaagtgatctgacctgtgctttagaaagatgatggtggcagagtatggaaaatgtgttaaaggtgatagattggaagcagtggaaccaataggtggccatgggctattgtttggataagtgatggtaagggcctgacctctcaaacagcaactactgtggtggtgggtatggaagttaccatgcagtgaagaagaggaactgataaatataatgttgcttactttttttagatggttggtatggagggtattagagatgggatactctcccactgtggagtaaggataaaggaaggctgttctactataggggaatatataaatgaattcaaggatccttaaagatccttgaaaataaagaggaggtaaaaactaaagagaaatagaaacaaagaatggatataacagatgttgggagctggtgaactttaaggacaacagtatccaacagaagtatcatgtaagtacaggtaattttaaattatctagtagctatgttaatattaacataaaaataagcaaatataatgaattttaacaatctattttatgtaacctaaaatatttaaaatattatgtcaggatgtattcattataacaaattcttaatgagatattttacatttttttttttttgagacagagtcttgctctgtcacccaggctggagtgcagtggagcgatcttggctcactgcaacctccacctcccaggttcaggcgattctcacaccctagcctccctagtagctaagatcacaggcgcctgccaccacgcctggccagtttttttgtatttttggtagagacggtgtttcaccatcttgcccaggttggtcttgaactcctggccttgtgatccacccgcctcagcctcccaaagtgctgggaccacaggcatgagccaccacgcccggctttacatttttttaaactatttgaaattcagtgtgtatccgcatttatagcctatttcaatttagagcaaccacatttgaagtgcagagtagcacacatcctgagtgaccaccatttgggcatttgagatttaagaagtcaactttttaaagtcctctatgatttttatgtgtatattaaaaatgttgaagctaggcagtttaagtgtgcagtcagagtgccatgtgagaactaacttcataaagttgtagcctaagagaaaagcacacagactcctgagtgaacagaagaaacttactctcgacattaccctaaaaatcactcctgtaattgagtgtcttgttattttcatgaactgtttccagttgacagtattattaagtcagcaatgtatttttgaggggaaaatgtaacttaaaaattatagtgaaaagataagtgaccccttcaagtagtataacaaatttaagttcattctattatctttaagaatgtgtacccggctttattaactttgttatattaatcaacttccaaattattttgattttctactaagaatggactgtctccttactcccacccccagttagaattttggaacaaataattaaaactattacttcatagatttcgaggatagagagtggggctcaagtgagccacaactccctgtgccttacctgactttcgacttgagaatggtggaagttataaacattgctttgagttaggcttctttttactgacttttgaatcatgaggaggtaggtggtccactccatgaaaatgaactctgaacgtctttatgctacccttgacactttgaaaggagccgtatttaataggaaatagcacttacggaggagaatctgagacatgcaagccgccatacaaagataaatcaacaggggcttgaaaaataaattgcaggcctcataaattcacagcctctcttctaaaatactaagcctaaagaaataagtagaagttccataattattctacaaattgtgtattctgaatctcttgggtaattatcacaactaaaaattacgtattacaataatcacaaccagcaaattacctgaaattgcttgctcagtgaatatctttcagtaaccaaaatttgtacataaaatgactatgcatgagtgctatttataatgagcacttgttaatattggctgtgtaaagacttctccgatttctctaatttaattttcatcaacactacgggtttaaatagtacctgcttcagtatctaagcaaaggggttttatttttagaataaagcagttaaattacactggtgtaatggaacactgtatcaaaatatatacatatatgtgtataaattctaagaaaatactatttaattcatatgttaatccctttgctaacttcattacaattttttccattaaatgaaaattaagtgtcttctaaatgttttatggagttcaaattaagtagttgaatccactgatgactataaaatgtgtgagctgcttcaatagttgtatcctgaagtacagattgaaggtgtgtctagatggagccagctgtacatcttcctcttctaagcttctctcccaaaacataggggcaagtaaatgatttttataggtagatgcttttaggatttgaatatttttgtcatgtattggtgttagctgtgggtccgttacaaatcccgtgggaccttcattggtgtgaacattagtgtatgggacttccagtctgatctttggcacccatgcaactttctcttatgtttttagttggcaactctggagaatactggttcctaagctcttttggctcacagatacctttaagagtctaatggtgacctagggattctctttaccggagagatgtacataagcatgtgagcgattttgcataaggccaaggaggaattagtgagactaagaagcaaactgggaactgttccagctggagaggacactggtgtctgcctgtttaggtaactgcaaatcttatgcatgaaagctacagtaaggaaagaggctaggggcaggggccgagtaatgaagaaaagaatctgacatccaggatgctgcaggtatctttggatgtatgagtttctccctggggtatttcagatttatttcatgttagtggcttgcttgtgagatttttcttgaaggttattacagtgattcagtgagttattgttggctaattgatgtttttatcatttctggtttaaatgtatatatactttcattcacgcatttgaacaaggctgttctcagtcccttttagaatccaggcaacttcctcatctttccccaccatcctgtggatctttcaggatttttccaaggttacagctcaaagtgtcgtgttgacaggctggagcttgtcaccatttctaaagtatgtttttgaatgaattcaatatccttgattcttatcccaaggagttttaacacactttttgtcattgttgttgttgttagcagtaggctagcttagaattgtaacttgcccatgccaaagcaacaacaacagattgcaaaggaatcttcaaaatgggcggtgggcaaggcccagcctgttttaggaagtcccctagctgtttgctgtaccacagtgaagaagaaagtgaagaagaccatttcatgtaaatgaatgcagtgatgtgggtaagcacatcaaagttcttcgtaaaatgaaaaaattgttcagacctgtcttaatggttaatgcttacttaaaagtctaaatgactaatttgactagccatttgttagaggattactcattttaacttttgagttccttatacttaaagtagctaaacacattttaaataatctgtaatacattaaaatattaaaaatatgcttttctcattaaaaatactatcttttaccacttttataagtagggtattttacacagtgattaagtatcattttagtttcctctacctaggttttataagctattaaagttatagaccatttatctgttaataatggctgctgctgctttttatgctattttcctgagtgcgttaaaggttatttttattaattagccaatatttcagcccacagtaagggtaaatcctctctaaaaaatttggttttcagcctgctcaactcacaagcgattaagaaaaaaacagttgtgagaattcattagtgaaatatttaagaaacatgttttaagtctcttcccgagatttttttttttaaagcatgctgagatagccttctctcactttctccctcttactttcacatgacatataagccaaatggatcagttctttttgacaaagtgtgcattgtactgtcataatttgttttcatgtttattatctaaattcgatttgtgtagcagtttggtaaaacccagattatatgtatgtcttttcactttgttccttctttcttcctgtggtaatttactataattgtattttcccagtaagtcagatcatattagagacattattccttaattatcagtaggtgtccttggaatattgcttagctcacaggaagtgggtgggggggtttgtgtatattttgttttcctagaaatttttatgtaattctaggttattgtcaacaacctgcttgtaaccagacaaaaccttgctatttcaataagtctaaggctagttgttctgtcaaacacgtgtcttgacagtgagtctcagtcatggtgatgcatcagaaatcatctggcgagctcttaaagcaataccagtgctttggtaccgctaaggctgtgattgaattgagccaggatgggccctggtaatggtaggctctacaagttcctcagatgattccgatatgcagctcaccttgggattcgttgtgtgttagaaggaactaggaagcatcctcctactcagtggttttcagccttggctcacattggaatcatctgggggaactgaactctctaatagccatgtcccttccccagagattttattgattctctgggatactggtctgcacatgcggatttaataaaatagccaggtgattgtaacatacaggtaggtttgaaaaccactgaccaccgcttctgtttttcagatgaggaatttgaggcccaaaccaaagagattaggtgacttgtccaagatccacagaaccacaactagaactcagatcttcagacttgttatcaaatgtttttaccaggacatcacatatttttaagaaaatgtgggctttggtatttcagatgtacttttaaaattatgtccctggctgcttagtctaaaaaacaatgtttattttatttattttaatactggaaatatgaaaggacatgactaagatccagccattaccttagttatgacataagagaccttaggagctgagaattagaaacgttcatgtatcctataacttgtccacattttctagaagcaaatcaggcaaataaataacccaactgaaattttcttaggttagattaactccatgtcactatttctatagcaggaattgtaaatgcctgtgtttataacatgggtcattctctaggagaatgtttaaaaaaacattttattgttttctgggttatagggtgaatattatagaaattttggaaatatatgagtataaataaaaaaatcacttgtatgtaatcttatcatccagagataacagcacaaacgttatgttgcaaatcaaaatggtgaaatgcctttttaatgcttgttatgcgggtgttttccagttgtgcatagaataactttatatgaaaggcactgaaacacacttcatgatgcaactgttgcctcatctatttaacaaatggtactaattgtgcctatttatgtagttaataaaaatgtaaagatagctgaaaagtagcttttccatgctctaatgttctcttacataacgtctacccaaaatatccttgtttgttttttcctgccctttgttttgctaatttgcttttttattttgataataccctcttaatgtttttacttttttcttcattttcttaactgcagggatttctttttttttttttttttgttctttgtcattatttaccatgtgtttctttctctcactgctattcattattttcccatttctatttttattattctggtcattgttaaacatttcatttttccatctagatactttgttagatcttgaaggattagcaactaattgtcatccttgagatctcatcttaacttttaatcagtattgatatttcttttaaaagaaacaagaccctttactgttctggagtccaacttaattagtcaccttttgtctttctctgtgatgctcctgccatccagaacaacagcataatgcctgaccccctctgtctcacacacatataaggcaggcagttaggagattttgacatctaggctgcagcttttcattgaaaatcttgcagaaaatcttgtgtttgcataaatgcatgtcactcaaaataagcagcaaaggggaaaaaaaagagtagctcttgggttttaaaagaggaagtttacttcccctacccccagtctcaatatttaacatctggttctgactaaagatggggaggaaaaacagacttaaatctccattcatcctgtcttaccaccaaacagtgtgatcactgatagcatttctatttctgttgtggtctgtatgatcagatgtttttgttaatgatttgtaagggaagataatgccttatatttctctgagtggttataatgtaacgtgccaacatttctaatctctagatagggctagatttaatcattatttaatgaaactttgaaggaaataagttattgctggacctcatgtgtttctgtcttatttcaaagagaaaacaaaaaacagaaacctttaattaatctactgataatatttattttattttaaaattttaatttatttcaacatttgaataagtaatacattcaaatgggcagaagtgaaacagtcttgtgagatacctgttttttaaaactccattttagaaaaggaattaactaggactttatatctcttcgttcaatataattttaaagaagaattgttgaaaagtagatatgttttgccatttgctgagaatattttgtacgttagcttgtctcttacaaaaactaaatgtgttcctacttctgataatgtatgtcaatttaaaatttggaatggccaaggtaagttgctctgcatctgcccttcatatggctcctgagcacagctcagtgactggcccttgcagtgcttctaccgttgtaatgggtaaaaccaggggaaactgccttgctcttgagcttgagcaggttaatcaaaaggatagagcttccacttctcattattctcctttctactttgatgggcagaataaggggatgctcatggaagagaaattcacacccatgaaaagcaaattgccctgatgtctgaatcacattacaaatttgaattaaattacaaacttaaatcaagtctaatgttacctacattgttgtacatgccatcagaaattcaaaagcattttattggacaactgaatatttattgaagtccagaatttattatgtaaacttggtggttagatttttctttctctttctctgtcttttaacctttattttaggttcaggagtaaatatgtaggtttgttatacaggtaaactacatgtcatgggggtttggtgtacagattatttcatcacccaggtaataagcatagttccccatgggtagttttttgatcctctctctcctcctgcccttcaccctcaagtagcccctggtgtctgttgttcccctctttgtgtccatgtgttcttggagagaactaaacatcaagtgagagctaaacaagaggatatatggtatttggttttttgctcctacattagtttgcttcagataatggcctccagctccatccatgttgctgcaaagaacatgacctcattcttttttatgacgtgtagtattccatggtgtatatgtaccacactgtctttatccagtctaccattgatggcatttaggttgattccatgtctttgctattgtgaataatgctgcagtaaacatatacgttcacgtgtctttatcgtagaatgatttatattcctttgggtatatacacaataatggaattgctgggtggaatggtaattctgttttaagtactttgagtaatcaagattgctttcacagtggctgaactaatttacactcctaccagcagtgtataagtgttcccttttctcagcagtgtataagattcccttcccagcacctgttattttttgacttttcaataatagccattctgagtgctgtgagatggttcccattgtggttttgatttgcatttctctaatgattagtgatgaacattttttcctatgcttgttggttgcatgtatgtcttctttggaaaagcgtttgttcatgttctttgcccactttttaatggggttgttttttgcttgtaaatttaagttccttatggattctggatattagacttttgccacatatgtagtttgcaaatattttctcccattctgtaggttgtctgtttactctgttgatagtttcttttgctgtgcagaagctctttaattaagtcccatttgtcaatttttatttttgttgcaattgcttttggcatcttcttcatgaaatctttgctaagtcctatgtccagaatggtatttcctaggttatcatccacacttttgtagttttgggttttacatttaagtctttaatcctcatgagttgatttttatgtatggtgtaagggaaagggtctagtttcaatcttctgcatgctagccagtttgcctagcacccatttattgaatgggaagttctttctccattgcttgtttttgtcaactttgtcaaagatcagatggttgtaggtgtgaggtactatttctgggctgtctattctgttccattggcctttgtatctatttttgtaccagtaccatgctgttttggttactatagccctgtaatatagtttgaagtcaggtattgtgatgtctctagctttgttgtttttgcttaggattgctctggctacttgggctcttttttgattgcatatgaattttaaaatagatttttttctaattctgtgaagaatgtcattgtggaaggaagataaatttttaaaaaatcacggtgttggcctggcatagtggctcactcctgtaatcccagcactttgggaggccgaggtgggtggatcacaaggtcaggagttagagacaagcctgtctctactaaaaatacaaaaattaaccaggcatgttggtgggcacctgcagacccagctactcgggaggctgaggcaggagaattgcttgaacccgggaggcagaggttgcagtgagccgagatggcgccactgcactccagcctgggcgacagagcaagactcagtctcaaaaaaacaaaaacaaaaacaaacaaatcacagtgttatggtattcaccataaaagatgaaaaacaaaaaaccataagccaactttctgatattagcctcagttcatggacttcggctatttcacattgagctttgggcgacaagttttgacagaaagtagaggttcctctcctgtggtcatgagcttgtgtttcaaggagttaatgttaagggtgcagaagtctctttcttctttatccagagaataagggggaaataaatacattttatacacacatactcaacatatatctatccacatatttatgcatatttatagtcattaatatgtaatatttccacatatctaactaagtatctgtttcttagataaaggaaattatatgatgtatacttgtgtttttggtttctttcgatcaacattatgatggtgagattcatccttattgctgcatattgttgtactttgttaattttcactgctgtttaggtttctagtttgtgactggttcataattgatctgcttattgatggacatttgttttgaataatgttgccattggctcacgcctgtaatcccagcactttcggaggccgagttgggcagatcacaaggtcagcagatctggaccatcctggccaacatggtgaaaccctgtctctactaaaatacaaaaaattagccgggcgtggtggtgcgtgtctgtagtcccagctacttgggaggctgaggcaggggaatctcttgaacccgggagacagattgcagtgagctgagttagcgccactgcactccagcccggcgacagagtgagactccatctcaaaaaaaaaaaaaaaagttgccattattcaaaagaaggttctgtgcatgtcttttggtgcatatatgcatgcatttcttctggatatgtggagtaagattgctgggttgttgggtatgtatatactctgttttggtagaatgtatgtatatatttttaaatgtagttttatgtatttatgtggtatctgttattaacattttttagtcatttaataatgagttgtggcttgttaattgtttagctcacaagaacgtcttgcctacttgtattggggataatacgataaggtacgcatagtttcagtggaacttgtagttcaaagactcaattctagcccctagttggtttgtgacattgggtctgtcacagctttaggtttgcagtttcacctttataagtaagatcgaaattaataaatgatctctgaggtgacttccatctgtacaattctgtgattatttcaaaatatcctaaaagactgttacagaagtatgctttttaaaaatatgctgctgactctggggaaacaaactgctccgtaaggttaatgtcagattgtatcttttttacagtaattaagtagtattaggttaggcagaaatctatttggatttttcagtagcaagcctgacttttagtaataggctagttcacataagaccgatattaaaataacagtttttttctttacatggtaacagttgtcaagaaatttcacagaaaatattcataattaaattaaagcactacaagtgtattacttactgttaagtaatttgatgaccgggaattttatgcccaaataaatctttgtattttgtgttacattttcagttgagttcattgagtcagatgggcatcatcagtcttatttttagtgttgcatgggtatattttaataattcattctttttctcttggccttttcccatatataaactctatttgcaagttattttcagagttggaaaataatgagctaatattttcatacttcatattcatagatgaataactagttattagcaggccatatttaatttatttttgtaagaagcttctttgtcacccttttttgcatttctttttcaaagtttgtagtgtatattgagttgttcttcacctgtgattagaggcatgtggatgtactgcaaagtggaacagctacacctcggattttcctgagagttgctcctgatgagttccttagcacccatttgtgacgtggaactggaagttataataggcattttagtgaatctttcatgaaattcagtttatttcatttaaatggatactttttttttcctgagattcttttccttgttcttcttgtgtgaaaatattaatgacaggtaaataatagattttgtgttaatggtgttatttgatgaactaaataagagtcctatgtcaatccctcccccttcctttttttttttttttttgttaaacattttagcatctcttaaagcatctggaaaccagaggtttatggaaggaacaccttcctccatcagttccttatgaaaagacttgcttcctgcatcccagcttttcagagccccttagttcattttacccttggcttattactccagggccagcttcccagagagtgctgggcatgcttgtgtgctccaccaagagttctgcctttcactttccttctctcaggtaccagaatgtagcggcatttcctgtaggattctgcagctgctcctttcaaatcactctctggccttgttctgtgatgttgcattcaacatcccaccacagactgtgtcaagatctttctcttttgattagaggttctcaggacaaaataagttaacagtgctcgactgacattttccaaggatatttatgctggtggaagcaatcaggggaggccctgattgctctttaattattaagttacaaggagatacataagaggaggaaaaatcagttccagaggataaagcatcttagtggtgcatttggagaaaatacttttttatttctttactggttggtcttacctttttaaatttgtttcctcctttcctgaccttaaggatagaactagacaccgcagatggattgtttgcatttaggaatctcagaggttaatggtgattgatggagattattgagggaggaactaatttgctcctttctcgaattaggcagaggtgagtcacacttagactgaacttgtgatgtgcctggtcctcttccatttcttgtagtgggaggaagcaaggaaagagccgccctctttctgacctttggcagtctgttgacccatcggaagaatttaatttttctcccaggatagaaatgtgggggtggactggggtgtggtgatgagggaattatcagtctgatgttgatatttacaaatgtgaaaagtacacgctgtcccaggactgggctgtcaaagactaggggatgagtacttcagggttattgagcaagagggatgacaaatgaaagtcccataagtagttgcccttgaggtctgacctgagagagaagctaactcactctaaaacaatgatgctccagtccagtagtgacgttagcagcctccccacttgggagggactgactgatagatatgtggtttgggacaacagagaggaattgctttcacaaatctttaagtaagtactgagatcttgaagatgctttagatgtagttttacctagaggcagagcaatgaaaaataaaggatcttttgagggtcctgctaaaccagtgctttcagacattaaacttgcataagaatcacctggggatcttattaaagtatatattctgagtcagcaattctggggtgggacctgagattctgcatttttaacaagctctgaggtaattgctatgctgatggtctaaggaccaaattttgagtgccattattagacacttatttaaataaaattcaaattaagattcaccactccgtgtagtttttccttctttacacttaaatatgaaaatgctgtaaacaagatattgtattattccttgtggcatatataaaatattatggaatgtacgtgataaaaatagatattaataaaatccaaattaaactcaccaactaacataatggaaataaatgctgatcagaaaccaaatgctaggccacccattgctatttacagatatccccatgtggtactgtattcatgtatgtgtgacaccagatgtctggaaaacagatttttgtgacaaattttgacacaggaaatttatcttgctccataaaacttaatggaaaaatgtaatgcttaacttcagtccattttaaaagaacggatttcagtgtagtccatagaatcaagtgggatttattcttgagagctgctaggagtctctttatcaagagaattacctaagacaacgtcagtgggtggcatggtatttgattggttattgataatatgtgttttgagtttgccatattcgctcagtattctattttagatcatgaataaaggatatatttctttactacactaaatatttatcagatgaatcatttctggaagtgagggaattaactgtggtattttgaaagagaaacacatcttttcagaagtacatcaacagtgtgatttctgaatgtgtttcaaacggttaatacaccaggggctgagactccgccaaagtagaacatctgcttctaagacataggcttctggtgcattcttctctgtgcactattgttttcttttgcttgttttctcaatttgttaaatctagattatatttcataaggatacagggaagaaacgctcctagctcatttgttatcttacaacagagaaatcttcaacagatcttttaaatttgggtaaaaatctaagaattcctaatgattaaattatgcctggtgataacgtattattgaaagtggttttctgttttgaactccccactcatgcagttctccctggtggaagcatggttgcacatgcagacctctgaattcctgagcagcatggatagaaggggtcctgtgctggtgcagtgggcagcctggatggggaggcagggtcctatctatgatgtgttcttgctcccctatcagcctctttcctgaatacacatcacttctatccctgtagtctgtcatattgccatactggtctacatctgtatgccttcatctgccaggcttgttgcctggcacgttcttctgcatttgctctgctcctcttaacagagttgaacacctctctttagttcctgtgttttggaagcaccttgaatgaccttagttgtacagttagagtgtatgtaattgtgtgtttttatatctaagtaaccactagactgtcgactcccagaaagcaaggactgaactggtctttatcttatatttccattacctaatgtagtggcagattttagcaggtcctcaaaaaaggttcattgagtgaataaactttattaaagtcgatggaactccatatatgttgattataattttcatgcatataaaatcaatctgaaaacagggaaaactgatagttgtgtaagtaagctatacttgtagacaaagaagcagcaagctgtgacatgagtaagatgtgtaagaaaatgtaaagattttctagtgctgctttgcagcctgatctttgtggattatttgtagcactttttcctctattttaatatttaattccttaagaaataattaagcaagaaatcattttaaaaaagtatacaatcttcctatcattttgatgtttaacagaaatattaaaatttgtttttaaaattattttcttatttttattacgacaaaataatcagtatcttcagagagaaataatggttgattctggtgcttaccatatagattaagtcctgcaagtcttgggtatagcagtgcatctattgaataaccatttcactaataacatttacacaggtagacaggtacagtgatctgttaagcaatatttttaaatatcttcttaaaagtattgtaatgaaaggtcaggtacagctggatgccgaaagaattacgctaaaaaaatcagtatgtatctttagtgttaataagtaggattattaatagtgcttgacttttaaacagagaccccattgaatagaactagacaagccaatatccaggatgatttcttatctagacatatgtcaaatctagttatgttaaattgtcttcagaaaacacataatttaaatggcagcgtaattaaaagacactgctgttaggtctataaacattctatgtctttgcttacattttaccacatacaatgtcaggggtttttctacctttgagaacttaaaacttgtagaaattaagtctattgtcagtagaaatcagtggattagcttaatggtattttcacagttcagttcattgtgtggttatctaagtaaatacgttttgggaattaattgcagtaaattccttctgattcaaatctgggtatgctgctttccaaataggatagtacaaattttttctaaaaacgttccctggttggtttttttatcagagaaaatacagaacttgtaaaatttataatttttaatgtaaaaactactttgtttaacaagaataaaattcttaaataagatgtttgatactttctacccagcttcatcactcatttctattctcagagagaaaagagccctatcctttcaagtgctagaattgagtatcgtgataatagtagtagattggtagctctaatagtgctaataacaattaacattattgaatgcatactctttatcagttttctaagctaattatttattgctgtatagcaaaccaccccaaaatttcgtggcttgatttaacagcaattattatttgtcatggttttgtgggtttaccgaatgttttttctgcttcatataatgttggttgggatgctagttcagctggaaggtccagtggctttactgtcatgcctagcatttggtacttggcagctgggaacatacttgggtctttctacgtgatttcttggacttcctcagaacatggcagctaaacgcaagagcatttttaaagaggcagaggcagaaactgccaaactttgtaatgcctagcctcaaaaatttatgtagcgtcatttctaccacattctattgagtaaagcaagtcacaggctacttcagtaaaaggggagggaaatagactcccctgtgttgggaagagtgccaaagattttgtggccatttttaattttttacagattgagctaagtactcaatcctttacaaaattagctaagtacttttacaatgtatgttttagggatcagaagactgagacttgaagaggttaagtcagtgtccgaggtcatctagttactaagtaccttaagctttagagcaagcttgtccaacccgtgacctggcccagtacagctttgaatgtagctcaacacaaattcgtaaactttcttaaaacattatgagattttttttgtgacttctttttttaagctcatcagctattgttagtgttagtgtattttacatgtggcccaaggaaaccaaaagattggacactcctgcttttttttttactttagagctggagcttttcaccgttgtaatatattaccttcaatggtataggcagataaatttgtccttctgctccaaagccaatatctgaggaagttttatttatgtggcgggataattgtctttgcagtcagtgagattataaagatatttggatcacattattgaggagccatcatgtggtaaaagtagcagtctctccaaatttggaaatttctgagttgttgggtgatatgaaaggtaaaatttttacatctgatgttatcctgcgtctactacaaatattagcacattgcctcaccaagagcaaataaataagtcagcttcttaaaccataagtttcccagtgggcatattccattctactacaatttatatttttcatatggtttcagtgttctagtgtttactattcattaacatgaaacacagattttaaattgcctttttaaaaattatttctgctgctttgtggttttctttgttttgagggccatgttattcatggagttttcattgttaaaaaaagaaaaaaagaaatgtcagtaatgcctgtgatggtaaagaacaaaataaaagtcaaggctttcaaatatactaattgaaagtttttatgcttatggtaatttgagttttctatgttagattcttggcatactgccttcaggatttcttcagtctaagactgaggcatattgaggaaagtctgtaagttgaaaatatcatatagcctattgtactttcatgttagctgtaagttgtgaaatggattcacagtttttgcttaaagcatatagacattttagcgccagagttatctagtctcctgctgatcagaataaaagtggtagatagccccatagaatgttatggggccatagaacgtatgtaccacaaccccctggttttatagaagaaggtttagataagagaagctttcactcttgttcatatcatggaatagtgtgaagggcagagttgtgtctagaaacctggtctgtcagcactgtgttttcttccactgtatcccagtatttccttaattagccttgttattagaaatgtaaaccaagtatccaagaaggtcctgtcaccaccactcagctgtagaataagttaaatccagcttaacttttgcatttgtgaaagtctgagacctttaaaaaatgcttagtcgtcattgtattcagaaagtttaagacttctatagtggttcagggaaattgtactgctgagaactagtaggttattacagccatgtttttctgtgattactttttatttttcattttgaaagaaaaaaactacctggcatagtctcaatgctttaaaaagacaggatttttctgtgagaccattgacatctaaattagtgttctcttgaactgttatcatgcatattaagacatatcatatatctaataaatggaagtaaaatggagttaattgaatcatctggttgggttcagctataccatttctttcatgacgttatatgacaaagaacacatgtgatgtgctaatttatctatataactattctgagatttcttgctcctaataataatttagttttagttgcattttttaggagactattataaaggaaggagataaatcatgaactagatttaaggatccagacccctccttctgtttaaagcaattttagactgtggatttaagtgtttgagctttctgttgtctggtttttattttaaagatatgaaattagaactaagaggtttttcttttggggaaggatatggaaaaaatactggtgtgccatttgtgaatgtactatgggggaaggactaacactttttagaagattggtaatcaatggtcatgcaagtggtttggcttgagtgccccagagatgccttctccgtttgaaagccgaagcagcgtggttgccagtggagcctttcagagtaccggagacattgcagtgagtctggaaatctattctgtctccagtaggcacatctcaggtttatcagagaacatcaatataatggctttttaatgacatttttgtttcttactgaaaatagggcaatttaaagaataaaagaaagaaaaaccatagtaattctactaaccagaggttactattgataatgcctttttgtgtatatcctgtatttgttttcctatttacaacacatcatctataatgtggtatttatagccttttaatttaatagaactaagattgtactgagcatagtatcgattaccctgcctttttcactctacactttcattttacccactttcccccccccgatcctctcacacagtgttatgtgtgagaatagatttccagactcactgcaatgtctcccatccttcaaagggccccactggcaaccagtctgtgttggctttcaaacggagaaggcatatctggggcacttgatatgcacttgcactaccactggttcccagttttctatgaggtatctatatcagatcatggctaaaatgagtgggaattgcttggcaaatttgtgtttgtttttatctttatatttgcacatactgaataaagataattttaatcctggggaagaggtagaaaaaatctcatgattttagttttctctgttagtattgcacgacataagcaccagagagatgacttttttcctcctttggttcatttgtattttacttgtcctattccaaaagaacagtattgaaatgagacacaggtatcaataaggaaaatttccagagccagaagaaggaaggaactgtcagggaacaattagtgtgacgggaaactatgttatgcttcctcagaaagtatgacaaatatgttacatttacttgagtttctggaaactgcacatgagggatatagcattaatttcctttgtcttgttggtattctgttttaccattcatgaagaaagaaaaaaaatgagagaaaataaaaggtctcagaactcacagagtaaagatatttttctaattgaaagtcaaacttcagagttgtaattctctgttaaagtgggttttggttaaaaattattttctgtaaactaattttataggctattcctatctgtctgttcattagcacagttttggggtcagcaaattattccttatagtttaaaaatattttttataatctttgaaattaacaaacttttatttgatagctattttaagaagttgttacaggtgtaagactttttcttctctaacaaagatgtgcgctgatagagctgaaacacaatatctatgtaaaataaccatggtgcccttcaaggaaacctttgtgccctggcagataaagtgttgtggcatgtcacatatgggaaggggtagggaaaagattccatgtgggtattttgacgtaaagctggtgcatttgagaattcaacaggctgagacatagaaacattgtatcagagtagggtgaacgaatggcttaactcacggaaagcagaccatttggatagcttggggagaaaacagaagtagtgtgagaaatggtggaaaggacactagaaatacttgaggagtttctaactatattctattgagtaaaaacaagacaaaagccatccttgatttgtcaaaagtgataaaaatggtgtaacaagtagaaataggattttcctgctgggtttggggtagaacttggaaaaaaaactgatcaattatgtctttttgagccatgtgtttaaaattaaggtggaggatcagaagcaataaatatggattaattgactccattcagattgccacttcactgttttatgatagactactgggaatagcaaataataagaaacttaatgtagatttgtttaaaaaatttgttactactgttgtttcaatgtcaagtttattttgcttgttgatgaaactggtcttttatcacataactctaggtcataaaagtatagtttgaatttccaacatttctaatgtttctttttcttttgaaaaataatcaaagaaaaagttctgctatgttctggttacttctgtatgaatctcatagcaatcatgattttccattgtgtttacagaactttcattgtgttttatcgtaaaggaatgaaagaatggattattttttcttttttgtctcaatggagtggtctacattttaaaggtttattttacaaaaggatacttgttacatattaggtttcatcaaaagaagttcaatactaaagataaagagttttccttattgaaatatatattgttaagcttttaaaccaaatgaaagtatgagtggtcatatttatactgtagtaggttttcataatagattttccggattaaaaccatttgttgaaatcatcaaagagatgttaatcctgctttagcattaatgcaaagttaaaataactgacattaaaaaaatcatttgggaagctttattgaatatattgagataaggtttattgtagtccttctcttcatgcatactgctgttacaattccaggaggtttcaaaatccatgtgaatgaccccattcaaccctcaaactgtccaattcctggcctaattcaccttcactctgctgtagctaccagttgcaggactagctgcattttggccagaactgcatcaccctggaaattttaaacacactctctctcttctgattgccctctttttctggttccatctcagctttattcccactgcattgttctttatctttggagctctctaaatcccttaaattcattgggtataggattctgtcatcttcatctttgtaatatcagtgcctagactaagcctggcgtataataggcactcagagatttgaagaataaatgactaaatgactgtatcaaatacttgcccattgtttgctgtttctgaattgtacaaggccatcatgataattgatgatcttaataatgtgagaatatgattctcttaccttagtaagagagccatcagtttattggatgatagttatatggaaaaagaagaaatgctactgtgataaatatttataattttaaacaagtggtaagtagaattaatgataaataacttacttattgaaggttttcaaaaaattagatgcatcagggtcttacctacatgtcttatgtatttttattcaaaatttttagaaatgaggaaacaggttaagtaaccttttctttatctttaaacaggctaaggatattaagccagtactaacaaacttaaggaccaaactcgtcaatatgtaagatactagaaaaattaatcagaatatattttgaatatcttgtggaagcttctcaaaggatattacattgagcatcataggctggcatgttatttcatgttttaagtaaggcatttgtaagttgtccatttaaatatatttattttagaaagcaaatatatttgtcctgtgaagagcatccttattttgagggatagcttcttgctgttaataaaacttcgtagataacatgttggaaagaataccacaggccgggctcagttggctcacgcctgtaatcccagcactttgggaggccgaggtgggcagatcacttgaggtcaggagttccaaaccagcctggccaacatggagaaaccccttctctactaaagtacaaaaattagccaggtgtggtggcatacaccctctggaggctgagacacgagaatcgcttgaacccatgaggtggaggttgctgttagcctagaatagggccactgcattccagcctgggcaatagagtgagactctgtatcaaaaaaaaaaaaaaaaaaaaaaaaagaaaagaaaagaatactacagagagatcaaagaatgcccaaagttacctctcccagtcagaattttgggtgtaacctccctagctgtgctagtggtggatgaggtgtttaacaaaggaaatgtaagatcttcctttagttgccagttcagtgtagttttttggaattagtttgctaaatttcttttaaagtgatcagtttagtatccaaaaagtaaaatggagcaagttgaaaaattgtcctgcctgtgtttcagtttaggttgttcctaggcgcactgctgacttctcttatgactttgggtagattcagtctttattggtatttgaacttttaatgtctgttgtgtactctccttagacacatttaaaactttaaagcatttgggcagggggaatgggtaaggatccagttttatttaagactgtcaggacccatctcaatagtagtatctattcccatgagagaaaaatgaaaggcaaggcttacatcagcctagtctcctcacttaggaatcacaacattcctaaggtcagaaacagtgtgaagtaagtctctgaacccattttggagggcagtacatcagaaatacagtggaaattgctctttagctggatgtgagtttcattttaagcaccactgtttatattctttatgtcttgcctttctttttatattgagtttgtttcaccttgtaaaattaagaagagcatgttctagcttttaaatgtgagaattgaattattaatagtttttgggtaaaagtatcacctcccatttcttctaccaaaagcagcacatcctcacagacttttcacctcactgaaaatggtcatttaaagtttttactaattcagcatttaaagctttttatttcctccttgatttataacacagctgtttcttgaatagaaaaaactcaaggactacatgcaatctaattagattacattctttgtgtgagtctcctgcatttagtggaggccactaatcaaatcttgatatattttctatttcttttatttatttattcatttaattcatcaagtcaagatggctataattagagcagattactcctaccacattgtattatgatgatctcttcatgtttttttgtttcatactatatgatattcattaagggtagcattgtgccatcctccatcaagcattatatgcttcctttgtgcttatgatgttgaccgtagcaaccttaaactgttaagacttgtttataaacggtgaagctccatcattccaacagagtcagcatgctaattcactctagttcaaatccacattctagttgaatccctgcaatgtaacttgacatttggagaaattgattcataccattttaaagaaatatgatataggtgacagaatccaggatgaagaggtaccttgttgcttagatgatgacgttgtggattcatttcacatctctctctctttttaatgtagctcttctccctccttggtttttattcctccctccttctttcttcttcaagcctcctctaatgttaacatctttggcagtttggccagtgttgacaactactttttgattatttggtattttaaaatatttggtattttaaaatgcttttattggccaggcgtggtggctcacaccttataatcccagtactttgggaggccgaggcgggcagatcatgaggtcgagattgagaccatcctggctaacacggtgaaaccctgtctctactaaaactacaaaaaattagctgggcatggtggcgggcgcctgtagtcccagctactcaggaggctgaggcaggagaatggcatgaacctgggaggtggagctggcagtgagccgagatcgcgccaccgcactctagcctgggtgacagagcgagactccatctcaaaaaaaaaaaaaaaaaaaacaaaaaacaaaacttttattgtaaaattatttattacaaacaaaatcccatgcaaagaaaatgaataaatgtgatggttaatggaatatatgtggaaataagtagcttctcttataagtgaaagaactgatatcgaaaattccatgtaaaattaattaaaattaaaattacatttgtaagattattgagtcttttctttctgggcagatttttatttagttagttgtttggggattgtttgggttttagtggcagaaatatattcttcataagtatcagattctttattaggcagtcatcattttaggtcttacaatgatgggaaatcccatgggcttagggaagattggctaagtatggaaaaattacgtaatagaaaaaattccatctacaaagatgtgtgattgtttcttttcttctccctatgtctagatggttagagatgactgtattcttggattgttttattttcaaaattgaaagtttttttttttttaaatgggtattaggctgggcacggtgtctcatgcctgtaaccccagcacttcaggaggttgagatggttagatcacctgaggtcaggagtttgagaccagcctgaccagtatggtgaaaccccgtctctactaaaaatccaaagttagacgggcatggtggtgcatacctgtaatcccagctacttgggaggctgagacaggaggattgcttgaacccgggaggcagaagttgcagtgagccgagatcacaccattgcactccattctgggcaacaagagcaaaactccatctcaaaaaaaaaataggtatcaaaggatgcatagtgggatggggaataaagaaagcacatactacatagcaaaacattgtaaacaaaaaagtcacagcgtcatataaaaaagaaaacgtattttctatgataacaaatttaaatgaaattagataccagaatagttttcagttctctttctcttttttttttccttttaagctccaacagaatgagtttgtcagttcttttcataccttggtgtcgattgcaacctctataaatatgttaaataatggatttctaagatgattatttacatatatatggagtgtgtgtgagtgtataagtataaagtaaatatatatattctggaatgtgtatgtatatatgcatatatatattcaactatttttaacaatatgagcttgagtttctttcaagactaaaggcaaaatgtattttttcattgccttattttgtgcaaaagccaaataaaattgtggtgatccatttgcagaagcaagaaaatagaagcatatacacattttatagtcaatcagtgagtattgatataggtcctggtgattattagagcttctagactataaaaaatgttactatgtttcttttaaagtttagtgtctgcatagccagtggatcagatacccagaatgggtgggtaatcactggcgttgtgtttcagactttagatctaacaagtaaaaattaaaaaaataaattggatatgattaaaggacaaagaatcaaggggatggagtgcagccaattcagaaaataaagaacagttggtttaccctaattatagccattttgatttttcttttgtatgtatcaattggaagtttggtaaaaattaaatctttagcccaattatagaaataaattcattgcagatttcctaaggtgactttatacttttcttaaactcaaatctttaaagtcggttgtagtttgtacgagccacaagtctttcctttgatatagtaaaacagtttactttttattttttaaaacaatatcttatatctaccatcatctaaattgaggatatgttaacataaatgacaaaatcatattgatatcaaagattcaactgattaagagaaattttacacttggattaggatgagttgtgcaaaagtggctgaatctaataaaatgatgtaacatttatgtatattataatgaattttccagattgtttatgggtacaggtcaagcattaaaataaggcctatgttatgccctcccccctttcatcaaaaagctttccgtagataagttatggaaataggtgactgtttttttttaaacttttttaaggaagatgaatttattgattttatagaatatcttattgcattcttcttattactacttttcccacttttgatcatgggtatcttttttctgtattcatcttatacaggaagttatgtatctcctttgtaaattagaaagagtgtcatccaatgattttattgtcaggtgcatattaaatgatctccttggtaaaaaacatgagttattattattatttgacagttattaaacacagatacatgagtaaatatggagcaaagaaacacaactttcaatgacaaaggtctgttgatacactgtatggttttatatgttgcaaatttgatgactttctctgtgttaatttccctctatatttttattgagtcctaacacacttgcctccactcacatcccaacacacacacatacacacacatgcacacacacacactctctctctctctcatatatacaggtgcacatacacatgcataatagctgaagcttcaaaatgaaaacatatgcagaatgtacctattgatttctggtcattctgagcaaaagggaatataaagaaataaagtttgtttgaaactggggaagttatttagtattttgtgttttaattgcttcttaactatctaacactagtagttttatgccgattaacgtaatattgaaaatttaagaaatgttagcaaggattataggtgattgatagtactgttttgtagtatgaagcatggtttaaatcttgtcagaatccccagaacttaacatggaaattagaatagagctgacacctgtatctccattctaaaccatttagtcatattactacaattttttccttctaaaacagacttcatcaagtcttcagaattctgaatccattaatccatttgactctccatttatttcttttctctttcattttttgtttttgagacattgtctcaccctgttacccatgctgaagtgcagtggcgtgttcatgtctcactgaacctctgcctcctggctcaagagatcacatcagcctccctaataactggggctataggcatgcgctcccacgccctgcttattttttgtagagatgaggtcctgccatgttgccgaggctggtctcaaactctgggctctagccatttggccacctcagcctcccaatgtggtaggattacaggggtgagtcaccacacctagcctgactctccatttcttatagcagtacctcttaatgaggaaagcacattagaattgcccacagagcttctaaaatatgcatatatctagaacccttcctcagaaaatctgatatttaggtaggagtggcaagcatgtataacaaaaactgtgcattgattcagatgcctctttatgtcatgtcccaactaaagtttcttactctgtctcatgttattttctcctgttaccctctgctctgctttcactgtgccaaaaatgatataataccaatacattttcattttatacttttgtattagtacttttgtacatgctgtttctactgcctgaaataccttttacttccctctgcatcctccctttctctcctctccaggtcttttatctgtcaaacaatgagttttcttcctagattctcctctgatggcaaatctttccttactcccccaggtagaaatagtcactctctcttttatgtttaccatttatatttgattattaattttttaagtgtccatctcttccattaatctgtgagctcagtgaaattatgtaatttatcttatttgtgtttatgttaaatgaatacatggataactaataagttgacagctttgttaggaatttaaatgtacaatgttttcataaactttttttaagggcagcattaagttgaggttttcgtgatcaaccaccaagacatacgttgatacatacataacattgtttgtcttaatatatattaccataaaaatttgcatgcatctaggtgtttatttcttatcttcatttttttcccttgtgcctacagacgaaatctcacaaaactgtcccttatcttcagtcacatgctggcagaaatcaaagcaatctttcccaatggtcaattccagggagataactttcgtatcacaaaagcagatgctgctgaattctggagaaagttttttggagacaagtaagtaaaaatcatttttatagctgtggatctatctttcaagggttactgtatcctccctccagttggatatggtatatattttacttgagtgttttgtgaatgtattttattatttttgataagcattttgattgattggtaatttttatagtcaccaaaggtatgcctcaataatgcaatagttttttagtcactaagtaaatattatacaatgttatttatttcatgagcattcattgagtttgttctgagaagcagaggtatgtaagagtctttatacatgagaagctgataacttcttataatttaatatgtacttaatatacaaaataaagggtagaagggcatagatactttaaaaaatggcataagtaaattgctgtgacaatttagaagggaatagttgctttgggatcagtgatcagggactgcttcctggaggaggtggtatgttaagcttggttaaggtctgaaatcgaggtgagagaaggccctggcaccctgatccagcctttctttgttttactgcatagagattatatatgtgtgaacaagatatgaggataatgttatcacaaagatgtagtctgttgcaaagtagttaaagatgtatgccctacagagacacagattctggtgagcttggtagataatggaattgaatatctaaggtcagcataatttatataaaagttgcaaaacttttgtcataaatgtctcagtaaaatgatcaagaatgagcatcaggaggcaagtggtttccccagtaaccttttaagaattacctttaatgagagcgcgtcctcagaatgtcctagaatgtaggcaaagccctggctgagtaagtaaggaggaagtgtgactgcttacatagattattgttttaatcttgtctcaaaggtctgtatttgtcctttaactgatgaaattagacataaaaattgttagtatacactttttttagtactttgatatgttgaactgcgatattggttttcctttgtttgtttttgtttttgtttttgttttttctttacaaagtactatagtttactatagtcaccaccaagattggaatatacctacatagagtaatgtacatttcattaagcacacaatttttttggcatccaaaatattaaagcctatttcccacattgtactaaaatgtattctgaattatttttaggcactaaaggagaaaatgccattgttgccattaacatggaaattctgtgttgtagcaatctagttatttataatgttcatatattcattcagaaatgaagaatttagaaaaaagtttatccttatctgaattagaataacacagacagctaagactatactaattaaatatttattaattgtagaaagcagaacaatcttgccaggttcagggtgtaggaactgagagggtttgtttcctgtagttgcagttcatgtattaacattgtaagcttagttcaaacagtcggtaaccttcaaaataaactttctcttaaagttttaagaaaatatataggacaaattgattatggtatttctttttgaaaactgaaaattctgatgagttataaaattgtttacttgtcataaggtcaataaaaaatggaaagattgctcacaggttttttttaagagaggagtttactttgctggtacttcaagccacagaaaattatgcatttgagacatctttatttccctttatttataaccacagacatcaaggggctgaaatttctgttcaactgctacatgagtgaaatcactactacatcaaatgagggattcttgaaatagtgattagatactttccctacctcccaagtcttttcacttttgtctgctgtttttactgaattgagaaagctgcttaatttttctttaacacttttaatttgtggatgactgccaaatttcatgctccttttggaacttatatgcattaccatagtcatacaaattaattattcctttattagataattggagaaagtatttttttattcttcctaggtttttaccttgtggttccataatattttattgaatgaatggacatatgcatgaataatgtgtatcctttcgaaaggttgaattttttttttccattatgctctaatactgtttgtattttcccaaaacttagttctcctgtgggagcatgcctgtcatttatctcactgttcattgaggccatgtaagaatgctttcatttagcccaaccagtgcctttaaacttgaaaacagcattctttcctaaaacagaaccattaatccattcatgagggcagcgccctcaagatctaaccacctcttgaaggtcctacttcttaatacagtcgcaatggcaattaaattttaacatgaattttggaaggggcattcaaactatagcactgttgtttttgaagacatggtagtgctaccctctctgctcagggcccaagcccagcagccaagaggcactgcctgccttgtggcccaaggggaacagtaacacatgtatcatggcaaatttgttgggggattctgtttatggtcaatttttatgcaaatgaatatattttaacaatgcagtcatgcttttcctttcatagtctgttgaccaggagttagaaatctgtaggaaaagaaaagtgacattattgaataaatgtgtctaagtatgttcagcaaaatgagaccaatactaaactgaatcttcacactctggaataataacttataatccagaaactttaagcatgaagctcgaaaaatatattactagcgttaaaaaaagaagcacatcaatgtgattagactctgggtgacagtcatttttcttagcgagtcaggtagcagcttgtgactcatttaagtactttttgacattgagaaaagtgattgattgaagtggtgatttataagattttagtgcactggtcacctgagcagtgcacactgtacctaatatgtagtgtagtcttttatcccttacccctctctcacccttccccaccacgaccccaaagtccattatatcattcttatgcctttgcttgctcatagcttagctcccacttataagtgagaacatacgatatttggttttctatttctgagttacttcacttagaataatggtttctagctccatccaagttgctgcaaaagacattatttcattccttcttatggctgagtaatagtccatggtgtaaatatgccatgttttctttatccgcttgttggtctatgtgcacttaggttggttccatatcattgcaattgcaaattatgctgctataaacaagcgtgtgcatgtgtctttttcatataatgacttcttttcctttgaccaggtatctggcagtggattgctggattgaatggcagttctacttttagttctttaaagagtctccatactgttttccatagtggttgttactaattcatactcccaccagcaatgtagaagtgttcccttttcacaacatccacgtcaacatctattgtttttaaagatgtttaacaacatctattgtttttaattatggtcattgttgcagaagtaaggtggtttctcactgtggttttcatttgcatttccccgatgattagtgatgttgagcattttttcatatgtttgttggctgtttgtatatgtcttttgagaaatgtctattcatttcctttgcccactttttgatgggattatttgttttcttcttgctgatttgttttaggtccttgtagattctagatactagtccgttgtcagaagcatagtttgcaagtatgttctcccactgtgtgagttgtctgtttactctcctgattatttcttttgctgtgcagaagctttttaattaggtcccatttatttatttttatttttatgcatttgcttttgaggtcttagccatgaattctctgcctaagctaatgtctagaagagtttctttgatgttatcttgtaggattttcatggtttcaggtcttagatttaagtctttgatccatcttgagttgattttttaataaggtgagagatggggatccagtttcattcttctacatgtggcctgccagtttttccagcacagtttattgaataagggtatccttttccaaatttatgtttttgtatgctttgttgaagatcagttagctgtatttggctttacttctgggttctctgttctgttccattggtctatgtgcctgtcattataccagtaccatgctgctttggtaactatagccttgtgttgtaatttgaggtcagatagtgtgatgcctccagatttgttcttttcgcttagtattgctttggtgatatggtctcttttttggttccatacgaatcttaggattgttttttctagttctgtgaagaatgttgatggtattttgatgggaattgcatggagtctggattgctttgagcagtgtggtcattttattcttcccatccatgagcaggtgatgtgtttccatttgttgtgtcatctataatttctgtgtttcagtgttttgtagttttccttctagagatctttcacctccttggttaaatatattcctaagtattttattttatttttttcagctgttgcaaaggggattgagttcttggtttgattctcagcttggttatgttggtgtatagcagtgccactgatttgtgtacattgattttgtgtccagagactttactgaattcatttataagatctgggagctttttggatgaatcttgaggcttttctaggtatatgatcatatcatcagtgaatgatgacagttcgacttcctcttttccaatttggatgcttcttctttctttttttttattggattgctctggctgtgacttccagtactgggttgaatagaagtggtgaaggtggggatctttatctggttacaattctcagggggaatgctttcaactttttgccattcagtatgatgttagctatagatttgacatatatggcttttattactttcgggtaaatctcttctatacatattttgttgagggattataaaggggtgctgtatgttatcaaatgctatttctgggtctattgagatgattatatatatttttaaaatttctgtttatgtgatgtgtcacatttattaacttacatatgttaaactgtccttgtatctctgaaacccacttgattgtgatgtattagttttttgttgtgctgttggattagttagctagtattttgttgaggatttttgcatctatgttcatcagagatattggtctgtagtttgttgttgttatgtcttttcctggttttgatattagcatggtagtggcttcatagaatgatttacggaggattccctctttctctgtcttttagaacagtttcagtaggattagtaccaatttgtctttgaatgtctggtagaattcagttgtgaatctatcttgtcctggactttctctgtttgcatttttaaaattactgattcagtctcactgcttgttattgttattgggtggcagtaaaccttacccagctctcctgcagttggtgaggctgatctcactcccacagtgctctgctaacaatgcttggtttagatccaggtagtctatagacagaactcagacctgccccaggtcataagcttctccacatagaaagctgccatggctttcaaaaagccatgcccctcttcgtcttcccaggcatggccaggcacccagctcctatgctcctgtctgcagcacacttcccacttgccgcaactcctccctccccattctgttcaagggaatttgttccactcgagattatattacaaaattcagttgggaggttctttcaccctgtgtcctttccctgagctcattggctgacttccagagggtccctgtgagatatattcaggaatagcttccctgggttcatgctggagattgggaagtcctgctgcggctcctacttttttatttccctaaatctgttccagctattggaagggtgaagggcttctccagtggcctggattttcagattccctggtggggatgtgtacttggaggcagactcttcccctctcgcactctgaggacttaggagtttttcacctgtctcacgtagtaggctatagcctcccaattctttcacagggtctgtggattctgttttcctaagttcctgtgttggttctttgggaaaaaattcacactgaatctctgcacattattctgtccttccaagtgggagaagcatgctgacactgcctccagtctgccatcttgaaaaaaaaaaaaaaacaaaaattagcattgagaagatttagacataaatttccaaacaaattataattcaattagaaacacagattctaactcttgaatcttcttacatagcctctcctctccatacataaaattctcttcttcaaatttaaattcatgctctgcattaccccaacaaccttctgattagtctccctatctccaataacaacctatcctattgttcttaggggaccagtgagctgtctaaaaagcagagctaatcattccagtctttcgcttgaagtgtttgcatgaatattaattctaggttaaagtctaaaatcacattaaacatcacaagctttagaaactttttcgctggcttctttttttctacttatcatttcggtatacttaactcatctgcaaagaaacaactcaatactgttcatatttcccaatgtgtgtctcattttttactcctcattgtctttgttttcgtatgttgcattcactgcttggataggcagtcacccatgctctatccagtgttgccactttgaaagctttactttagtaccctaatggatatccctactctgtgctgccaggtacctgagctcacatctgtcaaaatatattacagtgaactgctgacatgcccccatttctcaattgagaggtttccaatagactggaaagtttatttggaagcagagactgcatcttacttatttttgtgtttttgagcctgacacgtttcctggaatgtttaggcaataataattttttttttttaggtggagttttgctcttgttgcccaggctagagtgcaatggcgccatctcggctcactgcaacctctgcctcctgggttcaagcgattctccctcctcagcctcccaagtagctgggattacaggcatgcaccaccacgcccggcaatgttttttttgtatgtttagtagacgtggagtttctccatgttggccaggctggtctcaaactcctgacctcaggtgatccacctgtctcagcctcccaaagtgctgggattataggcatgagccatcacgcccggcggcaataattgttaacagactacatgaataattgcatagatggacgatgtctatcctctactctcaatttccaatgacttcattatatataaattgatctctttttaaatgatcagttcctacatttctattccttagatgcctctcttccctctttcttttcatctgtctcagaattttgacacctttctttaagttcagttcataagtccacttttctgagtttttctcattcatatacacccttctacgtgatctatgttaattactctatactttggttgctgttgtattttagacacatttatattgtaaaatgtgtttactatgtaggtctttcttgcttgattgcaagagtcttcaatgactttctatgcaaaagttggtgaaaacagacacatttattagagttattgtatgtcagattttgtgttcagtgctttcacatatgctagcctcattttcaatataatctttaaactactttgtaaatggttgttgaatgagtcttttacgtaccatatacattgtagacatttagtagttattagttgaatgtatgagtataggatgcaacttttctgccatttaagctctctcttcctttacttatgttctgttcttcagtcaaattggcttatttggctctttctggacacatcagcaattataggtttctatgtctttgcttatattgctccttctacttgaagatttttataatctctggcataacaattcttactaatccctcaaagataatattgctcataatttttcccattagtgttattactagatctcttcttaaacttcgctaagctctggcttatcttctgcatttactatttttatggttttattacaagtatttgtctagttgtcatacttccccctagactatatgctccatgaaatcaagcttatttattcatctttgtatttctaaagtagctcagcacaatgttttatatacaggctttcaggaaccattgtacatttaattcagatgagttgagaagtagaggaatgtgcattagcatgtgtatgtgtgagatggagcctgcttgcttgattgattgggagacagcatacttagggtttggcatgcaggctctgcagccagattatctgtccctggacaggttctgactgcaacatttactcactttgtgactttggggaatctagttggtttttctatgcatctttttcctcatttataaaatgtagatgatactaataatacctaccatcaatggttgttatgacggcttaaaatagctaaaccttacctgtaaggcacataaaactgtgtctagcatataataaatattctattttgtggaataattaaccaattgaaacctttaaaagtggtccaaattctctaaataatgtaatttacatttgactttgtatgatggaagtttcatactctgtagcaatctaaaagcaaaaaaaaaaaaagaaagaaaaagaaaaattttaaaaattattttatgtgtatcccgagatgaaggtagaattgaaaataagaaactgtcttggttaccacagggggaaaatggtttatttatagctcaatttattgcctctatgattgatttcctgcaaaagcaataagggccttctttggtttcccgtattttcctttcacttttaatatgaaagaaaggaaaatactttctttacctttccagcgagtgagatatatattcttctagcatattaactagctcagttttccctctaggtaaagaaaacacaacctaattatattaaattaaaagaagtggtacacagtagaaattaagagtttttacaatgaaaatttttagcagatactctaaagagaaagaaaaaagaactaaagtaggattcaaagaatcactagttaatatttctactagttagctacctccctttgttgcctgttaaaaaaaatttcaggtatctttatgtcaataaggaaaataaaaacttacatatctattggaggcctattttgttcaattcattgtaacaagatacaaaacaactaagatgccaattgtaattttctacctgtaagcaatattcagtttggggaagacaggatataaaaagaagataaatacttaaaatcagctctgtaccattagaaaatcacataaagaatatcacaaataataaattaatttactagtacatgcatacctcagagatattgtgggtttagtttgggaccactgcaataaagtacatcgcaatgaagtgcatattgcaataaagcaagtcacttagattatttttgtcttcctagtgaatatagaagttgtatttatactatactgtagtctgttaggtgtgcagtagcattatgtctaaaaaaatatatgtaccttaactaaaaaagaattgctaaaaaatgctaacaatcagctgagccttcagtgagttgtaatcttttagctgctggagagttgtgcctcgatgttcatggctattgactgatcatatgctgaaagttaaggtggctgtggcaatttcttaaaattaggcagcagtgaagtttactacatcaattggttcttcctttcatgtaatatttatctataacatgcaatgctgtttgataatattttactcacaatagagtttcattcaaaattggagtcagtcctctcaaaccctgccactgctttatcaaccaagtgtctgcaatattctaaatcctttattgtaatatcagcagtgttcacagcatcttcacctggagtaaattacatttaagaaagcacttttttgcttttctataagaagcagttttcgtctgttcaaattttatgagatttgcatcaatttagccacatatttaggttctacttctaattctagttctctagcaaattctacatctacagttacttcttccactgaagtcttgaaatcctcagagtcatccatgagatttggaattcactgtttccaaactcctgttaatgttgatattttgacctcctcccttgaatcataaatgttcttagtggcattagaaaggtgaatcctttccacgaggttttgaattttctttgcccagagctatcacaatatatgacagctatagccgtaggaaatgtatttcttaaataataagacttgaaagtcaaaatggctccttgattcatgggctgcagaatgagtactgtgttagaagacatgaaaacaacattaatctcctcgtacatctccatcaaatcttttgggtgatcaggggcattgccaatgagcagtactattttgaaaggaattgttttctctacacagcatatctcaacagtaggcttaaaatattcagtaatccaggttttttgttccatttgtagagcacaggaagagtagatttagcgtaattcttaaaggacctaggatttttggaatggtgaatgaacattgtgttcaacttaaagtcatcacttttattggccactaacaagagtcagcctgtcctttgaagctttttgtttttttaaataaagaacagaaatttatttctcatagttctgaaggctaggaagtccaagatcaaggtgctgacaaattgtctggtaaggacccacttcctaatttacagatggccaaacatctttttgcaatatcctcacatggtagaaagagtgagcaactatcagggggcttttgttttttctttttcaacctttattttggaggttatttagggggtacatatgcaagtaattgcatattgcatgatgctgaggtttggggtatgattgaacccatcatgtagctttgaagctaggtattgacttctccctagccctagcattttctagatagcattttctttcatcttaaggctgtttcatctacattaattatctgttgttttgtgtagccacattcatcaatgatcttagctagatcttctgtataacttgttgcttctacatcagcacttggtgcttcaccttgcacttttatgttagagagagcttcttttcttaaaccccataaacaaatttctgctagtttcaaactttacttctgaagcttcctgaattctcttacccttcacagaattgaagcgagttggggccttgttttggattcggctttggcctaacagaatattgtggctagtttgatctaaccaaaccaccaaaactttctccatatcaccagtaaggctgttttgctttcttatcattcatgtgttcactgaagtagcatttctaatttccttcatgaacttctcctttccattcacaacttgcctaactgtttggtgcaagaggcctcacttttgtcctatgtcagatttagacatgcctttctcactaaacttaatcatgtccataattagagacttgtgactattcctttcacttgagcacttgaggtcattttagggttactatttggcctaatttcagtgttgttgtgtcttagggaatagaaagtccccaggagagggagagagaaaagagaacagtgttggtggagcagtgagaacatataccattaagtttgctgtcttacatgggcatggttcatagtgcccaaaaactatttgtttcacacaaaaaatactgttttgaaactgaaaaactgggaatgatggcaacaatagatatgaattgtgatatgacagcaattggctagacctgtgtgccagctgtttttcttagataatctctcttctaattcatcacaatcctttcctggacatctttactaatttatattatatacctggtccccagatatttgattttttaatatctgaaagctatttggaaccactggactaatagtgtgtgcccctggatttatagaggtcctggtcttaatcctctaaacatttgttctcttctctgctggatgtgtctacttgggtcaataaagagagagaaggtctccttctagagcaaggatcttaaagccttgtaaaatagaggtagtatttctccctgggtcaaaggatatgcattatccattacaaaatatttgggttccttaagttctaggttctactccagaatcaaacccacagtgtatgcagatgtcacctggccctctttgcatcttttaggagaatttgtgtttagggaacaagggcaagaaaatgctggtactctagccactgctcttgctgtgagtaacaaagtcctttgactctgacccagtcctttgacagaaacccagtatcttctgccactatacatgaaactgtgataggccaacttcttagcttgcaagaagagtagaatctcattctttggttttttaggatcatgttctgctggtagagaaagtgtgacttaatagcaaagtaaataaaacttacttatattagtctgttctcatgctgctgataaagacctacctgagactaatttataaagaaaaagaagtttaatgcactcacagttccacatgactagggagacctcacaatcgtggtggaaggcgaaaggcacttcttacgtggcagcagatgagagaaaatgagagccaatgtgagatttattcactaccacaagaatagtttgtgggaaaccactcccatgattcaaatacctcctattaggtcctttctacaacatgtgggaattatgggagctaacaattcaagatgaaatttgggtggggacacagccaaacgatgtcattctgccactggcccctcccatatctcatgtcctcacattccaagacacaatcatgccttcgcaacaatcccccaaagtcttaactcattccagcattaactcaaaagtccacagtccagagtcttatctgagataacaagagtctcttgcacttatgagccagtaaaatcaaaagcaagttagttacttcctaggtacaatggggatacaggcactgggaaatacagccattctgaatgggagaaattggccaaaacaaagggtctacaggccccacacaagtccgaaatccagcgggccagtcaaaccttaaagctccaaaatgatttcctttgactccgtgtctcacgtccagggcacgctgttgtaagaggtgggctctcatgctcttgggcagctccaccccgtggcttcgcagggtacagtttccctcttggttgctttcatgggctgatgttgagtgtctggcttttccaggtgcatggtgcaaaatgttggtagatctactattctggggtctggaggacggtggccctcttcttacagctccactagtcagtgccccagtggagactctgtgtgggggcttcagccccacatttccctcccacactgctctagcagaggtagggctctgcccctgcagcagatgtctacctccacaagagccctgcccctgcagcaaacttctacctggatgtccaagcatttccatacatcttctgaaatctaggcagagattcctataccttaacttcttgacttctgtgcacctgcaggctcaaacaccatgtggaagctgccaaggcttgaggcttgcaccctctgaaataaaagcttgagttcaacattggccccttatagccatggctgggatgcagggcaccaagtcctgagactgcacaaagcagcaaggccttgggtacagccaatgaaaccattttttcttcctaggcctccaggcctgtgatgagagaagctgccgtgaagacctctgacatgccctggagacattttcctcattgtcttggtgattaacatttggctcctccttacttatacaaatttctgcagccagcttgaggttcttctcagagaatgtgtttttcttttttatcacattgtcaggctgcaaattttctgaactttaatgctccgcttcttcttgaaacataagttccaattccatatcatatctttgtgaatgaataaaacttaatgcttttaacagtacccaagtcacctcttgaacgctttgctgcttagaagtttcttctgccagatgccctaaattatctctctgaagttaaaagttacacagatctctagggtggggcaaaatgctgccagtctctttgctaaaacatagcaagagtcacctttacttcagttcccaacaagttccccatctccatctcagtccaccttaacctgcacttcattgtccatatcactatcagcattttcgtcaaaaccattcaacaagtgtctaggaagttccaaactttcccacatcttcctctcttcttctgagccctccaaactgttccaacctctccctgttatccagttccaaagttgcttccacagtttttgggtatctttacagcagcaccccactctactggcaccaatttactgtattagtacattttcatgttgctgaaaaagacatactcaaaattgggtaatttataaagaaaaagaggtttaatgggctcacagttccatgtggctggggagacctcacaatcatggcagaagataaaaggcacatcttacatggcagcagagaagagaaaatatgagagctaagcaaaaagggaaacccctataaaaccctcagatctcatgtggcttatttactaccacaagaatagaatgtcggaaactgcccccatgattcagttatctcccacctggtccctcccacaacatgtgggaactatgggagctacaattcaagatgagatttgggtggggaaacagccaaaccatatcattattaaaatcagtgtttagggggtgggtgcagtggctcatgcctgtaatcccagtattttgggaggccaaggcaggtggatcacttgagtccagaagttcgagaccagcctggcaacatggcataactctgtctctacaaaaaatacaaaaattagctaggcatggtggcgcacactttaatcccagctacttaacgaggctgaggcaccagaatcacttgagcctgggaggtcgaggttgcagtgagccaagatcatgctactgcactccagcctgggtgacagaatgagaccctgccacaaaacaaaatgaaatgaaaaacaaaaacaacaacaataaagtcagtgtgtaggtatttacatttcggtaatagactgctaatgattgatctttctattcttccttattttaaatgttggttattaataggaaatgcagtagttgtttttcttgtatagatttgtttttgttttgttaatctttaataacaaaggagttatttaaaatatcagtagttttattaaaaggactttttgatggatttaattatgcacaacaatatatgcagagttttttctttcagggttttagaaatgttatcagaagctctttattttttgcctcagtttatcttatacatctaggagacttcactcattggttgttaatttaattggctcatgttgagagacttacaaggaactttgttttattattgacttttttttttgtttgtaattctgagctctcattgctaaagggaggatcattttaaagccttacttaaacctctctttggtagcactttctagagtatgaaatttttttattttatttaatctgatttcagcatatcaaaggtatcttgcataacccatgagctgctgtatctcatgaatttatcctgtattggtgaactttcagagaatatatactgaagaacagttgtactttagaaagaaaagaacaagtattcattgtttaaaacctgaatctttctattttttacagtcagtggcttctaagtggaaaaatttttaatggaatttttacacattattatggattatacttgcagcttctcttcctgttcctttccactaatgaactttattttctaagttggaaaataaatagaattatgtacaaataattttatgactatagcaaatttattgtttattatgttccaaggttaggatgtgagaccagaagttagaaagcattaaatctatcatgtgaacaaaataattgagaagattagaaataatactaaagacactatttcctgtgttcctgcaatcttataaaaatatgattatagatttaggctgcagaatatgaatatgtgaaagatacttgtccactatacagagaagtaatgatggccccagctgcccatctggagtggctgctgcaaagatgctgcagtaggggaggcatggccagggctgtgtgctctatggagcaggtgggatctgggaacaggcaggagccccaccctcttccgagttggtggggttggagccccatgcttccaggagcagctgcagctatggacctgggcatccctatactcttgggggcccaggaagtccccctaccccagcaggcttggaagtgcctgctcctgctgtctggcctcaccctgctcacagtgcctgctctgggatggagcaaagctgtggccaagcctgggcactgttgtgacctggctgggtgtgcacacactcagggtggcgctgacatgccagccccctgccaccgcatccctttccagactttgggaccaaggagcactggagggaggccgacggggttgggctgagggtagctccgcatgggcctgcaggtgaccctccacagaaacagcctgtagatgacatgattggtggcagcaggaggcagacaggctcatgggtggaaaggggcaggtccccattgaagccctaccttcaagacagggacagcctacagcctcagggccgggctgtcagttcctggtggagttcacagtgggagtgaaaacttatggagctttttctgggcctgcccttggaccagtcagcacacacttccttccttctgagcccatgaaaaactccagacccagccagactcacacagacatcagactaccagctacaggaagaagctacccaatttgagtctctttgactccttgggatgatctgcctgcagaaaggagctacccactctgggtttcctgagtgctgttctgctgctcagtaaagctcctttctgccttcccaccctccagttgtttgtatacctcattcttcctggacacgggacaagaactcagggcctgccaaatggcaggactgaaaaaacagtaacacaaacagggctgaaatgtgcccaccctccacccccttcacgccccacattgtgggcaatgagaaggagagaagagctgtggccctttggggagcccacacctacaggctccctgagctaggctgtgaagccccatttgggagactgcagtttctggagtctccaagcttttgggtgccactggttcccctcctccagacacaggtgcccacagtggaagccacttgctgtgcatcagatccagccactggcttgcccagagccggtgcctgtgctggtgcctggagctgcccgccctgctacagcagccagtgtgcttggctgtgcgcagtggctggactccatgctcacttgttcatgcaccacttgctgctccgcatctgactcgtccttggcaggtgtgggatctgtgctggtagtgcatggtgagcgtggcccgccaggttgagtcagtggaatgagcccagcaggcctgagcaaaactcgggtaatggcgcaaccaatcacagaggtttctggctggaaaagtgacacacaaacaataagcgacagtagtgcttatctgcactagagacaaagtgttatgagctttttgacaaataaaaattatgcagcataaatacccagcatctgtgtctttatcgtgtcaggaattttcatgtataaggaagagtggttcaatctaattatgaatatccttggtaactatggttgccatcctatcaaagatgacttgctgtatggtgactatgtaaccagagtgttttgatatgctgtttctgtgggttgctggaaaacaattgcttttcccttattgatagaacagcaagagtaataatggtcccataagaatctatcttcatattcttaaaaaatggagtcatgataaaagaaggttggatttaaggaataactgtatacaaaatgtcaacagatttaggaatcacagcgctaatattttgtgtccataatgtaatcaaaacatatcaaatatgttacaaatatcctattatcttctgaacaaagcttgccctctgtttaaagatactgacattgaacatatcttagttgatcaaggtgaattttctgacaacatttacataaaattagaaatagagttgggctgctgaatatagaacatattttctttttgtttaaaatgaggatgacagtatatattcctgagctacaatggatcttaaacacctgccaccactaataccacagcaacaaaacaaaccaacaataaacaaacatgttgatttccttgtttgtattatggtagttactttgttcattcccatgtatctgatttttagtcttagatttttagtgtaatattttttaaaatttaaatatatgattgaagtttaatgtacatggggaaaagtacacaaatcatacatatagagtgagaaatttttatgaagtaaacataatccatgtttacagtatgtatccatgtttatctcctacccagataaagtagtaataccttgctgcctgcatcttggaagacgccctctctcagcgcgtcagaaagtcgcacatagtcaccacccagtagttctgatacatgccatcataaatcatacatagttgaaatcatactaattatactcttttggaaaaaactggcttcttcctctctttatgtttatggaattcaactctttacttgcatgtagcagtactttgttcattctccttccttcattgtatagctgtaccacaattaacctattcatttgattatttatttataaattacccagtctcaggtatttctttatagcactgctaaaacaacctaacacagaaactggatatataacatcaaggagaagaggtttattgggcccttggttatgcaggctgtacaggcatggtattagcatctgcttggcttccaggcaggcctcagagagcttttgctcatggcagaaggtgaaacagagcctgcatgtcacatgtggtgagagcaggagcaagagagagcaaggggaggtgccgtgcacttaaacaaccagatcttgtaagaactcattgtcgtcaggatggcaccaaggagatggtgctaaaccattcattggaaatatgctcccatgatccgatcacctcccactgggctctacctccaaaattgaggattacagttcaatatgaggtttggggggaacaaatacccaaactctatcatccagtgtttatatatgggtacataagtttctaaaattttagttttgttccatctatttgtttatgcttgtaccaatgccacattgtcttaattaatgtaaagagtacatacttgttaatgttcctagtgaagattaccttttttgttcttgactcttgcatgtcccataaagttcagaaccagtttttagtatctactacaaaaaatacctcctgggattgtttatttgagttacattaaatttttagactaatttgggaagaactgactctttataatatgaatctaccattaaagtgttacatacctttatttatgttttttatatctctgtgtatgggtgtatgttgatatttttgccttgctcatctattgttagctttatttctatttatttgggacttttagatagcattataaaaattaaatgtttaaagtttcattttctagttatatatagccaatataaaactacaactgatattttatatcaattttgtgttcagcaaccatgttcaattttcttatttcctatagtttaatctgtaaaatttttgtattttctacatataaagctatgtcataagcagacactgctttgtggttttcattgattcttctacgattatatacatttaaaagttttagtacatcttctattcttttagtacactagagcatattagacatataaaaagttaaaaaccatgaacaaatacagaaaatatatttttgaaatcacttataatcccttccacaagccagttattaaatttgtggcatctccagacattatagtttaacattggatcataaaggacaaaattttaaaaatactatttccatttagtagtatcccatgaacacttttccacgacaataaatatttttctgcacaaaattgttactagtgaacgtatttgttttcaaatattttgctcttataaacaacactgcatataaaagacttgtggatgggcctttggctgctatgtattttttggacataatttccttaggtgaaatccctagaagtagatgttcttagtcataatgtataggcttttgaaatttgtttccaaattgccttctagaagtgctgtgtaaattctcattcacaccagtagtatacagaagttcctgtttgcaaacaccctggcttctgttgatgttcaccctttgttgtatgaaatgtttttcattttttcctatcagttgaaactaaaaccctgtagccttttatgaactagtggtttcatgaaaatattttacctttttttttttaaaagaaatcattctcctcacactttctattttccattcttcttaccttttcttaaaattgcaggattttcaaattagaatatctaatacacttctccacatttacacaggagaaaacaagcctggaagattaagtgatttgtcaactactgaccagctgacacctttccagatttatttccctgcatctgattaatcccattgtctttgaaaagatactgtttttccaccgcatattctcactcataggtgggaattgaacaatgagatcacatggacacaggaaggggaatatcacactctggggactgtggtggggtcaggggaggggggagagatagcattgggagatatacctaatgctagatgacacattagtgggtgcagcgcaccagcatggcacatgtatacatatgtaactaacctgcacaatgtgcacatacatgtaccctaaaacttagagtataataaaaaaaaaaaaaaaaaaaagaaaagatactgtttttcaagactttaaagaggattactattgttttttggtagacagactagaactcattatctaataacaggattataacatagtcttatttatcatgttcttatctctcatgatattatatgaacagaactatcactttaacaaaaagtcattaatagatttctgtgtaatagataggtggtagtaagccatcaataaatggcagttcttactcttatttgaaataaaaagatcctgaaattatattattattacaggtgcatggctatccattggtcatgtattctattctttgtagattgacagttacttatacttgttcactgtattttaaaagcgattcagcctgcaacttaagtggtttataattgtaagggtcaatagaatatattgaaagatttttggattagtgaaaacttggagtttagttggcctttttattttctctctctctctctctctctctctctctctctctctgtctctctctctctctctctctctctctcttcccttttcttcttcccaggtctctgccagcaccctgaaatttctacatttgtaaaacaatttgctattgatattttctctcatttgcttcatctctaaaggaagaggtagtagaaaccaccactgtctttttgcagaagtttaaatggaggtgtgaaaattcatgctttctgggttcacttcctctcatatacctcagagaagaatggtagccacttagtcttaagaaagtttagagccttagtgcaacagtttagaataatctaatttttttgtttgtttttaaagactttgttgttgtactctatttctattcttcattcttgaaatccccttccccctacctctgttccccagccccacctcctacacaataatgggtgttttgctgaagtgatcaaatgagggttatctgttgcgcaaacactcatatatgaaaagttaacaaaatagtgccctggctccctaatccctattactaccactaattaagattccagtgaatgcctctttggatttgaggtttatcacaagtaatctgacaaagtttcatttacacaagtactgtggaaatgtagcttgtccttttgtaaattgttattatgaaggggtacattagactaaaattttcttaagttcatacatctcgtataactggctctgggaaatcactgagtgctcaaaagagttatgaattgaattcactgtaaatgctagattagaataatttttatagtaacagatgtggaagatgtctatttggcctttttaaaaaaactttatatgttatacacaaaataaatattgacaatttgaccactttacgttgtatactttatatacgaaacaaaaatgctttaacttcttcatacgaaaaataaaatttatactgttctgaagtcagtggttccctgatcatattttttttactctgagtatcatgatcaatgtcccttttgtgtgttgagtatttgaaactttaatttggaaaagaaaaatgtacttcttagacattagttttatggtaattataatgaagctggacataattgcatatttatacttcattgtaaatcaacagcttcctaatattcttaaaagtgttccttttttttttaaaaaaaacagcagcattttgttaaccttaatcagagcctgacgtgtgtttcttctaaatggcatctgatgtggtatctctcctcagttctctgtggtttcctgtttttttcatacatagagaaacactttttgaggtattaagcctcatatttttcttcttttaatggtacctgaaacctaaactctcttataaatacagcatagtgttgtcaacctgtctcaccattaatttatgccacttcccacagactgcttctctggaatccctccagcccttgctgtgtatccatcacccagctagcattgtccttacataccccactctagattctttagcattacaggttttacttctgtccctaaagtgtgatcttcagagacaaagatgttatctttatttctataattgtatttccccaacagtgccttgttttagcaatgaatagatatagtaggctctcaaagtgttgtctctagagcagcagcagcagcagcagcctcagggaacattttatggctgtcagtgttcaggccccaccccaatgctactgaatcataatatcttgggttgaggatcaggctatgttaacaagcccttcagataatttggatgcatgctgaagtttgacaaacactgctatagaaaattattgaatgaatatgaaagttgtacctttatggtgatatctcattgaagaggattgatatagcatggggccaggcacaatttagtttattctgcctgagtcttgtatttcagagatactggtaaaggaaacctaagatttaaatatttgctatcttataagaggatgttatcttataagagggacggcttttatgctattctgttgtaaaaattgtagtttaagttttaaaaactgaatgatttctttcttctgccgaattaggaataggcattattctgaagcaggaaggttaactaagaaatgcatcctgggtgtcactaattttcatggtgggaaagagggtaggtgagttggaaactggtcaggtggggataggttgagagacgtttccctagttaccattctgtgctcttctaaattttttgaatcatgtaactgcataacatgttaaaattttgtcgaatgatattcagtcagaataaagtaataaatattgaataaactcccatgatataaccattgtggttggtttaaaaattaagtttttcttttgtattaatatgaaaagttagataacgtatgtttttcatctctgcagaaaacatcagggttgtggcagaaatgtcttgatggctttctaccttagccttcttgaattggttgatacatattactcatttgtgcaatgaataaaggatttctattattatatggacccatcatcttatgactgtttttagaaaatgagaattaaatcttgattcattattaaataaccttgtatacacacatgaagaggtcatgacacataagactttgacctaataccttttttcagatatgtttctatagctatcttgatagcctaggactgtttgagagaaaattaatttaaatttttgtcatattcttagcaaatgcaaaataattgagttaaacagaacccactacatttgttctgaaatatgaacaatattctcaaatatttgtctggtagtacaaatacaattatattaattttattattgaattttgctgcttcaaagggaggtatttcttaaatgatacttgattcaattatttccctttttttccaccttgcacagaactatcgtaccatggaaagtattcagacagtgccttcatgaggtccaccagattagctctggcctggaagcaatggctctaaaatcaacaattgatttaacttgcaatgattacatttcagtttttgaatttgatatttttaccaggctgtttcaggtaagtttatacttgcttagtctatccattcccttcttctctctccctgtcttttctctctgtttctctctctctctgtctctctctcacacacacacacataaacaaacatatggaaaatagtgtcattcctattagtctgtaattttattggtatgtttggtcattgttaaaagtttagagtgagccatttggcttaagtttggagttatcagatgctgtgagcctggtggctgcttgttagctttgagaggatccatccaaaagagctttgttttaaactctctacttagtttcttactttattttgttgatcttttagcacaatgatggatagaatgcaacaactggaggtgggcatttgccacctagatctaaaatctttccctttgccttctgtgtttattaggtcaaaagggaaatagtaaacttagaaatatcagactgttctgtataggacagaactgtgtcctgtacttattttgtaaattgctttgcacattcaaccatgtgtaatgaacatttttcaaagtcaataaatattcttcatcagttcccttgtcatattcattcaaaatagtatgtcagtattaacaaagctctcttttgaaaaatttaggtcttttcccacattttctctaatgtaaacaactttaagatgaacatttttgttgtgcttattgtttttccctttaaattatcttccataattgttttcacatagcaaattagaagaaggtagcattatatggatactatcattttcttcttttgcgtgccttaatggaattatcttaaacatgaagacttgaaatttgtgttttgagattgactctgccactaaaatgtgtatcatctgtgggattcagttcaagtagtcacaaaataaaaattttctgcttaacatttttatgatttggtaacataaattatatagtaaatgaggtaaattgatcagactgggcttacttatgttctgatattattttttatcattttaatctgaagtaacattgattctctttgaaacatgctttcaacaaatatcagagtataatgttgatttcatgtttttcatgttacatgaacttctctgatttataattctgcttggcactagctagcaaggattggatgactggtttttgtaataaaaaccacaatgcacaggattagcttttaatttatgaaggaacccattattagaagagagattaactcctgtcaacagtacatataggatgtgaaaccccgcctctactaaaaatacaaaaaattagctgggcgtggtgatgagcacctgtagtcccagctacttgggaggctgaggcaggagaatggtgtgaacccgggaggcagagcttgcagtgagctgatatcgtgtcactgcactccagcctgggcaacagagcaagactctgtctcaaaaaaaaaaaaatacatataggatatatttcacttcattacaattacagaatcataattctaccttctatatagggtaagcaattacataaatcactgatgaaagtgtgcttttgattattttaaattttttaaattcgtgattattcccttggtgtatgtattacagtttgacagtttatgtttttttcaaagattttaagatatattttagaaaaaaaagttgtctgatattggtcattttataatggaacctctcctttctttaaaggtttctgataattttacagaatatcttatttatttaacatgatacaaagacattctaaatagtaagagctttgcttcttcaccttgtcaaggaagagagtaaaagtaatcttcctacattaaacctaactgctcattctaggaagtgagtatatttttttgtggtgagcctggatgactttttactgtgggtattaaatgctagaattccttgcagttatatattgaaagtatgagggcaacttaatgttggttaaacgattacctaaactgctatttagtatgtggacatattttcttataagctgttttactttaaaacagcacaagtcaaccttaatactctaaggaatatatatcttatttactttcactgggatgggaaaagaaagggctatttttaagtcaagctactgtgaaaagttttatatcataaaatgaaaatcttacctgggctagggaatgcctaatcattttaaaaacatgattacagaagcagctgaagaactcttgttagcccgtgtatttctgagattcatcattccatttatttctatgctgtaatcaaatgctttgaatataatcgtatgtgtgccttaaaatatgataaaattgattctgtttatagagaagtagatggtttaacttttgctgatcttttatttttataaaactttaaaaattaagtgattttcttaaatgtattatatttattaaatccttacatttatatattctgtatgaaaatatgttttgctaatactgtgtgactgcgggaacatacattcagctatagccatgatattagtagttaattggcagtgacttctgactaaattattgggagtggggtatttagcagaaagggaactgaaggtgagcaaggtacacttagaatttggggtctgaagtagagaaactagcagaagaaggagccagtgggggggcaggagatgaagttgagaattgcagagggaatgaaagaagtaaaaatgtttgcttcctcttggctttcattcttatatgcaggcagaacctgaacaaatacttgggagtcactggagggatcttactgtggtcaccttcctcatttcctcttcttccttctttccttctctttatccttctcttttctctcaaagtcacttagagatgctggaatcagcacatgctaatattagtcctggaaagaaattatagggattttgcttctcccttccttctccctggggctgttgttccccttttcctctatatccccactgttagaaaaaatggggtcactgagaaatggggcactacatttgggggtagcaggataaggacggagtattttagtagaatatcctattgactatggaatgtgtagcatcaatggaatagtgaggaaaatcaaagaagaggaaagtcctcagttgtagtaaagaattatagagagtgtctaggcagaccagccaaggaccctcttccaacttaagttgtaagtcagtaactggctcttcttgaaaatagtagcatttgggagaatggagggacttgtctcctttattcacttcattaatttctatagtcatttaaaagtatctagataattttatgttcttgtacacaaaaaaatttcttcaatctgaaaaacaaaataaaaaaaagttactctccaaagcactttgttttgccttttttaagccagtagttgtcgaaatactttaaaaagaaaaagcacataagtactacatgtttctgttttgaatgggctgcatacaaggaaaacaatacacagactcattctattacataaatatttttgcaattagaagctggccctctggattatgtactaagaaatggttgctgaatactgtagcagttggctcaaaactccaacttaatttttttacttgtattttgatctgtgcattggtgctctataaagtttagcattttttttcaaacactcttccactaatttgtgtggactttaatggaagtgacccaaatatttgtttggtatttctttcatccttgtccttcatagatgggtagaacaaaacaaaacaaaaactagggtctagaataacagttattgctcagttttttaaaatatatgctgacattttccaagtaatgtttaatgcagtttttagtcatgcattaggaagaaaattaaaaacatgcatttttttcaacctctcatattattattctctagagcgcctttcttgcccaattaggtattttagtgtgatggggaaataccccaaagatttttcttgttatgttgaaaaaggctaaaaaacttcattattactaaatcattctttgttatcctcatctcaagggatataatccttgcagaattctgccttgtaaatttgtaacttgcttaacaataatgaggaacaagtctcaaaacaaacaaaagcaggaactttctaccagtaatcatgtctaggatgactgctttaacagctttaacatggcaacgtaaaaatactttttttttcttttatgtagaagatttaataatctggcacatcagatagagtatgctgccagttccttggaataaatgtagcgtcagccttaaaaaaatcgcagagatgattcatccacacccttgagtaaagttgtctatcactgactgccttttttctcttgccctggtaactggagagacaactaatcagattaaagagagaatgatacagtggtacaaaccaaatacttctgtgaaaagttttgtttttatgtaactcacttttaaaccctaaacctttcctaagcttatagttctgtacagttaagccagatgtgactactgtagtgttaacactagccttttacacaagtcactatttagaggatctatctgttcaatgacaattatccctggctgattactattatctgacacttgcattggtcctggcatcccatgggtgagcaagacaggcatgtcccttgcccttatgtagcttatggttaatgtgtaacctttgccaagtcatgcaagttctatgatatggtttcttactgtgtaaaatgaagcagttgttaacagttaacagacatacagggaggtgaagaatatggcaagaggtaacaattttaaatacactggtcagagaaggcttcattgaggaagtgacatttggagacaggaatgagatggatatttgggggaagaaaattcaagtaaaggcatcggcaaatacaaagtctctgagggggaagagggcttttttgttcaagacaatagcaggctggagaagatgcagaaagtgggagagttatagcagatggagacaaaaaagggaactgctggggatcaaaccttgtaaagtccttctaagtgatatatgaggactttgacttttattcaaagttagaaggctttgaacaaaagaattatttcatttgtgttttacaatggtcacaagtgccgtagcgagaataggctgtgtagagaagagattttgtggttttcttttttgttctctgtattctcttcctgaaaaatctgttttctgcctaatcatatttctgtaaagaacaatagacttgaacgtctgctgttaagtaacaaaggttgacctaaaccacataaggtcagattaacttttaaaatatccaagataatgttaagtgtattaaatatggttcagtatgtaatcaaaatatttgatattctctagatactaatctcttttaatttttttattttttagccttggggctctattttgcggaattggaatttcttagctgtgacacatccaggttacatggcatttctcacatatgatgaagttaaagcacgactacagaaatatagcaccaaacccggaaggtaagactttttagcagtaatattatgtgatatatccaaagatgaaaaattctccatggtttgttcctaagtaagtcagcaatacccgctgatggcatgcttgggagggggctagaaaaggtaaaagaccaaaatggtgcttcttaacataatgttttaaattgatacattgtgcttactctgaaacttggtattcataaagtgaaaggggatatggtctcctttctgttttcatgatatccgtgtttatgtgcaaaattgaatgtatatatttgaatacttgaatactgtgctaaagaacaaataaatagaacagtcttatgttcattactttagggtatttttaagacattcatttaggctctctgataaatataagtacatatacatgcacatgaacacataaatatacatgtaagtttatgaacaaatacatgtatgtacataagcatatataaacctattgttataatcaaaaccaatttcgtgtccttgaaaacttttttcataatgcctcgtgttttattatagcctcagaatttatattttattttccacatgatgaaactaaatgagacttggagaggttggtcagcccctgtggctttggccaagaaattacaccttattaatggcaaatctggatccagaaaatctgtctcttccatggttcagtgcattttcccatgtatttcatctaaaaatgcattaggcccatagtaggcatttgtgctatggcaggcagtatgctgagtacggcagtagggatattagagacctgcttgtcagagtcctcacatttcagatgctattctggtattcattagtgctctgattttcatatagagaacattccattttagggatgaatttggtgctggtttttaagactgccttttttttctagtatcatttttatttggtttatggtgtatataatgtttgtactttataacatttttgttttgttttgttttgctttgtttttgagatggagtcttgctctgtcaccaggctggagtgcaatggcacaatctctgttcactgcaaccttcgactccctggttcgagcaattctcctgccttaacctcccaagtagctgggattacaggtgtgtgccaccacgcccagctattttttgtatttttagtagagatggggtttcaccatgttggtcaggatggtcttgatctcctgacctcgtgatctgcccaccttggtctcccaaagtgctgggattacaggcgtgagccaccatgcctggccaaaacagttttttttttaagtgcaagccaatagtgctgattttttcttctctgccattatgaaaatttccatatttttgtctttttaagaatttatatagtgtatttgaaaaaggagctagtgaatacaaaaacagtttggcttccactgaaatcttctatttagactaaggagagttagttctcattgtaaccatgagagaaggtcagttattttgcatattgacagtggtttagatgaatgattttctttcattcaagaatctctcatgaccagatttttccatagtttttactctcttgaatgacatcatataaagtatctttcttgctcttggcgtatgatgtgaaaaatgtgttgtggttggaattatgcagtcatacaaaatttaacatgtgagttatttgataaattttcttgtccaatcatagaaataatgagggaactgaggcctttatttttggttttgttttcttgttcagtatcatggagaacatgaattacaatattgagaacaacagccaggtctcattactatttggaaaagttctcttagtaaaaaactactgccctctgataagaatatcataagacccaccagtaaacttatcttcaaatgtgtacaatatatacattttatacagcttttttgtatatcagtcataccttaaaaaactagaataatgtcctctgagaaaaaaatccttctttccttcctaacttgccatgtaatgattttttctactaaattttaggtagctaatgatgaaataaataatatataacaaattataatgtgtaaagtatgacatgggtactaaagatgtgtttaggaaaacacccttaatgaataaaaaaaaaactgtaacaaatatttgctttggagaatatctgtgtttaacattttttttttttttatcttggaaagtatttcatgtctttgacttcatagtatagaaaacaattggatttacttcaaggacattttggctccttaggttgtgctttattattggggatccatctgacaaaatatgagttatggggtgaaggtagtaacatttggaaatctaagtgaagtattcattaggtgacaagagtggcattgaaccctaaaccacctcgtcaacatgaaagatacttcattttatatgcatatatatatttttttttaatttttttttttttttgagacagagtctcactgtgttcacccagcaggagtgcagtggtactatctgggctcactgtaagctccacctcctgggttcaaacgattctcatgcctcagcctcctgagtagctgggactacaggcatgtaccatcatgccttgccaatttttttgtatttttagtggagacgggttttcgccatgttggccatggtggcctcgaactcctggcctcaaatgatccaccttgctcagtctcccaacgtgctgggattacaggtgtgagccaccactcctggccccaatttgattaaatgaagcatgttgtttaagagagcaaggtgctaaaaataagccaaaaattcacttgaggttgggaaggggtagccaccagccaaagggtcagcagcaaattaaatggaataggaaaaaggtgcatgagaggaagaaattgttctaaaatgcccttggctcttaccagattgctggtattactatctcttatatacatgaattgcttgaaccttctgtataataatgagttttagaaacaccatcccaaaacaatgtttagagcaacagagaaggacatgaattaaaaatgtgagcaatttcacaagtatgtatttcagtatctactagattcttactatatgaataaatttggaattacatttaaaattactagcaatgcagatgtgtaattattttcttcatgttaatacactgtaaactttgctgagattcatagtacacagatttttaagattttaacttttgacttaatttgaaggaattttatcattaatttttttatgtaacattttaataatgtccttctgaattataatttatagtgactttctattatttttggcacttctcataatatcatagccgtaggtctctgtggcattattttatctgattttgattaagaatgccaatagttgacagattgatatcctattttcagtttctctagggatatatgtttttagacttttttgaaaagttctttccaaagattctatcaccaatataaagtttttaatatttaaaaatcacttctttttttaaattctagtttgtccaaagatgaaattacactctactactaaatatacaagttaattaaatccagtgactgcagccattatgtatgttaaagtatatctttcaagttacatttatagtgcttggtctatcaatgactaaagtatattttgacatacttgtgtctgccacatacatacagcagctcgaatattaaaatatagccaacagatttaagttggtttgtttttgatctgcttaatgggtaataaaatccagaaggatatcctgatagaaagattgaatgcagtacactcaaataaagagaaatgatctaaaatgaaataagtacatagatagctaaatgaatacataaatgataatgatggatttaaagccaagttgctgcctccttttgttggatattagtttcaagggacttgatttgatttatatttttgaactatcttaacttccccttgtaagatactgaatttgacatggttagatggcaggcatgaaattacctagtttgcacttttgagctttagggatgaatggcaggaatacctgaaagaaaagttactatttttttaacagttggccgggtgcggtggctcacgcctgtaatcccagcactttgggatgccgaggcgggtggatcatgaggtcaggatatcgagaccatcctggctaacgtggtctctattaaaaatacaaaaaattggccaggcgcggtggctcatgcctgtaatcccagcactttgggaggccaaggcgggcagatcaccaggtcaggagatcgagaccatcctggctaacacggtgaaccccatctctactaaaaatacaaaaaaattagctgggtgtggtggcgggcacctgtggtcccagctactcgggaggctgaagcaggagaatagcgtgaacctgggaggcggagcttgcagtgagccgtgattgcgccactgcactccagcatgggcgacagagcaagactctgtctcaaaaacaaaacaagcaacaacaacaacaaaataacaacaacaacaaaaatacaaaaaattagctgggcgtcgtggcacacgcctgtagtcccagctactcaggaggctgaggcaggagaatcgcttgaacctgggaggcggaggttgcagtgagccgagatggcgccactgcactacagcctgggtgatagagtgagactgcgtctcaaaaaaaacaaacaaactaattttaatgtctaaattacaaaggaatatatcgaattctaagcctgggtgacagagtaagactccgtctcaaaaaaaaaaaaaaaaaaaaaaccctaatttttatgtctaaattacaaaggaatatattgagttctacagtagaaactggctcagtttttgtctgaggtaggcaaattaaatttaaatttttacttatttattttatttttacattccaagagtgatttattttgtagctattccatgccatcagcatttccatacaccgcatcctgatgatgatggtaaaaaaatccctagtttacaatcttgtttattttcttatatatgtatctggaagtatttaaataaaaagaaatgatcagtgtttctcttgtaaattgataaatggagaagtacattgttgctaaaaataagttagaagacagaatatgaggagtattcaggaattctaaatgattgtttattcttttgcttggaagaaacctaacaaattgtttagtattgatttatattctgaatgtaattagatgtaattttcttatgttttgcagctatattttccggttaagttgcactcgattgggacagtgggccattggctatgtgactggggatgggaatatcttacagaccatacctcataacaagcccttatttcaagccctgattgatggcagcagggaaggattgtaagtatgagaaatggaagttggagtgcttatgattcatgaatttgaaaaataaagattgctttgtgatggcgaaaatggatagcaaggctgctttttatgttaagactgagaaataatggatgttccttagttttttgttcatctttttatataatcattttaaggtaacctaattatttgaaactatgttatagcaaatacatttcaacttcaaattacaacatgaatttttatctttcatatgataaaaacttgggacttaaaggtagttttaaaatggatgtaaaactgtttatatattaagagaaaattatttagaataattgacttctgtttcttcatataacccttagaatactagtttcactttttctttcttttttttttttgagatggagtctaactctgttgccaggctggagtgcagtggcacaatctctgctcactgcaacctccgcctcctgggttcaagggattcccctgcctcagcctcccaagtggctgggactacaggcgtgcgccaccatgccctgctagttttttgtattttagtagagacggggtttcaccgtgttagccaggatggcctcaatttcctgacctcatgatccacccgcctcggcctcccaaagtgctgggattacaggcatgagccactgtgcccagccagtttcactttttttattttgattgttagtgccagattttcttttctctttcttatctgtttctccctccaccccttttcttttctctctgtagatctttcctttttcacactaataataactccatattcaggactatatgttaatagctaagtttagtttatctcaagctattcctttttaaaatttaaaaataagttaagtttccctttgttttttagattaataatgaattctcccccactcactccacacacaaagtatggacaatctgttgggccccttcatagtaaatatgtctgattcttcttatttgttttattcacatgcctttccctttgtcctctccttgtattctcagtgcacttgatcatgctgctttttcttcccctctgccttgctggataacttagagatgctcgtgatcaaactcgctgcagtatgaattctggtagaaacctagaaggaaaaagtccaccagagttaacaaagagcaaatgatttgagttttacctttatcaaagtaattgcaaaaaagaaagaaagaaaagaaaaaaagtgatgagatttatacaaagaaaaattaggaatttctaaaaacctatgagcttttgggtgggagtggtggcttacgcctgtaatcccagcattttgggaggccaaggcaggaggatcacttgagcccaggagttcgaggtcaggctgggcaacatggcaaaaccctagctctacaaaaaatgcaaaaaaattagctgggcatggtggcgcgtgacggtagtcctcgctactctggaggctaagatgggttaatcacctgagcctgggaagttgaggctacagtgacaacatcgtgccactgcactccagcctgtgcatcagagtgagaccttatctgaaaaaataataataataccaacacacacaaaacctttcaataaattaattacatagtaattgtatggcaagttatggtaccaaatgtgtatttctctttctttttccttctaattttcataaatttgctagtgtcggttatttatacctttcttctaatgcatcaaagctttgtgtatagattttgatttaccatgattacagattttttttaagaaaatatactttaaagtttgaatttataagtagaacctatcaacaacctcctgataggttaaaaagaacctttaacctttcctatggtaccctaatcatgatttttgtaatctttaatttctgaattcttgctagaatttcttcatattatttgaaatgtgatacataaaattttatataatatttttttaaatggccaatttttagggtatataaaaaagttgcttattcttctttggattatcattcctcatttaatgtttgtcaaattttgtaacacacccataaggacattctaaatttgtatcatactcagatgtagtaaactccttttgctaaatcataaaaaaatcatctcactgatttgtcccttactatgtaagaattatctgtcctttaagttcatttttctggttttcctgggtaaattctaacacttaacatgtttgtagactcctcattcgaggataaatattttttgttattttaaagaatagatttgttatagttttaccctttaaaatatacatactacagtactatattcttattgttttatttaagaaatttagtatagaaagatgttaaacagaactaaccataatttaataaactgatgaaatacagcagtgacattaccacttttttcaattaaaaataagtatttaccaacacagtaaattttattttttaatgaatttttcaataacaagtacaacttctgatttaacacagcaagaatgcattattactacaacttttagttcagatttcagttttccctgataatagaaccttgtatctatagaccattttgtgtttgtagtgttaagtcaatgaaacatggaaacgtaacattttctaattttttaaaatgtgtatgtgcctgtgtgtctgtgtgtttgcatatgtgcacatgtgtttgtgttaggtgaggggaagatgggagaagagatatggaaactgtagaaacaggaataccacagaagtgtttgagtaacttacaagaaaactgtggcctttaattatatttttctttccatgtggtattaggcatggtgtaaaatgataccattattacctgtgcctcagatgaaggatgtaatagttgcaaataggtatatcttcaaagtgttttggaatacaacatgattgaccagtgaaataaattcttaacttatataagagacatatattaaaggtatttcattgaaaatggttgcttagtgggttaggctatattcattatgtatttcttatatgaaaattctacattttaagtgtcctaaaaacaacatagtaatttggaaacattgaagtaatatgttattgatatgttacatgaaaaggaaacatccaaatgtatgtttgaatatagaaaaatttgattaaatatccttaaatgcaattaaaacttgtatatttaaggtaataaagtttctgtttaaattcagttatctttatcctgatgggaggagttataatcctgatttaactggattatgtgaacctacacctcatgaccatataaaagttacacaggtaaggataattgctagaaaagtataattaaataagtatccataatgcccttgaaataaataagactctcttagtgtagttaaaatcatgcacaggtatttaatacaaaatatacttaaggaattctaagagtgtttaaggagaaattatattactttcttagattttttgatacaaatttatgctgttaaaattcttgctttccatatagtattggcatttatatgggggaaataaataaatccataagcaagtaggtggatgtaaggagagagccatagtttagaaagctaccaaaaaaagattagaataattatatgccttagattttagagtcaaatgggtaattgaagcctgttgaagataacaattatgtttttctaaatccattgctggcagcctcatagcttatgaataggaatgtaagttggtaaaacctttcttgagagtgatattcagtgattataaaaatctaagtgaaaatatcttttcactttaaattctatatatgtaatagatcccatatatatcacttacacatgcacacaaaaatctatacaacaggtatttactgtaatattttttgtaaaagcgaaaaattacaaaatactaagaactttagtaatagattaaataaaatagactactgttctcatactgtattgtgtgaagtcatgaacaggagggaggtagctctacatactcacatgggaacttctgcatcttgtactgtaaaataattccatttatgtaaagtacatgtaggtaacatttcatttggtcaagttgcattatgtacgttcaacttctgtaaggaaataattaattaattgctgtatatgaacatgtgagtatagtttttaaaagatcaaaaaaacaccaaactggtaagaagatttaactgaggaggtgaaaagggattgagcagtgagggaggggatgaagggagaacttgaatattttattttatattcagtctagttttaaaatattttatatttgagatatgttcacgccttgtgtaataagtaataaaaatctaagggaaaaaacaagagaagaattacagctttttttaagacagtagataataatttaccttataattctccagatttcctttcccatggtctttacattattatgtgagacatacacattgacatttatatgtacatattgtacgtgtaaatttaataaatgattgccttgtttttctttttttgagacagagtcttgctttgtcgcccaggctggagtgcagtgacgcgatctcggctcactacaacctctgccctctgggttcgagctgttctcttgcctcagcctcccgagtagctggcattacaggtgcctgccaccacttccggctaatttttgtatttttagtagagatggggtttctccatgttggtcaggatcaaactcctgacctcaggtgatccacccgcctcgccctcccaaagtgctgggattacaggcgtgagccaccatgcccggccatgattgccattttaaatttaatatatgattgccattttaaaagttatgtggtgatcctgtattttcaaatatgtgtgaaaggatacttaggaaagacagttcctctttataaagccattaaaaaaataaatctttggaagtaataacatccccttaaattactcataaatactcttactgtttaggttgcattgagatctgcatctttgattttagcttaatgtaattttttttataattgcatatttttcctttggacagtcataaccagtatcaatacttatctattatttcagggacatcagcaatttataacttactttgaattactttagttttatttcttctccatttttatgcattctctcgtctctttccttaagttccccacaaaatttttgtatattcacacatcataatagacacagtttatgtgtacatgtgtgtacctgcatgtacatgcatatgtatccatgtatgtttatgtatatgtatatttttctcaccaggattataatatattttagtagtctgtggactaaatattattatttatttatcctactccgtgccaggcacattgtagtcacttactgaactaaaagttgctttatttacccaaatattggtttcataattccttttctttgtattcctaacatcttttttcattatttaccaatttatttgtattttttcaaatttaccaatttatttgtatttttgtcacgaagatcttttcagtatttgaattttgaaaattttactgtgtcaaatgttttaagctaaaaacccaatcaatcaagaacattgcagttttaataaaatggtttttggttactgaagtgatttgttttcttgacagtgttagggaaagcgtatgcactttttagtcagaggcttaatttagagtttgaagcaaacaagttaaactgattaagccccaggcctaacttttgttaagttcaatatatcttcttgtgctttggagatgaagtgaaatagtaagtgtggaccacagtgcctggcatgcgggaagtgcttggtaaatgtttgtcacttttcttgcattcagggatttctaaagatattgaaacagtaaaatttaatgtttagccaagtatcatttttttttcaatttgacagttaaattatatgagcatgtggcatttataaaaaagataaagtcattttatattcctattttaaaaccatagattttgtcttttgagtcaccatgactattgaagcaagtcccttcctaaaggaacagtaaaggaaccttaaataatatgaatcattaagaagcatgtaagatagatgataaaagttcaaacctttttaaaatgaattttgtagaccaaattgtgattatcaattcaaaacatgtctcaaatggtaacaaaattctgacaaggtcaattgtgagatattgactgtgcgtcattttatttgggaaaatttgatactaacaatttgtgctggaaatacattaacattactttctccctcctccccaaattccaggaacaatatgaattatattgtgaaatgggctccacttttcagctctgtaagatttgtgcagagaatgacaaagatgtcaagattgagccttgtgggcatttgatgtgcacctcttgccttacggcatggcaggtataatcattagcaacatggatgtctttcttatatgtcctactaatatttctcttttcagttttcttaatggtttaggaagtaatgctggtaagtgctttgtttgaaatgattgtgtaaagattttatatgtttgagttgctatatataatttgatttttaactatcttatattttatctttaaatgtataaattttattatattagtcatcagtagttctacttctgtagtaccatggatttcagtatgttcacttataggtattaccaactagacatttttatgccatttattgataggtcagaatctgtttgtagtaggtcagtttctgaagccaagtaaaccatagtagataaatcttattttgatattttcaagcaaacttctttactcctgaggaggcatggttcctgtctcacatatgctccgtagtgctgaaattggagaatcagacaggaagtatctctcaaacacttatagttcccacagaaacaggcattaaagttccctatttactgaagcataatctcaggcaaattgccttcccatctgccgcaggcattcaataaatataaattgatttgaagttttataggaaggtctggagaataactggttttcccaaggactttccttttttttttttttttgagacagagtcttgctctgtcgcccaggctggagtgcagtggcacgatctcggcacacagcaagctccgcctcctgggttcacgccattctcctgcctcagcctcccgagtagctgggtctacaggtgcccgccaccgtgcccagctaattttttgtatttttagtagagacggggtttcaccatgttagccaggatggtctcgatctcctgaccttgtgatccacctgccacggcctcccaaagtgctgggattacaggtgtgagcccaagggctttcttatatttcttcatctgtaacaaagattggggatttttaaaatggatctttggacagatttccagttttgagtatttggaggagtcatggaatattttgatcaagccattttgaaactgtttgacatagttatattgaagttagaagtatattgatagggtttagaaagcaaataattaaattttctagtctttaaaaaagattttcggtgaagcataaagttgacattcctaccctatttctaagccctctcagtccttgattttctttgtccaaccctgcatctctgcatgagttgaggaaggatggacattgaggctcatggaagggggctgacagactcagaggggcattgatgcctgccgagctccttacttagtaataaaatataatgattttcccattttcatagtcataaaagggcctgcttcttcttttgtgaatatttttttaatggatgaggaaaagaggaagaattaggaaatgaattacagttgatttatattttcaaagtcctttctgaatgcccagagatctttgctacctcagtaacgttgttcaaaatccctatacttctcttcttcatatgcatttggcttatattttgttttacgtttttctttatatcctctcccttatttccttttatcccttcatgatgttggcgaaatttgcatattacagggagatgttgaaggagatcattctcaacatagatacttgattcctatttaaattacattaacagtaatagttctgcaaacttagcagtgccttttaaactacattcaataaatgtagatatgttggtagaagacagaattgtttgcaagtttatttcaaatgtgtcagtaggaggtattatagagatattgactcagtgatagattgtctgtaattggactacttaggattggtgtacctgataatcattcagtgtttcaggatacgtgttaaactaacaaagaaaacaaattgtatttgtcacccttctttgccttcagggtggtagtgtggtttttacaagtacaaagaaactaaaagccttgcatttctccctgaagaaaatcagaataataggctgatacaaattcctaacactctgcctccatgcagggcttgtgtatttcagccatttttcttggggctccagtcttagagcatacacatgcatacatgttgcaagacttcacatctgtaactcaatactgtgcttagtatttggaggttaagttggttaaaagtcagaagctgctatgagtttgacagaaacatttgtttagtaaaatgttttgttcaaagcatcatacaaaatagtttactggccaggcatggcggctcacacctcttaaatcccagtactttgggaggctgagacaggaagatcccctgagcccaggagttcgagaacagcctggacaacataaggagaccccgtctctataaagtaataataataataataaaatagttgtttgccttgcagcccagggtgagtagaaaagtatggctgaatataaagcaacaaatatttattttactttctatatacttcttaaaagaaagtttattcttagtaaataaatactacctttacttaagtcctaacttcacattgtcagtacattcttggaaactgcaactttgagcaaagtgacatactgtatgctgtaggaaccttaactcttgtttctatcaattagcctagggtaaaatggatttcattatacagtaagttgtttcacttaaagtctcaatttccaagaactaattaacaatattaattgaggacttactgtatttatttgttaggtttgtgaactatttgcagtaggttcattatgtgcaaaaggcataggaacatttcccagttgtctttctgtattactaatgctgtgtaatcaccacaacattttacatcaccacaacatgtctatgtgttactgtgcattctccaaattggtgtagttcctcttgcttgttgcttatcaattccttgaattatttcatgattttttatattggcctaatgaccttatatgaagatatgttcagtaatgttagtaagttgttaacatttactatttctctttttcattctcagctacttctcagttttacttttataaattgaggaagtacataattgtcaagctctgaaaattgtgtttagctaaaattttaaaagttatgtgagagtaatagtttactaagtaaagtagaaacaaaaatgacaaattcaaaactattcagaaggttgaaataataaattacatttatgttactagacagtaatgttattgtcttaatctgatcattgaattttgcacctaaattaatgaaaaatatgcaaacctttattttcagtattgtttttgaattctcctcacacaaagcagctcatcattaataagcatcctcctgatacattttgtatatgtttctgtaagctcttacctcattatgttcatattgtaaatatttccatatttatgtattttctttctagaatgtggattactttacctattttagaatctctctgctagcatgttatctgcacattggtcattacgcagtaaatacttatagattgcatgctaggtaatgtaaaagaaaaggaagtctaaaagtacattaaataaaaagacagtattgtaggttgtgaaaagctcttacaatacttttctgagaattatttatgatcttttcaaagaccttttatcatacttagtatctgagaacgaatcctagaatatctaaggcttagttccctttgttccattcactagctattgtgtgatttccttaacattttacattgtgatttttttccacccattcttgaaccatgttattttacgtttattgtatgcagtactaccagcactaacacagaagcagtaagactgcatttttatcgctgttttacaccaaaaaaaaaatatgcacagtaatccccggttcatgttttctctccccctaaagaaaattattcttaggtcaataattctaaattgatatttcacagtctctgtatctcagtgggatcttactggtcaaaggaagtttcaacttttgaacatttctacttttggataatctaaatatttttacatttagatacagtcttcacgtagtgcttcctgccaactagcgtatctgttttggcagcttttgtattattgcatgaacaggaatgtttgttgatcattaaaatatgttattgcaaaatgcctctggtatttaataaaaataaaagtcctataaagttaatctagttaaagcacagttgtaaaacattagatatgctgaaggtctcgtatctgtaatctcacctagctgtgctcacttgacttaacctaattgatgataaatatgtcagtaagaagaacaaaattatagcacacttcatagttccacaaaatattgccaaaagtggtatctaagcttgtacatatatagagaaatgttctcaaggtattaggtgtctggacttagattaaaataacttacgtctctcttgttcaattttttcctcattcaaaaatggtaccttaaagacattgtttttgttttatatgattaaatttgcaagcttaactgtgcacaggaagaatttgttattttgcttcttcctcttactggtttggtagaagcaaaccaagatggtttcaactatcattttccatttatttatttatttaggaaacattctttgagcagctgcaatatcccatgttctgtagtatgctctgaactgaaaagaactctgagacacaatccttagctccaagaagttaaagttaaaattaaacctgaaatttctattaaggggcatggtgttaataattacttaaaatgagtatcctgttatccttcaaatttaattatggctgctagacattttttcttactttctactacgttggttgggcatgctgatatgaacatgattttaaaggctgcgtgtctgccaagaagctatgataaatcagttagaatccaataggaaatattaaaccctgaccataaatcatactctgctcagcagtgtgcatttttagcaagtcattcttcaccaacttttccttccatctgatgccctactttggtttaattgaaattcattagagacttaaattttatgtaattaacagaaaacttaagaaatgcgcagtagctgtgtcagtagttataggctgcaagtggttactctttaattgctgtcataatggcccattagtcatgcctctgaagaggtgtcagatgcttttttctctttctgaaacatctgtgttatttaataaattgaataagcaagcagagtttgcacagaatactaagtaagttgaataatcacctttctagagacattctttgaagtaatactactctgtgtgcaacatttttgtgctacccttagtaataaaacttggagagttgtctttcacatccacgtgttaatgggacattaacaaaacgtttatcttctcaaccctgaacttctgcacatcttcctttgtgaggtagtatattatgcagcagtgattctgcagcattaccatgtaaggttaaaagttaaggaattgttttcattctgtgcttatttcaggaatctagaaaatggcttttgatttgtagttctccttgaagaggttcttcacatcccttgtaagttgtatttctaggtattttattctctttgtagcagttgtgaatgggagttcactcatgatttggctctctgtttgtctattattggtgcataggaatgtttgttatttttgcacatagattttatatcctgagactttgctgaagctgcttatcagctttaggagattttgggctgagacgacggggttttctaaatatacattcatgtcatctgcaaacagagaggacataaagaaagaatcaatatcatgaaaatggccaaaatgcccaaagtaatttatagattcaatctatccccatcaaactaccattgactttcttcacagacttagaaaaaactactttaaatttcatatggaaccaaaaaagagctcgtataaccaagacaatcctaagcaaaaagaaaaaagctggaggcatcacgctacctgacttcaaactatactgcaaggctacagtaaccaaaacagcatggtactggtaccaaaacagatatctagaccaatggaacagaatagatgcctcagaaataatgcgacacatctacaaccatctgatctttgataaacctgacaacaacaaacaatggggaaaggattccctatttaataaacggtgttgggaaaactggctagccatacgcagaaaactgaaactggaccccttccttatatcttatacaaaaattaactcaagacggattaaagacttaaatgtaagacataaaaccgtaaaaaccctagaagaaaacctaggcaataccattcagtacataggcatgggcaaagacttcatgactaaaacactaaaagcaatggcaacaaaagccaaaatagacaaatgagatctaattaaagagcttctgtacagcaaaagaaactgtcgtcagagtgaacagacaacctacagaatgagggaaaatttttacagtttatccatctgacaaagggctaacatccataatctacaaggaacttaaacaaatttccaagaaaaaaaaacaaagtcaaaaagtgggcgaagcatatgaacaaacacttctcaaaagaagacatttatgcagccagcaaacatgaaaaaaagctcatcatcacttgtcattagagaaatgcaaatcaaaaccacaatgagataaccatctcacgccagttagaatggcgatcattgaaaagtcaggaaacaacagatgctggagaggatgtggagaaataggagtgcttttacactgttgatgagagtgtaaattagttcaaccattgtggaagatggtgtggtgattcctcaaggatctagagtcagaaataccatttgacccagcaatcccattactgggtatatactcaaaggattataaatcattgtcctaaaaagacacattcacatgtatgtttattgtagcactgttcacaagagcaaagacttggaaccaacccaaatgtccatctatgatagactggataaagaaaatgtggcacatatacaccatggaatactatgcagccataaaaaaggatgagttcatgtcctttgcagggacatggttgaagctggaagccatcattctcagcaaactaacacaagaacagaaaagccaacactgcatgttctcactcataagtggaagttgaacattgagaacacatggacccaggaggggaacatcacacaccagggtctgtcagcggctggggggcaaggggagggatagctttaggagacatacctaatgtagatgactggttgataggtgcagctaaccaccatggcatgtgtatacctgtgtaacaaacctgcatgttctgcacatgtatcccagaacttttaaagtataataaaagaaaaaaaaaaaaaaggaaaatggcttttgagctcctgaatggaacaaaccccaaactgtgttcttcatatcttccttccaaaataaaaatgtatatctttgtcagttggatatgcagctatgttttgaattactgccaaggactttaattcaattaaatgtaaaattaattttcctgtggttattcttcctgtatattgtttgatataatttaaaagaaagatatcagcgtattcaaaatagtaatttacaaaaaaatcaataaactctgattttaatgtggaatatgattaaggagatttatagatgttccatatgtagagagtgttgttgggaaaatgtatatagttgtctgagagaggtgagaaacaagttcaaatgatcacttttaaggcaataagaaataatctttggccaggcgcggttgctcacacctgtaatcccagcactttgagaagccgaggtgggtggatcacatgaggtcagcagttcaagaccagcctagccaacatggcgaaccctgtttctactaaaaataccaaataactgggcgtggtggcgggagcctgtaatcccagcagcttgggaggctgaggcacaggtgaatggcctgaaccctagaggcagaggtttcagtgagctgagatcacggcactgcaccccagcctaggtaacagagtgagattctgtctcaaagagtaaaaaaaaaagataaaataaaataaatgttttactctatcttccttcaaaggcgaccagtcttcacatttctagcctcagtgaaattttataacttaggtactacaaatagaaaataatacttgcctttacgaacgttttaaattggcacttttgtctttaaccacccgttgtgtcttttgtgtaaatgagtgattaaatgttggagcttccctttcacgttgggagtaataaagaagctgcgagagttgtccaaacacgttatagtaacattatgaactagagcgatgcactagaaaaggaaaggaactgatggatatgagaaaataaaatctggtagaataagtgcttgattcttaagtctaatgactaaattagatggatgcccactatattgtagagtgtgtggaccgtaacagatactggcactttggaaacattttcagttgactccaacatggatcttttataggtgtttttaaagttgacaccatttttaaaaagtgtttagaatgtagactcattctaaatgaccagctcattctagaccaacttcatgcattaaaattttaaattggattcaagcaacaaatgtttaaaacaaaaagcttttactatgtaaaaacgtgtcctcagaaatggtataattattcagatacattttatgtcttcctgttaaagagctgaaacttttcaattagagcgaacagaatattcattgattgtagtagtttttttaattttgatatttgcaatttgaacaaaatagttttaaattttttacatttatgaatgatcagtagtagcaaccggctatttttcacctttattttaaccttgactatgaatctgagattgaaaaaccactacatgctgatcctatagtgtagtgttgaaatgattagactcttgaaatagatccccttgttgtgatttcctacttagtattttttgctatgtgaccttcatcaaactagtaaacccctttttcttgttggggaacaataattgtacctaccttataggattaaatgaattaatatttagagagtgcttacaacagtgcctggaattttagttattattgtttctaataaagtgcttaggagatatctcatatttccagttatgattaatctatgcacattgataattctgcaggtttaaaattgtattcattacattacaaaatggaattcgtatagagatacgagttttttcaaaagttaagatatgtttccttagttttcttattatatttattcttgagactatttttatactaattatgccaactggaatttttatagcactttataaagctttaaactaatttatctcattttatctttatggtgtctctaaatgtgcattatctcattatgccctgtaaataagatgcaatgaattgcccagtgtcacacagctgtaattggggaaggtagaagtccagtccaagtatcctaattcagaatgtctgtgctccttttctgatttactatgcaggcttttatgggcatcccaaatcaaaactaaaagagtctagattcaccaatgtcttttttccttgtccatcataataaaccagaaccaaagatctctcctgctctggggcattttgtctaagattactattaactatccttccttattctttcattcgttctttcagccagaagttattgagtttctgtgggagatactgtgctttgaaaattcttggtatggatccaacctattctcagtgcatttatatacatataatttttccagttataattgctatgtttaaaaaaagatttgtggcaacctcatagctacatgaaatatagcaaggtaatataaattagaagttattgataaaggaaaaaatacatacatatgcatacacatacagtaaaaatataaaatgcactccaaaatgagacttaggaggccctatgtatataccctacagcccttgatgtatattacatatgtgggaaggcataaagtgattgaccttaagtcaaagtatgcctctaataatcatgctgagagcaaataatttatactatatataatgctatcttgaaaaacatttaaaagaaggtatttaagatatagtatgtctgtcttagcaatatttaaacaaagtatggaaggtgattgatacatgatgatgatgagatggtactaacaatattcatttgggtttttacatagagtatttgtggagaatgtagtattatgaaggtttaaaatttttttctatttgtttgtcaatgttaattatctgagtttacctaaagttatattttaagtaatgtcactgacttacagtattaatggttgaaatgtgagtctgaattattttactgtattatgagaaatgacagaatttagtttcagctgtgtatagtattcactgataacttctgtatcctttggtttagaagtatttcttattcgaggaagtgaaacctactgagtcatttagaatagacggagtcctcagtacttcctcttaacattcacagaatggtggaaactagaaaatgagttatttattggtctttattctccattaatgcatattatttgatcttgcattaagtatacattgacagaaacattattttaattattgtattgaagacattcttcatgtgtatttaatcaaccacctgacagaaactagggcagagtaaccaaagtaacatttgagactttatggttagacgtctaatttgcatgtgacagtagtccatttataaagtgtgatggatgaagtcatattagagtaagtaccattagagacacattcattgtgatctgtgtttttgcaaatcaaaattaatggtcattgcagctgtttgttgatctgttgagtaatgtaaactctcaacaacaaagctgaagagaagatgtattttgtgatatctactcttataacaggcactcttaacaacatcagtaatttgtataacattaaatgttcaagtagcatagagaaaaatctaaattcagttttagatgaacagttctttgttttttcttgtgctataaagacacagttattgcctttcacctcctcccccttcaaaactcaatattttcagttatctcttgtgaggaaatatatcacataaactggaagtcatccttaaccagagcaatatgtaagttgaccctggaggttcaaagagaaacttacgtagcctaatagtgtggcttgcacattctatgcactgtactaaaaaataatgtaactttcatgatttcgattgggtggaataatttttagttacgattaggtctttaaaaggttcttggtttttagttatgatactaaaatatttaccagtgaatttatatgatgtgtaggaggtacttcaaaacgatctaagagtgaaaggaacaagagaacatatagatgaaactggactggggatgaattgataattgttgaagtcgggtgataggtacatagaaatttatagttttccctcaaattttatgtgtatctgagattttctataataatttttttaaatgtatgagactatgtacgtctcaaagtactgctttccattcttcatgtgttaaagacagagggaccatcagatcttcagatcttgctttctttttttcttttcttttcttttttttttttttttttttggcacggggtcttacccttacacctaggctgagggcagtcatgctgtcgtagctcactgcagcctcaaactcctggtctcaagcgaagcccccagctcagcctcacgaccagctgggacttcaggtgtgtatcacaacacctggctaattttttaaacatttattttttatagagacaaggtcttgctatgttgcccaggctggcacagcttgctgttaaataaaactatctttttgaaattgtcttatgtaaatctgtaaaagtattccactataggcacctaatatggatctcttttgacttttataacaaggggagttttgcctaaatgataatatatattgaatatgactttaaatttaatactctttttataggagaaagattttgcttttcttaaatatttactcctgtgttagaaaatatcattgcaaggaggttatcaaaaatagaagaattaaatcttggttttgttatctgtaaaatgatggagcaaataatatttaatgtgttttgaagccccctggacatgaacatgtatggtggtaaaccttgaaggattctatatttatatgtctgtatttctttataccaccttagttcaactgttttaccattccaagtttcttttccaaattatgtacaagcagatgaataagaatatgttgcaattgatttttcttaagcaagctcagtgttttttgttttttgacactttgtttggcaaaggaggtgttcaatttgttgagctgactgagacttagaaagaataaactatcacaggatctgccacaaagtagatgtgaggtagttgttcagtgagaagagagtgagagagttattccatccttgatccaaacatcactgttgggcaggtagatcactaatttcctattaaaccaagtggacaggacagtcctgaattaattatggaacagtttctttaaagacgtcagctctctgcatcccttgtcttgccactgaaatggtgctatttcatcccatttctcccttaaagtaattttgtgatttttgccacttttaaataacacattgctaaagtcagggtataatgcaaaaccaaaaatcgggtggagaatatcatataccatcttatctctagaaagcaatgttgtctgtagggcaagaaaacagtctttatttccctgcaaattgtgaatttttaatgtagttagttttgagaaatgaatgtaggaaactttgagcaaaacatccaataatgaccagtaaatacatttccagcccaaaagtatttgcacaaatctttttgtatcatattgtttaaaagcaagataagggcaaaacttagtatctcacagagaactttatatttgagctgtgtgtcaggatgacaggctttcaggacagatgatggcttttgaatataaatgtacacattttaatttttcctaattgtgtataaagattttaaaatacacaggcagttaaaacaaatatttagccacagttagatgcacaagtattgtatttttctcaccgatactactgctttattatgaatttttaccaaaatatacttttttatggaatgtttggttagtactgcactcttttcataaggagagatacgattcaatttccagttttcttcaggactctaatgccttgatacattagaataataagtgaacagtcaggaaaataaagaggtttcttctcttgggaaaatcaaactttcttaaaactttatttttccttcggaaactgaagatactgaccatttattatacattttgttggctccctgttctatacccactaccacttcaagagaaatcttcccccacctcctaccccaaaaagtctttttgggggtgtgacttttatagttgtagaacattattagcatttctcggtactgtttattttgaacaaaaccaatccatgcattttctatttgtttaggagtcggatggtcagggctgccctttctgtcgttgtgaaataaaaggaactgagcccataatcgtggacccctttgatccaagagatgaaggctccaggtgttgcagcatcattgacccctttggcatgccgatgctagacttggacgacgatgatgatcgtgaggagtccttgatgatgaatcggttggcaaacgtccgaaaggtaaaactagaaacatgggtttagttaaggtgatgtgaccatatgaaaaggaaatgatgccgtcataaatggagtatgactcagcgtatttgagtttagaaaagaggcacacacacatactagcctaggcctgtacagggtcaggatcatcaatatcactgtcttctacctccacatcttgtcccactggaaggtcttcaggggcagtaacacacgtggagctgtcatctcctatgataacaacgccttcttctagaatacctcctgaagaacctatctgagactatgttacggttaactttttttcataagtaggagtatactctaaaataatgataaaaagtatagtatagtaagtacataaaccagtaacattcatttattatcattatcacatattatgtactgtacacaagtatatgtgctatacttttatgcaaccagctgcacagtaggtttgtttacaccagcatcacacaaacacgtgagtaatgctttgtgctatgacgttaaggtggctatgatgtcacccaccaggcaataggaatttttcagcttcattataatcttataggatcaccatgatgtatgtggtccattaccgaaatgtcattgtgcagggcacaactactataatgttgacaggctgatataatcatagtttgagagagaactagattctcctcaaggtactctcattggttttaaacatcataatgagaggtaatgagagtgtttgtataaaattaaacgtcatctaacactggcaatagcaaatctgtgaagaaaaaatttaagaaaggaaattattaaatttggaaatggtgaaactgaatttgattaaaaatattctttgttcaaatctagaagaagtgtctgatagagttttttcttaatgggaaggctgtctcactgtttggcttctaagaggtcacataagatttacttttgtgaatttaccagaatctgagtttgctcattaatgttgttgactcacaaaaattgaccttgtcagttcactgagaatggatgttactgcccagtttaagtttatataaccaggtacacaacagactagtcaatgaaccagaatgccaactgattaatttacagaccagaaatacagtggtcaagagaggtagactcttggatatcaggctattccctgtacaggaagatacatgaaatcccaattactttcacaaattttttgctctcaaaatcagagaaatgggattgagtggtgtgaaaggtcttcatatatggtgccacccttcctcctgacttgggcagacatggctaatcagtcatgatacttaccgccgctgagttcagacttggccaacagagcctttcaacacagccctgaagatcttccactgtagttcattaggatttggcaggtagcatgaaacctatttgccattgtcactaggtggagtgatgataatgtgtaatggcatcagattgtattcaccgtattcatctatctctgtaggaaggaaatgcttggtagtggaattttgacaaggcatttctttgggttacttcatttttagggctgtgtgcgagttttcctcttgggaaactcctgcattttctcccttccaatgttccatgctgaccaaatacattcccttaggatattttacatacaaatttttacataggaagatatgttaacatcaccaacatggctactctcaaattttgcatttttctggaatgatttttattatttatcttattggtttttcataaatgttataatttaacactcagagaaactacacgatttcactctttaatcctgaaagatacagtttttattaatagtttgaattatgtgattttttttgacctaagaaatggaagaaagtagttattcctcagaaatcctgaaggatgaacaagaaaaaataatcttagattgaaacagacttgagacttgagtctcaaaacaaagttaacttaaggaagacgtggaataaagcaccagttctgctacagagtagtaatggcatgctaggcaagttatataacctctttcttcctcagtttaccccttcgtaaaacagaaaaataatagtagcttctctgttgggtgttgtgatgatcaaacgggatcaaggacctaagaacctagaagaacgtcaggtcaccaaagtcataaatgctcactatattattaccaatgttaattaataatactatgtaatacatattctaggatatactccataatctgagttagtagaaatcatttcatcactatgcatgtgagagagctacatatgcggagctggtgatattgatgtactgttccccagaagacataagtttgaatactggctctacaatgtacaaatacaaacaaatcacatgactagtctgagcctgatttcctcttctgaaaaatgagaagaataatacctccctaaattgttgaaagaatgacatatgttattaatgctggttttattgatttgtgaccctttttactgcaagttaaaaatgattttacattttaagtggtgtatatttaaattcctatataagtacctacaaaatgtccttgattttacatcttgtcctaaaatattcactacctggccctttaagaaaaagtttgcagccccaggtctaggacatagtaagcatttaaagaatggttattgaatggatggataatggagaatgacagaaaagtatttgagatacaacagctcaagtagttgaagtgctccaggtagcagtgttttttttagctgtgaagacatttcaattttctttactaaatgggttttcatcttcagtgatccatgtgagagggtaggtctaggctttaagagaaattttaaaaagaatttttgtataccttactgcttgggagaaaacagtatctgccctatgtgaatttgggaaaagttgcagtctcaacctgctttccattgaattaagttgaagcttgagcctcttttcagtagtggcactaacaatcgaaaggaaatgctgggaacataacatgagaagcagaatgttccaaggtgactctagtgtttctccttgatcttatcacccgctcccaagaccattcatatttcattctctctggcacactggtgagctttctgtcaagggagctgagacattttcaaattgatcctttctgttttttaccccacaaagagtagaatgatgatctcaagcattattgtcctagaagtcacacttcagtagcttatttggcagtcagcagtgtcatttaataaagctttacttcactacaatcttgttatactttcttatgggagttagatagtttagaaatttatatcacttgtaattatttgtttttaagttgaagaggtatatttcagttaaatgtctttatgtctactaatctgcctctagtcaattaaactgattgtgacttttatagcatttcataaatgtgtaccacttaattttcaataaaacctaaatttttatatagatacaagcataattctttctgaataatttggaagaatattgctatgatgttatgttcttatagtattttatatgtacttctatatcaggagttacactaaattgttatttaaaggactgtctttaaggccagggacaatgccttgtatttgtgttcccaggcacctaaatgcactcaataaatatttactaagtgaactaataaatgcacactcctgcacacatgttcctttagggcaatgacctaatcttatgcatattttaaactttgttccaacctgaatactaaatatatatatacacagagcatcaaactgatagattttatccagttttttcttataggagtggttataaacaaatgtcacatggtcattttgacacagtagttcttctacctgaatgttcttcctctgttttttaaatcattcttttgtcattcattcatgcattttcattcattcactcattcatttattcatttattttttaattaactgatgaattaatttagagctggagtctcgctttgttgcccaggctggactgcagtggcactatcttggttcactgcaacctctgcctccctggttcaagtgatttttctgtctcagcctcccgagtagctgggattgcagacgcctgccaccacgcctggctaatttttgtatttttagtagagacagggttttaccacattggtcagcctagtctcgaactcctgacctcaagtgatccagccgcctcagcctcccaaagtgctgggattacaggcatgagccaccgtgcctggtgcctggacttactcatttatttttaaaacattttattctctcagttaaaagagtgactgccagtgattgtccaacagaatggttccattgtttctatctgccattttgaatactcatctttttcttcttttatgagcgggccaatgataatattgtgaaattgtttcttcctgtcttcagcatattacattctttaaatgagtaatgctgccatcgtagcatggaaacctgctttgattgtatattcctgttaccatggtgatatcccacagctggagttcataaatagtagcatactaaacatatagtgtatttctattatttcttttatagttaaatgtgtttaataatgctaatccttacagaagcaatagaacctaaaagtctataatacatactttttgattccctctctgatagaaagtattttgtgatactaactagaatatgttttagattttgcccaagtctgtatcgttgcctgagttttcaagctgtcagaatcctgggataattagatctgatgtgagccctcagacaaagtccaggatgtgactggtaagatcacagccttctctctgacttcattcagatcagagctaaaaaacagtatctgttgctacccagtgatttgcttcattttgccagaggattaagaggaatcttaacagtttttcacagtattagtcaccaaatacataacatttacatttgaaataaataaaaatttgcatataaaaataaaatgttgacagactgaactagtgaatgagcccagattagttcttattgtcttaaacattgtattttcttaaaataatatttaaaatcttaatggccacattagaacaaaaatgggtatgaacaaaagtcaaggaagttattcttttttatgcattctcctttgttgccttcttcaattaaaatacatcttgctaattagaaatgtaacatgtcatctaagtattgcatttcctaataagtgtgaagaacgtctctatagcaggggtccccaaaccccaggccatggaccagtaccagtctgtgatctgttaggaatcggctgtgcagcaggaggtgagcaacaggctagtgagcgaagcttcatctgtatttacagccactccccacagctcgcattaccgcctgagctctgcctcctctcacatcagcagtggcatcagattctcaaaggagtatgaactctattttgaactgcacatgggagggttctaggttgtgcgcttattaggagaatctaatgcctgatgatctgtcactgtctcccatcacccctagatgggactgtctagttgaaggaaaataagctcagggctcccactgattctaaattatggtgaggtttataattatttcattatatattatagtgtaataataatagaaataatgtgcataataaatgtaacacatttgaatcatcccaaaaccatccccttcccctccctgccaccgttcgtggaaaaaatgtcttctacgaaaccagtccctggtgccaaaaaggttggggactgctgctgcatagtattcctgatcctaaggaaaggggtgtgtgtgcatatcacagtgttttgcagctatttttaggaggatgttgcctttttacgttgtctatttaaatctgaatcacaggattagagtccgtgttggagccgtaagatcaaaaattacaacagaagtgaaatacaaaaataaaaatagacattcgaaatgcagaacatggttctcaactataggcaattttacctcttgagggtattcagtaatatatggggacacttttgttttttgcaatgactaaaggatggcaaatactgctggcattgagtgggaagggccagagatactaaatatcctgcaagcccatgggtggtcctacccaaaagaattattctgaccatagtactaatagtctagtcctcaccactgccttcccacaacatttaggtagagccgtgagtgacagtaagcaatccgaaggcacaggtttgttgttcatcgtttacttgcactctgtttactgatcttctaaaagctcttcgtgcaaaatagtggtattttctttattagcatgtgattagcaaaagaaacaactgcatcatccaagtgctgtgtgatgtcgtaggataccttgaagacattgttacttatttctcaaaaataacttgaatacatagtatattagaagaagcaacacttgaactctaaaagttttgagtccactgcctcaggtagattgtcagctccttgaggacacaggctatgctttattcttctttccattcttagaggtttgcatggtgcttgatacacagtaggcccttgagaaggatttttttttttttaatgatcagtggtgacactgttcttaattgttatattcttaagtagagctgccctcttggagtgaaactgcctgggctcatcttctagttttcctacttagtatctatgtgccttgttttatttaacaccccctcccactccccttcccccaagtacttggtccataaaactctgcctctttaagattaaatgagttcatatatataaaaagccattagaacagtgctggcacatggaaaatgctaagtattggttgttattaatattatttttaaagtaaattaacaaagatggggtctcactctaatgcccagcatagtcttgaaatcctggcctcaagttatccttccacttcggcctcccaaagtgccaggattacaggtgtgagccatcacacctggccacattttttatactagtgttatttaagactcctgaaatattgctgtacttctaagaattaagttattagtatttttgagttgtctatggaaggttttcttttagatgttgatagcattccattaggaagaattagatgccaaaattttattggttaccaagcaattggttgtatttctttaacatattaacatctatatagttataatttcattgcatgaccacattaaaataatttactgttaaaccatgcttcaagtttgaaactcagaaaatgcaaacttgtcaacatgtcagctgaaacactattttgaggattaaataatttagataaggttacaatgttcttattttctgtataactaagttgaaatgattaagtattatctaaagtagggtgaagaactacagtaaccaaatagcaattgcttgttccaaccagctttagctggcatttggagagcaataagggttttcacatgaaagcattttttcagtgaccaaaatttattcctttaattctcagaatgttcttgtttcttggccatttttatggaaatatttctgaattatatgtcacccttccctgccttctcaaaattggttatgcagctgagcgaaccaaagggcataagaagccactgtagtcaacatgacacatttctaaaagcttacattttgctacaagatttgagggtagcttgcataaagagaagagaaccataaatttaaattaaaatcacacatatatttggatcaggatataaaatttgtgtaaatgtttaaagtaaaatacaaaactttaaagaaatttcaaggttatggaggctgatcataggccaggagatcaagtccctctcataagaaggtacttcaaggagaagcagtatcatcacttgtgttaacagtgtttaatatttcagtagagcagtgcccttaaggttatccccaagtgtgagactgcactactttaaaggagacctgtcggcaactttattgaccacttctctgccagacctcaaagagaaatcagctcttattccatttggtgtttcttaatatataggtttctgttttcctccgcttctttattgcttctaatttgaaaatttccagaagttactagtggaacatagacagtaattcagcctttgaattagtgaacttcattttacacagaaatcaaatgtccatattcttaggaaggtagcagggtgcctacattctgtttctgtattaagaagtagaaaaaaaaatgttcctcctacttcagaggactttcctgtttcattctgggtttctccccaaattattgtaggttgagacttctggaagtatgaccatgggtaaccaggaaatgattgtattgctctaacacaatattcttcatataagcttttgtttattatctgtggaggtactctaaaatagataacacaagtcatttacttaagctataatgccatatgaatatgtgagtatgtgtgtaagcatttttgtagcatataccctctctgaataatattctgtataggctactattttaaatactcttgatttttgttttgttcacaaaataaagtgaaaaaactgagaatagtagcaagtaaaatatgcttagactattttatcatgtgataaaagttttatcaatgtcttcattaattttcacgcatttatagaaaaacattgactctctgagtccttctcatcatccttattatagtaactgtatatacctttttcagatatggaaattcattaagtaaaaatcttgttaaaagtattataaaattgactttgattatctctgggactgtcagaactctcatcttattagtaattggagaggttttctcttgtcctgtttttataataagcctgactgtgcttgaagtttcttaccaaatgttttgccatttccataataatggatcttattaacgtttttataaaatggctctttaaaggtgtggataaaatgaagagtgactaaaatagaatgagttcccaattattattggtaattgctgaacatagtgagatgggcttttgactactttacttcttagggtttttctgtcaggattttctgttaagattgaacatgtggatttctctgttcctcgtggtagttataaaggattctgtgcattttgtcagaaaaataaataatagtatttattaatttccaaagcaaaaggatgagtatgtagacgtaaaagaatccaatgtggtctttaaatttctagaacaccaggattctagtttgtaaaagatgttctcatctgttactaggattgaagaacttaagagaaattgttagttagacaaatatactggtgtgatgataatacaggaaggaatcaaggctggctcagcctttggatttggaaagaagctgaaacagataatttatcataaagttttagaaacaagtgtaatactagaaaaatgtgagaatcacctaaaatcaagataaaggaatgaagtgatactgatcagtatcttttaacttgaaaggatgtttgtgtctttcccaacttttgcagttacctctacataaggattttggatattgtgaataagttctatacagttaattctgtgttcacattctactttctatgctttatgacttaatttttccatacttatatttgtttgtcttaagattagagagatgaacatgaaggagtgacaaagaaaggaactcatttaagcagtgctcaaaaggacatgatactttagtcattcattaacacatgaaaaccagaagagcacaggcgaaatctctaccatgaaaaagtcagaaaaatttaactgcagaaaaaatagtgacagcaattataacagatagaaagaggttgactatcctaatatataaagagcactttcaaatcaacaaagaggaaattagtggatatggggaccaaaaaaatgtaaaaagctagtgacccaaagaagaagtacacatgtccaacaaagattgcatttctttcaaattatttgtcaaggaaggaagggcaaatttaaacattggtatggcccattaaaatgacaatcaagcatcaaagcaaacaaaatattctttgcatttgagttttgtgtaatggtaattatcacacatggctggtgagagaataaattggttttaccatctggacagcaaaagagccttaaaaatattcataaattcgacacggttacatgtctagtatcttgtgctcaaaaaaattgtatgattctttccttaaaaactagcacatatatggttaaagatttctttatgatactactttataataaagaaaacttgaaaacagcataagtgttcattgcacaggaatggttttgtaaatccatatatgcatactttaaaattatattcatgaagaatgtttaccgctaatttttttttgtataatgctaataaacattgagtagacttaacattgtgtatgtgattaggaaccatcaccaatggctatatgtaggtgttctttaaaatatctttatatttccctataattccaaaatttcctagagtgaaattatgttctttataatgaagagagaaaaagtgaaataaacagaaaattgaaaagatacaaggacagaactgccaaaaccagtaaaacgtattttttatgggaaacagtttgacaacatgcatacccctaaatttataagcctaatccaattcttaaactacttttcttgttatcatttgttgtttttgtgtttaagaccctttctaggttaaaaagaaagcctaatcatgttaaattaaattttaagtttgcttttttaaaggtcagtgctaataatagcaaataatccaagaggcatttctatttgacttttgaatgcatttttacttaatttaattatttggttagatatttgattttgttactattgcattaagagctgatagtgggaggaattgtctgggacaaaataagaaataacaagggtgggcattttgtttaacagtaactttaatgggattaccttttggcagtgtagatgggcaagctttgactagaaatttggagataatataatagattacattctgtcccttttacatcaaaataatccattttcctaacaatttcatctaaataaaaattaaatattttacatcagcatttattttacataatgtttataaatgttttacatcagcatttattttataatgcattatttaattttattaacccataatgttctgtcaaatgcattaattttattctttaatctacagataacaccaccaataggtggtgacattttcaagactgcatggcctctttatattataactgaaatttaaatataagcaatttgaatataggaaatttaaatataggagatctgtggtttgaaatcaggttatacatttatttatatgattactcaaatgcaaaaaaaaattgttctttataaccagttggcattagagcttagatagtcatacttgaaatttacattttctaatcaagagtatccgtatgaacaaaatactaactgttgttattcagaatttaaatgaatcctttgctacttttctacccaagatgttttatacttatgtatttggtctgcatttttatacacaatggaagtagtagtgattaagaggaacagtggacaaatcagtaagtgataattagcacagttcacagtcttagaatttaaacctgagccaggggattggctctatgtttgtttgttattattgttttaccccaacagctccctttatcacctatcacatgtagaaaaagataaatggcctgggtgatttgagctggggattagaactgtggaagaaagcccagaaaagggtggaggaaatggctgataaggagtcaatttaggggataatatgaactggtgggctaaagatgaattcccacattcaggtcccagctatagaggcatttgaattacctggtgattacctgtacaggggaaaagtctgttactctagaatttagtcttttaaaatgcaaaaataaaaataaaaaatgcagctattacaaaaagacagcattgtcttttaaaggagaaataattttggtctacattgaattgatgtactttgtttttttttattttcagaaggataaaaaagagcagactttttattctttaaagtgagctgataaattttgatgtcctgaatttttaatatctcatccaataaattaattatggactggattgtgcaacattgtgctagatttcctttttttccgtagactttcaaataggaaggaaaaaacacaatttgtgatgactcaaataaattaaaagtgaccttctatatgaacattacctactaaaaatgtttacaaatcttcatagtgtttgaattttaccagcaatgaaagttaaggcaaagcttcagtacactaactgcataattgtttttaaaaatagtaaaaggttcagcaaagaataatgcattactatcaaaagagcattgttaaattatctactcttaggtaggaattttactctaatttaggagaactcaggaaaatgaggtttggtgagtttagtacatataatcaataaagatgaaagatattttttttcttcacatactctccataaaatccttctaactaagccaacttacagatactgtgtagcttctaaatgaattaattcttcaaaaggacagtacataatcaccttcagaacacagtatgaattgcgctaagcaagatatgcttccaattacattcacaaagcttcagagagcctgctcgtctggaagtatctaaaagttcaaggttattactttgccacacacattggtttggatgtctgttctgaggattttcattatactctttcagtatccaacaggctcttcaacacatctgaaatcaaatttaccccatttaaacatgctagaaatttactaactgcttaaatattagtaattaatccaatctagggtgaaagtcaagtcctaatactgataatgggctgttagctctatcacaattctgtgtcttgtttcagttaatgagagaagtaggaaaatatttgcagaaaaaaagtgcttggaggagcacaggtctcattcagcagtctttgcagtgtgtacatctagccctgaaaagtgtttttgcaagtaactaaagaactgtgcattctttctatatataaaaaaatatattgtttgggctagaaacagtattgatagctttgcttcttgattcttctttgtaaaggagagtagccatatcaacaaagcatttttttcaggtaatagcttttctagttctcagagaatataatttatcctaataggaatggtggagaattagatgtcctgtgacaattgctgatctacagtgtcaagatgctgtgatgcaaatagctttacatgaggaattttttctgatctatgatgattttgggattagttttgctccagaaggttcaaatttatggaacatgtgaggccctgtgactttctgggtagaactattcttttgttaagaatcactggctgtactcaagtcagtttgtgactatagagattctttattttctccttcagtgacaattgaatttttttgacttaccataaccgagctaaaagaaatgaaaaagaatcattaatttttaaaaaagaaatgtttctaaaaattgtaatgtaaaattatgttgaattttgttgaatgattttgttattcggcaccaacagagacaaagactttgcagtttagtaatgtcatgacaatattcaggcatatcggaggtatagtcttctgtttagctactcagatttatttattttattatttttttagagacagggtcttgctctgttgcccaggctggagtgcagtggtacgattatagcttactgtagagtcaaactcctgggctcaaggaatcctcctaccttagcctcttgagtcgtgggactggaggcgtgtatcaccacaccattctaatttaaaaaaaataaaaaattagtaaagaggaggtcttgccatgttgcccaggctagtctcaaactcctggcttcaagcagtcctcctgccttggcctcccaaagtgctggcattacaggcatcagccaccagggctggcctcagatttatttttaaaaagtctttacaaaggacgcataaatttccaactatataaatgtgggctgtgcagtgacttccttgcaaagagtatactatgggaaggaggagaaaaaaagaaacttaacagtgaagaagtctgataaacaataccacggtcaggtaattaaggtcagcaacagcagtgatgaatcatgttgatagcatggatccttgatatgatgtggtcaaaatgacactttacttccgtggtcctactcctcaaaacttttaaccccaggctaattatttttaaaaattagacaaatcccaataaagaaacattctgtataatctctaaccagtactttacaaaactgtcaaagtcatcagacacaatggatgtctgagaaactgtcagagccaaaaggagagtagggagacgtaataataaaaactaatacagtatcctgcatgaaatcctggagaagaaaaaggacattgggtataaactaaggaaatctgaataaactatggattttagttaattaacaaaagccttatacaatgtaactaagtgtgaatgtgaacgcaaatccattatctcagttaaagacagactaaatgataaagctgctgagttcatattaaattgtcaaatgatgacatcagcaattcgtacaaaggtaagtaatgttaggaggaaaatggcttacaaataagtttaaaactataaatactagcattcagaaacagaacaatatcaaagcatgatagatagtgttgtaaacattaagtctaaatcttcaaagagctagtaacttagacttggaaactgcccagtattttagaacagaatattgtttatcctgttaaggctacttgattaactgtatggcttttataacaaatagaaattaggttatgctgcattaaaacacatagacacacacacacaaacacacaacattatgcaataggaagtgttacgtacaaattgaaatacttactggcttttcgatgctactttatctaccagacaatatggaaaacttgtagtaacagaaacttagtttttgccagaagtacagacagactttcctagatatagttcattgtgtttcaagaaaaatgtttaacttgagaatcttttccatgtgtagtattaaggaaaaaaaaataggcttaaatcagtttgttttaaacacacaattatttttttaaaaccccacctgatttaattggtttgcaggaaaatagactacatacaagatacatgtttggaacaaagcaatagctaagtattagaatgtacaaagctcgagcaaaaaaatttataatatagagatccaagagcaatgtgcattattgcattatgtgtttctggattcctcataaaatatagatggttttcttgaagaatatctgcatttgattgtctttatactatacttttgctgttcttttttcttagcccctttctgcaatatgtaggaaaaacttatcagtcttttcttttcgtaatactacccttgagggacagtatgaactatttgcttctattaatcagggtaaactaagggaattcattttaacaaatgtatggtgccagaaaccttcttccaaccaaatatattctttaagaacagagaacactctgcacagctaataaacagcggcatctttctctttttctgttctagtgcactgacaggcagaactcaccagtcacatcaccaggatcctctccccttgcccagagaagaaagccacagcctgacccactccagatcccacatctaagcctgccacccgtgcctcctcgcctggatctaattcagaaaggcatagttagatctccctgtggcagcccaacgggttcaccaaaggtagatcaattttattagaaacacagatgaaaattttgtaagacaattattgattgctcagattcttttaaattccattactcatatttttttgcttttaattgtgaataagtttgatgagtttctcgtttagtttatggtgagcccacattcatatttcttgttttattactatgagagttacacataatttttgcagagagcatatgtctgtagccccatgatttgccagaataagaagcagagtaagtatagttattttgtaaaatagatttaaaaggtcattatatgcattaaaataatttgcatgctgttatcaaatgatattttacgctcatttttatagtgccatgacaagaaaaatgtgatgggagtatatttgcaaagggcttaaggtatgacaaattatcacccttgtttcaaggcgactcagaagagggaattatatgtagtttttaaacctggagaaattaattacaatgaattgcaatatttgcagagtaattatattactgtttaatattaaagacataatttttactctacagttaaacctacagtttattttactgtgtggattttttcctcgaatttgataagtataagattttaaaaattaattttatttggtaacatgctctgttgtctaaaatttgttggaagcacaatatatttagtagtgtttgattttcctgtattaccagttaacctggttttaagatcacacattattagaatatcatttagaattatgtaaacacaacagattccttttttctctgctattttctaacattaaaattacaaatcaatcatcagattaaccatgtaaataaaagtcaaaagctacgaaaagcatactcattcaattcattgatctatacctttatatttctatgtaagaatcagcctgatcttttaaaatactgtaagaattcataaaattgaagtaattccatatgtagtgctgctctgttacttgtgatcttttactattctggtaataaccgataaagaagcagagcacatttgcccccatatcagttttgctgtatcacagtgatgaaaacaaaaataaagaaaaagcttttaatcttctaaaaatattacaaatattacaaatcaagttactctttaaaaaaactgaaaccatgacaacttttctcctctctcctaaatcatagagatgtgcaaataaattattgacatattcttttaatgtcaaagtcattgtatgattttgatcttctacttgtttctgtatcatttctataacttaacatttggatttatgtactcatttctactttttcctaatgaaatgtatttatgagttttttattctcatttaaatttcattaactagtaataaaagcaacttattaaaagatggcacaggaatctagtaaaactccttgtttggcttagaaaattaaagcaataatacactagtttggaagttctgtcagtgcatggtacaacctttagtttttttttttttttttttttttctcttctgtttctttagtcttctccttgcatggtgagaaaacaagataaaccactcccagcaccacctcctcccttaagagatcctcctccaccgccacctgaaagacctccaccaatcccaccagacaatagactgagtagacacatccatcatgtggaaagcgtgccttccagagacccgccaatgcctcttgaagcatggtgccctcgggatgtgtttgggactaatcagcttgtgggatgtcgactcctaggggagggctctccaaaacctggaatcacagcgagttcaaatgtcaatggaaggcacagtagagtgggctctgacccagtgcttatgcggaaacacagacgccatgatttgcctttagaaggagctaaggtaagaaatacgcaaagcagaagctagagaatctgcttataaatgcttctgatggtcagtagcataaaaggttgggaagtgtttttttttgcctagcatggcacttccctgtaagtctttaattatccccaagttataaataatcagaagctcattaaaacttgaacttaagagaagtcaggaggttctaagtaatgatttaattttaatgtgaagacactagacaatttgccagagatttatttcaccttcccgtatttcatttgccaaaaatttcttctattaacaacttagtggtggtataatcagcctgcatttttttttttttttgagacgaagcctcgctcttgccccccaggctggagtgcagtggcgtgatctcggctcactgcaacctctgcctcccaggttcaagcgattctcctgcctcagcctcctgagtagctgggattgcaggcacctgccaccacgcctagctaatttttgtatttttagtagagatggggtttcaccatgttggccaggctggtcttgaactcttgagctcaggggatccacccgcctcagcctcccaaagtgctgggattacaggcatgagccactgtgcccagccagcacggtatttttaacaaactcctttagttccatccaatttttacaagttgtgttactatacatgtcataaatcagttgttggagttagttattgttgcctgggacagtgcacattaggcagccaggtaaaatgaacctcctatatacttaaaaacactaaaccaagtttactcttaaaaatcaagttcttttcccaagttcttttcccctaaaattgagagattctcacccttgttgacttctcacttgaaggatgttactactatcatttatatgtccaggaaggagtttatgtgtgcaggaaactgaagagacagcagcagaaatcatttactttagaattcttggtagttccttggcagaggtatactgtaataatttctggtatttggttccagggtgcttttgactctgagattagatccttgccatcttccctctgctgatggaggccttggctcttctgaagactataatattaaagtgatctcatctctgtagaccttaaattgatattttacagaagtagtctagtccttcaatataatggttaaagttggtgagttcatcttatattcaggatataaggaagaagcgtgacttgcactcagctcctctgtgtgtggaggtacagtgataatgaacactttgtatgacagtatttaacctcagtgatatggggtagccaggaaaggaagagtcacaactcagtagcacagcattcaaaaccaaaggcaaggagtggtggcagtttgctttgactctttgctggtgactcaccttggtaccttcagtctcatctattataaatatgaggatctctaaaggtttcctcaggttcacctaaatataatgtaaatgtaagagaacatactaataaaaccaattttataataatctctgttagtggggagctcctcctcaaaagagtttttccacaacctagaatgctgattggaaggacctgccattcattttcaaaaattttcaggtttttttttttccccctttagctttactttttattcatatgtgatcacacttgtcttgggaagagtgttagaagaagctgtaattaactagttatatctcagcagcatgttataatttattttctaaacggacttcaaaaatacttgaaatataatagttgttatagccatttcattgaagatatcttgtctcggggttcttttagataacatgttatagttttaatgctattataaatttgccctcctaagctctagaaaactggctgttaatttttttatgagatgttaccccaatttcttaggttattggtatgagatactgtttttattttattaactataggtatgaaatattgtgagttcagttataaaaacattcctttaaatatttctgtgtgaactttcatgttgtgatcattgaaacagattagtataatagagaacactgagtgaaaattttatctcttattgaaagctgaactttaatattgcttagttctctctaaataattctcattaagatttcctaagttctaatctcatatattgtgggtatttatattgcaatgctaatatcctccagttaatttcatgttactcttagatttggtgtttcaaccaaaattagacttctgagatttctaatttcaacctaattgtttaacttctggagtgagctgtaaaaaagaatttatatatagttgattcattgaatgccacaaaactataaaacagaccatgtctcctcttttattactgttataaaggaaaaatgattcacttcattagatcatagggcataccagaacttagataaattagaaaaggcctaattcctcttctcatttcattgttgggagacttaagaggttggcttcaactaacaactgacataatcacttaggatcctgtaattctcagtttctgtgatcctgggttggagtttgttaccaggctccaaaatcaggcctggatttcactttttgctctagaatatactggctttgtccccaaccttacttattttccttataaataaagtggaactttattttattcctttatataaaatggaataataataatacctggtatctaggataatagttagcttccagctatctaaatttctaaaccccttggttcaggaataaaaccaaaaataacagctgtacatagagtgtcaaaataagttgtaacacttggcatttttattcaaaatagttgtctgttttttaaaccgaaacatcttctctttgtttcactccttttttcttttatttaacgtagttcctaatgaatgcttggtaagcactcaataaccaagttaataacttggttaagcattcaataaccaagttaataactcggtgcttataatataatacttacttggtgctagtaatctaacactaaagccagtggtggagacaaacagtatatgtgcttgggtttgagtgtgggcaggtgtaggccaggagagaaaaggtggagggagaggatggctgtgatatgcaaggagttacaaagggacaggtttttatgtatcttgcatagttttcttatttaaaataggatttttgttcctctgcagaagagtagtcattttaaacatgtagatcatatacaacaagaaaaggagagcgggtgaagagaaaggggagatattttgattttttaaaaagactaagatttttaatgaaaatagtagatgttaacgtattttcacccttcatacctaatagtgccattttgttttgtcttactttatcattggattaaagagtgggagatttaggtatttggaggctgttttattcacataatttatcaggcccctaatataagcaaatgcgctttttttttttttttttttttggtcaaatggtaaaaactgccgcttaggtttagtagttttcataagcggcaaagcttcaaatctagtagcatcttcccattttagagataagaaaatgtagataagtactaaaaatttaagtgaaaactaattatacatgtggaacacaagggaatttataaaagtcaataccctgtcaatgggcgaggactcgcttcccttcattactcgtcctgtcattatgtcagttcccatcatccagaagaaccatttgccttttctgtttgcattgtcactagaacaatgaatgataggggaaatggaaataaattaagctctaattactccatttggtaattgtagagagctctgaggaaaaaattattctagctcatagttaaagtgaagtctaagaagtttttgtgatattcatttactccctttactcttcccctcctacctagcataatcaaaagctagccaaggtaatactttactttaaatgtggatctacaagctaagagttattgtttatttatctgattttgatggtaagagatttctatcctatagcattgtttccattctctgaaagtctcagattttagtcaccttataaagctgttccttaataaacatcttgttggctagatatttattgttgttggtggtttgttttaattatctgagcaaaataattcaccaaaatatattaaaatatgactaaaaacttgcaatgtatctacttatttctagatattaacattatacccaactgtcattatttgttctcttgaggcagtaagaatactgctgtttcttatatgataaaactatctttaactggtcatgtaacttacggtgacattgtcctaaagatgacctttgcatgataatcttttgaagatgctgcaagataaatatatcaaatgatttccctttttgcagttgtggcttgttgtacttttggtggagaggcacatgtttacattcatgaccttaaagttatctataaatttcaagggctgtctttcagaaaatcagtcctgtgtgtgtgctggtttcatgtacagtctgacgttgaaggacaaattatagggatacttttgaggtagcatgtgtaggggtttacatgtgtaggataaggtaaaggcttgtgttttgtaagatttttaaaagatgtttttcttaattgtgctaagattttcacaaaattgttttctctgtatctcaagggtcccatatgttttaatcttcagtttttaaacaatatctgaatcacaagaaaacattttacactttaggattagatgataccttgaggcatttagagaaatattactctaagataacctagatttctaataaattatacaagccagtttatacttctttcacaagtctacacttccactattttcttagttcttcaacctcattacctaattcagctatatgagtacctaggatgttcaagacactctggggaatacaaagaggaataaagcacagaaaagctagtcaggatgagacaggcatgtgattaagaggtaaggcatatgagaagcatccagaaaatgtgtgggatccccaagaagaaaaaaaattccaagatgcaggactgaaaaaggaataaagaaagcttcatgcaagagggtcactggctataaacattaaaagattggcagctgttgtttttgtaggtatgacagaaagaaatagcatggaggtgggagaatatgtatagtaataatgtagtcagttttggatagcctatagcataaaaagagtaggcaggtgaggtaagaccatggcaaggaaagttaaaaccaattatgaagtgtcttgaattctacattaagtaatcataatttggtaggcaggaaagaatatttaaagttttcttttctggcagaggaatgctagaaacagaattgggaatttagaagatttaaattggtagcatttccaatgcaattgtgatagatttcagacagtgagctaagaatgcagaggacaaaaaaatgtttcttagttgaggattcagtgaggtttcagtgtgaactgcttcagttgatactatcagtctgtgcatttgaggaactatagttcttttttaccatttgcaaacaggtttttcagatatctggaaaatatataaagtatttatttttagcccccaaagttgtgatttattgtactcactcaggtagtggcagctaaaatgatttttgggtgtaaagaaatggttgcaggtgagaacatgaaattcagaatgacataatgttaaatcttgtcctctagccaccgtcaataaatatgaacagccattttagatgaacggaaacatgatcatcattttataaagtacctgcgcatttaggtcctttgctatctcaatctactaattaggaaagaactatacagttctattccaagttttgtattaggcgtattgtcaactctcagtacatatttatgaacagtttataggaataacagccattaagcaaattatatatatgtgtgtgtgtgtgtatgtgtatatatgtatgtatgtatatatatgtatgtgtatatatataacatatatatactcacacatatttgctgagagtatttcagtaataatgaatttctttcagctaaacctaaggaaagtgatttcactagttaatacttccattaataagtgcatctgagccaatcagcagtcaatattaatttaagccttactgtgtgtaaagcactgtaaggcatatgctataaaatggtaaaggagtaatagaatttttgtcctgaaagaggcccagtgtggcctaaattctccttgacaaaacgtaagtacacagaacatgagcaaaatattatcagatgccaaacatcaaatgaatggttcagaaaagacactacagcatttcagtggccatagaaatcctttagttactatacatcagtaacaaatggtatttctgaaacagttagaaaaagtgacatttgcattttaaagcagattaatggattgtttcaaaaaattaggaataagtcacttaattttcttaggtattgccctccaagagtaagttacctcattaattgtactttgtggctttactagtgatagacatcattgcctgtatgacttgtcttcatgaaatttaatagtagtacataaaaatatgcaaactctgttattagtattttattctcatttacctaatgtgcaagatgaaagcaatttcaataccagtaaatgtttgagtgtattggtattttttaaagatacgttcataatgttctgattataaaatcatagttggtttattgttaaattagagccaatacagctgaaactattattcccatatctgtcttttattttattgttgctgaaattttattaagcatctatctatgttggatgctttgtcctaaaataacaaactattgcatataaaatccacattacattttcccaaatgagttttatatgtatttgtaatatgaattaaggaaatttgattgcagatatcttacattgtgtctattcctgcagcatgcaaagaattccaatgaagaatgacttccaatgctcaatatcacaatgagcgtttgtggtttgatatgtgtgtttgtctgttaaaaggcctttttttatgttaaaggattttattaaacttcaaaatgattaatatctattaggaaatttcttctttctggcacacttcagaacactttgccttgttctgttatttggccacagtcaaaagcccagataaaatcattgtatttaaaggtaatattttgtcatggtatggctacagtatattgagtctaatgaagggaaacatgaaaaaagagaaattggtactaaagcataagaccagtctctagtgaaactttaaagctataaaatttgattttgtagctttgaaatacgaacgttgatacatttcaaatatgttctttaaacttgtttcttattttaagttgataggtaagggtatggtcttttaaattttgtattctaaagcttccacatcattttagaaatatgatgggggaaagtaattggtaagtccttgactgaattatctagcactgctcaaattactttacatgtatttagccccaccatcagtctacctattaaatatgaattaataagagtttgcaaaataactccatgataaaaatctaatgattgcttataggtcttaaaatgtgttcaattttttcttgtgaatatgtttctgcaaaattatcctctcataaccgcccccctttaagttgctgtaatttcaaattgtaggagaaacgaacagtttttcagacatttcctgtagtgtaacagaaatctagacatcagaatctcctaattcagagtaattgaacactggctttggatggtcccatatgagcaagttagaaattgtggtttgtgtgttcacaaactgaaaattttgtttaaatttgcctgcacttggaaagccagaacaggtctctagttatcattagcacagaagcttcaaccatgaaagaacaaaggcacttctgattatattaatgtactgtttcatcagaaagtttaagaatgttccgttcaccatggagaaatataatttagagcactttaatttggaaggaattaaaaaaagaataacatcagtgctgcttttttctttaccccttatagagaatgctaacttttcaataaaaactgatcatagttggtgaattttactgtctacctatttagccattaatatctgtatctggtgtatataaattatagtattccctctcagtgtttaagaatcatggtgtaacatttttattttttgtgtaagcttgaacccctttgggcagtggtgaatcatcagtgtggtgctactgactcctgaataaattgtatgacatttcacttatttaaatatactgaacttacatattgtcttcagaatggtagagcagctatggctttaaggtccttcagaagttaaattcttggaaactaacaatttgatttactttcaggtcttttccaatggtcaccttggaagtgaagaatatgatgttcctccccggctttctcctcctcctccagttaccaccctcctccctagcataaagtgagttttgttgaatttggagatcacttgtctatgcatcaagatatggtagagtggttttgagaggttctcagtacagacactacatgatgaatagaaagtcattgatagtaaacagccagctgtatttacaaactaaattaccataaattaagatccttccctatactttgctggtaaccacaacatctgtgagacttacctaaactcttgaggttacatagagcaacagggagagaaagctacgcattccttgcatcaaagagggtagagtagcacaatgaggtatcccttcaaatttacctcttctcctcagaggtatcccattttatctcagcttcccagtttgacatcataaaataatctttggttccaccaggtttctcatttctcacatccattcagctactaagtccttttgattattcttttggagtttttcatattcctctaatcttctccattgctctagctttgcctcagagctggacaactgaaagagccttttatctgataatctcttatgatcggagagttagtatcaatgggtttacggctagtaaaatgagataactgggtatctcatttgaaaaatggggccccagaggcacactagatatctctcattgatatcttcccaagttaaaccaggataaactggatatcccaattctaattttcctgctctttatatgaagtcaggatgtctgcctttcctccggtatattttgttcactgtaactgagttcatttaatttaacttctgccttccccaggtcaccttcccactcaaaaactagcattgtttccctttttccattaaattaactctatgcacttctgctaacagtaagatcctacagaattttacccaactaaaatattttttagtgattcctaatacttgccctgtaattatatctggattgttttttctgactttgtgagatttttatgctgttcctcctgtgcttcaccttttcctaatctttatcctaaaggtccatatctcagactctatttctttgtgaaatgttctgtgacttcttcaattgtagtgattaagactgatttacattcctcttcctgcagtttatttcctactcatttaattatatactctgccatactgttttctaattttaatgtgactctccaattgaaaagattacaagactttgaggacaaaaactttgtgttctctatctcccataacatcttactgtatttgatatatactaagtaaatgatttgcatatagtaagtaagtacacagagaatacgtggatgcttggtttattgatttaaattttatctgctgaggtccttatttacaatagaaggtcttagagcttcttcattccttcatctgcagttcactgaactcccactatacatatcaatgcaaaagaaaaataaggtagatcccagccccccagaaatttacagtctactttagaaaataacattaatgtatatgaaatcattagagaatggtgtgcactaacatttataatgctaacttagtagtttcaaatgagtttggaggagaggctgttatagactttgtgaatgaaggatgtcatggagggatgggcatttcatgactcatgaagtacaggcacaattgggaaaggcagaatgtgagaaaaaacaagggtgtgaaatttggcatcattcaggggacggtgggaaactcagcgaattagatcattctccagtgggaacttctcttggaactgcgagatatttagcaggcataggagttggaagcaattatacatactctgggtagtaggctctaaatcagtgacaacatatgattatattctagtgctaaatgaactctggatatatgtgcatgtaataaaacatctaagctcaattacctttgataactcagagaacattagaaaatgaaatcttgctaattagagattaaatgttcacattctgtttattaaattcatattggcaagcgagctggaagttgcagtgtgcttcatttgtatttaacattaaatacctgcttctcctgtaaatgaagcaagaataacaaattgatagtaaaatgtattttttctaatacttcagttggtaagtgcatctaaaattagtcatactttctccctatttactcttgatttttccctcagaaacagtgcaattgtttcacttaactagagaaatattttaacagtgcatgcttgccagcaaattagttttcaattttgcatgctgcaagtcagccatagttagcagtacaaatgctagcttttaatggcacagttatctctccagtttatttttgtttctgactctgtgggtagcagttgttcactgtagacaatatgcaaagtatataagagaaagaaaaaagtgagatacataagcacaagcatatgcatatgctaaatatcaatgtccagagttttaacattttgacattttttatttaaacttgtttttaccttatttagattatatggtagagataattttatgccctcctttcttaacattatcataagcatttttcttaattgtttagttatgcattgtgtttattacaaaaccagcatggaatgctgatgtgaacagtagaataaaatcagactgtgtaccacacctgactccgccattaagtatttgtcttttttttttttttttttgagatggagtctcactctgtcgcccaggctggagtgcagtggcgcgatctcggctcactgcaacctctgcctcctgggttcaagcggttctcctgcctcagcctttcaagtagctgggactatagtcgtccgccactatgcccggctaatttttttatttttagtggagacggggtttcaccatattggccaggctggtctcgaactcctgaccttgtgatccacccgcctcacactcccaaagtgctgggattacaggcgtgagccaccatgcccagcctttgtcttatttttaacaggaaaacttctctgggcctttccccatctggtagaagagaggatcagtttaacttaaattatctccaaaatccttttgagattcagctttttaggttctatacagtgtgtttttttaaaggatactgatggcaaacctgacaataacagtgagtcaagagtcttggctgtggatggtcttagaacagaggtcttagcaaaatgccactcacagaaataggaatgtcagatgttcgagagttcatttccatcttagactcttggtatatgtttcaaaatcaggccaaagcaaaattacgcattcctaccaaacatacacttctgtagttcttacaaatgttgaaaactccaatgttaaatccaagaatgtcaaagctaacatttaccttacatttatactatgttgttataaatatttactgaaatagaattgttaaatatttgatattgtttactctgattcccatttacatgatgtcagaggctgctgtcatagtgtttcctgttaaactgtatctttgcaattatttgacttttattgtgtaatttgtcttgagacctagaagagtatgaaatttacttttaaaaaacatactgttgtaaaagcaatctgaaattatttgtatctatatacttaatttttctaaattaatgtttttttctaaatggtttctgcttaacatctttgacacttagctataaatataaaattttaagtaaatcctagaaacatgggtaattatttttttggagggagaacaaaataatcctatgtggattctttttactttatagtttaaattatttatttactaaattatatttattttgaaataatttgatttttcaaaattaaaactaaatgatactctgaatagttaaggaaatatttattttacttggcacacaatagattggcaaatgaacagaggcattattacaagtaataatggagtaattacttccaggtaagataatgttctgaaatgctgaggaatatctttttttttttttaagaattttttttgtgtgtgtgccttgtcttttagagatggaccttttgtacttttgatatattaaacttaggctttcaaatagccatgaatcaatattacttggttttgcatttttgtaagaaaggtttttttgttgttggaggtggtattatctatttttatgaatatatattcttataatatatatttttctaaagagataagtccttgtatttttggttccataaacagagaatacaaatttcacctaactgtcctgttttcattcagttgctttacttactagaggttcagtttgtacaagataaaatcctttatcctctttggaatttaagctctagttagggagaagagaaagtaaacaaataactatattgtatattgtcagctagtggtaagtgctaggaggaaaaataagcagggaagaggataaataatgatagagaggtttatactttagatgaggtcattaggaaagacctctctgaaaggctaatatttgaacagaaacttgaaggaagtaagggagcaaaccatgaggttatctgtgtgaagagcacagaggctggatagaacacaagtacaaagcaacttcaaatctgtttctccgatttacaaacaagttagtgaaaatgcttataagtaaagcttatcttaaagcttgatagtataccacacagacttttttttgatgtcatgaaagacccttgaaaattagcatgtatatgataaaatttagtgttttgaggaaatgatttatatgtggcacatgtgcaaaaatcatttcaaatatagctatattttgttttgtctcttctctggactatttatttgagttagacatttttccctgttaggattgggataatgtaatataagaaatatggtgtaatgtatagggcaagctctaaagattctattttaacagaagatattttttcgttacttgccttcagatactattaaaggaagatagattcttatatgtagaaaacaagcttactaattaagcatagcattggatactatctcctgtgctggcgtgaaaattcacttgtttcttggtttgtttcaagtgtgcatatgtagaagatgaatgtatttatcactgaaggctcaaataactaacacaatgaatttaataaaatgtccaggttgtggggggggtggtgactatgcatctttgagagtgtatgtccctatgtattaccagtgtattaactcagtaataacagcatcagtgcagaacactccgggaatgtaagatgagaatataaagggattgcagtaataagtgtaatggtaggctttaatgtggagtcagccaaatacaatttgttcagctttattcctctggtatcctaaaaatttcccgtaagtttagtcaatgactgaaaattgtcccatgccccataagatagaaattttttctactgccaaatgtcacatacatggatagataatgactaaatataaatctaattattttacctgttatttagtagtaagtaaaattaaacctgttttttgaatgagtttgtagaggaaaataaaaccccacatgtaattccagatggtataattagttacatgactctagttttttattttgctattttatatacaacttctttatctcatttgtaccatcctcttctgttattttttttgatagaaaccagttttttttttcagaactggataagattaatttttaataaaaactatgtaatcttgaataaaatacactgtaatgttattcttttggtttatttctaatccaaatagactattatactgatattaaacattttcattaaaaaacataagcagaaaaataaaatctgaatctttagaactttattaaaatggtgataaatggaacacctaagtatttttttcatatttagatacaaacaccacttgaaaactattttttcacttctagatataattctctaaaatctgtgtaagttgacaatttatactagtaagttttagcaataagccttattattccatgaagtattagatgagctttgcttttcacttcattctgaaataatctactgatttgtggcaaagcaaaagagtgggggaaattactcgagaaattctctatcaatgaaggtaaaatatcaaattagccacaacgtatgtgatttaaaaaatggtccattgttgactcataatgaactgcttctttggaaacttaaatctttggagggaacctgcaacaggcagatcttagaacatttctgatattgaatagttgttgaatgtttagtgaaaaatactgttttctttttacatagtgtatgcttgaattgcatagaaagggattcattataaatgaaagtatcttaggcatagagaggatgtacagtatatatttctcatttcatttggtaatagaacccattctggtccttgacttagctaaattgatggctgtgtttacaggaatttatggaaaagtaatattataagtctctcccagttccagtgccatcagatcaccattttgcagcccatatacttaacttcctttaattggaaagtaaatcctccaaagttaaaccataataggtgtaataatagattattattaaggcccttagcttgtgctgtaggtagtgacactggaccttttgtcactatgcttttgtctctgtgcttttgtctccctgtaacgctcatgtgaattctaaagttgctcaaacaacaagaatttaaagatcagtattgaaatttttttcaaagataggtatcaataaacatacacttgaaaaatcttagtttttctttgatctttgaatgtcctatctaatgacttagataatatggttgaaacttaaatctttagatttttcaaaatgccttcttgtaatggtcacaaagaaaagcccctaaaattgtttctgttctaactaataaaaagaaataaaaatgaatgttaattcaggcatgtttgtttaatataccaaaggcctcttgctccctctatagaaggtgctgcaaaattgcagtcacttagtttttttgttttttttttttccttcctttttaaattgaacagatttctcatgaggttcatgatgattaacaaaaagatttggatccatgtgtgtattcccaatgccttgcaaactagttggtcgtcctaaagtgaatcaggatgagtttgtgcatttctgccttggcatttttcattggcttcagccagggttagattaaactctggtagcaacagttacgaaaaacattgtttatgtttgcccatttgtttgggtttgttttatttggtttatttactcttatggataagtgaaatgcttaatgtttttgatattaggtgatggttttactcattttaattctcttttatttagccagggaatttaaaccataaaaataatgtacttcaaagacttaaaaataagttctctcattatctacatctaatcattccattcatttctagctctcagttgttggtaggaagaaaaacctaacaacttcaactataatttcacaggatagttaagttttacagaattaaactggatatttttccaactatagataatattcacaggagacaaaaagtttgaccattctataaagacttctttaatatataaaacaaccatgcttattattgtagcatcatattttacccatttaatttaaattggaacaaaatattaatattttaatagatttggtttgaataaatttgtacattttgtcttacaaatccaacatctttttattttattcgacaggttactagtttttcctattgtgccatttaaaattgtgaggatgaaaattcttttatcccatcaagggtcttaacaaaagattgaaaattgttttcatttgtatttgatataagaacagcttctctgatgataccctgtaattgaaaaatgaacataagtgataaagtagcttctttgacttgaatatgtcacatcagacttgcctgttttgaatattatgcttaaactagattggattttttttcccctggtaggtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtaagagtatgggcagatttttgccacttacatctgcataagcattatcatttgtaatgtttgtttttaaacaattgattcacgtctaattaaaaatccttaatttccagctcaaatccttctcttcctattataccatttccagcattacttttaaagaaaactaaaagatgcagatttctcattaatcaggaagtcactttattttatcagttctaaacacctatctcatgaagaaaaattagtgctcagaaactaaggtattattctgaccttccttagaatttttcttcatgatgtacatactagtataatttaaattcattttccttcttgatgtacttgctacctacttctattcagattttgaagaagtgggaaaaaaataccaaggtgtttctcttaactttttgtttttctatcaagtgctatttaccttcctttagatcaaataagaatgtgaggaataggccaggcgtggtggctcacgcctgtaatcctagcactttgggaggctgaggcgggcagatcatgaggtcaggagtttgaaaccagcctgaccaacatggtgaaaccctgtctctactaaaaatacaaaaatgagccgggtgtggtggtgggcgcctgtaatcccagctactcaggaggctgaggcaggagaatcgcttgaacccgggaggcggaggttgcagtgagccgagattgtgccactgcactccagcctgggtgacagagcgagacaccatctcaaaaaaaaaaaaagaatgtgtggaataatgggggttttttccttacttttgtggcttattctcacttacatggggatagccttggtctttgacttggaagctccatcgaggtaaataagagttattaataatgggttgtttgtattgaaatattactttatctttaagtaaaggcatgagtctgcatttatattattgaatttataaactgaaatctattatttctgggtttgtttgttttacctgactgaaatatatgaggtactcagtactcccagctacaaaacttactttctgagttctggtttcctttgtataacattcctggggccatgcttggcacatagaatagggctcaagtactgctggtggccctacattccagcatctctccatgactgactgagggataggggagatggcaattacttcagtcatctgtgccatctttacttagtgtgccatttccttttgaggtcaagtcaatcaactgaaaacattttataagtacagtgttcccttcttttccaatgaaaaactaaaaaatgttttaatgtgtttcttcaggcatctcactgaggtaggaaatgcagcacttggaaagagaagcacattttcattttaagcagatgtctgtcttaataagtgaccctggttagaattatcaacattctgctgaattattacccagaattgttaatagtctacagcgcaggactttatttttttaaaagcaagaacctaaacactccttgctattgctggtgtttagataagacatctgttgataaaatcatatcattgagaagacaaagaaataacgaggagctaattttatgtatcagaagaaccaagtagctagtgatgtatttgagaacaaaagatattccttctcatcaaatataagttgttacaattttcggttctttggctcttacaaatgtgcagtaatttttaaaaactaaagtaggatgagctttttttggtctctttgggagtcttattttttttcattgctggataagtgaggagtataattttaatacgttaaattttaattgttacatcatattggatgtgattttttgtggcagagctcccaaatcttgaaaataaatttgaattataaccatcattaccatttttctattgttacagaaaagtaataaacgttgataatccctaaatgtaattggagtgaattctttatctaatttgttcttgaaagctgatggatgaagtatcttcttttgtgctctccaaagagctctttaagcttaacactttctttccctgtgtgtatttttccccctttggaacatcaggagaagtgaattgtcaatatgtagaaaaagtgacccagtaaatgagtaattcaatttaatttcagtaaaaatccatcattattatgaagattcaatacatgataactatttttaaatgaactatatctggccccttaaagagaagtggccaccagagagaatagaaatgaatttcagatgaagcattcttgcattatctattcaaatatccgtctcttccattacatacgcacttttactttgctctaaaaattcagagtaaatcagagaaaagtaaaatgtaaactctgatttctatagctctttgtcagttgattgttatagagctatatgctgaaggcaaaatatgctccttatcttctcttcctgctgctatcttcccatggctcaattttaaccattgtgttcatagtatgattccgaagaattaataccaataatttggctgggttcggtggctcatgcctgttatcccagcactttgggagggtgaggtaggcagatcacttgcggccaggagtttgagaccagcctgggcaacatggcaaaaccctactaaaaaataaaaaaaattggctgggcgtggtggtgcacgcttgtaatcccagctacttgggaggctaaggcatgggaattgcttgaacctggtaggcggaggttgcggtgagccaagatcatgccactgcactccagcctttgcaacacacagatacacacacaacaaaacaaaaaaagccccaacaactcaagcatacttttctggtaagtagtaccctttacaaacacattaaaaactgtacttgtcgtgtacccaaggcctttgctaaagtcattttctgtagtgtcagagggccagaattttgttcttggcagtggtgaaatagtaccacaggctctagttctttgaatgttcctgtcagaattgtaatggaattattgaaagttcaataatttttcttcttttatgtgctgtagcttatttttattacaaggaaattttaagttacagaagcaaaaatatgatattaaatactatcaacaaataataaagtcattgagactgctatatgtttttgccttggcacatacatctttttttacttcaaaaacttgaccaggttctattgaattcttccacagagataagttaattttacatagtgtttaggatatcaacaatttttgtggcccttgtaattcttggttatagtttaaaaaaagagagactgtgttacttgagatatttacttctacattttaaaataagggatgagtatcttgatgttattactggtaattttgagaaaagaaaaatatatgttcaaactttattaaataaacaggaatactagttccctctacctctcaagttacttttaattggaaagtattctccttatataattttactctgaactgtcctttaggtcttgtgataatggtcactgtatgctgaatggaacacatggtccatcttcagagaagaaatcaaacatccctgacttaagcatatatttaaagggtacgtatagaatataatttcctttgtgatgtacatcttaatggtcagaatttaaaggcaaaatttcatgccattgtactgaaaatacattaaggttttgtgttatcctctaggagatgtttttgattcagcctctgatcccgtgccattaccacctgccaggcctccaactcgggacaatccaaagcatggttcttcactcaacaggacgccctctgattatgatcttctcatccctccattaggttgaaacctttaaaaaagttttgaacaacccacccctccttcttttaatttcagaattttcagaattcagagttcagtataacacagactcactgggttgtgaatttgcctgaaatttgaatgggttctccaggtgccggtgactcccaagttcacgagaccattactccatgtagatgattaaggtagtagtgtagtagttgggcatcagtcaggttttaagcaagttgttttgtccatactaaatgtagtctaaaaacacatgagagctttgtgctctagtagttttgaagtgatgacttgaagtgttgagattttctttaagtataataattcttaataaatatgaacttgcttttcttgcagcatgagcaccagttccacttacgctaattaaattatgcaaaattaaatagttgtatgtagagaactgataataaattctgttttattctaatcattacaactgtaacacattcaaaaaaatctcaatttccttgtttcaaattataataatgaagaaacatcctatgtgtatttttgtttgtatccacaggtgaaggtatatgtaggtgtgtgtatatgttgaagttggctggtgccactgtcactgttaatttcattaattctaattaggaagcatctcaccctgtttttagaagacagaatgtctgcacaccttacataacgcttcacaataacaagtcactgtaacataaaataatatatgatgaaagtcaatttacaagcttttctgaactggctgttaggccaccagaaatacgaaatgaacctttccattttctcacaagtaccactgactagtattttgaatgacacaattgcagtatcttttgctcctgttctctctgtagttcaggactttattctcacctacatcaattacaaaaggctcctaactctgttacctacagtctttctctcccttccacttcttcctgcacacttgtttttgacatattacccagatcttcatcacctggataagcattgttcaataaagctttctgtggtgatgtaaaggatctgtatatttgcatatgggttaagtgtgattatggaactgacttttcattaaatttaaatagctaaatgtggatgggtgactactttatatgacaaagtagacagattttccaacctgtctttttaccttgacctttttctaggtcttcttatgcgtgtcatttttttcttcatactgtttactctacttagacgatcctgtccttgcatttgatgaataaatttttgagccttctgatatcccaggacttcctgttcttcaacacttcactgaataccacttgtctacatttctctttcctcatccaatgcatatgtctcttttatgggaccaataaaacactttcacttgtaaatcatacctatttgtgagcttttttttaagctcctttcatggaatctcagctccttgagtcagagccttataattttgattttcatatcatgcatgagcctggcccagtgtcagatgtttaagggctgaaaaaagatttgaataaatatgataatgtaggaaatagctgaaggttaaaatgatagcaaaactctgtcttgtgcatcttcagatgtgttaataagaaaaatatgattaaagaaattagtgtgccaattttttctaaagcaagttcagtaaatagtaatatctttttttttttttgagacagtctttctctgtcaccaggctggagtgcaatggcacgatcgtggctcactgcagcctccgcctcctgggttcaagcgattcccctgcctcagcctcccgagtagccaggattacagtcacgtaccaccatgcccggctaattttttgtattttagcagacatggggtttcaccatgttggccaggatggtctagatctcctgacctcatgatccacccaccttggcctcccaaaatgctgggattacagacatgagccaccacgtcaagcctcatttttctttaaatatgccattatgtttaaatcattcaacttaaaagtgtgaatttttattattagaatatgtgtttatgtataccctgtctcattcctaaagtattttgagatagcttaccaaacatgtgctttttttttttttttttttttttttaaagactcaaggtaatgaaaactatgagtagaatagtaaggtgtgacaggggacaaataagtagatataaaactatgctgcaatattctagttattaaagctgggaaatatgcaaatgtaagtagtgcttggaaccagagaaggttctatatttagctgttcttctgtagctaaatctgacaaattgaaaaatatcatattctctgctctaggtacattttatgtatattttgacagcatatcaaatatatgaaacattaggttaaataaattaaaatccagtggaataaactatatgggataatctaaactattctttgacattaaattatatgtatttgaggtgattttccagttaaaggctgtgaactctttttgctacacagaaaatcctgttttcatagtgtgactgataataatttttagttcaaactctgagtcctaatattataaaggttacaagtgaactatgagctgtttcataagttacaatgctgctttttataaaaaggcctgatagaaaaagtttgcatgggtgtgtttgtacgtatgtatgtatgtatatgtgtgcatgtgtgtgtttctctccagactctcccacaaaataattagatcccattatcaaagttggtgaactgctggataaaccctgcgagtgaaattctttataagcagcaagagaaaatcaattattacttttgttagatgctgtgtattttgattaatgaagttctgctgctgatatcgacctttctttaaatgcaggtgaagatgcttttgatgccctccctccatctctcccacctcccccacctcctgcaaggcatagtctcattgaacattcaaaacctcctggctccagtagccggccatcctcaggacaggatctttttcttcttccttcaggtaggagggaaaagccaaagaaaactattcacttaaagcaaaaatatttccaaaaccatactatcaaataaatgttagtgattcattagtctctacctccctagcagaacagaaaaatccctgggtggtaaaagtagcaatcgtgggtacgtttgtttgtcagttgattggtttaatgccaacataattacagagatggaaagagcaataaggaactacagtagaaatgatgctgtagagattgtccagtccagaaacctccttatgttacaaatgcaaccctgagaaccagaactgctaaggggttaggctaatagtaatgggaacaatatttagactttgtcttccgattcccaatgcaatgttttttaataggtttttaaaattttactagatatgccttagtgtcataattttgttcaaaacataagattttggtaaattatgttgttataacaagaatagagaagtttttctgaaatagctgcattttattgttttgttgtgatatttttaatgcctcagtgtgtatctactcactaattattggtattctaaggaaaactaattttttcttagtaactatttaatattattatttacatatgtatatattatataatattatttgtttaatattagtaaactaattttttatattagtatattttaaaatttcaaaagaggatctgaaataaaaatgaagacttaataaactatctgttgcattagttagaattatcaggaaaaagaaaaaaaaatgttatatggaatctgctgatactaattaacagttccagtctgtcttgttagtgatactgtgccctgaaacctttgcctccttggacttgatttgggaaaatacagggtaaagatgttctttttttcatagttccttacatttctacatgagataaatcatgaaaatctttaaaaagtgctcagtttgtcctttagctacgtatatgcctcatggaaacttgagaatctttattcccaactgattcccttatcagcttcctcagttgattattagtcatgtctaccattagtttttcttagttttttttttttttttttacttggtttgatagtttagaaagtttgaagagatttgataagacacacaagttatggacgtacaacttggaatacacagcttcaatttcttaaagtggtttatttttcaagaataatcttctgcttttacatcagtaaatattgattgagccattcactgtttacccatttgtgatagatgcctcctgacctgtggaaccttatgagtttactggattctgccaatgaaagcactagtgtcataatccattccggtccaagcaggaagtatagtgtatgagtccgttttcacactgccataaaagcactacccaagactaggtaatttgtaaagaaaaggggtttcattgcttcacagttctgcatgcctggggaggcctcagaaaacatacaatcatggcagaggtgaaggggaaccagggcatgtcttacatggcagcaggtgccagagagagtgagcaagcagggaaaatgccagacatttatcaaaaaccagatctcatgagcactcactatcatgagaacagcatgggggaaccacccccatgatccagtcatctcccatcagttccctccctaacacgtggggattacaactcaaaatgaaatttgagtggggacacagagccaaaccatatcataactgcttgggcttttattcatatagttagttacatgtaaacactgctgtatttcgctattttttttcacttattcaaaactttgtcttatacatagactttaacatgatttttagaacaatgagtcattccaaatcttagttacttaaatttcctgatgctaagttcaagatagtttctggctctcctattaccttttatatgcgtaacttaagccccaatagatggaactttgatgatgtggcttttctgacaaataaagtcaagctttccaagagctataataaaagagaacattttacacaaatacacacttttacctgtttttgtcaaaagaattgtaaaaggttttagttatttattttatcttaattgttcatttccttttatataaaatcttcataaagaaacccctcaagattaattgctaaaaaaaattactagtttaaaatgaaaatgtcccctcttattagctaaaatgcaatttttataaaccgttgtatttattttatagatttgcctgttttgtgtttgcttggctgccagggagctcaacaacacttatccttaacttagagtgtcaggctagcatgggagtcagggcaatgtttttcaagcctgttgactctctcttgactttcattaatagatggacatattctaattctcttcccaataatatcaacatcccttataataagtatttcttaaaaaaaaaaaaaggcaacttttttgtttccttttccagtctttcataatcatttacatgacagatttctagtctggtaccagatcctttatgactttttttttttttttttttttgagacagggtctcactctgttgcccaggctggagtacagtggtgcgatctcagttcactgcaacctctgcctccaaggttcaagtgattctcctgcctctaccccctaagtagctgggattacaggtgtgcaccactacactcggctaattttttatatttttagtagagatggggttttgccatgttggccaggctggtctcgaactcctggccttatgtgatccccctgcttcaacctcccaaaatgctagaattgcaggcgtgaaccactgcacccagcccctttatgactttcattgtgacaattattttgattcaaatttaaaaccgtgtgtattagctttcctccttttcaaaagaaatattttaatattttaatgctattgcatatcatagagcataaagatttttttggctgataaatgagcatttgagtattgttttgcaaagtatttaatacattatactgtctaaagaaatgtaaaagattcattatagcagttctccctaagaattgactttattaagaatatgctataaatgctaaagtttaactcagtagttttagaattataaaaggtaaataaaagggactgcaaaatttaaacaaactctgtgagcattttaccgttgttttcgttgttcagtctgtaaaatcttagtctggtgtaatgatacatcttttctaaacattattgagatttggtttcctgaggaaataattatttctaattaaagacaaatgataggtgtcttaggaagagaggttgaattttctcatgatattatactaagtacctaatcaagcctacatttttattccagcattcagtgccctgcacagttgggccaggttcagcttttcagccttatagctcattgacttctatatagtagtgattgtcaaattgcagtgtcctatgtctctgcaaaagtgcctcagtactgttgtaggatcaagggggcagggcaagcagaggcagggctctcattcttcttttatctcttttctattttaggccttcatgtaagatttcttttggcagaatagtaatgcctacagctgtacatgagccctcagttttggacaggccaatcatatcttaacaaaacctgaagtccccaacaaaagctttacactcttttgtctccaatcccaaggctaccactagtgtatatgtgcttttttccatattacaaaaaagtacttcctttttgctggtagattgaagtagacacccctacctctactttgtgccaggtcagggctcaagaaacaaagcaaagactgtatttaagtcctgctcataaccataactaggtgtctttgtgcagtgcacaaaatacagtgtttttgtcagtctttatgctacctttattttgctgtttctctaacctgtaatctcacttgaagatcttatctaatcttaaagtcacagttcaactctgaaagctagccaaattgactttattaaagataaatggaagttttattcttggatccagaaatgcaactgcataagtaaaatatgaagaaaatccagatcattagcaacaattgttgaaaaggttttaggggttttgttgactacaatctttataaagcatcatcatgatgagaaagcgcatgccatttggggcttgaactcacagaattcaaatctgaggagtgaagtcctccacagtactcagcctttgtggagaccactgtgttcagatgttgaagcattaatgggctagagcagcaattctcaaattgtgttctccggattcacaacattagcatctcctggggatttgctttaaaatgaaaattcaagacttaataaatcagaaaccctgggaatgggactcagtggtcacaagccttcctggtgattctgatgcatgttcaagtttgaaatatactgggctggggtattctcaaaaaagtgcaaccaggatgataaagtaaactagagtggattaaaggaactgagaattggctcagagaagagaagaccttggatgtagacgagagttgtcttcagttacagtatttgggaaagaaggattatattttttccatgtcacttcagaaggtaggagtaagatcaatgggaaatgttaagaatgaaaattgcattcatcctacaaagaagtttcctaaatcattacgaattgttctaccttgagtgtagtgagttatctgacactggaggatttaaaccaatggtagatcattacatagtagagatatgaacatgagctaaagcatgagggcagcaaagaggggagaactttgtttcacctaaatggctcttcaaaccttttttagcattagtttctgctttgatatactctattaaaatttgggggataattgataaatcttccaggtggtttatggatggccagggaggaaatttgccattgttttaattatttaattttatttgcctctgtttttacacttggtaaaaatttggaagtgtttcagatagtgaagaaaaaaaagaattaagaaaaacattccaggccaggtacaggctcatgcctgtaatcccagcactttgggaggctgaggcaggcatatttcttgagctcaggaatttgacaccagcctggccaacatggtgaaactccagctctacaaaaatacaaaaattagccgggtgttggtggctcgcgcctgtagtctcagctacttgggaggccaaggcaagagaatggcttgtgcccgggaggcagaggttgcagtgagtcaagattgaaccactccactccagcctgggtgacagaacaagacgctctctcaaaaaaaaaaaaaaaaaaatccatacttcctcctcctaatcattgataggtatccttcctttttttttgttgtttttttgtttagttttgttttgttttttgcctacgagtatgtaagttcacatttatgtaaaatttatttctcatagtatatgctatttataaccattttccagttaatatgtcatgagcaatgtatatagttgttaaatatttgtacacaatataatttttattgatgtatatttcctcatttcttttttaagtagcaaaccatttcctcccagtcttcccttagttttttataatctctcaaataagtactaaattttcagttttgcagtgctgtactataaatcacaggcagtagtactttgctttttacaacagattagtctctgtatgtgtatccatttatttctcaaacacttatataagtgataaattggctgtgatcatttcagtttgttttaatcctgtggatataatatttttctttagtttttgaagaatttattttagaaaagctttattactttacaaagttttcttatcaggggacaattggtaattattatactctgtgtagcaattgtgttatatctaatctctttccctttaaagcctgaatgcttccaactttataatttgctttagagagcccagtttaccctatggctgacaaatagcaaacttaggatatgatgcaagatagaacatgataatgattcaaaacagaaccataagcaggtatttttgctatttataataatattttgagtgtatctagtaccttttaaataatatagtttaatggaagaatatagtacagtggtatattttatagtctatgtatggctctgaagtgtatccagagtttactttttaaccttaaataaatagatgattagaaatagcattaagtgtatttgacaaacctataaatttcccgtaaagaaataagcaagtgtatgtattgtaaaagacagtctttcctctgacttaatgttgtatcaggggcatttagagtctacaagaacctaaagtaaattttatgaaatatctagtgcaaaatctttgggaaatttcaaaatttatgctgaaatcaccatatttgtttcctttggtttctctaacaaaccaaaagatcaacattgtctcatgcgatttatcttctcattccttcctaaaaatatttctggaaaattaatggagctggaccaccaattcttcaaatattgaaaactcaaaatctaagagttattttaatcttgaaattaatgaaaacaagttaaagagccttgtctaagtcaaaaagtcctcagcaaagctggtgataattattatactccaaaaaaggtgaaaagataatggcttcaaatcctgccttacattatccagtcaaaaactaatataaaaatagtcaatatttaagcctatgttttcatcgtaagtaatcaaatacttacatattattaatgtctaagggagtggaagttaaaacaatgaaattcaatcgggtcttctttaatatttacaatttttgctttacattagcaattcaggtttcactgattttaaaattgtgaacaatctgaatttgtgtggatttatttgtggtagttaaaagtaaatattaagtgcaagctatttgccaagagtgttctaggtgctatgtttgatttaggaggtataattcaaacatgagaagtaggaaaagatccctcagatccctgtcctcagaagaattgacggtactttcagagatatcgacaacataaaagtactatgagaggaaagcagcggtgcctgaggtgggagtgatagctagataaattattagtgaaccaaggttttcagatgtaatgtttgtctactcaaaaagggaatcaattctctaagaatttaaacccatataatactatgaatgttaaaccagttgggtttatggtattaatgatttaatacagcaacactgacaaaatctctttttctccctttagttccagttagtaaatatcatattttttactatttcagatgccccgtctctgactgccaaggtggtaaattatcacattttactgtatattcaaatgttaattactaaatggcataattttaacaattaaatttactattttgtgagccaactaacaacaatgatgttaagcataggatgcccaatataataataagcaccttgtatgtacagaagattttacatgcacaaattcatttaatccacacaaatccgaagtttcaggtctgttttacaaattttgtatgcccaatgcttagagagatcaagtaatttgataatactcttatagggaggatccaaacccatggttttatgtatatttgtttgtttgttttttgcttttactttagaacagtttctatacattgtacaggttggattgggacagaaaataaaatttagaagctgtttttattgtcaacaatcttataattaaatatatctgcaaaatataaatataaaacaagtagtctttattatctggaaataaatgtgaagagcatatttaagcaagtgatgaataaattagaaaatatgcaggcagtaaaaaatggttaatatcagtcatgaatttaattttctaatttatttttgaaagccataatcttcataaaataattctagaatttttttcttcagccaatcaacagagatcatccttttaatttgaaacttgtttttatttttaagatccctttgttgatctagcaagtggccaagttcctttgcctcctgctagaaggttaccaggtgaaaatgtcaaaactaacagaacatcacaggactatgatcagcttccttcatgttcaggtgagttggctagtaacagtaacaataataccttattgttatatagtgctttttgcttttcagaacaccttcttattcattatataatttaatgaatgttgtccacccaagaaagtattttgatcatcattgcattttgcttataaagctggcattgtgagaggaatggctacagaactgcctcattgaaaagaaaaatattaagcttaatattatctttgatcactcagtggagccacttctttgattcaaaatgtattcattatctaactttcattttgtctcagatttaggaaatgaggaaaatctctttgtacattgcaatgctgttttatagcaatctctatggaattaatactggcaccaaactattcattttcttagcttatttagggctccaaccagtgctgcactgacagccatcaaatagaaggatgtaccatttttgtaaatacaagtcatactcacaaccgttaataatggaatgctgcttctttgcttcttgaggaggctttccctgaacatatctaaatagctcctatcttctgcagttattttctttccttttttgctttatttttcttcatagcacccgtcactacttaaaattttgttattcagaattcatttggttgattgttggttatctgtctcgtctgctaaagtattaacactttggagataagaatgttgtctggttcactgcttgtcctagtcagttcaggctactataacacaataccatagactggattgcctaaacagcaaacatttatatctcacagttctggaagctgggaagtccaagatcaagctaccaacattcagtgtctagtgagggccctcttccaggttcatagataactgccttcatactgtgtcctcacaagggcaagggagtgctctgggatctgtttcataaggaaactaatccctttcatgagggcttcccctgcataatctaatcatctcccaaaagcctcccctccaaatatcattacaaaagggattaggttttaacatatggatttgcagggaaaaggggacagacattcagtccatagcattgctttatcctcaagtgtccaacatagtgcccagcatatgtctctttgatggatagttggggatggatggatggatggatggatggatggatggatggatttgagtggacaaataggcaaaaggagggaagaatagctgatgtctgtattaaactaaatgcaaatatctgataagcaatatttttgtcacatatttgtgaatatcagaagttttcctatatgaacttttgtcttatttcaaaatttttttaagctaaaacctcagttcatagaagacttgctaatactattagcaatacattctgaaacttctgaatcaagataattaatgagctgttttaagaattgtcagtacggtagttcttacgtgctcgatgccatgcatgggagatgcagaaatccctgtgtatttatatgtttaactgcttgcatgtaactcaggaaagcatagaaaatctttttaatcttttcaaattttggccagtgttctcttatcagtttatttctagtcattctaaggtttcatatgtatccatgagtagcctgtaattccagtcaaaataatttgatgacaaaacatgaaagtcaagaaagaaagacaatatctgacagcttctatataatataaggaagcaattttttttaaagaaaatagtattaaaatataactttgagcaaagtgctatgactgatttaaaataacatattcagtaccaccagctctggattaaaagaaacctatactttagttaggggaaaacttgtagccaaaacacagtgttttcacagcttctttacagttttctccataataagtgggtgttgtagagcccagaggaacatcattcacatatatgatttcaagcagtatgaatccctattagcattacactagaaatgtctggaagaacttcaaatcacaaaccttgttgaaatatccccgaatagccatttgccagaagcctgaactggcttttacctaagtaataaaagactcctgactattactctgagggcccagctagctgtaacatcaaatatttcaatctactccctaaattctctttacaaacacagtcaaccacagaaaataagatacttttttccaacatatgtttgttcacatgaagttcacaggcctaccaggctgttcactagtttattttacatcagcagagagactgagcttgccttctagatgaggtctttccattcaacccttgagactgaatgcatatgcgcctttaggattctaatagctcttattttgcagtaattaatctgtttttattaaggcctttttaagatgaccaaatggggttctggtatgattgctctgccttatgaattcaagttctgttatcacttatggctgtctaaataattgatagtcatttatgaataggcttctatggaaagtatatttctaaagaagtttaaaatgtagtttcattgattttaaaatatctcccttgttttcttatctaaaaagtatgtctgcatgtatatgtatgtctacatatataaagagccttctaaatgtttattcctcccaaagaaaatattttatgcttttcttaaagtttttctaaatgtataattgaaatttaaatatatgaacgtatcataggtagtccagatataagaagagcttaccagcccatgttaaattcacatatatcggtatataggacaaacatttttgctagtaatctgcttgtttctttacatggatgtattattcacatggggcacatgtctcttctatctatgtgtgaggttttacaaatttctcagtttgcttttctcttgttgtaagacatattgtactacagtttgaataccattctttgaaaactaaaaggctgtattaatgaagtgcaccattcactcttgaggtgatttttcatattgttagtatttagaaggaagttcaaggtaacacttgaaaacaatgcttgattttagcacaactcaaatgtgtttgaaaccattaaaagaccttattgccaaataagccaatctcattataaaataaaggagatctcattacaaaatgaattaatttcaatactaccattagtttactagcttaaaggagaattgttggaatgttgaatacattttatattaggaccaagtattcaatagtttatctctgttaagatctcaattggaaatcgtggcttcacttcacgcaagggttggaattttaaggtagattatgtgtctttaagattttgtgtttcaacaaaggaaatggcaatgttgagttttcccacagtttagctatgtgaaatactggggcttacagacagcttcagtttgaaagccagagaacctagagttgttttccttacaaatctaagattaaataaccattttctggatatatttaaaacagtgtatgtgaagatttttttatttgtaatttagtttattaaatcatgtccttctataggcagtatttctttttttttgagatggagtctcgctctgtcgcccaggctggagtgcagtggcgtgatctcagctcccagcaatttccacctcccaggttcaagtagttctcatgcctcagcttcccgagtagctgggattacaggcatccaccatcatgcccggctaatttttgtatttttagtagagatggggtttcaccatgttggccaggctggtctcgaactcctgaccttgtgatctgcccacctcagcctcctaaagtgctgggattacaggcttgagccactgcgcgtggccagcagtatttctatagaggcagtaattcaaccttatggcataaaaatgaatcccagaattaattgatccaacttctagtagaataacaaagttaatcataatgtgtttgagatttcttttattccttgatcaattaagtgtatttatatttttttctaattctgttattgttatagttgagaaggtcagtagtctaagagttttgatatctgtcccaagaattgtcactggtttactaagtaaatttagacagtatgtttcagataatatatacaatattattttgtctaattaaaattcgttaagtttgttaagcatttactatagtcctgatgctttgctaagtattttagagacagatatataattttttttttttttgagacgagtctcactctgttgcccaggctagagtgcagtggcacgatcttggctcactgcaacctccacctcctgggtttaagcagttctgcctcagcctcctgagtagctgggactacaggcgcccaccaccatgcccagctaatttttacatttttagtagagacggggtttcaccatattggccaggctgatctcaaactcttgaccttgtgatccgcccacctcggcctcccaaagtgctgggattacaggcatgagtcaccacgcccagccagatacagatatataaattttatataagttaatattatatataattgtatttatatagttatgcagtaaaatataaatattcatgttattttatttaataatgtatattttatattatctgtttataattatacatttatatatttgatattgttatacaaatatatggttatataatatggtatagatataaatataaatttatttttactgtgtaatatagaaatagatataattttatttctcctaaccactctgaaatatatatatgtgtgtgtgtgtgtatatatatgtgtgtgtgtgtgtatatatatatatatatatatatatatttatttatttatttatttatttatttttttggagacagagtctcgctctgtcacccagcaggctggagtgcagtggtgcaatcttgtctcactgcaacctccacgttccgggttcaagcaattctcctgtttcagcctcccaagtagctgggactataggcagtttttatatttttagtatagacagggtttcaccatattggtcaggctggtcttgaattcctgacctcaggtgatccacctgcctcagcctctcaaactgctaggattataggcgtgagccactgcgcctggcctgaaatagatactattaatgtcaatttatagaagaaagaaagacataacaaagtttagtaactaactcaagctgctgcagaaagtgtaagcctgatagaatttacatcagtttcagttttttaaaaatttgttaataaagtttatgttgttatcccatcatcatgccagaattaagttagaaaaaaatgtacacatgcatctcacgtctccagactgagtttggagatgttcagtggtaaaatcatgataatagtgatggaatagtagtgatgacaaaataatgaagggttttagaaatagtaaaactgtactagaaatttcaaaatctagattctgattatttttcacatgaccttaggaaagccactcactatgtacttcctatatttttcacatgagcgaaatcgatctctggttctcagtcttagatacttgtgcagctgaggcctttaagagtatcagaggagatctgagactattttgttgttggtggtggtttttctaattatgcgcatattatacacgcattggaaaataaaaggaaattacagagaaaacaattaataagaaaaagcaatcacttaaaaccctatcttcaataaggaacattattgccattttcaagtgcagccttctaaatgttttgtctatgcacattttcacttacgatcttaagatcatactcaatatgtcaattctatctcagttccatttaggagtattataagtattttaccacattattttaaaaaataaatgtcattttaaattattgtatgacaatccattatttttatgtactatcaaatactatttttaacattttgaaagtctaacagaaaccctaactttatatactagaaaactttcacattaaatccctttgctgtgacctagatatacagaagctcagtgttatatttattgttgaacatgatccagtataatagaaaatgggaaaatgtattcatcaatacaatagcatttgcttattagacacatctttcagaatatagaaaaagaaaacccaacccaatctttagatgatgaggggagtaataaagactttttgtggcaaaataattgacagccactgaaatgaaatatcctcagttctttaaatggaggttatgtagatatacagcagtgcgtctcacttaaaaaaaaaaaatatggtctgcctacgggccttttggacactttttttttttttcctaataagctttctgtccccagcaataatgaaagtttaaactacagatgtgcttagtagacatagagatagaggtatatctctatgtaccatggctctttggaaggccacaaaccattttaccatctaaaattgttttctttcatcccacaagaaccaatgattgcccccgtctagaatgtatgacctagagtaatcattctgggtattattccaaccatgttaggctaattcccatgcaatccaggaatgttctgtttgtcactctgttggaggctgaaagaatgagggtcgtgatcaactcactataccactggaggctatatgagtgaacagcaaactgttctcataaaagcagaatgttgacaaactgacaaactgcatctgctgcccaggaggaatgctgagggcagtcacgacccaggcacaagtgttgcttttgattaggcacatctgaagcctgttaacaataatgtgaacctgtgatcagtcaagcagctgaccaatcgttacctcctcatccctgctctttctacccaataaatacaaagggctatggaagctcagggctgctgcctttgctcacttcttcttcctcagaccctttccttaaaacagtttcttttgttttttgttatcatttctaggttcttcccttagttcagtcttgtaatgatggtctcaggtagtaacagaagtaactgttgtaatgactgtctcaaatagtaacagcagtaactgtgatagtgaaggtctcaagtagtaacagcagtaactgtgatagtgaaggtctcaagtagtaacagcagtaactgtcatggtgaaggtctcaggtagtaacagcagtaactgtgatagtgaaggtctcaggtagtaacagtagtaactctcgtagtgacggtctcaagtagtaacagcagtaactgttgtagtgaaggtctcaagtagtaacagtagtaactctcctaatgacagtctcaagtggtaacagtattatagtaactgtagtattgatggtctcaagtagtagcagtggcggtcggccacatcactctcttcttttatatgctcagaaaaggaaagctgaatcaaaatttactcctaaaccagggcttctcaaatctttctttctcaaagaagagttgacagtgaacaacacaagggctgggacattaggtaaaagaggacaatatttagttttttagtgcaagacattgtttctattttgtctcaaaaatttgtgcaaactttatattttactccttagtgtatctgtattcatttcagtgctttaaaatattaatgaccttatatcttttaagaaaattggtaacccagaagatgtactatttatattctgtggctcacattcttgtacttattttgataccactgcaattactcataagagttcaaaaataccatgaacatggatggtagcactagagcaaacacatgcttttccaaaataactatttttaaggatgatgtatattcaggtacagaaattccacagggttgataaaatttatagttatttcagtgtgctcccaaactgcaatataataatcttaggggaccagaagatagtttcatcttcatttcattgcttccttccactaccctacacattcgaaagtgctaaagtaaatgaggacctggtgtaatccacttttaggtactgtgaaaaagaaaagtggagaaagtgaaggctgcgtctgggctttgggaagagacagaaagagtaatcggttctaagcaatttgctctaatggtcagtgtggaaatgatctcctcctccccactttttaacagcttccactagtagttcaggttgcaccagatcttgctttttaatattctgctagaaggctttctgtagaatcagatactcacaacaaattttgtgttctttgttctttcacacttctctggcattcctgttatagtaattgctggtttatagcatttgagtatagcacttatcattataaatattaattcattgttctttctttttaatggtctcctcagtccatatactgtatattacattaattgttatactacctggttgaaagggataaataatacctctaaacacagatatataaagtagatttggagaatgagaattgtgaaagtaaattgcttagcccaagatcttagcggccaaactgagatttaagattcaattgccagttataccatttattagaggcattcgtcatagtattcatcccacttgtatacacagctgttcttttcaaacatgcctggcattcatgtgcacatctcagtatcctggacagacaaaataacaatattaaaaatctcttagaaaattacccgtagaaaacaagttaattgttttctaagtctaggtacttttgtatcaaagtctaggtacttttctcatctaaaagtgtctcagaacagacatgatttaaatgtatgaattttttggtaattgattaaaggaaagctttcaataatttctaatagttttgaaaatgattaaaagcaaaacataatttattgtccattcctgtatgacataatgaaaataataggaaaataaatggaactacaaaacatactaagaaacgcagtttatcagacgataatagtttgagatgtttaattacagatgcaagaaacagtacagatattagcaagaaccagaaattatatgttcttatatatgggcatactttaatatacattgtctacagctataatattttctaaaactggacattttgttacaaaaaaggaataatttaaaataagactgtcatcatggccggacgtggtggctcacgcctgtaattttagcattttgggaggccaagatggaaggattgcctgaggacaggagttcgagaccagcctggtcaacataatgagacttcatctcatttaaaaaaaaaaaaaaaagaagaagacatcctatttgatgtggaaacaaatgcttttatcctagaggctttgttttatgtatgttatattgatattgctataattgtatataggatgatgtttttacaagttaccttctcctggaaaaatatcatacatcttttgatttagaaaggaaaaatattgaataatacatcagattagcttttattatttacgtttattattattgaaaatatcagcttacagtatctcctgaagccattgggaaagttgctgaggcagcaggagtggggagacaagatgggaacattaagcatgagacgtttggcttttaacctcacatcaccattcacttaactctgtccttgaatttattgtttggcatttgtcagtggtctgagagaaaggtattaaatattgttgtctagatagtcttaaatccataagtgtatttcatgacataaagatgacattttaaaatgtatatatgggccaggtgcggtggctcactcctgtaatcccagcactttgggaggccaaggtgggtggatcacttgaggtcaggagttcaagaccagcctgatcaacatggtgaaaccctgtctccactgggaaaaaaaaaaaaaattagccaggcatggtggtgcacgcctgtaatcccagctacttgggaagctgaggcggtagtagaattgcttgaactcgggaggtggaggttgcagtgagctgagactacgccattgcactccagcctgggcaacaagagtaaaactccatctcatgtatacgtatagtgtgtgtatgtgtgtattttttgtctaggcttttgagataaagtgctatacataaccaggaaatacatgatgaaagtgccctgtcaagttagctagcccatgacctgggaccttttcttttttcttaaggttactctccattcatgggtgccaatgtcattggtttaaattggaggctgtattatcaccaagggactaatctagaacccaattctctactcctaatatttcttctaggtttgctttttcttgagcaaaacttcctacattgcaatctgtataaagtatttgagcaaagcactgagtttccttccgttcagcctgttgactaacagtagagtcaaagcatctactattttgttttttttttctcctagcatcagtaaggcacagattattaacttgaaaaatcagctaggaaaatgctacagagcatctgttttagagcagttctgctgtgacatggatgggactcaggacctgtggggacgttctgccatggctgtttgtgagcttgtgaacaaagtttcctctcatagaaacctcccaagattgttcctctggagaaccatcaaccagagcctgtgctgtagtagagtgtgaactgtattgctaaacaaagtgaaaggcttccctttttagatagttattttaattattttttcctttctctatctctctgaccacatatggcttgaagaataaaatgtggaatagagaaggagatgtgtaaatttctcagtggaactaccaacacactgtaccttaattttctgttgttcaaaatgaggataatactacctatctcacaatagtatggaaaaattgaaaaaaagtttatttgtaaagaaaacaagaaataatatttccataggaaatgagataatgctgttatgttatcttgccttcatttttatttcactgtttaaatagtatctctctttaaaaatctacacagatggttcacaggcaccagccagaccccctaaaccacgaccgcgcaggactgcaccagaaattcaccacagaaaaccccatgggcctgaggcggcattggaaaatgtcgatgcaaaaattgcaaaactcatgggagagggttatgcctttgaagaggtgaagagagccttagagatagcccagaataatgtcgaagttgcccggagcatcctccgagaatttgccttccctcctccagtatccccacgtctaaatctatagcagccagaactgtagacaccaaaatggaaagcaatcgatgtattccaagagtgtggaaataaagagaactgagatggaattcaagagagaagtgtctcctcctcgtgtagcagcttgagaagaggcttgggagtgcagcttctcaaaggagaccgatgcttgctcaggatgtcgacagctgtggcttccttgtttttgctagccatatttttaaatcagggttgaactgacaaaaataatttaaagacgtttacttcccttgaactttgaacctgtgaaatgctttaccttgtttacagtttggcaaagttgcagtttgttcttgtttttagtttagttttgttttggtgttttgatacctgtactgtgttcttcacagaccctttgtagcgtggtcaggtctgctgtaacatttcccaccaactctcttgctgtccacatcaacagctaaatcatttattcatatggatctctaccatccccatgccttgcccaggtccagttccatttctctcattcacaagatgctttgaaggttctgattttcaactgatcaaactaatgcaaaaaaaaaaaagtatgtattcttcactactgagtttcttctttggaaaccatcactattgagagatgggaaaaacctgaatgtataaagcatttatttgtcaataaactgccttttgtaaggggttttcacataacatagaggagcttcccttttttgtttaagttttgtaacctttaatcctccatattctcatgtctgtcatcccaggggtgtcacaactgtaaaaatctacaatattagaaagcagctactacagatgtggaagagagaacacttgtataagacaatgctgtactgaagttatataacaaggtcccctgatacttatgcctgcattactttgagggatgctgaatgagaggaccatctccctgaaatatattaataattttcagataaaattatgaacataacataccttttctcaagtggaattagagttcatttcccatttaagtgattacactctatctcctaagagctgtgggaaccagttcatattgcagtggaaacattaactcttcgttgcactgttgagtgtcaaggctactgtggcaatgttttttgcaaagttctcagtaattttctctctgcaatcaaattagagttcatatttaatttgcagattctctcattcattgctccacactgctttatcaaatcaggtgaaagaaattaatgtagttttgcctattttacaaaatggtggttggttctttaaacaccatgttattactcttaataaatctacaaatatttattgagcatctatgatatgcttggtgctgttaggcactgtgtttttaaagcacaaactctcaaaagctgattccagcgtaatgttttatttttgtttttctgtgttttttatgacaaagtctgtctgatagacaaagcctggaaggtttcccccagggctttagaatttagaattagctggttttcatttcaaatgggaaattaatggagaaatgagataaaatcatctctgtgtaaatgcttgaacccacagaaagttttctaaatatggactaattgtgggtttgtgcatatttctgtgtctcctcttttcacttcacacccaccatgatataaaaagatgcatcagagagtgagcctgccttcaggacctgaaaggacatgactgaacatctagtagatctggtggaaattaatgttatacaaaagacacgtagactaagttgaccctgggagaggagttaatacattcctttgcaaagtgacatgtgccctttttttgacagtctggctttcatggtatgagatgaggactcacaggccttgggtaatttctgcttttgtatgaattttttcagatggttctgagctggttatgtccctggaggggaatttttcatttgccttagatgttcttcccatgagtaggtacatgagtgaagtttcttacacttttaaaattgcattggcaaattttcttttgctttatgaacccctgttttgtcattatgatttttcacttgttttaacagtttcagatgtcaccttggaatagcactgtttattttggcctttttatgattgctgatgataggtacagtctaaaggccaaaagttgccacttcattgtgataacattgttgcaacattcctaatatattagagaatgatgtaaaataagaatgttagagaacatagcacattctcattcagtttataaacaatatttgaaaatttccagtgagaattaaaaaaggactagtgatgatatagcaaacctggacctgaaatttgggggtggggggtggggggatatctgttttgcagagaaaatttaccagcctccagcaaaatctctgcatcttgtactcaatagcaaccattagtattgtcatttaaaaagaatgaatcaaaaattcatacgtaatggaatgaaaagaggaaaactttctattaaacactgtcaagaaagacccattgctatctatcacctatgaaattaaacctaaaaaaatttttttttattgtaaaagcgactgctcgttgatgtctggaagtgatccagactttgtttcactgtgatccaaaaagttagacattccttccatttctcaagatttggtaacaatttttttttaagtcagcatttcatataatacataaaattaagacaaattaacttactgatatgtggccaatatcaacccttctcacccaacccaaacatgttctggagatagaaggacttatcctcaattatattccagtaaatttatctccataatctgttttatcccctaatgtatgatatatctcaccaaatttttctagaattgaataaaagcagtttttgcttaacttgaatttccttccacaaatacaaccttcagctcttccctctctgtttcccttttgcccacattgcaaatagtcttcataatagaaccctcactgcaacttaaaagggtcccatttcttaactcagcatttcatataccaagaacacttgcaaagatttcttcataatgtgattttttaatttttcagttccagaagcatttcctacagttcgaacgttattttttctctcagattttcaacttgaagctttgatttgcactgaattaacttggataaaattttagtttctctcttcagctaacagatttggaagcaaattacagtatacttgaataattcaggtactacctgttgacctggaattttagtgattctgggaaagttttccagacaaagacaccaatagagtgaagggttttttccccaattactattttattgttttttaaaatgcctttttaaaacttgtgtgatgtggcaaaatagtcatatgttcaattaatttatattttattcattccttttcaattctggttattatgtaacctcaagtactttatctgttcttttaaagtattttataaaatgaacgttaactaaa

SEQ ID NO: 4 - protein, 982 aa, Homo sapiens [sequence omitted]

SEQ ID NO: 5 - protein, 1004 aa, Homo sapiens
MALAPGPDAHAHLPPLIELKFQMANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGDVFDSASDPVPLPPARPPTRDNPKHGSSLNRTPSDYDLLIPPLGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 6 - protein, 955 aa, Homo sapiens
MALAPGPDAHAHLPPLIELKFQMANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGDVFDSASDPVPLPPARPPTRDNPKHGSSLNRTPSDYDLLIPPLGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 7 - protein, 960 aa, Homo sapiens
MALAPGPDAHAHLPPLIELKFQMANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 8 - protein, 933 aa, Homo sapiens
MANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGDVFDSASDPVPLPPARPPTRDNPKHGSSLNRTPSDYDLLIPPLGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 9 - protein, 933 aa, Homo sapiens; identical to SEQ ID NO: 8

SEQ ID NO: 10 - protein, 722 aa, Homo sapiens
MAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGDVFDSASDPVPLPPARPPTRDNPKHGSSLNRTPSDYDLLIPPLGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 11 - protein, 889 aa, Homo sapiens
MANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKSCDNGHCMLNGTHGPSSEKKSNIPDLSIYLKGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 12 - protein, 933 aa, Homo sapiens
MANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKSCDNGHCMLNGTHGPSSEKKSNIPDLSIYLKGDVFDSASDPVPLPPARPPTRDNPKHGSSLNRTPSDYDLLIPPLGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 13 - protein, 938 aa, Homo sapiens
MANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 14 - protein, 982 aa, Homo sapiens [sequence omitted]

SEQ ID NO: 15 - protein, 678 aa, Homo sapiens
MAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 16 - protein, 542 aa, Homo sapiens
MKYSKESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 17 - protein, 889 aa, Homo sapiens
MANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 18 - protein, 629 aa, Homo sapiens
MAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKSCDNGHCMLNGTHGPSSEKKSNIPDLSIYLKGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 19 - protein, 673 aa, Homo sapiens
MAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKSCDNGHCMLNGTHGPSSEKKSNIPDLSIYLKGDVFDSASDPVPLPPARPPTRDNPKHGSSLNRTPSDYDLLIPPLGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 20 - protein, 889 aa, Homo sapiens; identical to SEQ ID NO: 11

SEQ ID NO: 21 - protein, 933 aa, Homo sapiens; identical to SEQ ID NO: 12

SEQ ID NO: 22 - protein, 938 aa, Homo sapiens; identical to SEQ ID NO: 13

SEQ ID NO: 23 - protein, 982 aa, Homo sapiens [sequence omitted]

SEQ ID NO: 24 - protein, 539 aa, Homo sapiens
MPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGDVFDSASDPVPLPPARPPTRDNPKHGSSLNRTPSDYDLLIPPLGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 25 - protein, 722 aa, Homo sapiens; identical to SEQ ID NO: 10

SEQ ID NO: 26 - protein, 917 aa, Homo sapiens
MGYLCVNFIWFLGITTHRVDLKKELKFQMANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 27 - protein, 961 aa, Homo sapiens
MGYLCVNFIWFLGITTHRVDLKKELKFQMANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGDVFDSASDPVPLPPARPPTRDNPKHGSSLNRTPSDYDLLIPPLGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 28 - protein, 966 aa, Homo sapiens
MGYLCVNFIWFLGITTHRVDLKKELKFQMANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 29 - protein, 1010 aa, Homo sapiens
MGYLCVNFIWFLGITTHRVDLKKELKFQMANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGDVFDSASDPVPLPPARPPTRDNPKHGSSLNRTPSDYDLLIPPLGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 30 - protein, 961 aa, Homo sapiens
MGYLCVNFIWFLGITTHRVDLKKELKFQMANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKSCDNGHCMLNGTHGPSSEKKSNIPDLSIYLKGDVFDSASDPVPLPPARPPTRDNPKHGSSLNRTPSDYDLLIPPLGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 31 - protein, 673 aa, Homo sapiens; identical to SEQ ID NO: 19

SEQ ID NO: 32 - protein, 894 aa, Homo sapiens
MGYLCVNFIWFLGITTHRVDLKKELKFQMANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGDVFDSASDPVPLPPARPPTRDNPKHGSSLNRTPSDYDLLIPPLGEDAFDALPPSLPPPPPPARHSLIEHSKPPGSSSRPSSGQDLFLLPSESPVYPMADK

(19) United States
(12) Patent Application Publication
(10) Pub. No.: US 2024/0299542 A1
(43) Pub. Date: Sep. 12, 2024

(54) NATURAL KILLER CELLS ENGINEERED TO REDUCE OR ELIMINATE CBL-B AND USES THEREOF

(71) Applicant: City of Hope, Duarte, CA (US)

(72) Inventors: Jianhua Yu, Duarte, CA (US); Michael A. Caligiuri, Pasadena, CA (US)

(21) Appl. No.: 18/271,026

(22) PCT Filed: Jan. 5, 2022
(86) PCT No.: PCT/US2022/011319

§ 371 Date: Jul. 5, 2023

Related U.S. Application Data
(60) Provisional application No. 63/134,152, filed on Jan. 5, 2021.

(51) Int. Cl. A61K 39/00; C12N 5/0783; C12N 15/113

(52) U.S. Cl. CPC A61K 39/4613; A61K 39/464499; C12N 5/0646; C12N 15/1137; C12Y 203/02; A61K 2239/48; C12N 2310/14; C12N 2510/00

(57) ABSTRACT

Described herein are compositions comprising human natural killer (NK) cells engineered to reduce or eliminate Cbl-b and with or without a chimeric antigen receptor (CAR), methods of making such compositions, and methods of using such compositions (e.g., killing cancer cells, treating a subject having cancer or viral infection).

### CLAIM OF PRIORITY

This application claims the benefit of U.S. Provisional Application Ser. No. 63/134,152, filed on Jan. 5, 2021. The entire contents of the foregoing are incorporated herein by reference.

### TECHNICAL FIELD

Human natural killer (NK) cells lacking expression of Cbl-b and human NK cells expressing a chimeric antigen receptor (CAR) lacking expression of Cbl-b are described.

### BACKGROUND

Natural Killer (NK) cells are part of the innate immune system and act as the first line of defense-against infectious pathogens and tumors through cytotoxicity and cytokine production (1-3). NK cells were first described for their ability to spontaneously lyse target cells without any prior priming or restriction to target cells expressing major histocompatibility complex (MHC) molecules (4, 5). The genesis, survival, proliferation and activation of NK cells are regulated in large part by IL-15, which binds the IL15Rβγ expressed on NK cells or its precursors resulting in activation of JAK3 and the subsequent the phosphorylation of STAT5 (6). NK cells express both activating and inhibitory receptors that receive their signals by engaging with target cell ligands, which then activate or inhibit NK cell function (7, 8). NK cells themselves can express non-MHC class I specific inhibitory receptors or “checkpoints” including programmed death-1 (PD-1), T cell immunoreceptor with Ig and ITIM domains (TIGIT), lymphocyte activation gene 3 protein (LAG3) and T cell immunoglobulin domain and mucin domain-3 (TIM-3) that can suppress NK cell function, and may represent new targets for checkpoint blockade-based NK cell immunotherapy (9).

The Casitas B-cell lymphoma (Cbl) protein family, which includes Cbl-b and c-Cbl in mammals, represents RING-finger domain-containing E3 ubiquitin ligases and provides critical inhibitory signaling for the proper regulation of protein tyrosine kinases (PTKs) (10, 11). Cbl-b also regulates CD28-dependent T cell activation by selectively suppressing TCR-mediated Vav activation (12). Additionally, Cbl-b regulates peripheral T cell tolerance, and the loss of Cbl-b results in the onset of autoimmunity (13). The Cbl family plays an important role in the induction of B-cell immune tolerance (14). Deleting Cbls in germinal center (GC) B cells abolishes antibody affinity maturation via the early exit of high-affinity antigen specific B cells from the GC (15).

In spite of the advantageous properties of NK cells in killing tumor cells and virally infected cells, they remain difficult to work with and to apply in immunotherapy, primarily due to the difficulty in obtaining sufficient numbers of activated NK cells for adoptive transfer. Further, it may be challenging to maintain their tumor-targeting and tumoricidal capabilities during culture and expansion. Thus, there is a need for NK cells with improved functions, such that they may be effective in targeting and eliminating tumor cells and virally infected cells when used in vivo.

### SUMMARY

Described herein are compositions comprising human natural killer (NK) cells that do not express Cbl-b (Cbl-b^{neg} NK cells) or in which Cbl-b expression has been suppressed or reduced (Cbl-b^{low} NK cells). In various embodiments: the Cbl-b^{neg} or Cbl-b^{low} NK cells express a chimeric antigen receptor; chimeric antigen receptor is targeted to a cancer antigen; and the Cbl-b^{neg} or Cbl-b^{low} NK cells are activated. In some embodiments, the Cbl-b^{neg} NK cells or Cbl-b^{low} NK cells are genetically modified. In some embodiments, the genetic modification is deletion of all or a portion of the Cbl-b gene or an insertion into the Clb-b gene. In some embodiments, the genetically modified cells express Cbl-b at a level that is 50%, 40%, 30%, 20% or 10% or less than cells that are not so genetically modified. Also described are methods for preparing activated Cbl-b^{neg} or Cbl-b^{low} NK cells, the method comprising culturing Cbl-b^{neg} or Cbl-b^{low} NK cells on feeder cells. In various embodiments: the feeder cells are K562 cells; the Cbl-b^{neg} NK cells are cultured in the presence of IL-15 or IL-2 or both IL-15 and IL-2; the K562 cells express membrane bound interleukin 21 (IL-21) and 4-1 BB ligand (4-1BBL); the ratio of human NK cells to K562 cells is in a range of about 0.1:1 to about 10:1; the NK cells and the K562 cells are co-cultured for a duration of about 5 min to about 6 weeks; the IL-2 is present at a concentration of about 10 IU/ml to about 5000 IU/ml; the IL-2 is present at a concentration of about 50 IU/ml to about 2000 IU/ml; the IL-2 is present at a concentration of about 150 IU/ml to about 900 IU/ml. Also described is composition comprising the population of expanded NK cells produced by any of the forgoing claims.

The gene encoding E3 ubiquitin-protein ligase (Cbl-b) in humans, CBLB, is located on chromosome 3 at 3q13.11. The CBLB gene (GenBank Gene ID: 868) can comprise SEQ ID NO:3. Human CBLB encodes Cbl-b, which can comprise the amino acid sequence of any of GenBank Accession ID XP_011511559.1 (SEQ ID NO:4), XP_016862884.1 (SEQ ID NO:5), XP_016862887.1 (SEQ ID NO:6), NP_001308719.1 (SEQ ID NO:7), XP_016862888.1 (SEQ ID NO:8), NP_001308725.1 (SEQ ID NO:9), NP_001308735.1 (SEQ ID NO:10), NP_001308726.1 (SEQ ID NO:11), NP_001308723.1 (SEQ ID NO:12), NP_001308720.1 (SEQ ID NO:13), NP_001308717.1 (SEQ ID NO:14), NP_001308737.1 (SEQ ID NO:15), NP_001308749.1 (SEQ ID NO:16), NP_001308728.1 (SEQ ID NO:17), NP_001308745.1 (SEQ ID NO:18), NP_001308740.1 (SEQ ID NO:19), NP_001308727.1 (SEQ ID NO:20), NP_001308724.1 (SEQ ID NO:21), NP_001308722.1 (SEQ ID NO:22), NP_733762.2 (SEQ ID NO:23), NP_001308751.1 (SEQ ID NO:24), NP_001308736.1 (SEQ ID NO:25), XP_016862889.1 (SEQ ID NO:26), XP_016862886.1 (SEQ ID NO:27), XP_016862885.1 (SEQ ID NO:28), NP_001308715.1 (SEQ ID NO:29), NP_001308718.1 (SEQ ID NO:30), NP_001308742.1 (SEQ ID NO:31), or XP_011511561.2 (SEQ ID NO:32). In some embodiments, Cbl-b comprises the amino acid sequence of SEQ ID NO:29,

| Cbl-b (GENBANK ACCESSION NP_001308715) | |
|---|---|
| SEQ ID NO: 29 | |
| 1 | mgylcvnfiw flgitthrvd lkkelkfqma nsmngrnpgg rggnprkgri lgiidaiqda |
| 61 | vgppkqaaad rrtvektwkl mdkvvrlcqn pklqlknspp yildilpdty qhlrlilsky |
| 121 | ddnqklaqls eneyfkiyid slmkkskrai rlfkegkerm yeeqsqdrrn ltklslifsh |
| 181 | mlaeikaifp ngqfqgdnfr itkadaaefw rkffgdktiv pwkvfrqclh evhqissgle |
| 241 | amalkstidl tcndyisvfe fdiftrlfqp wgsilrnwnf lavthpgyma fltydevkar |
| 301 | lqkystkpgs yifrlsctrl gqwaigyvtg dgnilqtiph nkplfqalid gsregfylyp |
| 361 | dgrsynpdlt glceptphdh ikvtqeqyel ycemgstfql ckicaendkd vkiepcghlm |
| 421 | ctscltawqe sdgqgcpfcr ceikgtepii vdpfdprdeg srccsiidpf gmpmldlddd |
| 481 | ddreeslmmn rlanvrkctd rqnspvtspg ssplaqrrkp qpdplqiphl slppvpprld |
| 541 | liqkgivrsp cgsptgspks spcmvrkqdk plpapppplr dpppppperp ppippdnrls |
| 601 | rhihhvesvp srdppmplea wcprdvfgtn qlvgcrllge gspkpgitas snvngrhsrv |
| 661 | gsdpvlmrkh rrhdlplega kvfsnghlgs eeydvpprls ppppvttllp sikctgplan |
| 721 | slsektrdpv eedddeykip sshpvslnsq pshchnvkpp vrscdnghcm lngthgpsse |
| 781 | kksnipdlsi ylkgdvfdsa sdpvplppar pptrdnpkhg sslnrtpsdy dllipplged |
| 841 | afdalppslp ppppparhsl iehskppgss srpssgqdlf llpsdpfvdl asgqvplppa |
| 901 | rrlpgenvkt nrtsqdydql pscsdgsqap arppkprprr tapeihhrkp hgpeaalenv |
| 961 | dakiaklmge gyafeevkra leiaqnnvev arsilrefaf pppvsprlnl |

In some embodiments, any of the NK cells or CAR NK cells of the disclosure are genetically modified. In some embodiments, one or more genes are knocked out or down regulated. In some embodiments, the one or more genes comprise a gene encoding Cbl-b, e.g., CBLB. In some embodiments, Cbl-b is knocked out. In some embodiments, Cbl-b is down regulated. In some embodiments, genetic modification is achieved by methods described herein and those known in the art. In some embodiments, genetic modification methods comprise gene editing, homologous recombination, nonhomologous recombination, RNA-mediated genetic modification, DNA-mediated genetic modification, zinc finger nucleases, meganucleases, RALEN, TALEN, megaTAL, CRISPR/Cas technology (e.g., CRISPR/Cas9 gene editing; see, e.g., WO 2019/090202), or CRISPR/Cpf1 (briefly reviewed in Alok, et al. (2020) Front. Plant Sci., doi.org/10.3389/fpls.2020.00264).

In some embodiments, the NK cells or CAR NK cells of the disclosure comprise a nucleic acid to suppress or reduce the expression of a protein. In some embodiments, the nucleic acid is an inhibitory nucleic acid, such as an antisense oligonucleotide (ASO), e.g., locked nucleic acid (LNA), an shRNA, or an siRNA, that targets Cbl-b. Methods of designing and making inhibitory nucleic acids to target an endogenous target are well known in the art.

Also described herein are methods for preparing activated Cbl-b^{neg} NK cells or activated Cbl-b^{low} NK cells, a method comprising: obtaining a population NK cells, reducing or eliminating Cbl-b in the NK cells, and culturing the NK cells. In some embodiments, the step of reducing or eliminating Cbl-b expression comprises genetically modifying the NK cells; in some embodiments, the genetic modification is deletion of all or a portion of the Cbl-b gene or an insertion into the Clb-b gene; in some embodiments, the NK cells are obtained from peripheral blood, bone marrow, cord blood, induced pluripotent stem cells (iPSCs), cell lines, or cytokine stimulated peripheral blood; in some embodiments, the culturing comprises co-incubating the NK cells with feeder cells; in some embodiments, wherein the feeder cells are K562 cells; in some embodiments, the Cbl-b^{neg} NK cells or the Cbl-b^{low} NK cells are cultured in the presence of IL-15 or IL-2 or both IL-15 and IL-2; in some embodiments, the NK cells are cultured in the presence of presence of one or more cytokines, wherein the cytokines comprise thrombopoietin, SCF, Flt3 ligand, IL-7, IL-15, IL-2, IL-18, IL-21, and combinations thereof; in some embodiments, the K562 cells express membrane bound interleukin 21 (IL-21) and 4-1BB ligand (4-1BBL); in some embodiments, the ratio of human NK cells to feeder cells is in a range of about 0.1:1 to about 10:1; in some embodiments, the NK cells and the feeder cells are co-cultured for a duration of about 5 min to about 6 weeks; in some embodiments, the NK cells and the feeder cells are co-cultured for a duration of 5 min, 10 min, 15 min, 20 min, 25 min, 30 min, 35 min, 40 min, 45 min, 50 min, 55 min, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 8 days, 9 days, 10 days, 1 week, 2 weeks, 3 weeks, 4 weeks, 5 weeks, 6 weeks, or 7 weeks; in some embodiments, the IL-2 is present at a concentration of about 0 IU/ml to about 5000 IU/ml, about 50 IU/ml to about 2000 IU/ml, or about 150 IU/ml to about 900 IU/ml; in some embodiments, the IL-15 is present at a concentration of about 0.1 ng/mL to about 50 ng/mL, about 1 ng/mL to about 20 ng/mL, or about 5 ng/mL to about 10 ng/mL; in some embodiments, the expression Cbl-b is reduced or eliminated by targeting a gene encoding Cbl-b or an RNA product of the gene encoding Cbl-b; in some embodiments, the gene encoding Cbl-b is human or the gene comprises SEQ ID NO:3; in some embodiments, the RNA product of the gene encoding Cbl-b is the mRNA transcript of the gene; in some embodiments, the gene is modified, edited, or knocked out using gene editing, homologous recombination, nonhomologous recombination, RNA-mediated genetic modification, DNA-mediated genetic modification, a zinc finger nuclease, a meganuclease, RALEN, TALEN, megaTAL, CRISPR/CAS9, or CRISPR/Cpf1; in some embodiments, the reducing or eliminating Cbl-b comprises administering an inhibitory oligonucleotide to the NK cells; in some embodiments, the inhibitory oligonucleotide an antisense oligonucleotide (ASO), an shRNA, or an siRNA; in some embodiments, the inhibitory oligonucleotide is complementary to at least 8, 9, 10, 11, 12, 13, 14, 15, 16, 17, 18, 19, or 20 continuous nucleotides of a gene encoding Cbl-b, an RNA product of the gene encoding Cbl-b, SEQ ID NO:3, the complimentary sequence of SEQ ID NO:3, or a nucleic acid sequence encoding a cbl-b, e.g., a sequence encoding any of SEQ ID NOs:4-32; in some embodiments, the inhibitory oligonucleotide comprises about 5 nt to about 80 nt, about 6 nt to about 60 nt, about 8 to about 50 nts, about 10 nt to about 40 nts, about 12 nt to about 35 nt, about 15 nt to about 30 nt, or about 18 nt to about 26 nt; in some embodiments, the inhibitory oligonucleotide comprises SEQ ID NO:1. Also disclosed are compositions comprising the population of expanded Cbl-b^{neg} NK cells or expanded Cbl-b^{low} NK cells produced by any of the methods described herein.

Also disclosed herein are populations of human NK cells harboring a vector comprising a nucleic sequence expressing an siRNA targeted to Cbl-b. Also disclosed herein are populations of human NK cells, wherein the cells do not comprise a nucleic acid encoding Cbl-b. Also disclosed herein are populations of human NK cells, wherein the cells comprise a nucleic acid targeting Cbl-b. Also disclosed herein are populations of human NK cells comprising a nucleic acid comprising a sequence that is complementary to at least 8, 9, 10, 11, 12, 13, 14, 15, 16, 17, 18, 19, or 20 continuous nucleotides of a gene encoding Cbl-b, an RNA product of the gene encoding Cbl-b, SEQ ID NO:3, the complimentary sequence of SEQ ID NO:3, or a nucleic acid sequence encoding a cbl-b, e.g., a sequence encoding any of SEQ ID NOs:4-32; in some embodiments, the nucleic acid is an antisense oligonucleotide (ASO), a siRNA, a shRNA, a gRNA, or a crRNA; in some embodiments, the nucleic acid comprises about 5 nt to about 80 nt, about 6 nt to about 60 nt, about 8 to about 50 nts, about 10 nt to about 40 nts, about 12 nt to about 35 nt, about 15 nt to about 30 nt, or about 18 nt to about 26 nt; in some embodiments, the cells express a chimeric antigen receptor or further comprise a nucleic acid encoding a chimeric antigen receptor; in some embodiments, at least 30%, 40%, or 50% of the NK cells express a chimeric antigen receptor; in some embodiments, the chimeric antigen receptor is targeted to a cancer antigen or a viral antigen; in some embodiments, the NK cells are activated.

In some embodiments, any of the NK cells or compositions comprising NK cells comprise a nucleic acid sequence encoding a chimeric antigen receptor. In some embodiments, any of the NK cells express a chimeric antigen receptor. In some embodiments, the chimeric antigen receptor is targeted to a cancer antigen or a viral antigen; a chimeric antigen receptor can be targeted to any known cancer antigen (e.g., CD19, CD20, CS1, CD138 and Her2) or any known viral antigen (e.g., Spike protein of SARS-CoV-2)

Also described herein are methods of killing cancer cells comprising contacting the cancer cells with a therapeutically effective amount of a composition of the disclosure or any of the population of human NK cells described herein. Also described herein are methods of treating cancer in a subject in need thereof, comprising administering to the subject a therapeutically effective amount of a composition of the disclosure or any of the population of human NK cells described herein, thereby treating cancer in the subject.

Also described herein are methods of suppressing or reducing the proliferation of cancer cells comprising contacting the cancer cells with a therapeutically effective amount of a composition of a composition of the disclosure or any of the population of human NK cells described herein. Also described herein are methods method of treating a viral infection in a subject in need thereof, comprising administering to the subject a therapeutically effective amount of a composition of claim 1 or the population of human NK cells of any one of claims 27-31, thereby treating the viral infection in the subject.

Also described are methods of treating cancer in a subject in need thereof, comprising administering to the subject a therapeutically effective amount of a composition of Cbl-b^{neg} NK cells, thereby treating cancer in the subject. In various embodiments: the cancer is selected from a group consisting of lung cancer, breast cancer, ewing sarcoma, central nervous system neoplasm, skin cancer, head and neck cancer, ovarian cancer, colon cancer, anal cancer, stomach cancer, gastrointestinal cancer, fallopian tube cancer, endometrial cancer, cervical cancer, vaginal cancer, vulvar cancer, esophageal cancer, endocrine cancer, thyroid cancer, parathyroid cancer, adrenal cancer, soft tissue sarcoma, urethral cancer, penile cancer, testicular cancer, brain stem glioma, pituitary cancer, adrenocortical cancer, gallbladder cancer, multiple myeloma, cholangiocarcinoma, fibrosarcoma, lymphoma, liver cancer, kidney cancer, bone cancer, bladder cancer, colorectal cancer, endometrial cancer, renal cell cancer, pancreatic cancer, prostate cancer, thyroid cancer, mesothelioma, neuroblastoma, retinoblastoma, melanoma, rhabdomyosarcoma, leukemia and lymphoma. Also described are methods of treating a viral infection in a subject in need thereof, comprising administering to the subject a therapeutically effective amount of a composition comprising Cbl-b^{neg} NK cells, thereby treating the viral infection in the subject. Also described are populations of human NK cells harboring a vector comprising a nucleic sequence expressing an siRNA targeted to Cbl-b and a nucleic acid sequence expressing a chimeric antigen receptor.

Also described are methods of suppressing or reducing the proliferation of tumor cells comprising contacting the tumor cells with a therapeutically effective amount of a composition of the disclosure. In some embodiments, the tumor cells are primary ductal carcinoma cells, glioblastoma cells, leukemia cells, acute T cell leukemia cells, chronic myeloid lymphoma (CML) cells, acute myelogenous leukemia cells, chronic myelogenous leukemia (CML) cells, lung carcinoma cells, colon adenocarcinoma cells, histiocytic lymphoma cells, multiple myeloma cells, colorectal carcinoma cells, colorectal adenocarcinoma cells, prostate cancer cells, or retinoblastoma cells. The materials, methods, and examples are illustrative only and not intended to be limiting. All publications, patent applications, patents, sequences, database entries, and other references mentioned herein are incorporated by reference in their entirety for any and all purposes. Other features and advantages of the described compositions and methods will be apparent from the following detailed description and figures, and from the claims.

### DESCRIPTION OF DRAWINGS

FIGS. 1A-1D depict the results of a study showing that Cbl-b is upregulated when primary human NK cells are activated by IL-15, IL-2 or K562. (FIG. 1A) Enriched primary human NK cells were treated with IL-2 (150 IU/ml), IL-7 (50 ng/ml), IL-12 (10 ng/ml), IL-18 (10 ng/ml), IL-21 (20 ng/ml) or IL-15 (20 ng/ml) for 24 h followed by immunoblot analysis (n=5 donors). Densitometric quantification from five independent experiments shows the ratio of Cbl-b protein to β-actin protein. (FIG. 1B) Enriched primary human NK cells were co-cultured with the K562 myeloid leukemia cell line (E/T ratio=10:1) for 24 h followed by immunoblot analysis. Summary data are from three independent experiments. (FIG. 1C) Primary human NK cells were treated with different concentrations of IL-15 for 24 h followed by immunoblot analysis (n=5 donors). (FIG. 1D) Equal numbers of purified CD56⁺ human NK cells, CD56^{bright} NK cells and CD56^{dim} NK cells were sorted from peripheral blood mononuclear cells isolated by Ficoll, cultured and stimulated with IL-15 (20 ng/ml for 24 h) followed by immunoblot analysis. The data are representative of three independent experiments. Med, medium only; *P<0.05, **P<0.01 by two-tailed unpaired t-test or One-way ANOVA; ns, not significant. Data are presented as mean±SEM.

FIGS. 2A-2E depict the results of a study showing that JAK-STAT and AKT pathways mediate upregulation of Cbl-b in NK cells. (FIG. 2A) Primary human NK cells were pretreated with the JAK3 inhibitor CP-690550 and the JAK1/2 inhibitor AZD1480 at various concentrations for 90 min prior to a 24 h activation by IL15 (10 ng/ml). NK cells were then harvested for quantification of Cbl-b protein expression by immunoblot analysis. Data are representative of 6 donors performed in a similar fashion. Densitometric quantification assessing the ratio of the Cbl-b protein to β-actin protein levels for 6 donors is summarized in (FIG. 2B) and (FIG. 2C). (FIG. 2D) Primary NK cells were pretreated with the AKT1/2/3 inhibitor Afuresertib (10 uM) for 90 min prior to a 24 h activation by IL-15 (10 ng/ml) or IL-2 (150 IU/ml). Data are representative of 5 donors performed in a similar fashion. Densitometric quantification assessing the ratio of the Cbl-b protein levels to β-actin protein levels for 5 donors is summarized in (FIG. 2E). *P<0.05, **P<0.01, ***P<0.001 by One-way ANOVA or Student's two-tailed paired t-test. Data are presented as mean±SEM.

FIGS. 3A-3E depict the results of a study showing that downregulation of Cbl-b enhances the cytotoxicity of primary human NK cells. (FIGS. 3A and 3B) Primary human NK cells transduced with Cbl-b siRNA or Scr siRNA for 24 h were stimulated without (FIG. 3A) or with (FIG. 3B) a low concentration of IL-15 (2 ng/ml) for 16 h, then co-cultured with ⁵¹Cr labeled Molm-13, MV4-11 or EOL-1 leukemia cell lines for 4 h, followed by quantification of specific tumor cell lysis. Each experiment was repeated with 6 different normal donors. (FIG. 3C) qRT-PCR was performed to quantify the granzyme B (GZMB) mRNA levels (n=8 donors) in siRNA- and Scr siRNA-transduced primary human NK cells incubated without or with IL-15 (10 ng/ml) for 16 h. (FIG. 3D) The concentration of the granzyme B protein in the supernatants of Cbl-b siRNA- and Scr siRNA-transduced primary human NK cells incubated without (688.1 pg/ml vs 525.4 pg/ml; P=0.0232) or with IL-15 (10 ng/ml) for 24 h (8789 pg/ml vs 3397 pg/ml; P=0.0232) was measured by ELISA (n=8 donors). (FIG. 3E) The level of granzyme B protein in Cbl-b siRNA- and Scr siRNA-transduced primary human NK cells incubated without or with IL-15 (10 ng/ml) for 24 h was measured by immunoblot analysis. The data are representative of three experiments. Scr-scrambled; *P<0.05, **P<0.01, ***P<0.001, ****P<0.0001 by One-way or Two-way ANOVA; ns, not significant. Data presented as mean±SEM.

FIGS. 4A-4D depict the results of a study showing that downregulation of Cbl-b increases IFN-γ secretion in IL-15-activated primary human NK cells. (FIG. 4A) RNAseq was conducted using primary human NK cells transduced with either Cbl-b siRNA or Scr siRNA without or with incubation in IL-15 (10 ng/ml) for 16 h (4 donors for each group). The heatmap illustrates significantly up-regulated (red) or downregulated (green) gene expression. (FIG. 4B) qRT-PCR was performed to quantify the mRNA levels of IFN-γ (n=6 donors) after IL-15 (10 ng/ml) 6 h stimulation. (FIG. 4C) The percentage of intracellular IFN-γ+ cells in un-transduced NK cells or those transduced with Cbl-b siRNA or Scr siRNA, each activated with IL-15 (10 ng/ml) for 24 h (11.5% vs 4.338%; P=0.0008; n=5 donors). (FIG. 4D) Supernatants were collected after IL-15 (10 ng/ml) for 24 h and IFN-γ concentrations measured by ELISA (18115 pg/ml vs 882.5 pg/ml; P=0.0099; n=7 donors). Scr-scrambled; *P<0.05, **P<0.01, ***P<0.001 by Student's two-tailed paired t-test or One-way ANOVA; ns, not significant. Data presented as mean±SEM.

FIGS. 5A-5D depict the results of a study showing that Cbl-b interacts with and regulates phosphorylation of Mertk in primary human NK cells. (FIG. 5A) Representative intracellular flow cytometric analysis of the TAM family receptors expression in primary human NK cells. Summarized data are shown from 6 donors. IgG1 is the isotype for Tyro3 and Axl, while IgG2 for Mertk. (FIG. 5B) The level of Mertk phosphorylation and Cbl-b in Cbl-b siRNA- or Scr siRNA-transduced primary human NK was measured by immunoblot analysis. Data presented are representative of five independent experiments. (FIG. 5C) Primary NK cells were pretreated with the Mertk inhibitors UNC2550 or UNC2881 (5 uM) for 90 min prior to a 24 h activation by IL-15 (10 ng/ml), then the percentage of intracellular IFN-γ+ cells in NK cells was detected (20.64% vs 5.49%; P=0.0228). Data presented are representative of 4 donors performed in a similar fashion and are summarized in the panel below the representative flow dot plots. (FIG. 5D) Primary NK cells were pretreated with the Mertk inhibitors UNC2550 or UNC2881 (5 uM) for 90 min prior to 30 min activation by IL-15 (10 ng/ml). Data presented are representative of 4 donors performed in a similar fashion. Scr-scrambled; *P<0.05; ns, not significant. Data presented as mean±SEM.

FIG. 6A Enriched primary human NK cells were cocultured with Molm-13 or MV4-11 leukemia cell lines (E/T ratio=10:1) for 24 h prior to assessment of Cbl-b by immunoblot analysis. Data are representative of three independent experiments. FIG. 6B Enriched primary human NK cells were cultured overnight with an anti-NKG2D activating antibody (clone 1D11) (10 μg/ml) or mouse IgG (10 μg/ml) as control prior to assessment of Cbl-b by immunoblot analysis. Data are representative of four donors with similar results. FIG. 6C The expression of Cbl-b was assessed by qRT-PCR after primary human NK cells were treated with IL-15 (10 ng/ml) or IL-2 (150 IU/ml) at different time point. Data are shown summarizes four independent experiments. FIG. 6D The expression of Cbl-b was assessed by qRT-PCR after primary human NK cells were treated with IL-15 (10 ng/ml) or IL-2 (150 IU/ml) for 24 h. Data are shown summarizes five independent experiments. FIG. 6E The expression of Cbl-b was assessed by qRT-PCR after primary human NK cells were incubated with K562 cells (E/T ratio=10:1) for 24 h. Data are shown summarizes five independent experiments. *P<0.05, **P<0.01, compared to the shortest time point. Data are presented as mean±SEM.

FIG. 7A Primary NK cells were pretreated with AZD1480 (10 uM), CP-690550 (10 uM) or Afuresertib (10 uM) for 90 min prior to a 24 h cocultured with K562 cells (E/T=10:1). The immunoblot data are representative of 3 donors in a similar fashion. Densitometric quantification assessing the ratio of Cbl-b protein levels to β-actin protein levels for 3 donors is summarized in FIG. 7B. *P<0.05, **P<0.01; ns, not significant. Data are presented as mean±SEM.

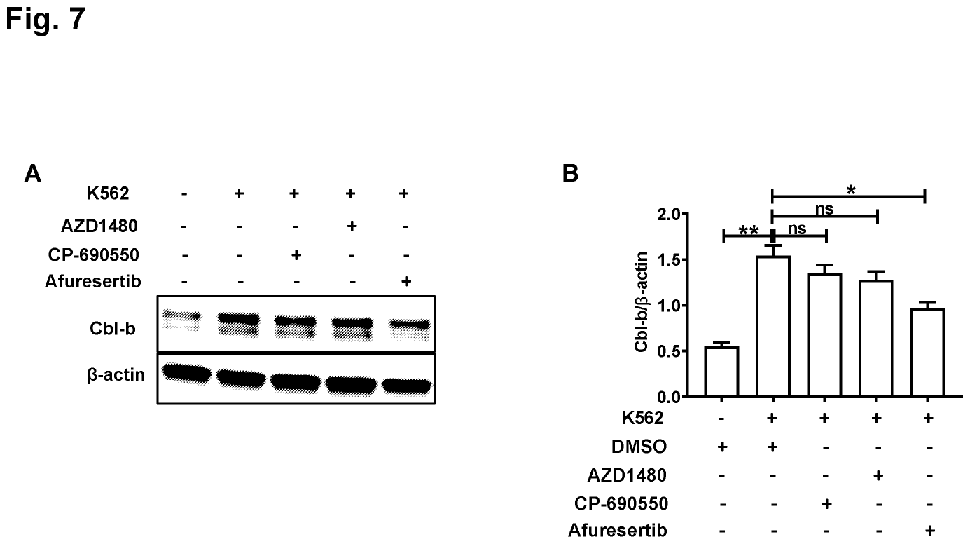

FIG. 8A Primary human NK cells were transduced with Cbl-b siRNA or a scrambled siRNA (Scr siRNA) for 48 h, directly followed by immunoblot analysis to determine the efficiency of siRNA (n=3 donors). Densitometric quantification shows the ratio of Cbl-b protein levels to β-actin protein levels. FIG. 8B qRT-PCR was performed to quantify the level of perform mRNA (n=7 donors) in Cbl-b siRNA- and Scr siRNA-transduced primary human cells incubated without or with IL-15 (10 ng/ml) for 16 h. FIG. 8C The perform protein level in Cbl-b siRNA- and Scr siRNA-transduced primary human NK cells incubated 24 without or with IL-15 (10 ng/ml) for 24 h was measured by immunoblot analysis. The data are representative of four experiments and are summarized in the right panel. Scr, scrambled; *P<0.05, **P<0.01; ns, not significant. Data are presented as mean±SEM.

FIG. 9A shows intracellular flow cytometric analysis of un-transduced CD56+ primary human NK cells or those transduced with Scr siRNA or Cbl-b siRNA, each without or with incubation with IL-15 (10 ng/ml) for 24 h. FIGS. 9B-9D show intracellular flow cytometric analysis of CD56+ primary human NK cells transduced with Scr siRNA or transduced with Cbl-b siRNA, incubated with IL-18 (10 ng/ml), IL-12 (10 ng/ml) or IL-18 (10 ng/ml) plus IL-12 (10 ng/ml) for 24 h. Scr, scrambled; *P<0.05, ns, not significant. Data are presented as mean±SEM.

FIGS. 10A-10B depict immunoblotting analysis of the phosphorylation level of Mertk (p-Mertk). (FIG. 10A) The level of Mertk phosphorylation and Cbl-b in Cbl-b siRNA- or Scr siRNA-transduced primary human NK cells was measured by immunoblot analysis. Data of four different donors are presented. (FIG. 10B) The level of total Mertk expression in Cbl-b siRNA- or Scr siRNA-transduced primary human NK was measured by immunoblot analysis.

FIG. 11A Flow cytometry of Cblbf/f NCR-Cre mice and littermate control (8-week old). Dot plots indicate the percentage of NK1.1⁺CD3⁻CD19⁻ cells which gated in lymphocytes in different tissue or organs including bone marrow (BM), liver (LV), and spleen (SP). FIG. 11B Absolute numbers of NK1.1⁺CD3⁻CD19⁻ cells were shown in different organs between Cblbf/f NCR-Cre Tg mice and littermate control (8-week old).

### DETAILED DESCRIPTION

Unless otherwise defined herein, scientific and technical terms used in this application shall have the meanings that are commonly understood by those of ordinary skill in the art. Generally, nomenclature used in connection with, and techniques of, cell and tissue culture, molecular biology, cell and cancer biology, virology, immunology, microbiology, genetics and protein and nucleic acid chemistry, described herein, are those well-known and commonly used in the art. In case of conflict, the present specification, including definitions, will control.

The practice of the present application will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al, 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al, eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, N Y (2001); Ausubel et al., Current Protocols in Molecular Biology, John Wiley & Sons, N Y (2002); Harlow and Lane Using Antibodies: A Laboratory Manual, Cold Spring Harbor Laboratory Press, Cold Spring Harbor, N Y (1998); Coligan et al., Short Protocols in Protein Science, John Wiley & Sons, N Y (2003); Short Protocols in Molecular Biology (Wiley and Sons, 1999).

Throughout this specification and embodiments, the word “comprise,” or variations such as “comprises” or “comprising,” will be understood to imply the inclusion of a stated integer or group of integers but not the exclusion of any other integer or group of integers. It is understood that wherever embodiments are described herein with the language “comprising,” otherwise analogous embodiments described in terms of “consisting of” and/or “consisting essentially of” are also provided.

The term “including” is used to mean “including but not limited to”. “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a”, “an” and “the” are used herein to refer to one or to more than one (i.e., to at least one) of the grammatical object of the article. By way of example, “an element” means one element or more than one element. Reference to “about” a value or parameter herein includes (and describes) embodiments that are directed to that value or parameter per se. For example, description referring to “about X” includes description of “X.” Numeric ranges are inclusive of the numbers defining the range. As used herein, the term “about” permits a variation of 10% within the range of the significant digit.

Notwithstanding that the disclosed numerical ranges and parameters are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.3, and ending with a maximum value of 10 or less, e.g., 5.7 to 10.

Where aspects or embodiments are described in terms of a Markush group or other grouping of alternatives, the present application encompasses not only the entire group listed as a whole, but each member of the group individually and all possible subgroups of the main group, and also the main group absent one or more of the group members. The present application also envisages the explicit exclusion of one or more of any of the group members in the Markush group or other grouping of alternatives.

### Definitions

As described herein, the term “modified to exogenously express” refers to forcing the expression of a gene of interest in a cell. In the context of this disclosure, a chimeric antigen receptor (CAR) is exogenously expressed in modified NK cells. “Exogenous expression” refers to the forced surface expression or “overexpression” of the protein of interest on the surface of the NK cells. In order to induce exogenous expression of a CAR, a sequence expressing the CAR may be cloned into an expression vector and delivered to the cells by, for example, lentiviral particle transduction. The CAR may be under the control of a “strong promoter”, which is a promoter that leads to a high level of transcription of mRNA. In some instances, the strong promoter may be a “constitutive promoter”, which is an unregulated promoter that is active under all circumstances, and allows for continual transcription of its associated gene. In some instances, the strong promoter is a retroviral promoter, or any other promoter known in the art.

As described herein, the term “expanding a population of cells” refers to the process of culturing cells in vitro or ex vivo by conventional cell culture methods known in the art. In the context of this disclosure, culturing cells or “cultivation” includes the step of “co-culturing”human NK cells with feeder cells (e.g., K562 cells), by the methods of the disclosure. Culturing provides the chemical and physical conditions (e.g., temperature, gas, pressure, etc.) which are required for NK cell and feeder cell maintenance, as well as growth factors. Culturing the NK cells includes providing the NK cells with conditions for expansion or proliferation. Examples of chemical conditions which may support NK cell expansion include but are not limited to buffers, serum, nutrients, vitamins, antibiotics, cytokines and other growth factors which are regularly provided in (or may be given manually to) the cell culture medium suited for NK cell expansion.

In one embodiment, the NK cell culture medium includes TexMACS Research Medium (Miltenyi Biotec GmbH) supplemented with 0-20% human serum type AB (Life Technologies) and 0-2000 IU/mL of interleukin-2 (IL-2) (Proleukin S, Novartis). In another embodiment the NK cell culture medium includes Stem Cell Growth Medium SCGM (Cell Genix) supplemented with 0-20% human serum type AB (Life Technologies) and 0-2000 IU/mL of IL-2 (Proleukin S, Novartis). Other media suitable for use in expanding NK cells are well known in the art.

Cell culture media or liquids providing the chemical conditions which are required for NK cell and feeder cell maintenance. Examples of chemical conditions which may support NK cell and feeder cell maintenance, as well as NK cell expansion include but are not limited to solutions, buffers, serum, serum components, nutrients, vitamins, cytokines and other growth factors which are regularly provided in (or may be given manually to) the cell culture medium. Media suitable for use to cultivate NK cells as known in the art include TexMACS (Miltenyi), CellGro SCGM (CellGenix), X-Vivo 10, X-Vivo 15, BINKIT NK Cell Initial Medium (Cosmo Bio USA), AIM-V (Invitrogen), DMEM/F12, NK Cell Culture Medium (Upcyte Technologies).

As used herein, the term “expansion”, “proliferation”, “multiplication” or cognates thereof refer to the increase of cell numbers during cell culture. During culture, the cells undergo a series of cell divisions and thus expand in numbers. Expansion, as used herein relates to increased numbers of NK cells occurring during the cell culture process disclosed in the methods of the disclosure. In one embodiment the term “expanded NK cells” refers to a group of activated NK cells.

As used herein, the term “primary NK cells” refer to NK cells that may be obtained from any conventional source such as from peripheral blood, bone marrow, cord blood, induced pluripotent stem cells (iPSCs), cell lines, cytokine stimulated peripheral blood, etc using techniques known in the art. See e.g., Fang F, et al. Cancer Biol Med. 2019; 16:647-54. doi: 10.20892/j.issn.2095-3941.2019.0187. The primary NK cells are immune cells with typical NK cell markers such as CD56 isolated from healthy donors or patients.

The terms “engineered cell” and “genetically modified cell” as used herein can be used interchangeably. The terms mean containing and/or expressing a foreign gene or nucleic acid sequence which in turn modifies the genotype or phenotype of the cell or its progeny. In particular, the terms refer to the fact that cells can be manipulated by recombinant methods well known in the art to express stably or transiently peptides or proteins which are not expressed in these cells in the natural state. Genetic modification of cells may include but is not restricted to transfection, electroporation, nucleofection, transduction using retroviral vectors, lentiviral vectors, non-integrating retro- or lentiviral vectors, transposons, designer nucleases including zinc finger nucleases, TALENs or CRISPR/Cas. In one embodiment, the genetically modified NK cell may express a chimeric antigen receptor (CAR). CARs are able to redirect a cytotoxic immune response against all cells that express the antigen they bind to. CARs may be used in the cancer immunotherapy, wherein NK cells, carrying a CAR targeted to a tumor antigen may generate a strong antitumor response against cells expressing the antigen targeted by the CAR (Sadelain et al., Cancer Discovery. 2013. 3(4):388-98). In some embodiments CAR-NK cells may be expanded using methods of the disclosure.

“Activated NK cells” refer to NK cells that are activated and acquire a cytotoxic phenotype and have enhanced NK cell function. A number of markers of activation, and cytotoxic function may be upregulated on the surface of the expanded NK cell population produced by the methods of this disclosure. The activating pathway of NK cells also includes a series of different receptors. Activating receptors do not directly signal through their cytoplasmic tail, but instead associate non-covalently with other molecules containing ITAMs (immunoreceptor tyrosine-based activation motifs), that serve as the signal transducing proteins. Thus, according to one embodiment, the ex vivo expanded and activated NK cells have an upregulated expression of at least one activating receptor, e.g., CD25 and/or CD69, and upregulate killer cell lectin-like receptor Gi (KLRG1), which is expressed on the most mature NK cells and is a receptor for NK cell maturation. In some instances, such cells upregulate the expression of IFNγ and/or CD107a, which are functional markers for NK cell degranulation and cytokine production, following NK cell activation. In addition, activated NK cells may upregulate PD-L1 expression, which is an indication of enhanced NK cell function. Activated NK cells of this disclosure may have enhanced expression and activity of cell signaling molecules such as p-STAT3, p-P65, pAKT, p-ERK, etc.

In the content of the present disclosure, NK cells that have a “cytotoxic phenotype” relates to cells that are cytotoxic, i.e. they induce the death of other cells such as, but not limited to, tumor cells, virus-infected cells or cells that are otherwise damaged or dysfunctional. Cytotoxic cells of the present disclosure are mainly toxic to tumor cells and virus-infected cells. The cytotoxicity of NK cells towards cells can easily be measured, for example, by traditional cell counting before and after exposure to the expanded NK cells of the disclosure. Such methods are well known to a person skilled in the art. Examples of suitable methods are, but not limited to, fluorescent cell counting assay, immunofluorescent cell counting assay, Chromium-51 release assay, cell viability assay, and flow cytometry-based cytotoxicity assay.

As used herein, a “composition” refers to a preparation of the expanded NK cells of the disclosure. The composition may have a physiologically acceptable carrier, diluent, excipient, and/or other components such as IL-2 or other cytokines or cell populations. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. A composition of the disclosure may comprise a) a population of NK cells, wherein said NK cells are expanded to therapeutically effective amounts; and b) one or more pharmaceutically or physiologically acceptable carriers, diluents or excipients. Administration of the NK cells in the compositions of the disclosure can be autologous or heterologous. For example, NK cells can be obtained from one subject, and administered to the same subject or a different, compatible subject. Compositions of the disclosure can be formulated for administration via localized injection, including catheter administration, systemic injection, localized injection, intravenous injection, or parenteral administration. When administering a therapeutic composition of the present invention (e.g., a pharmaceutical composition containing a genetically modified immunoresponsive cell), it will generally be formulated in a unit dosage injectable form (solution, suspension, emulsion).

Compositions of the present disclosure may be administered in a manner appropriate to the condition to be treated. The quantity and frequency of administration will be determined by such factors as the condition of the subject in need thereof, and the type and severity of the subject's condition, although appropriate dosages may be determined by clinical trials. The expanded NK cells achieved with the methods of this disclosure may be used in subsequent therapeutic or non-therapeutic applications.

### NK Cells

Natural killer (NK) cells comprise 5% to 20% of human peripheral blood lymphocytes and are derived from CD34+ hematopoietic progenitor cells. NK cells have the morphology of large granular lymphocytes, and are phenotypically defined by the expression of CD56 and the lack of CD3 and T-cell receptor molecules. NK cells function predominantly in direct cytotoxicity and antibody-dependent cellular cytotoxicity (ADCC). The function of NK cells is regulated by the balance between activation and inhibitory signals. Upon encountering normal cells, NK cells recognize their major histocompatibility complex (MHC) class I molecules and induce inhibitory signals to override activating signals (Gasser S, and Raulet D H. Immunol Rev. 2006; 214:130-42). By contrast, NK cells exert strong cytotoxic effects when they encounter cells lacking self-MHC class I molecules via multiple mechanisms including releasing cytotoxic granules such as performs and granzymes (Liao N S, et al. Science. 1991; 253(5016):199-202).

NK cells differ from natural killer T cells (NKTs) phenotypically, by origin and by respective effector functions; often, NKT cell activity promotes NK cell activity by secreting IFNy. In contrast to NKT cells, NK cells do not express T-cell antigen receptors (TCR) or pan T marker CD3 or surface immunoglobulins (Ig) B cell receptors, but they usually express the surface markers CD 16 (FcyRIII) and CD56 in humans.

### Expansion of NK Cells

Natural killer cell-based immunotherapy used to treat cancer requires the adoptive transfer of a large number of activated NK cells. Obtaining sufficient numbers of activated NK cells is important for an effective NK cell-based immunotherapy (Kweon S et al., Front. Immunol, 24, April 2019).

NK cells can be expanded in vitro by cultivating with combinations of cytokines, by supplementing cell culture media with small molecules (e.g. Nicotinamide) and by combinations of cytokines, antibodies and feeder cells. However, cytokine-based NK cell cultures result in only a minor increase in cell numbers that are not sufficient to manufacture NK cell products for multiple patients or from small NK cell subpopulations. NK cells may stop growing in these protocols after 3 weeks, and a prolonged culture does not result in higher NK cells numbers. Feeder cell-based NK cell expansion protocols generate higher NK cell numbers. However, there is a need for effective protocols that would predictably produce expanded numbers of NK cells that would be of therapeutic value.

The methods of this disclosure can take place in any container compatible with cell culture and expansion, e.g., flask, tube, beaker, dish, multiwell plate (e.g., G-REX®), bag or the like. In a specific embodiment, the co-culturing of NK cells with feeder cells takes place in a bag, e.g., a flexible, gas-permeable fluorocarbon culture bag (for example, from American Fluoroseal). In a specific embodiment, the container in which the NK cells are cultured is suitable for shipping, e.g., to a site such as a hospital or military zone wherein the expanded NK cells are further expanded.

### Cell Lines

Feeder cells can aid in the expansion of NK cells ex vivo or in vitro. Feeder cells provide an intact and functional extracellular matrix and matrix-associated factors and secrete known and unknown cytokines into the conditioned medium. Feeder cells are usually growth arrested to prevent their proliferation in the culture, but their survival is maintained. Growth arrest can be achieved by irradiation with an effective dose or treatment with an effective dose of chemicals such as Mitomycin C. In the context of this disclosure, feeder cells useful in the methods disclosed herein include but are not limited to cancer cell lines (e.g., chronic myelogenous leukemia (CML) cells such as K562, etc), fibroblasts, stem cells (e.g., stem cells), blood cells (e.g., allogeneic or autologous irradiated or non-irradiated peripheral blood mononuclear cells (PBMC) depleted of NK cells), genetically engineered cancer cell lines, lymphocytes immortalized by natural infection with Epstein-Barr Virus (EBV), etc. Preferably, the feeder cells are K562 cells. The K562 cells may be genetically engineered to express certain ligands, for instance OX40 ligand (Kweon S et al., Front. Immunol., 24 Apr. 2019). K562 cells expressing membrane bound IL-21 and 41-BB Ligand (APC K562) are preferably used in the methods of this disclosure. An APC K562 cell line can be generated from commercially available K562 cells by methods known in the art. Other cell lines useful in the disclosed methods are K562-s (ATCC® CRL-3343™), K562 (ATCC® CCL-243™), K562-s (ATCC® CRL-3344™) and cell lines described in U.S. Pat. Nos. 7,435,596, 8,026,097, 9,623,082, and 10463715, the disclosures of each of which are incorporated herein by reference in their entirety.

### Methods of Treatment

The methods described herein can be used to produce a population of NK cells, e.g., comprising NK cells lacking cbl-b, compared to methods known in the art. The cells thus produced may be used to treat cancer and suppress the proliferation of tumors due to the cytotoxic activity of NK cells. For instance, the expanded NK cells may be used to treat a wide range of cancers including by not limited to lung cancer, breast cancer, ewing sarcoma, central nervous system neoplasm, skin cancer, head and neck cancer, ovarian cancer, colon cancer, anal cancer, stomach cancer, gastrointestinal cancer, fallopian tube cancer, endometrial cancer, cervical cancer, vaginal cancer, vulvar cancer, esophageal cancer, endocrine cancer, thyroid cancer, parathyroid cancer, adrenal cancer, soft tissue sarcoma, urethral cancer, penile cancer, testicular cancer, brain stem glioma, pituitary cancer, adrenocortical cancer, gallbladder cancer, multiple myeloma, cholangiocarcinoma, fibrosarcoma, lymphoma, liver cancer, kidney cancer, bone cancer, bladder cancer, colorectal cancer, endometrial cancer, renal cell cancer, pancreatic cancer, prostate cancer, thyroid cancer, mesothelioma, neuroblastoma, retinoblastoma, melanoma, rhabdomyosarcoma, leukemia and lymphoma.

The cells of this disclosure may also be used to treat acute or chronic viral infections, such as infections caused by human immunodeficiency virus (HIV), Epstein-Barr virus (EBV), herpes simplex virus (HSV), cytomegalovirus (CMV), varicella-zoster virus (VZV), hepatitis B virus (HBV) or hepatitis C virus (HCV), coronavirus, etc. In one embodiment, the expanded NK cells of this disclosure may be used to treat COVID-19 caused by SARS-CoV-2.

Additionally, it may be possible to engineer human NK cells with antitumor and antiviral chimeric antigen receptors (CAR-NK), or genetically modifying NK cells themselves to be stronger and more resilient. The NK cells can then be expanded using methods of the present disclosure. Engineered NK cell therapy, such as CAR-NK cell-based immunotherapy may be used in cancer therapy or anti-viral therapy. For instance, CAR-NK cell line therapy may provide a favorable treatment alternative to relapsed and refractory B cell malignancies (Ochsner J. 2019 Fall; 19(3): 186-187.) NK cells of the present disclosure may function as immune adjuvants with the ability to boost the efficacy of anticancer therapy when combined with traditional treatments such as chemotherapy (Hu W et al., Front Immunol. 2019; 10: 1205). Further, the NK cells of this disclosure may also be used to produce extracellular vesicles (EVs) that be applied in cancer therapies. EVs are nano-sized vesicles with anti-tumor activity that are naturally secreted by NK cells and provide a cell-free immunotherapy avenue (Hu W et al., Front Immunol. 2019; 10: 1205).

NK cells of the present disclosure may be administered alone or used in combination with other cancer therapies, such as, but not limited to surgery, radiation and cytotoxic drugs. Similarly, the NK cells of the present disclosure may be administered alone or used in combination with other antiviral therapies. The NK cells of this disclosure may be administered prior to, at the same time as, or subsequent to the cancer therapy or anti-viral therapy.

The expanded NK cells of the present disclosure may be administered to a subject in need thereof by adoptive cell transfer (ACT) wherein the NK cells may have originated in the same subject or a different subject. Further, the expanded NK cells of the disclosure can used for adoptive immunotherapy in conditions such as cancer and viral infections. For instance, autologous ex vivo expanded NK cells, can be administered, either prophylactically or therapeutically, to patients undergoing autologous hematopoietic stem cell transplantation for diseases such as multiple myeloma which have in general a poor prognosis with high incidence of progressive disease post transplant. Ex vivo expanded NK cells of donor origin can be used for the treatment of recurrent malignant disease following allogeneic stem cell transplantation. Autologous ex vivo expanded NK cells can be administered, either prophylactically or therapeutically, to patients undergoing autologous stem cell transplantation for cancer. Other examples include using autologous expanded NK cells for ex vivo purging of malignant cells in the harvest, for treatment of patients with hematological malignancies, and as a cellular therapy for solid tumors.

The expanded NK cells of the disclosure can also be administered to a patient in order to prevent recurrence of a malignant disease. In one instance, a sample (e.g., from peripheral blood, bone marrow, or cord) is taken from a patent afflicted with a malignant disease. In one embodiment, the patient has been treated with conventional cancer therapies but the treatment has been unsuccessful or the malignancy has recurred. The method can also be a prophylactic treatment, for example, to prevent recurrence of a malignant disease.

In the method of this disclosure, the NK cells are expanded ex vivo for at least about 1 day, 4 days, 8 days, 12 days, 16 days, 20 days, 24 days, 28 days, or greater than about 28 days. Preferably, the NK cells have been expanded ex vivo for about 6-28 days, before administration to the patient. In another embodiment, the NK cells have been expanded at least about 50 fold to about 50,000 fold compared to day 0 of expansion, before administration to a patient.

The method of treatment of the disclosure may be performed once or repeated several times. In one embodiment, the expanded NK cells of the disclosure are administered to the patient as needed, about 1-10 times, about 1-7 times, about 1-5 times, or about 1-3 times. In some embodiments, a population of NK cells described herein is administered to a subject in need thereof at least once or at least twice. The administration route can be any suitable way of administration well known to the skilled person for example, but not limited to; intravenous, intraperitoneal and intratumoral administration. The dosage can be the same in all administrations or for example high in the first administration(s) and then lower in subsequent administrations. The therapies can be a monotherapy or combinational therapies with other agents.

Administration of the expanded NK cells of the disclosure may be alone or in combination with IL-2 or its derivatives, immunomodulatory drugs (such as thalidomide), proteosome inhibitors (such as bortezomib) may further increase the effect of administered cells.

### EXAMPLES

The invention is further described in the following examples, which do not limit the scope of the invention described in the claims.

Described below are studies showing that the E3 ubiquitin ligase Cbl-b is significantly upregulated in primary human NK cells activated by IL-15, IL-2 and the human NK cell-sensitive tumor cell line K562 that lacks MHC class I expression. It is also shown that pretreatment with JAK or AKT inhibitors prior to IL-15 stimulation reverses Cbl-b upregulation. Downregulation of Cbl-b resulted in significant increases in granzyme B and perform expression, IFN-γ production and cytotoxic activity against tumor cells. Thus, modified NK cells having reduced or no expression of Cbl-b and expressing a CAR can have superior activity compared to NK cells that do not have reduced Cbl-b expression.

The practice of the methods and compositions of the disclosure employs, unless otherwise indicated, conventional techniques of molecular biology (including recombinant techniques), cell culture, microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the methods and compositions of the disclosure. Particularly useful techniques for particular embodiments will be discussed in the sections that follow. The following materials, reagents, and methods are used for the Examples described herein.

Isolation of primary human NK cells. Blood leukopacks were obtained from City of Hope National Medical Center Blood Bank under the institutional review board approved protocols. NK cells were isolated by using the RosetteSep™ human NK cell enrichment cocktail (StemCell Technologies) and Ficoll-Paque (GE Healthcare). The purity of primary NK cells was confirmed with flow cytometry using anti-CD56 (Beckman Coulter, Cat #B46024) and anti-CD3 (Miltenyi Biotec, Cat #130-113-134) antibodies. CD3⁻CD56^{bright} CD3⁻CD56^{dim} NK cells and total NK cells co-cultured with tumor cells were sorted using an Aria Fusion sorter (BD Biosciences).

Cell culture. Molm-13, EOL-1 and MV4-11 cell lines were purchased from the American Type Culture Collection (ATCC). Molm-13 and EOL-1 cell lines were cultured in RPMI with 10% heat-inactivated FBS (Sigma-Aldrich), and MV4-11 in IMDM with 10% FBS. All cells were incubated at 37° C. in a 5% CO₂ humidified incubator.

Antibodies and other reagents. Antibodies to Cbl-b (#9498), Granzyme B (#17215), perform (#62550) and Mertk (#4319) for immunoblotting analysis were purchased from Cell Signaling Technology (CST) and p-Mertk (#ab14921) from Abcam. An antibody to β-actin (#MAB1501R) for immunoblotting analysis was purchased from Millipore-Sigma. For flow cytometric analysis, the anti-hCD56-APC antibody (#B46024) was purchased from Beckman Coulter, anti-hCD3 (#130-113-134) and anti-hIFN-γ (#130-113-493) from Miltenyi Biotec, and anti-hTyro3 (FAB859P), anti-hAxl (#FAB154P) and anti-hMertk (FAB8912P) from R&D. The scrambled and Cbl-b-specific Accell siRNAs were purchased from Dharmacon. Recombinant human IL-2 and IL-15 proteins were obtained from National Institutes of Health (NIH). Recombinant human IL-7, IL-12, IL-18 and IL-21 were purchased from Miltenyi Biotec.

Transient transfection. Primary human NK cells were transfected with Accell siRNA by using the Accell delivery media from Dharmacon. Gene knockdown efficiency of siRNA was determined with immunoblotting analysis.

Reverse transcription-polymerase chain reaction. Total RNA was isolated from primary NK cells with the RNeasy Mini Kit (Qiagen). Complementary DNA (cDNA) was generated by Moloney murine leukemia virus (M-MLV) reverse transcriptase (Invitrogen) and amplified by qPCR with SYBR Green PCR Master Mix (Applied Biosystems) and gene-specific primers. Relative amplification values were normalized to the amplification of GAPDH or 18S rRNA.

Immunoblotting. Cells were harvested and suspended in RIPA lysis buffer (Thermo Fisher Scientific) on ice for 20 minutes. Equal amount of protein was resolved by 5-15% Criterion TGX gel (Bio-Rad) and then transferred onto a Nitrocellulose (NC) or PVDF membrane (Thermo Fisher Scientific). The membrane was incubated with a primary antibody at 4° C. overnight and an IRDye secondary antibody (Li-COR Biosciences) for 1 hour at room temperature. The immunoblots were visualized with Odyssey CLx Imager (Li-COR Biosciences). Densitometric analysis was performed to quantify intensity of gel bands with Image J (National Institutes of Health).

ELISA. Primary NK cells were plated in an equivalent number of 100,000 cells/well in a 96-well plate in RPMI and supplemented with 10% FBS. Cells were treated with recombinant human IL-15 (10 ng/ml) for 24 h, and cell-free culture supernatant was collected and frozen at −80° C. for later use. IFN-γ concentration was measured by ELISA using anti-IFN-γ antibody from Thermo Fisher Scientific. Granzyme B concentration was measured by ELISA kit purchased from R&D.

⁵¹Cr-release cytotoxicity assay. The ⁵¹Cr cytotoxicity assay was performed as described previously (18). Primary NK cells were transduced with siRNAs in Accell delivery media for 24 h, followed by being treated with or without IL-15 (2 ng/ml) for another 16 h. The treated NK cells were co-cultured with ⁵¹Cr labeled Molm-13, MV4-11 and EOL-1 cells in triplicates in a 96-well U-bottom plate at multiple ET ratios for 4 h at 37° C. in a 5% CO₂ incubator. The supernatant was harvested from each well and transferred into 96-well Luma plate and analyzed using a Microbeta scintillation counter (Wallac, PerkinElmer).

Flow cytometry. To determine protein cell surface expression via flow cytometry, cells were stained with monoclonal antibodies at room temperature for 20 minutes and washed with FACS buffer prior to analysis using a Fortessa X-20 flow cytometer (BD Biosciences). For IFN-γ intracellular flow cytometric analysis, 1 mg/ml GolgiPlug (BD Biosciences) was added for 4 h before cell harvest; cells were then permeabilized and fixed using a Cytofix/Cytoperm Fixation/Permeabilization Solution Kit (BD Biosciences), followed by analysis with a Fortessa X-20 flow cytometer. Intracellular flow cytometric analysis of Mertk was performed similarly except that GolgiPlug was not added. Data was analyzed by using Flowjo V10 software (Tree Star, Ashland, OR, USA).

Statistical analysis. For continuous, normally distributed data, student two-tailed t-tests or paired t-tests were used to compare two groups. Two-way ANOVA was used to compare two groups with different conditions. P value of 0.05 or less was considered statistically significant.

### Example 1: Cbl-b is Upregulated when Primary Human NK Cells are Activated by IL-15, IL-2 or K562

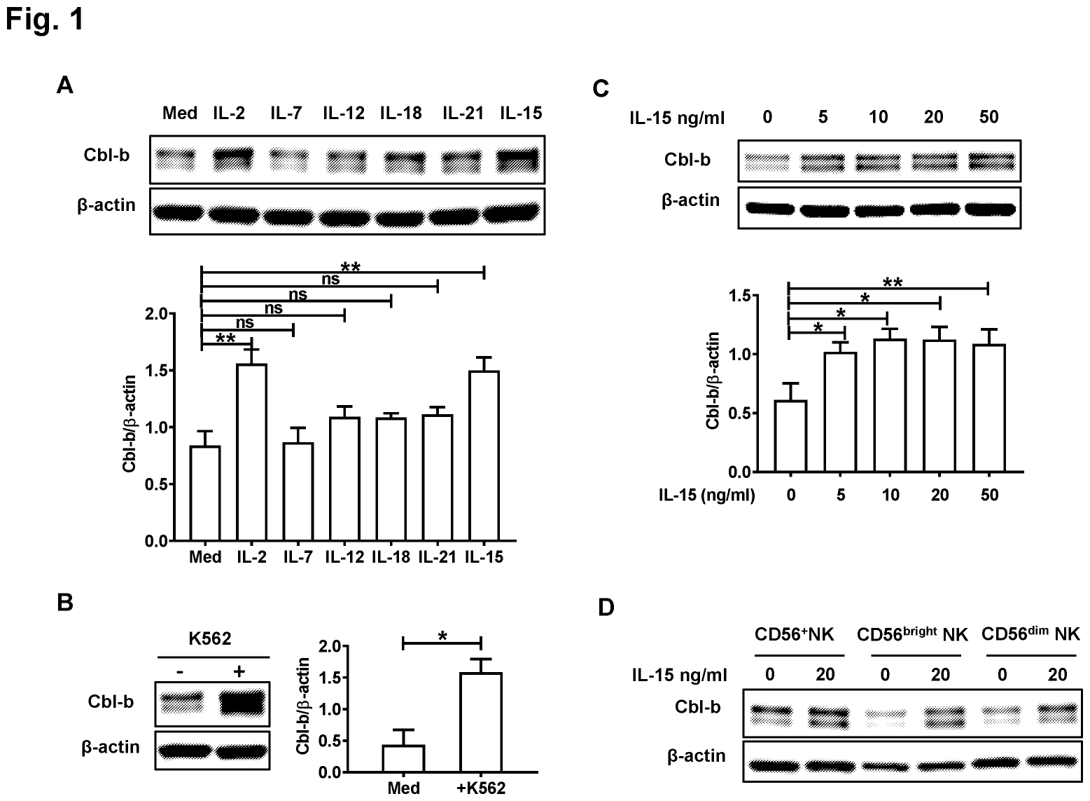

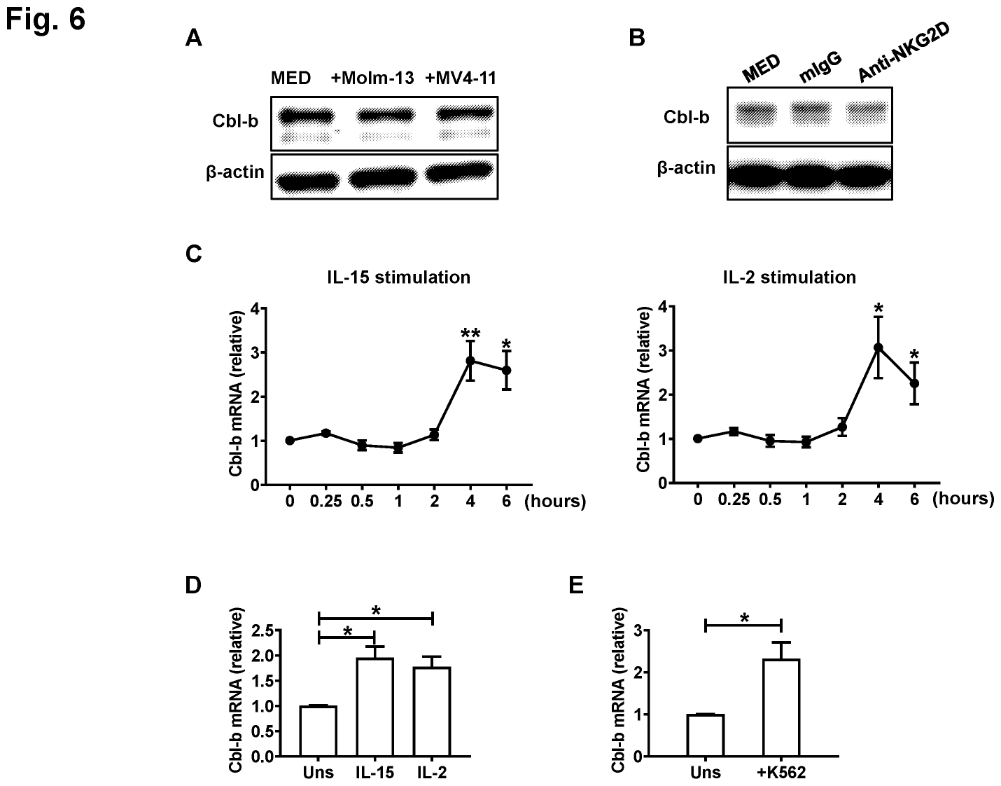

We assessed the expression of Cbl-b protein in primary human NK cells enriched from the blood of healthy donors in response to 24-hour stimulation by different cytokines. We found that stimulation with either IL-15 or IL-2 induced an increase in the expression of Cbl-b, whereas stimulation with IL-7, IL-12, IL-18 and IL-21 showed no significant change compared to resting NK cells incubated without cytokines (FIG. 1A). Since certain tumor cells can trigger NK cell activation, we co-cultured NK cells for 24 h with different tumor cell lines in order to assess this effect on the expression of Cbl-b. Our results showed a 3-fold increase in the expression of Cbl-b when fresh human NK cells were co-cultured with the NK-sensitive K562 myeloid leukemia cell line that lacks MHC class I (MHC I⁻) (FIG. 1B), whereas the more NK cell-resistant cell lines, Molm-13 and MV4-11 cells, which express MHC class I (MHC I⁺) (19), did not induce a significant upregulation of Cbl-b in resting NK cells (FIG. 6A). Moreover, primary human NK cells incubated with an NKG2D activating antibody (clone 1D11) for overnight showed no significant change of Cbl-b expression compared to those incubated with mouse IgG (FIG. 6B). When incubating NK cells with IL-15, we did not find evidence of a dose response change (FIG. 1C), nor did we find a difference in Cbl-b upregulation between CD56^{bright} and CD56^{dim} NK cells incubated in IL-15 (FIG. 1D). Then Cbl-b transcript was quantified at different time points in NK cells following incubation in IL-15 or IL-2; Cbl-b mRNA increased within 2 h and peaked following 4 h of stimulation with IL-15 or IL-2 compared to unstimulated NK cells (FIG. 6C). The upregulation of Cbl-b mRNA by IL-15 or IL-2 or K562 stimulation was also observed to be sustained at the longer time point, 24 h post stimulation (FIGS. 6D and 6E).

### Example 2: JAK-STAT and AKT Pathways Mediate Upregulation of Cbl-b in NK Cells

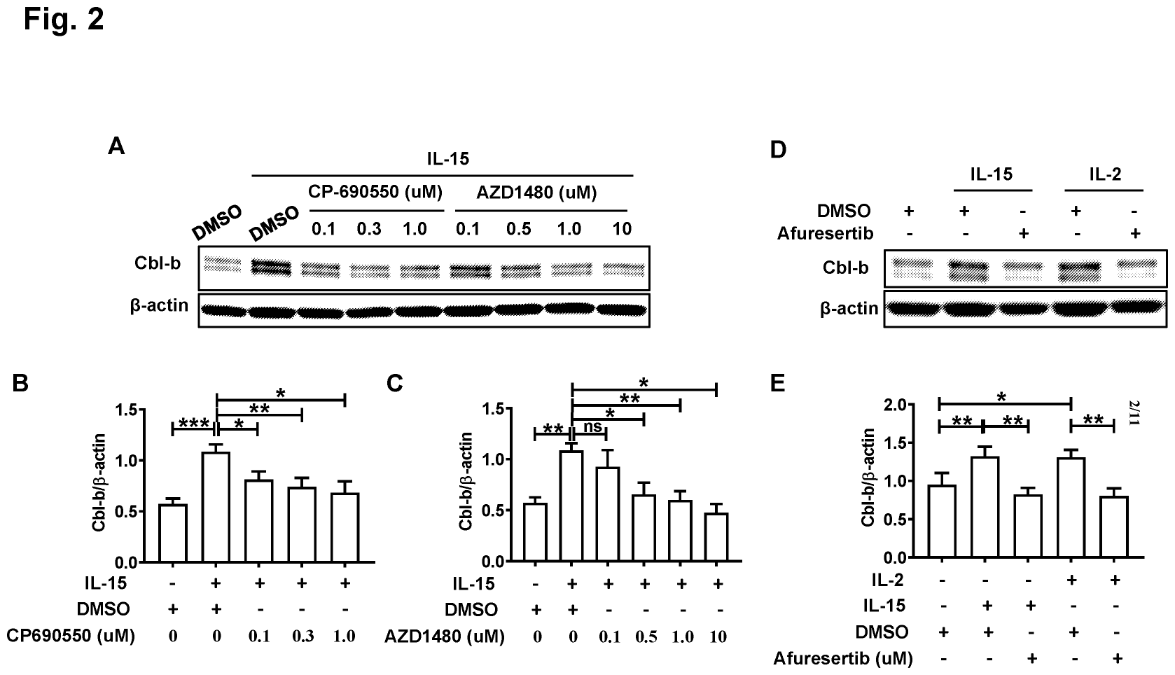

We next investigated the underlying mechanism by which IL-15 or IL-2 up-regulated the expression of Cbl-b protein. Despite the lack of homology in the amino acid sequence between IL-15 and IL-2, both proteins can bind to the IL-2/15Rβγ heterodimer, activating the intracellular signal leading to cell activation (20, 21). Since IL-15 utilizes select JAK-STAT proteins to initiate cellular activation, we pretreated primary NK cells with the JAK3 inhibitor CP-690550 (22) or the JAK1/2 inhibitor AZD1480 (23) in increasing concentrations prior to IL-15 stimulation then Cbl-b expression level was evaluated. As shown in FIG. 2A-C, blockade of the JAK-STAT pathway with pretreatment of either JAK inhibitor then stimulated primary NK cells with IL-15 resulted in a significant decrease in the expression of Cbl-b protein compared with IL-15-stimulated NK cells in the presence of vehicle control. Furthermore, we observed nearly identical results in primary NK cells pretreated with the AKT1/2/3 inhibitor Afuresertib (24) prior to activation by IL-15 or IL-2 stimulation (FIGS. 2D and 2E). As K562 cells were also able to induce expression of Cbl-b in primary human NK cells (FIG. 1B), we repeated the inhibitor experiments under the K562 cell stimulation. Blockade of the JAK-STAT or AKT pathway in NK cells showed that only the AKT1/2/3 inhibitor Afuresertib rather than either of the two JAK inhibitors (CP-690550 and AZD1480) resulted in a significant downregulation of Cbl-b expression compared to NK cells co-cultured with K562 cells in the presence of vehicle control (FIGS. 6A and 6B).

### Example 3: Downregulation of Cbl-b Enhances the Cytotoxicity of Primary Human NK Cells

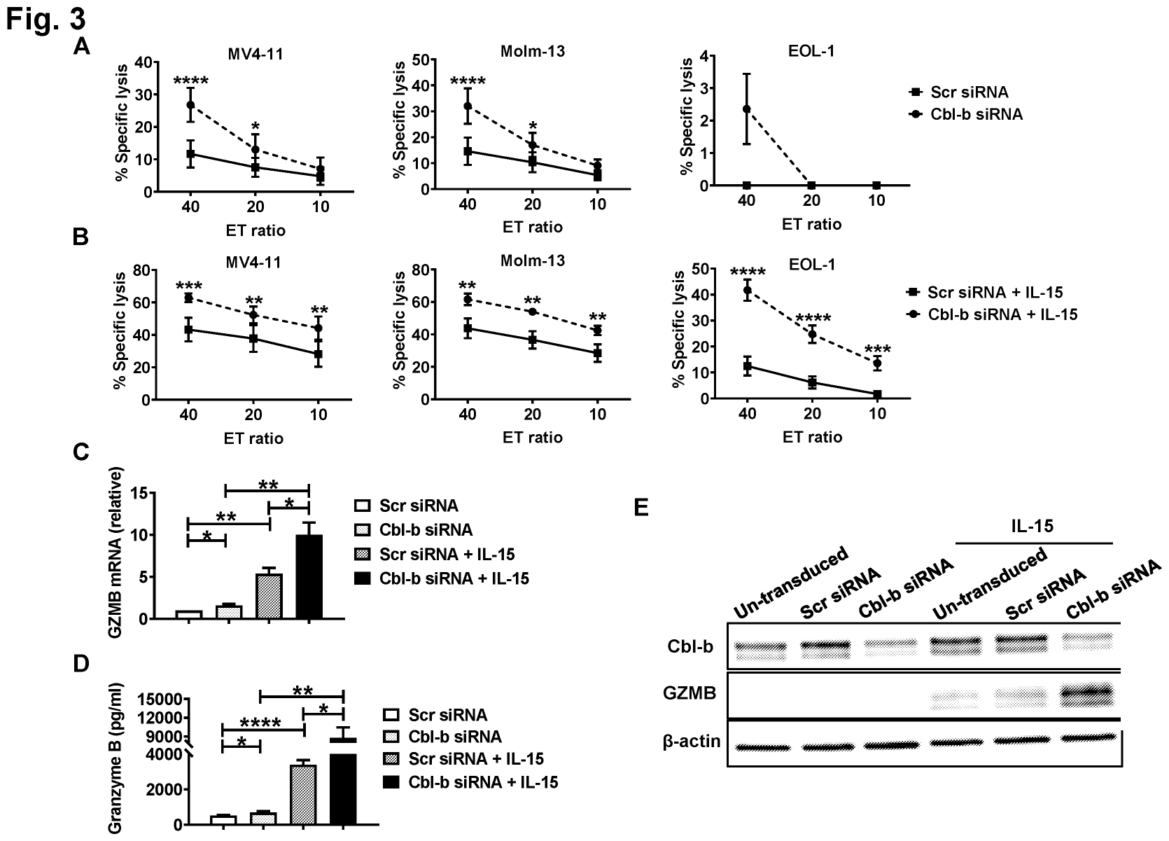

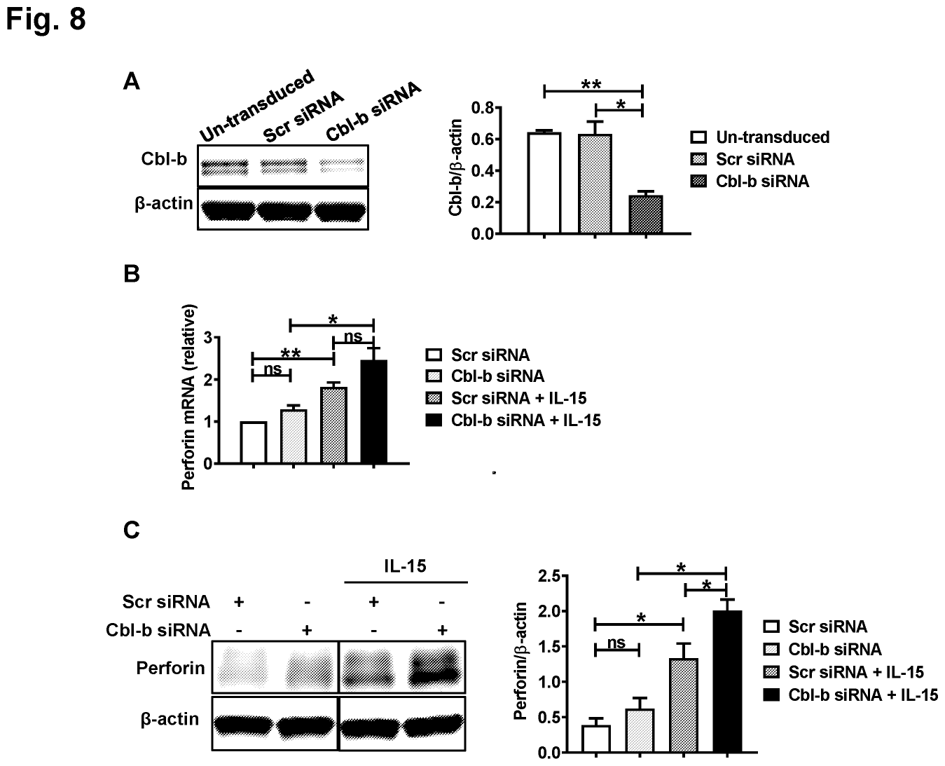

We transduced primary human NK cells with Cbl-b-specific siRNA (Cbl-b siRNA; SEQ ID NO:1, CCUUCAUGUUCAGAUGGUU) or scrambled siRNA (Scr siRNA; SEQ ID NO:2, UGGUUUACAUGUUGUGUGA) for 48 h and confirmed a significant decrease in the expression of Cbl-b in the experimental group (FIG. 8A). Both groups were next incubated without or with IL-15 and co-cultured with ⁵¹Cr labeled MV4-11, Molm-13, or EOL-1 leukemia cell lines, followed by assessment of specific lysis measured after 4 h of co-culture. In the absence of IL-15 stimulation, NK cells transduced with Cbl-b siRNA showed a significant increase in cytotoxicity against three of the four AML cell lines at the highest E/T ratio of 40:1, compared to NK cells transduced with Scr siRNA (FIG. 3A). When co-incubated with a low concentration of IL-15 (2 ng/ml), NK cells transduced with Cbl-b siRNA also showed a significant increase in cytotoxicity against all four AML cell lines at all three E/T ratios compared to NK cells transduced with Scr siRNA (FIG. 3B). Granzyme B mRNA was significantly increased in NK cells transduced with Cbl-b siRNA compared to NK cells transduced with Scr siRNA, either without or with IL-15 stimulation (FIG. 3C), while perform mRNA trended toward upregulation in NK cells transduced with Cbl-b siRNA but the upregulation did not reach a statistical significance (FIG. 8B). The secretion of soluble granzyme B protein was moderately but significantly higher in resting NK cells transduced with Cbl-b siRNA compared to resting NK cells transduced with Scr siRNA (FIG. 3D), whereas with IL-15 stimulation the secretion of soluble granzyme B protein from the NK cells transduced with Cbl-b siRNA was 2.6 times higher compared to NK cells transduced with Scr siRNA (FIG. 3D). Immunoblot analysis demonstrated an increase of total GZMB protein accompanied with the knockdown of Cbl-b by siRNA (FIG. 3E). In addition, the total protein level of perform was also significantly increased in NK cells transduced with Cbl-b siRNA compared to NK cells transduced with Scr siRNA with IL-15 stimulation (FIG. 8C).

### Example 4: Downregulation of Cbl-b Increases IFN-γ Secretion in IL-15-Activated Primary Human NK Cells

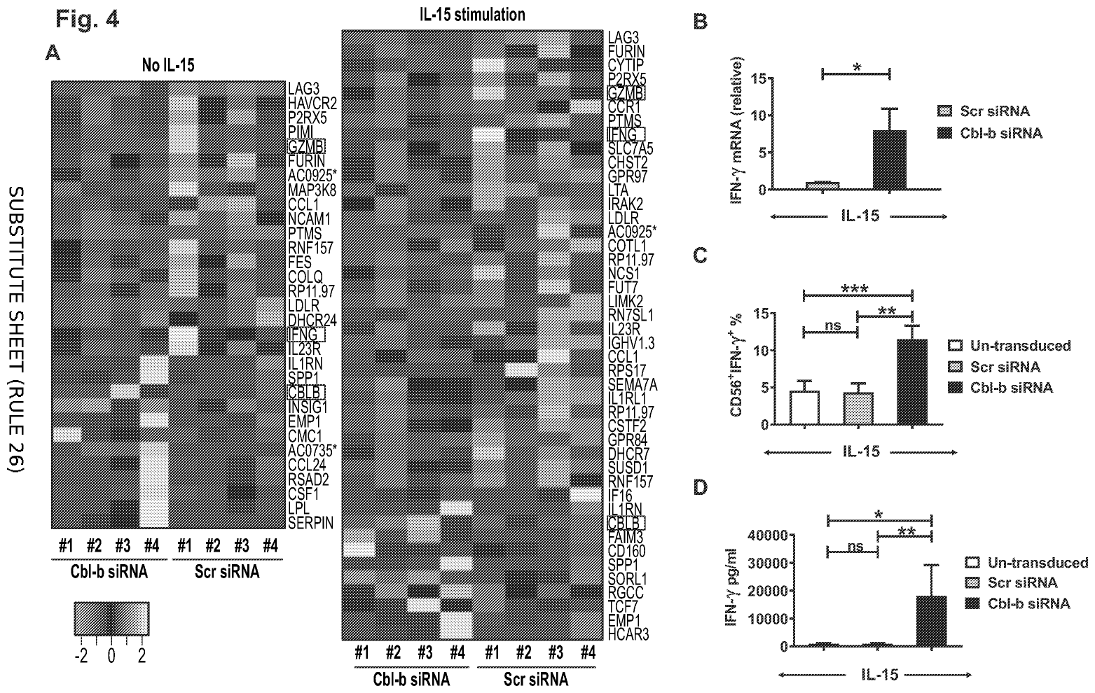

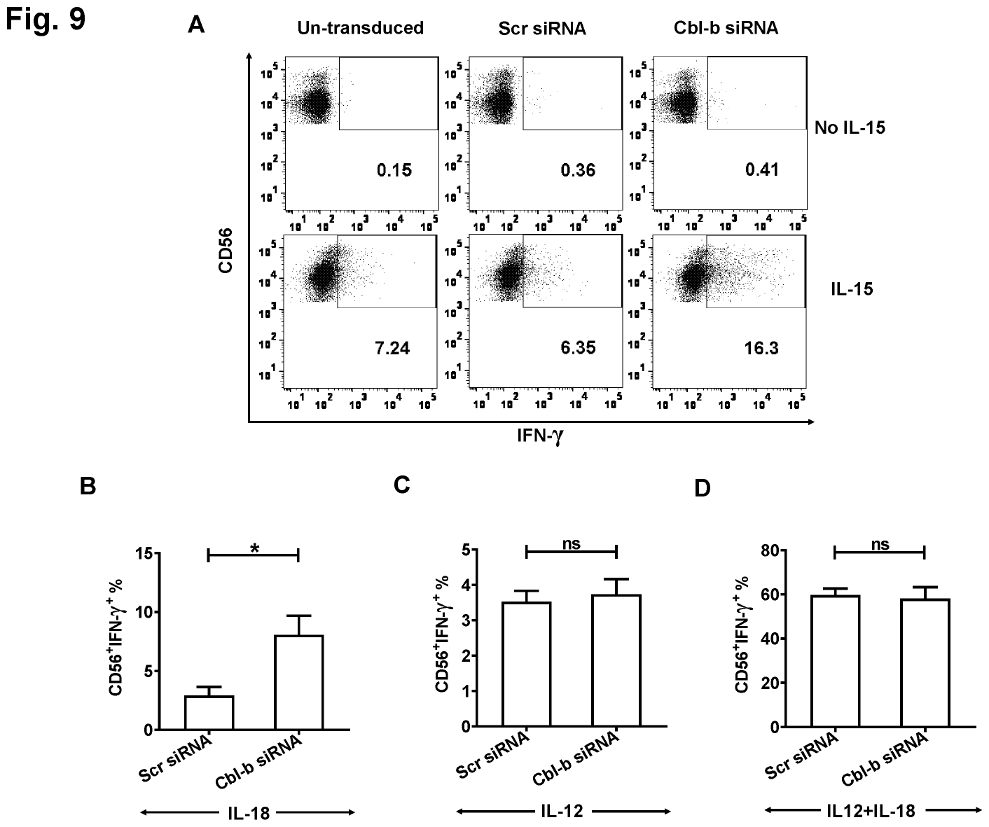

To further explore how Cbl-b regulates the functions of primary human NK cells, we performed RNA-seq analyses using enriched primary NK cells from healthy donors transduced with Cbl-b siRNA or Scr siRNA without or with IL-15 treatment for 16 h (n=4 in each group). After normalizing the level of each transcript in Cbl-b downregulated NK cells transduced with Cbl-b siRNA to their corresponding controls, we drew a heatmap quantifying the differentially expressed genes noted between NK cells transduced with Cbl-b siRNA or Scr siRNA and co-cultured without or with IL-15 (FIG. 4A). The RNA-seq data revealed that the transcriptional level of granzyme B was increased in human NK cells transduced with Cbl-b siRNA compared to those transduced with Scr siRNA either without or with IL-15 stimulation, which is consistent with our above results (FIG. 3C). We detected IFN-γ mRNA expression levels in IL-15-activated primary human NK cells, and the result was consistent with our RNA-seq data, with IFN-γ mRNA increased dramatically in the Cbl-b siRNA group compared to that in the Scr siRNA group (FIG. 4B). Using intracellular flow cytometry, we demonstrated that IL-15-activated primary human NK cells transduced with Cbl-b siRNA expressed 2.5-fold higher levels of IFN-γ compared to NK cells transduced with Scr siRNA (FIG. 9A and FIG. 4C). The intracellular results were supported by assessment IFN-γ secretion into the supernatant, in that IL-15-activated primary human NK cells transduced with Cbl-b siRNA expressed 10-fold higher levels of IFN-γ compared to NK cells transduced with Scr siRNA (FIG. 4D). Additionally, the percentage of IFN-γ⁺ fraction in IL-18-stimulated primary human NK cells transduced with Cbl-b siRNA was 2.5-fold higher than that in NK cells transduced with Scr siRNA (FIG. 9B), while there was no significant difference in IL-12-stimulated or IL-12 and IL-18 co-stimulated primary human NK cells (FIGS. 9C and 9D).

### Example 5: Cbl-b Interacts with and Regulates Phosphorylation of Mertk in Primary Human NK Cells

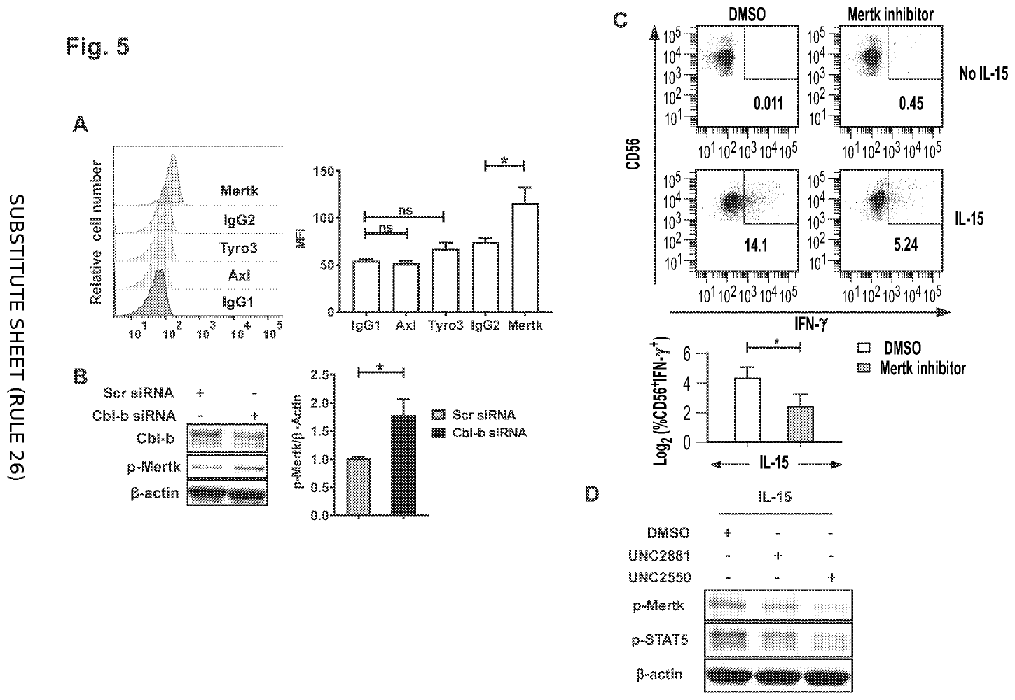

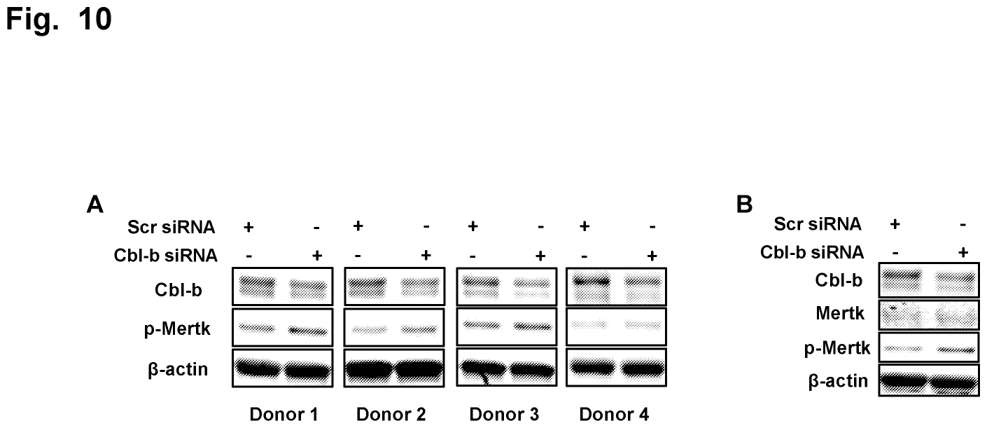

The TAM receptor family consists of Tyro3, Axl and Mertk, all of which are expressed in mature mouse NK cells (25) and are molecular substrates for Cbl-b ubiquitylation both in vitro and in vivo (17). To investigate the possible relationship between Cbl-b and the TAM receptor family in primary human NK cells, we detected the expression of the three TAM receptor members in NK cells. Intracellular flow cytometric analysis showed that Mertk could be detected in primary human NK cells while the other two-family members Tyro3 and Axl were undetectable (FIG. 5A). Immunoblotting analysis showed that the phosphorylation level of Mertk (p-Mertk) was significantly increased in NK cells transduced with Cbl-b siRNA compared to those transduced with Scr siRNA (FIG. 5B and FIGS. 10A and 10B). Previous studies showed that the TAM family receptors are the molecular substrates for Cbl-b-mediated ubiquitylation in murine NK cells (17). However, the level of total mertk is low in human NK cells (FIG. 10B), preventing us to characterize the mechanism associated with the p-Mertk regulation by Cbl-b. Next, primary human NK cells were pretreated with or without Mertk inhibitors (UNC2550 or UNC2881), followed by IL-15 stimulation. We found that in the presence of IL-15, primary human NK cells treated with Mertk inhibitors expressed approximately 3-fold lower levels of IFN-γ compared to control with no treatment of the inhibitors (FIG. 5C). Immunoblotting analysis showed that the level of p-STAT5 was decreased in IL-15-activated primary human NK cells treated with Mertk inhibitors compared to control with no treatment of the inhibitors (FIG. 5D). Our data suggest that Cbl-b regulates Mertk phosphorylation in primary human NK cells, leading to a suppressive role in activated NK cells.

### Example 6: Knockout of Cbl-b Show In Vivo Role Regulation of NK Cells and KO Increases in NK Cells in a Some Tissues

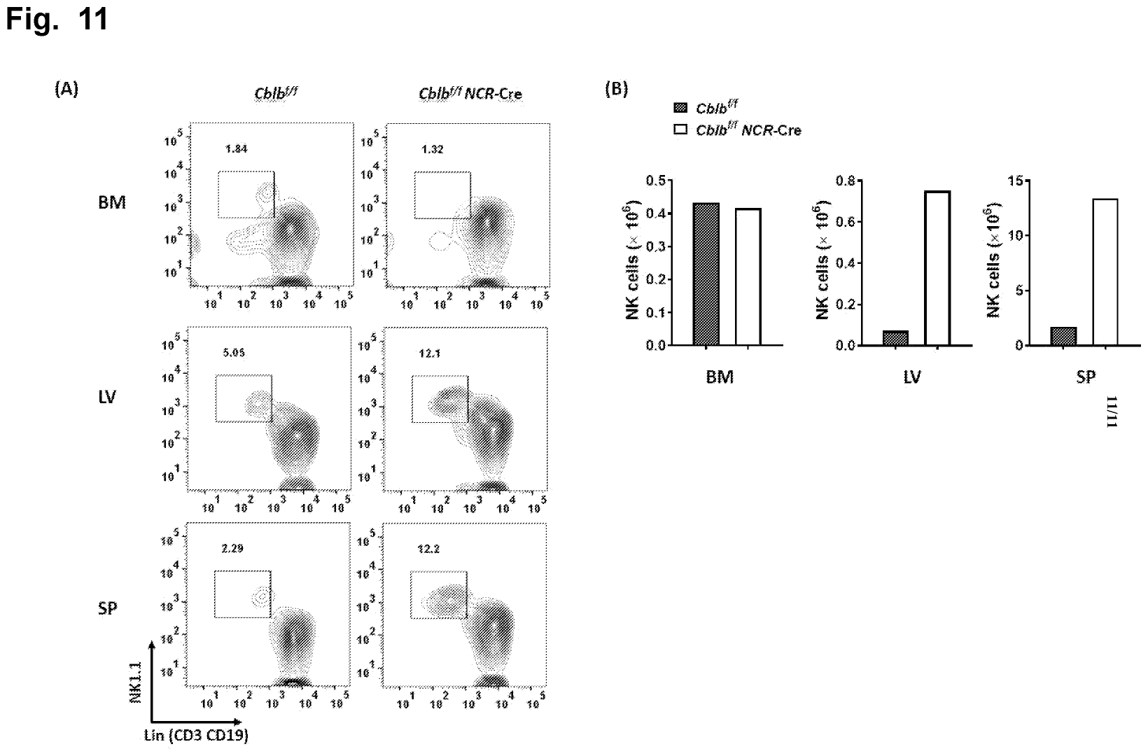

Cbl-b floxed (f) mice were crossed with NCR1-Cre mice to get specific knockout of Cbl-b in NK cells to create Cblb(f/f)NCR-Cre mice. Flow cytometry of Cblbf/f NCR-Cre mice and littermate control (8-week old) showed accrual of NK1.1⁺CD3⁻CD19⁻ cells in different tissue or organs including bone marrow (BM), liver (LV), and spleen (SP). As shown in FIGS. 11A and 11B, the number of NK NK1.1⁺CD3⁻CD19⁻ in Cblbf/f NCR-Cre Tg mice were higher than in littermate controls in both liver and spleen, but similar in bone marrow.
1. Sun, J. C., and L. L. Lanier. 2011. NK cell development, homeostasis and function: parallels with CD8(+) T cells. Nat Rev Immunol 11: 645-657.
2. Vivier, E., E. Tomasello, M. Baratin, T. Walzer, and S. Ugolini. 2008. Functions of natural killer cells. Nat Immunol 9: 503-510.
3. Spits, H., L. L. Lanier, and J. H. Phillips. 1995. Development of human T and natural killer cells. Blood 85: 2654-2670.
4. Herberman, R. B., M. E. Nunn, H. T. Holden, and D. H. Lavrin. 1975. Natural cytotoxic reactivity of mouse lymphoid cells against syngeneic and allogeneic tumors. II. Characterization of effector cells. Int J Cancer 16: 230-239.
5. Lanier, L. L., J. H. Phillips, J. Hackett, Jr., M. Tutt, and V. Kumar. 1986. Natural killer cells: definition of a cell type rather than a function. J Immunol 137: 2735-2739.
6. Muntasell, A., M. C. Ochoa, L. Cordeiro, P. Berraondo, A. Lopez-Diaz de Cerio, M. Cabo, M. Lopez-Botet, and I. Melero. 2017. Targeting NK-cell checkpoints for cancer immunotherapy. Current opinion in immunology 45: 73-81.
7. Morvan, M. G., and L. L. Lanier. 2016. NK cells and cancer: you can teach innate cells new tricks. Nat Rev Cancer 16: 7-19.
8. Vivier, E., J. A. Nunes, and F. Vely. 2004. Natural killer cell signaling pathways. Science 306: 1517-1519.
9. Kim, N., and H. S. Kim. 2018. Targeting Checkpoint Receptors and Molecules for Therapeutic Modulation of Natural Killer Cells. Front Immunol 9: 2041.
10. Duan, L., A. L. Reddi, A. Ghosh, M. Dimri, and H. Band. 2004. The Cbl family and other ubiquitin ligases: destructive forces in control of antigen receptor signaling. Immunity 21: 7-17.
11. Liu, Y. C. 2004. Ubiquitin ligases and the immune response. Annu Rev Immunol 22: 81-127.
12. Chiang, Y. J., H. K. Kole, K. Brown, M. Naramura, S. Fukuhara, R. J. Hu, I. K. Jang, J. S. Gutkind, E. Shevach, and H. Gu. 2000. Cbl-b regulates the CD28 dependence of T-cell activation. Nature 403: 216-220.
13. Jeon, M. S., A. Atfield, K. Venuprasad, C. Krawczyk, R. Sarao, C. Elly, C. Yang, S. Arya, K. Bachmaier, L. Su, D. Bouchard, R. Jones, M. Gronski, P. Ohashi, T. Wada, D. Bloom, C. G. Fathman, Y. C. Liu, and J. M. Penninger. 2004. Essential role of the E3 ubiquitin ligase Cbl-b in T cell anergy induction. Immunity 21: 167-177.
14. Kitaura, Y., I. K. Jang, Y. Wang, Y. C. Han, T. Inazu, E. J. Cadera, M. Schlissel, R. R. Hardy, and H. Gu. 2007. Control of the B cell-intrinsic tolerance programs by ubiquitin ligases Cbl and Cbl-b. Immunity 26: 567-578.
15. Li, X., A. Gadzinsky, L. Gong, H. Tong, V. Calderon, Y. Li, D. Kitamura, U. Klein, W. Y. Langdon, F. Hou, Y. R. Zou, and H. Gu. 2018. Cbl Ubiquitin Ligases Control B Cell Exit from the Germinal-Center Reaction. Immunity 48: 530-541 e536.
16. Matalon, O., and M. Barda-Saad. 2016. Cbl ubiquitin ligases mediate the inhibition of natural killer cell activity. Commun Integr Biol 9: e1216739.
17. Paolino, M., A. Choidas, S. Wallner, B. Pranjic, I. Uribesalgo, S. Loeser, A. M. Jamieson, W. Y. Langdon, F. Ikeda, J. P. Fededa, S. J. Cronin, R. Nitsch, C. Schultz-Fademrecht, J. Eickhoff, S. Menninger, A. Unger, R. Torka, T. Gruber, R. Hinterleitner, G. Baier, D. Wolf, A. Ullrich, B. M. Klebl, and J. M. Penninger. 2014. The E3 ligase Cbl-b and TAM receptors regulate cancer metastasis via natural killer cells. Nature 507: 508-512.
18. Mrozek, E., P. Anderson, and M. A. Caligiuri. 1996. Role of interleukin-15 in the development of human CD56+ natural killer cells from CD34+ hematopoietic progenitor cells. Blood 87: 2632-2640.
19. Dong, W., X. Wu, S. Ma, Y. Wang, A. P. Nalin, Z. Zhu, J. Zhang, D. M. Benson, K. He, M. A. Caligiuri, and J. Yu. 2019. The Mechanism of Anti-PD-L1 Antibody Efficacy against PD-L1-Negative Tumors Identifies NK Cells Expressing PD-Li as a Cytolytic Effector. Cancer Discov 9: 1422-1437.
20. Giri, J. G., M. Ahdieh, J. Eisenman, K. Shanebeck, K. Grabstein, S. Kumaki, A. Namen, L. S. Park, D. Cosman, and D. Anderson. 1994. Utilization of the beta and gamma chains of the IL-2 receptor by the novel cytokine IL-15. EMBO J 13: 2822-2830.
21. Waldmann, T., Y. Tagaya, and R. Bamford. 1998. Interleukin-2, interleukin-15, and their receptors. Int Rev Immunol 16: 205-226.
22. Kudlacz, E., B. Perry, P. Sawyer, M. Conklyn, S. McCurdy, W. Brissette, Flanagan, and P. Changelian. 2004. The novel JAK-3 inhibitor CP-690550 is a potent immunosuppressive agent in various murine models. Am J Transplant 4: 51-57.
23. Plimack, E. R., P. M. Lorusso, P. McCoon, W. Tang, A. D. Krebs, G. Curt, and S. G. Eckhardt. 2013. AZD1480: a phase I study of a novel JAK2 inhibitor in solid tumors. Oncologist 18: 819-820.
24. Spencer, A., S. S. Yoon, S. J. Harrison, S. R. Morris, D. A. Smith, R. A. Brigandi, J. Gauvin, R. Kumar, J. B. Opalinska, and C. Chen. 2014. The novel AKT inhibitor afuresertib shows favorable safety, pharmacokinetics, and clinical activity in multiple myeloma. Blood 124: 2190-2195.
25. Caraux, A., Q. Lu, N. Fernandez, S. Riou, J. P. Di Santo, D. H. Raulet, G. Lemke, and C. Roth. 2006. Natural killer cell differentiation driven by Tyro3 receptor tyrosine kinases. Nat Immunol 7: 747-754.
26. Kohrt, H. E., A. Thielens, A. Marabelle, I. Sagiv-Barfi, C. Sola, F. Chanuc, N. Fuseri, C. Bonnafous, D. Czerwinski, A. Rajapaksa, E. Waller, S. Ugolini, E. Vivier, F. Romagne, R. Levy, M. Blery, and P. Andre. 2014. Anti-KIR antibody enhancement of anti-lymphoma activity of natural killer cells as monotherapy and in combination with anti-CD20 antibodies. Blood 123: 678-686.
27. Benson, D. M., Jr., C. C. Hofmeister, S. Padmanabhan, A. Suvannasankha, S. Jagannath, R. Abonour, C. Bakan, P. Andre, Y. Efebera, J. Tiollier, M. A. Caligiuri, and S. S. Farag. 2012. A phase 1 trial of the anti-KIR antibody IPH2101 in patients with relapsed/refractory multiple myeloma. Blood 120: 4324-4333.
28. Tan, S., Y. Xu, Z. Wang, T. Wang, X. Du, X. Song, X. Guo, J. Peng, J. Zhang, Y. Liang, J. Lu, J. Peng, C. Gao, Z. Wu, C. Li, N. Li, L. Gao, X. Liang, and C. Ma. 2019. Tim-3 hampers tumor surveillance of liver resident and conventional NK cells by disrupting PI3K signaling. Cancer research.
29. Xu, L., Y. Huang, L. Tan, W. Yu, D. Chen, C. Lu, J. He, G. Wu, X. Liu, and Y. Zhang. 2015. Increased Tim-3 expression in peripheral NK cells predicts a poorer prognosis and Tim-3 blockade improves NK cell-mediated cytotoxicity in human lung adenocarcinoma. International immunopharmacology 29: 635-641.
30. Vari, F., D. Arpon, C. Keane, M. S. Hertzberg, D. Talaulikar, S. Jain, Q. Cui, E. Han, J. Tobin, R. Bird, D. Cross, A. Hernandez, C. Gould, S. Birch, and M. K. Gandhi. 2018. Immune evasion via PD-1/PD-L1 on NK cells and monocyte/macrophages is more prominent in Hodgkin lymphoma than DLBCL. Blood 131: 1809-1819.
31. Hsu, J., J. J. Hodgins, M. Marathe, C. J. Nicolai, M. C. Bourgeois-Daigneault, T. N. Trevino, C. S. Azimi, A. K. Scheer, H. E. Randolph, T. W. Thompson, L. Zhang, A. Iannello, N. Mathur, K. E. Jardine, G. A. Kirn, J. C. Bell, M. W. McBurney, D. H. Raulet, and M. Ardolino. 2018. Contribution of NK cells to immunotherapy mediated by PD-1/PD-Li blockade. The Journal of clinical investigation 128: 4654-4668.
32. Lino, A. C., V. D. Dang, V. Lampropoulou, A. Welle, J. Joedicke, J. Pohar, Q. Simon, J. Thalmensi, A. Baures, V. Fluhler, I. Sakwa, U. Stervbo, S. Ries, L. Jouneau, P. Boudinot, T. Tsubata, T. Adachi, A. Hutloff, T. Dorner, U. Zimber-Strobl, A. F. de Vos, K. Dahlke, G. Loh, S. Korniotis, C. Goosmann, J. C. Weill, C. A. Reynaud, S. H. E. Kaufmann, J. Walter, and S. Fillatreau. 2018. LAG-3 Inhibitory Receptor Expression Identifies Immunosuppressive Natural Regulatory Plasma Cells. Immunity 49: 120-133 e129.
33. Williams, J. B., B. L. Horton, Y. Zheng, Y. Duan, J. D. Powell, and T. F. Gajewski. 2017. The EGR2 targets LAG-3 and 4-1BB describe and regulate dysfunctional antigen-specific CD8+ T cells in the tumor microenvironment. The Journal of experimental medicine 214: 381-400.
34. Shamim, M., S. G. Nanjappa, A. Singh, E. H. Plisch, S. E. LeBlanc, J. Walent, J. Svaren, C. Seroogy, and M. Suresh. 2007. Cbl-b regulates antigen-induced TCR downregulation and IFN-gamma production by effector CD8 T cells without affecting functional avidity. J Immunol 179: 7233-7243.
35. Zhang, W., Y. Shao, D. Fang, J. Huang, M. S. Jeon, and Y. C. Liu. 2003. Negative regulation of T cell antigen receptor-mediated Crk-L-C3G signaling and cell adhesion by Cbl-b. J Biol Chem 278: 23978-23983.
36. Qiao, G., M. Lei, Z. Li, Y. Sun, A. Minto, Y. X. Fu, H. Ying, R. J. Quigg, and J. Zhang. 2007. Negative regulation of CD40-mediated B cell responses by E3 ubiquitin ligase Casitas-B-lineage lymphoma protein-B. J Immunol 179: 4473-4479.
37. Qu, X., K. Sada, S. Kyo, K. Maeno, S. M. Miah, and H. Yamamura. 2004. Negative regulation of FcepsilonRI-mediated mast cell activation by a ubiquitin-protein ligase Cbl-b. Blood 103: 1779-1786.
38. Konjevic, G. M., A. M. Vuletic, K. M. Mirjacic Martinovic, A. K. Larsen, and V. B. Jurisic. 2019. The role of cytokines in the regulation of NK cells in the tumor environment. Cytokine 117: 30-40.
39. Strengell, M., S. Matikainen, J. Siren, A. Lehtonen, D. Foster, I. Julkunen, and T. Sareneva. 2003. IL-21 in synergy with IL-15 or IL-18 enhances IFN-gamma production in human NK and T cells. J Immunol 170: 5464-5469.
40. Park, S. Y., K. Saijo, T. Takahashi, M. Osawa, H. Arase, N. Hirayama, K. Miyake, H. Nakauchi, T. Shirasawa, and T. Saito. 1995. Developmental defects of lymphoid cells in Jak3 kinase-deficient mice. Immunity 3: 771-782.
41. Teglund, S., C. McKay, E. Schuetz, J. M. van Deursen, D. Stravopodis, D. Wang, M. Brown, S. Bodner, G. Grosveld, and J. N. Ihle. 1998. Stat5a and Stat5b proteins have essential and nonessential, or redundant, roles in cytokine responses. Cell 93: 841-850.
42. Zhu, X., K. L. Suen, M. Barbacid, J. B. Bolen, and J. Fargnoli. 1994. Interleukin-2-induced tyrosine phosphorylation of Shc proteins correlates with factor-dependent T cell proliferation. J Biol Chem 269: 5518-5522.
43. Gu, H., H. Maeda, J. J. Moon, J. D. Lord, M. Yoakim, B. H. Nelson, and B. G. Neel. 2000. New role for Shc in activation of the phosphatidylinositol 3-kinase/Akt pathway. Molecular and cellular biology 20: 7109-7120.
44. Wang, K. S., D. A. Frank, and J. Ritz. 2000. Interleukin-2 enhances the response of natural killer cells to interleukin-12 through up-regulation of the interleukin-12 receptor and STAT4. Blood 95: 3183-3190.
45. Kennedy, M. K., M. Glaccum, S. N. Brown, E. A. Butz, J. L. Viney, M. Embers, N. Matsuki, K. Charrier, L. Sedger, C. R. Willis, K. Brasel, P. J. Morrissey, K. Stocking, J. C. Schuh, S. Joyce, and J. J. Peschon. 2000. Reversible defects in natural killer and memory CD8 T cell lineages in interleukin 15-deficient mice. The Journal of experimental medicine 191: 771-780.
46. Koka, R., P. R. Burkett, M. Chien, S. Chai, F. Chan, J. P. Lodolce, D. L. Boone, and A. Ma. 2003. Interleukin (IL)-15R[alpha]-deficient natural killer cells survive in normal but not IL-15R[alpha]-deficient mice. The Journal of experimental medicine 197: 977-984.
47. Carson, W. E., T. A. Fehniger, S. Haldar, K. Eckhert, M. J. Lindemann, C. F. Lai, C. M. Croce, H. Baumann, and M. A. Caligiuri. 1997. A potential role for interleukin-15 in the regulation of human natural killer cell survival. The Journal of clinical investigation 99: 937-943.
48. Carson, W. E., M. E. Ross, R. A. Baiocchi, M. J. Marien, N. Boiani, K. Grabstein, and M. A. Caligiuri. 1995. Endogenous production of interleukin 15 by activated human monocytes is critical for optimal production of interferon-gamma by natural killer cells in vitro. The Journal of clinical investigation 96: 2578-2582.
49. Kasaian, M. T., M. J. Whitters, L. L. Carter, L. D. Lowe, J. M. Jussif, B. Deng, K. A. Johnson, J. S. Witek, M. Senices, R. F. Konz, A. L. Wurster, D. D. Donaldson, M. Collins, D. A. Young, and M. J. Grusby. 2002. IL-21 limits NK cell responses and promotes antigen-specific T cell activation: a mediator of the transition from innate to adaptive immunity. Immunity 16: 559-569.
50. Fehniger, T. A., M. H. Shah, M. J. Turner, J. B. VanDeusen, S. P. Whitman, M. A. Cooper, K. Suzuki, M. Wechser, F. Goodsaid, and M. A. Caligiuri. 1999. Differential cytokine and chemokine gene expression by human NK cells following activation with IL-18 or IL-15 in combination with IL-12: implications for the innate immune response. J Immunol 162: 4511-4520.
51. French, A. R., E. B. Holroyd, L. Yang, S. Kim, and W. M. Yokoyama. 2006. IL-18 acts synergistically with IL-15 in stimulating natural killer cell proliferation. Cytokine 35: 229-234.
52. Michaud, A., R. Dardari, E. Charrier, P. Cordeiro, S. Herblot, and M. Duval. 2010. IL-7 enhances survival of human CD56bright NK cells. J Immunother 33: 382-390.
53. Yokohama, A., A. Mishra, T. Mitsui, B. Becknell, J. Johns, D. Curphey, B. W. Blaser, J. B. Vandeusen, H. Mao, J. Yu, and M. A. Caligiuri. 2010. A novel mouse model for the aggressive variant of NK cell and T cell large granular lymphocyte leukemia. Leukemia research 34: 203-209.
54. Fehniger, T. A., K. Suzuki, A. Ponnappan, J. B. VanDeusen, M. A. Cooper, S. M. Florea, A. G. Freud, M. L. Robinson, J. Durbin, and M. A. Caligiuri. 2001. Fatal leukemia in interleukin 15 transgenic mice follows early expansions in natural killer and memory phenotype CD8+ T cells. The Journal of experimental medicine 193: 219-231.
55. Sato, N., H. Sabzevari, S. Fu, W. Ju, M. N. Petrus, R. N. Bamford, T. A. Waldmann, and Y. Tagaya. 2011. Development of an IL-15-autocrine CD8 T-cell leukemia in IL-15-transgenic mice requires the cis expression of IL-15Ralpha. Blood 117: 4032-4040.
56. Mishra, A., K. La Perle, S. Kwiatkowski, L. A. Sullivan, G. H. Sams, J. Johns, D. P. Curphey, J. Wen, K. McConnell, J. Qi, H. Wong, G. Russo, J. Zhang, G. Marcucci, J. E. Bradner, P. Porcu, and M. A. Caligiuri. 2016. Mechanism, Consequences, and Therapeutic Targeting of Abnormal IL15 Signaling in Cutaneous T-cell Lymphoma. Cancer Discov 6: 986-1005.
57. Braiman, A., M. Barda-Saad, C. L. Sommers, and L. E. Samelson. 2006. Recruitment and activation of PLCgammal in T cells: a new insight into old domains. EMBO J 25: 774-784.
58. Caraux, A., N. Kim, S. E. Bell, S. Zompi, T. Ranson, S. Lesjean-Pottier, M. E. Garcia-Ojeda, M. Turner, and F. Colucci. 2006. Phospholipase C-gamma2 is essential for NK cell cytotoxicity and innate immunity to malignant and virally infected cells. Blood 107: 994-1002.
59. Upshaw, J. L., R. A. Schoon, C. J. Dick, D. D. Billadeau, and P. J. Leibson. 2005. The isoforms of phospholipase C-gamma are differentially used by distinct human NK activating receptors. J Immunol 175: 213-218.
60. Chan, G., T. Hanke, and K. D. Fischer. 2001. Vav-1 regulates NK T cell development and NK cell cytotoxicity. European journal of immunology 31: 2403-2410.
61. Yin, S., J. Zhang, Y. Mao, Y. Hu, L. Cui, N. Kang, and W. He. 2013. Vav1-phospholipase C-gammal (Vav1-PLC-gammal) pathway initiated by T cell antigen receptor (TCRgammadelta) activation is required to overcome inhibition by ubiquitin ligase Cbl-b during gammadeltaT cell cytotoxicity. J Biol Chem 288: 26448-26462.
62. Kim, H. S., A. Das, C. C. Gross, Y. T. Bryceson, and E. O. Long. 2010. Synergistic signals for natural cytotoxicity are required to overcome inhibition by c-Cbl ubiquitin ligase. Immunity 32: 175-186.
63. Correa, I., L. Corral, and D. H. Raulet. 1994. Multiple natural killer cell-activating signals are inhibited by major histocompatibility complex class I expression in target cells. European journal of immunology 24: 1323-1331.
64. Bhat, R., and C. Watzl. 2007. Serial killing of tumor cells by human natural killer cells—enhancement by therapeutic antibodies. PLoS One 2: e326.
65. Jewett, A., and B. Bonavida. 1995. Target-induced anergy of natural killer cytotoxic function is restricted to the NK-target conjugate subset. Cell Immunol 160: 91-97.
66. Sharif, M. N., D. Sosic, C. V. Rothlin, E. Kelly, G. Lemke, E. N. Olson, and L. B. Ivashkiv. 2006. Twist mediates suppression of inflammation by type I IFNs and Axl. The Journal of experimental medicine 203: 1891-1901.
67. Sen, P., M. A. Wallet, Z. Yi, Y. Huang, M. Henderson, C. E. Mathews, H. S. Earp, G. Matsushima, A. S. Baldwin, Jr., and R. M. Tisch. 2007. Apoptotic cells induce Mer tyrosine kinase-dependent blockade of NF-kappaB activation in dendritic cells. Blood 109: 653-660.
68. Cabezon, R., E. A. Carrera-Silva, G. Florez-Grau, A. E. Errasti, E. Calderon-Gomez, J. J. Lozano, C. Espana, E. Ricart, J. Panes, C. V. Rothlin, and D. Benitez-Ribas. 2015. MERTK as negative regulator of human T cell activation. J Leukoc Biol 97: 751-760.
69. Peeters, M. J. W., D. Dulkeviciute, A. Draghi, C. Ritter, A. Rahbech, S. K. Skadborg, T. Seremet, A. M. Carnaz Simoes, E. Martinenaite, H. R. Halldorsdottir, M. H. Andersen, G. H. Olofsson, I. M. Svane, L. J. Rasmussen, O. Met, J. C. Becker, M. Donia, C. Desler, and P. Thor Straten. 2019. MERTK Acts as a Costimulatory Receptor on Human CD8(+) T Cells. Cancer Immunol Res 7: 1472-1484.

### OTHER EMBODIMENTS

It is to be understood that while the invention has been described in conjunction with the detailed description thereof, the foregoing description is intended to illustrate and not limit the scope of the invention. Other aspects, advantages, and modifications are within the scope of the following claims.

1. A composition comprising human natural killer (NK) cells that either do not express Cbl-b (Cbl-b^{neg} NK cells) or have reduced or suppressed expression of Cbl-b (Cbl-b^{low} NK cells).

2. A method for preparing activated Cbl-b^{neg} NK cells or activated Cbl-b^{low} NK cells, the method comprising:
obtaining a population NK cells,
reducing or eliminating Cbl-b expression in the NK cells, and
culturing the NK cells.

3. The method of claim 2, wherein the step of reducing or eliminating Cbl-b expression comprises genetically modifying the NK cells.

4. The method of claim 3, wherein the genetic modification is deletion of all or a portion of the Cbl-b gene or an insertion into the Clb-b gene.

5. (canceled)

6. The method of claim 2, wherein the culturing comprises co-incubating the NK cells with feeder cells.

7. The method of claim 6, wherein the feeder cells are K562 cells.

8.-14. (canceled)

15. The method of claim 2, wherein the expression Cbl-b is reduced or eliminated by targeting a gene encoding Cbl-b or an RNA product of the gene encoding Cbl-b.

16.-17. (canceled)

18. The method of claim 2, wherein the Cbl-b gene is modified, edited, or knocked out using gene editing, homologous recombination, nonhomologous recombination, RNA-mediated genetic modification, DNA-mediated genetic modification, a zinc finger nuclease, a meganuclease, RALEN, TALEN, megaTAL, CRISPR/CAS9, or CRISPR/Cpf1.

19. The method of claim 2, wherein the reducing or eliminating Cbl-b expression comprises administering an inhibitory oligonucleotide to the NK cells.

20. (canceled)

21. The method of claim 19, wherein the inhibitory oligonucleotide is complementary to at least 8, 9, 10, 11, 12, 13, 14, 15, 16, 17, 18, 19, or 20 continuous nucleotides of a gene encoding Cbl-b, an RNA product of the gene encoding Cbl-b, SEQ ID NO:3, the complimentary sequence of SEQ ID NO:3, or a sequence encoding any of SEQ ID NOs:4-32.

22. The method of claim 19, wherein the inhibitory oligonucleotide comprises SEQ ID NO: 1.

23. A composition comprising the population of expanded Cbl-b^{neg} NK cells or expanded Cbl-b^{low} NK cells produced by the method of claim 2.

24. A method of killing cancer cells comprising contacting the cancer cells with a therapeutically effective amount of the population of human NK cells of claim 2, wherein the NK cells have been transfected with a nucleic acid encoding a chimeric antigen receptor or the NK cells express a chimeric antigen receptor, and wherein the chimeric antigen receptor is targeted to an antigen on the cancer cell.

25. A method of treating cancer in a subject in need thereof, comprising administering to the subject a therapeutically effective amount of the population of human NK cells of claim 2, wherein the NK cells have been transfected with a nucleic acid encoding a chimeric antigen receptor or the NK cells express a chimeric antigen receptor, and wherein the chimeric antigen receptor is targeted to an antigen expressed by the cancer, thereby treating cancer in the subject.

26. (canceled)

27. The method of claim 24, wherein the cancer is selected from a group consisting of lung cancer, breast cancer, ewing sarcoma, central nervous system neoplasm, skin cancer, head and neck cancer, ovarian cancer, colon cancer, anal cancer, stomach cancer, gastrointestinal cancer, fallopian tube cancer, endometrial cancer, cervical cancer, vaginal cancer, vulvar cancer, esophageal cancer, endocrine cancer, thyroid cancer, parathyroid cancer, adrenal cancer, soft tissue sarcoma, urethral cancer, penile cancer, testicular cancer, brain stem glioma, pituitary cancer, adrenocortical cancer, gallbladder cancer, multiple myeloma, cholangiocarcinoma, fibrosarcoma, lymphoma, liver cancer, kidney cancer, bone cancer, bladder cancer, colorectal cancer, endometrial cancer, renal cell cancer, pancreatic cancer, prostate cancer, thyroid cancer, mesothelioma, neuroblastoma, retinoblastoma, melanoma, rhabdomyosarcoma, leukemia and lymphoma.

28. A method of treating a viral infection in a subject in need thereof, comprising administering to the subject a therapeutically effective amount of the population of human NK cells of claim 2, wherein the NK cells have been transfected with a nucleic acid encoding a chimeric antigen receptor or the NK cells express a chimeric antigen receptor, and wherein the chimeric antigen receptor is targeted to an antigen expressed by the virus, thereby treating the viral infection in the subject.

29. A population of human NK cells harboring a vector comprising a nucleic sequence expressing an siRNA targeted to Cbl-b and a nucleic acid sequence encoding a chimeric antigen receptor.

30.-35. (canceled)

36. The composition of claim 1, wherein the NK cells have been transfected with a nucleic acid encoding a chimeric antigen receptor or the NK cells express a chimeric antigen receptor.

37.-39. (canceled)

40. The composition of claim 1, wherein the Cbl-b^{neg} NK cells or Cbl-b^{low} NK cells are genetically modified.

41. (canceled)

42. The composition of claim 40, wherein the genetically modified cells express Cbl-b at a level that is 50%, 40%, 30%, 20% or 10% or less than cells that are not genetically modified.
